(19) United States
(12) Patent Application Publication
(10) Pub. No.: US 2024/0246955 A1
(43) Pub. Date: Jul. 25, 2024

(54) TROPOMYOSIN RECEPTOR KINASE (TRK) DEGRADATION COMPOUNDS AND METHODS OF USE

(71) Applicant: CULLGEN (SHANGHAI), INC., Shanghai (CN)

(72) Inventors: Jing LIU, Shanghai (CN); Jialiang WANG, Shanghai (CN); Xiaoran HAN, Shanghai (CN); Ting YANG, Shanghai (CN)

(21) Appl. No.: 18/555,139

(22) PCT Filed: Apr. 12, 2022
(86) PCT No.: PCT/CN2022/086258

§ 371 Date: Oct. 12, 2023

(30) Foreign Application Priority Data
Apr. 21, 2021 (WO) PCT/CN2021/086732

(51) Int. Cl. C07D 417/14; A61K 31/506; A61P 35/00; C07D 401/14; C07D 403/14

(52) U.S. Cl. CPC C07D 417/14; A61K 31/506; A61P 35/00; C07D 401/14; C07D 403/14

(57) ABSTRACT

Heterobifunctional compounds (e.g., bi-functional small molecule compounds) and compositions comprising one or more of the heterobifunctional compounds are provided. The heterobifunctional compounds are used for the treatment of certain disease in a subject in need thereof. Methods for identifying such heterobifunctional compounds are also presented.

### TECHNICAL FIELD

The present disclosure belongs to the field of medicine, and specifically relates to tropomyosin receptor kinase (trk) degradation compounds and methods of use.

### BACKGROUND OF THE INVENTION

Tropomyosin receptor kinase (TRK) receptor family comprises three members, TRKA, TRKB and TRKC that are encoded by the NTRK1, NTRK2 and NTRK3 genes, respectively (Khotskaya et al., 2017). TRKs are receptor tyrosine kinases primarily implicated in development and functions of the neuronal tissues. The main ligands of TRKs include nerve growth factor (NGF) for TRKA, brain-derived growth factor (BDGF) for TRKB, and neurotrophins for TRKC (Vaishnavi et al., 2015). The binding of ligands to the extracellular domains of TRKs induces dimerization and activation of the receptors, which activates downstream signal transduction pathways, such as PI3K/AKT, RAF/MEK/ERK, and PLCγ pathways. These pathways have well established roles to support cellular proliferation, survival, and promote oncogenesis (Hanahan and Weinberg, 2011).

Like many other oncogenic receptor tyrosine kinases, TRKs are aberrantly activated in a variety of human malignancies. Interestingly, the primary molecular mechanism activating TRKs in cancer is not point mutations but in-frame fusions of NTRK genes (Vaishnavi et al., 2015). Typically, the 3′ regions of the NTRK genes are joined with the 5′ regions of a partner gene due to chromosomal rearrangement. The resulted chimeric proteins always retain the kinase domain of TRK proteins, indicating that the catalytic functions are crucial to the transforming activities. Loss of the 5′ regions of the NTRK genes that encode the self-inhibitory domains renders these fusion kinases constitutively active. Additionally, expression of the chimeric proteins is driven by the promoters of the fusion partners, which often result in overexpression. The most common TRK fusions include LMNA-TRKA, TPM3-TRKA, and ETV6-TRKC (Amatu et al., 2016). Hence, genetic events lead to overexpressed and constitutively active TRK-fusion kinases. These fusions are oncogenic, as shown by their ability to transform mouse embryonic fibroblasts and normal epithelium (Russell et al., 2000; Vaishnavi et al., 2015).

TRK fusion was first reported in a human colon carcinoma, which was named as oncD at that time (Martin-Zanca et al., 1986). Recent advances in high-throughput RNA sequencing greatly promote the efficiency of identifying chromosomal rearrangement events in patient samples. Consequently, TRK fusions have been found across a wide range of human malignancies (Amatu et al., 2016; Khotskaya et al., 2017). The frequency of TRK fusions is relatively low. For example, approximately 0.5% to 2.7% colon cancers are affected by TRK fusions (Creancier et al., 2015; Lee et al., 2015). However, for certain cancer types, such as secretory breast carcinoma, TRK fusions can be found in the vast majority of cases (Tognon et al., 2002). TRK mutations and deletions have been observed in additional human cancers (Khotskaya et al., 2017).

The never growth factor (NGF) and its main receptor, tropomyosin receptor kinase A (TRKA), have long been recognized for their roles in central and peripheral pain (Denk, Bennett et al. 2017). Nociceptive neurons express TRKA and mediate pain sensation by transmitting pain signals to the central nervous system. Multiple NGF-neutralizing antibodies, such as tanezumab, are undergoing clinical assessment in patients with osteoarthritis, lower back pain, cancer pain, neuropathic pain, and other pain conditions (Miller, Block et al. 2017). The efficacy of NGF antibodies in pain relief has been clearly documented in clinics. However, administration of NGF neutralizing antibodies has been shown to result in rapidly progressed joint destruction in some patients that leads to total joint replacement (Schnitzer and Marks 2015). These adverse events may be related to sustained exposure to NGF antibodies. Targeting TRK represents another promising therapeutic strategy blocking the NGF/TRK signaling pathway for pain management. However, currently available pan-TRK kinase inhibitors may induce significant on-target adverse effects through modulating TRK family members in the central nervous system. Peripherally restricted TRK bifunctional degraders are expected to selective block the NGF/TRK pathway in peripheral nerves while spare these targets in the central nervous system.

Most of the well-known TRK inhibitors, such as entrectinib (RXDX-101) and larotrectinib (LOXO-101; ARRY-470) target to the APT-binding site. However, because of the similarity of the APT-binding site of TRK family members, these inhibitors are generally not selective within the TRK family members. In addition, drug resistance to TRK kinase inhibitors trough TRK mutations, such as TRKA G595R, TRKAC G623R, TRKA 667C, and TRKC G696A, at the APT-binding site has been reported. Small-molecule TRK inhibitors targeting to the allosteric site have been reported, such as AR-256 (CAS #:1923837-34-6), AR-618 (CAS #: 1923837-35-7), AR-786 (CAS #: 1923834-82-5), 1-((3S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea (CAS #: 1824664-89-2), 2-(4-(6-(1H-imidazol-1-yl)pyridin-3-yl)-1H-1,2,3-triazol-1-yl)-N-(3-(tert-butyl)-1-(pyridin-3-yl)-1H-pyrazol-5-yl)acetamide (CAS #:1614229-04-7), 5-(2,4-dichloro-5-(pyridin-2-yl)benzamido)-N-(2-hydroxy-2-methylpropyl)-1-phenyl-1H-pyrazole-3-carboxamide (CAS #: 1821484-84-7), and 5-(2,4-dichloro-5-(3-fluoropyridin-2-yl)benzamido)-N-(2-hydroxy-2-methylpropyl)-1-phenyl-1H-pyrazole-3-carboxamide (CAS #1821485-70-4). These allosteric TRK inhibitors showed excellent selectivity for TRKA over TRKB and TRKC. However, TRK degraders derived from these allosteric inhibitors were not reported.

There is a need in the art for tropomyosin receptor kinase (trk) degradation compounds, compositions, and methods of use of the compounds for the treatment of diseases in a subject in need thereof.

### SUMMARY OF THE INVENTION

This disclosure relates to tropomyosin receptor kinase (TRK) degradation compounds, compositions comprising the same and methods of use of the compounds for the treatment of diseases in a subject in need thereof.

This disclosure relates to heterobifunctional compounds (e.g., bi-functional small molecule compounds), compositions comprising one or more of the heterobifunctional compounds, and to methods of use of the heterobifunctional compounds for the treatment of certain diseases in a subject in need thereof. The disclosure also relates to methods for identifying such heterobifunctional compounds.

According to the 1st aspect of the present disclosure, a heterobifunctional compound disclosed herein comprises a Tropomyosin Receptor Kinase (TRK) ligand (TRK ligand) conjugated to a degradation tag optionally via a linker moiety, or a pharmaceutically acceptable salt, hydrate, solvate, prodrug, stereoisomer, tautomer, or analog thereof, wherein
i) the TRK ligand is a molecule that binds to a TRK non-ATP binding site;
ii) the degradation tag is a molecule that binds to a ubiquitin ligase (preferably, the ubiquitin ligase is an E3 ligase; more preferably, the E3 ligase is selected from the group consisting of a cereblon E3 ligase, a VHL E3 ligase, an IAP ligase, a MDM2 ligase, a TRIM24 ligase, a TRIM21 ligase, a KEAP1 ligase, DCAF16 ligase, RNF4 ligase, RNF114 ligase, and AhR ligase; more preferably, the E3 ligase is selected from a cereblon E3 ligase or a VHL E3 ligase) or is a hydrophobic group or a tag that leads to misfolding of the TRK proteins; and
iii) the linker moiety is a molecule that covalently connect the TRK ligand moiety and the degradation tag moiety.

In another embodiment, the heterobifunctional compound disclosed herein comprises a compound of FORMULA I:

M_{TRK}-M_{L}-M_{DT} (FORMULA I),
or a pharmaceutically acceptable salt, hydrate, solvate, prodrug, stereoisomer, tautomer, or analog thereof, wherein M_{TRK} is a TRK ligand; M_{L} is a linker moiety; and M_{DT} is a degradation tag;
and wherein
a) the TRK ligand is a moiety of FORMULA 1:

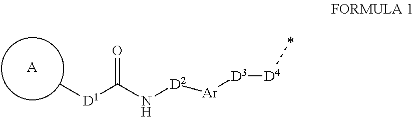

wherein *, Ring A, Ring B, D¹, D², D³ and D⁴ are defined as hereinafter;
b) the degradation tag is a moiety of FORMULA 5:

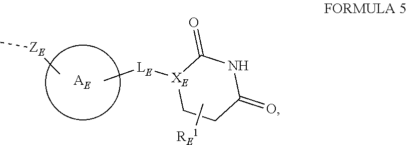

wherein *, Ring A_{E}, Z_{E}, L_{E}, X_{E}, and R_{E}¹ are defined as hereinafter;
or, the degradation tag is a moiety of FORMULA 6A, 6B, or 6C:

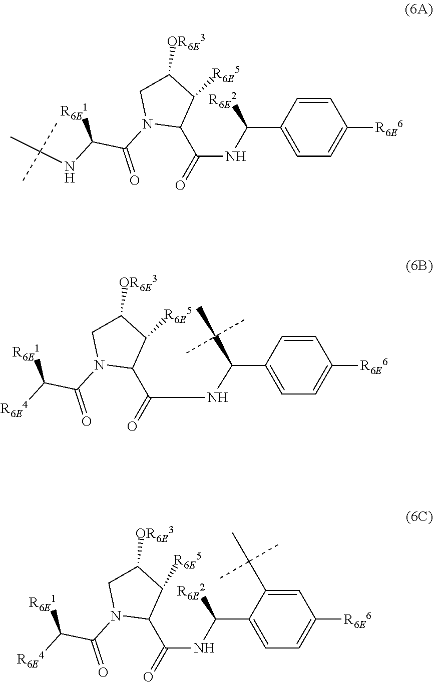

wherein R_{6E}¹, R_{6E}², R_{6E}³, R_{6E}⁴, R_{6E}⁵, and R_{6E}⁶ are defined as hereinafter; and
c) the linker is a moiety of FORMULA 9:

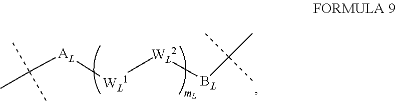

wherein m_{L}, A_{L}, W_{L}¹, W_{L}² and B_{L} are defined as hereinafter.

In some embodiments, the compound comprises any one of the compounds in Table 1 or Table 2.

In some embodiments, the heterobifunctional compound is selected from the group consisting of CPD-001 to CPD-033 or a pharmaceutically acceptable salt or analog thereof.

In some embodiments, the heterobifunctional compound is selected from the group consisting of CPD-002, CPD-003, CPD-006, CPD-007, CPD-008, CPD-009, CPD-019, CPD-023, CPD-026, CPD-027, CPD-028, CPD-030, CPD-031, and CPD-032, or a pharmaceutically acceptable salt or analog thereof.

In some embodiments, the compound comprises CPD-002, CPD-003, CPD-006, CPD-007, CPD-008, CPD-009, CPD-019, CPD-023, CPD-026, CPD-027, CPD-028, CPD-030, CPD-031, CPD-032, or a pharmaceutically acceptable salt or analog thereof.

In one embodiment, the heterobifunctional compound is 1-(3-(2-(4-(4-((2-(2,6-dioxopiperidin-3-yl)-1,3-dioxoisoindolin-5-yl)amino)butanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-002).

In one embodiment, the heterobifunctional compound is 1-(3-(2-(4-((2-(2,6-dioxopiperidin-3-yl)-1,3-dioxoisoindolin-4-yl)glycyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-003).

In one embodiment, the heterobifunctional compound is 1-(3-(2-(4-(8-((2-(2,6-dioxopiperidin-3-yl)-1,3-dioxoisoindolin-5-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-006).

In one embodiment, the heterobifunctional compound is 1-(3-(2-(4-(6-((2-(2,6-dioxopiperidin-3-yl)-1,3-dioxoisoindolin-4-yl)amino)hexanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-007).

In one embodiment, the heterobifunctional compound is 1-(3-(2-(4-(6-((2-(2,6-dioxopiperidin-3-yl)-1,3-dioxoisoindolin-5-yl)amino)hexanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-008).

In one embodiment, the heterobifunctional compound is 1-(3-(2-(4-(8-((2-(2,6-dioxopiperidin-3-yl)-1,3-dioxoisoindolin-4-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-009).

In one embodiment, the heterobifunctional compound is 1-(3-(2-(4-((2-(2,6-dioxopiperidin-3-yl)-1,3-dioxoisoindolin-5-yl)glycyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-019).

In one embodiment, the heterobifunctional compound is 1-(3-(2-(4-(2-((2-(2,6-dioxopiperidin-3-yl)-1,3-dioxoisoindolin-5-yl)oxy)acetyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-023).

In one embodiment, the heterobifunctional compound is 1-(3-(2-(4-(3-(2-(2,6-dioxopiperidin-3-yl)-1,3-dioxoisoindolin-5-yl)propanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-026).

In one embodiment, the heterobifunctional compound is 1-(3-(2-(4-(2-((2-(2,6-dioxopiperidin-3-yl)-1,3-dioxoisoindolin-4-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-027).

In one embodiment, the heterobifunctional compound is 1-(3-(2-(4-(2-((2-(2,6-dioxopiperidin-3-yl)-1,3-dioxoisoindolin-5-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-028).

In one embodiment, the heterobifunctional compound is 1-(3-(2-(4-(2-((2-(2,6-dioxopiperidin-3-yl)-1,3-dioxoisoindolin-4-yl)amino)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-030).

In one embodiment, the heterobifunctional compound is 1-(3-(2-(4-(2-((2-(2,6-dioxopiperidin-3-yl)-1,3-dioxoisoindolin-5-yl)amino)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-031).

In one embodiment, the heterobifunctional compound is 1-(3-(2-(4-(3-(2-(2,6-dioxopiperidin-3-yl)-1,3-dioxoisoindolin-5-yl)propyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-032).

According to the 2nd aspect of the present disclosure, a pharmaceutical composition is provided herein comprising a compound according to the 1^{st} aspect of the present disclosure, and one or more pharmaceutically acceptable carriers.

In one embodiment, the pharmaceutical composition further comprising one or more additional therapeutic agent.

According to the 3rd aspect of the present disclosure, a method of treating and/or preventing a TRK-mediated disease provided herein comprises administering to a subject in need thereof an effective amount of a heterobifunctional compound according to the 1st aspect of the present disclosure, or a pharmaceutically acceptable salt or analog thereof, or a pharmaceutical composition comprising the same.

In one embodiment, the subject in need means a subject with one or more TRK-mediated diseases and/or a subject with elevated TRK function.

In one embodiment, the TRK-mediated disease results from TRK expression, mutation, deletion, or fusion.

In one embodiment, the subject with the TRK-mediated disease has an elevated TRK function relative to a healthy subject without the TRK-mediated disease.

In one embodiment, the subject is mammal, preferably, human.

In one embodiment, the heterobifunctional compound is selected from the group consisting of CPD-001 to CPD-033, or a pharmaceutically acceptable salt or analog thereof.

In one embodiment, the heterobifunctional compound is administered to the subject orally, parenterally, intradermally, subcutaneously, topically, or rectally.

In one embodiment, the method further comprises administering to the subject an additional therapeutic regimen for treating cancer, pain, inflammatory disorders, or immune diseases.

In one embodiment, the additional therapeutic regimen is selected from the group consisting of surgery, chemotherapy, radiation therapy, hormone therapy, targeted therapy, and immunotherapy.

In one embodiment, the TRK-mediated diseases are selected from the group consisting of cancer, pain, an inflammatory disorder, an immune disease, or the combination thereof.

According to the 4th aspect of the present disclosure, a use of the compound according to the 1^{st} aspect of the present disclosure, or a pharmaceutically acceptable salt, or analog thereof, or the pharmaceutical composition according to the 2^{nd} aspect of the present disclosure in preparing a drug for treating and/or preventing TRK-mediated diseases is provided.

In one embodiment, TRK-mediated diseases are defined as before.

In one embodiment, the invention provides use of the heterobifunctional compound according to the 1^{st} aspect of the present disclosure, or a pharmaceutically acceptable salt thereof, in preparation of a medicament for treating a TRK-mediated disease.

In one embodiment, the invention provides use of a heterobifunctional compound according to the 1^{st} aspect of the present disclosure, or a pharmaceutically acceptable salt thereof, or a pharmaceutical composition comprising such compound or salt, for use in treating a TRK-mediated disease.

According to the 5th aspect of the present disclosure, a method for identifying a heterobifunctional compound which mediates degradation or reduction of TRK is disclosed. The method comprises:
providing a heterobifunctional test compound comprising an TRK ligand conjugated to a degradation tag through a linker;
contacting the heterobifunctional test compound with a cell comprising a ubiquitin ligase and TRK;
determining whether TRK level is decreased in the cell; and
identifying the heterobifunctional test compound as a heterobifunctional compound which mediates degradation or reduction of TRK.

In one embodiment, the cell is a cancer cell.

In one embodiment, the cancer cell is a TRK-mediated cancer cell.

In one embodiment, the cell is a neuron cell.

According to the 6^{th} aspect of the present disclosure, a use of the heterobifunctional compound, or a pharmaceutically acceptable salt, hydrate, solvate, prodrug, stereoisomer, tautomer, or analog thereof, is provided in combination with one or more additional therapeutic agents.

In one embodiment, the heterobifunctional compound is of FORMULA I.

In one embodiment, the TRK ligand moiety of the heterobifunctional compound is a moiety of FORMULA 1 as defined as in the first aspect.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows immunoblots of TPM3-TRKA fusion protein expressed by KM12 cells after 16-hour treatment with heterobifunctional compounds: CPD-002, CPD-003, CPD-004, CPD-005, CPD-006, CPD-007, CPD-008, CPD-009, CPD-014 and CPD-019 (A); WH-1, CPD-001, CPD-010, CPD-011, CPD-012, CPD-013, CPD-015, CPD-016, CPD-017, CPD-018, and CPD-020 (B); and CPD-023, CPD-024, CPD-025, CPD-026, CPD-027, CPD-028, CPD-029, CPD-030, CPD-031, CPD-032 and CPD-033 (C).

FIG. 2 shows an immunoblot of wildtype TRKA protein expressed by HEL cells after 16-hour treatment with heterobifunctional compounds CPD-019 and CPD-002.

FIG. 3 shows graphs of KM12 cell viability vs. concentration of heterobifunctional compounds CPD-002, CPD-003, and CPD-019.

FIG. 4 shows an immunoblot of wildtype TRKA protein expressed by HEL cells (A), TRKB protein expressed by NIH3T3-TRKB cells (B), and TRKC protein expressed by NIH3T3-TRKC cells (C), each after 16-hour treatment with heterobifunctional compound CPD-031.

### DETAILED DESCRIPTION OF THE INVENTION

In the present disclosure, a novel approach is taken: to develop compounds that directly and selectively modulate not only the kinase activity of TRK, but also their protein level. TRK degraders derived from these allosteric inhibitors have the potential to provide more selective TRK degradation among the TRK family members. In addition, these TRK degraders could also overcome the drug resistant induced by the TRK inhibitors, which target to the TRK ATP-binding site.

Without wishing to be bound by any theory, the present disclosure is believed to be based, at least in part, on the discovery that novel heterobivalent small molecules which degrade TRK, TRK fusion proteins, TRK splicing, and/or TRK mutant proteins are useful in the treatment of TRK-mediated diseases, particularly cancer, pain, inflammation disease, and immune disease.

Disclosed herein, in some embodiments, are heterobifunctional compounds. In some embodiments, the heterobifunctional compound comprises a chemical structure or formula disclosed herein. The heterobifunctional compound may be or include a TRK degrader. TRK degraders may be characterized by the ability to degrade or reduce cellular protein levels of TRK. Some embodiments relate to a composition that includes the heterobifunctional compound. Some embodiments relate to methods of making the heterobifunctional compound. Some embodiments relate to methods of using the heterobifunctional compound or a pharmaceutical composition of the heterobifunctional compound. For example, the heterobifunctional compound may be used to treat a disorder or a disease. In some cases, the compound is used to treat autoimmune diseases. In some cases, the compound is used to treat inflammatory diseases. In some cases, the compound is used to treat cancers.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound does not include any deuterium atoms. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound does not include any fluorine atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

### Heterobifunctional Compounds

Disclosed herein, in some embodiments, are compounds. In some embodiments, the compound comprises a TRK allosteric-site-binding moiety disclosed herein. In some embodiments, the compound comprises a degradation tag disclosed herein. In some embodiments, the compound comprises a CRBN-binding moiety. In some embodiments, the compound comprises a VHL-binding moiety. In some embodiments, the compound comprises a TRK degrader. For example, the compound may result in TRK degradation. The compound may degrade TRK as a result of hijacking CRBN ligase function. The compound may degrade TRK as a result of hijacking VHL ligase function. The compound may bind to or modulate TRK CRBN, or VHL. In some embodiments, the compound comprises a heterobifunctional compound. In some embodiments, the compound comprises a linker.

According to one aspect of the present disclosure, a heterobifunctional compound disclosed herein comprises a Tropomyosin Receptor Kinase (TRK) ligand (e.g. TRK allosteric ligand) conjugated to a degradation tag, or a pharmaceutically acceptable salt, hydrate, solvate, prodrug, stereoisomer, tautomer, or analog thereof, wherein
i) the TRK ligand is a molecule that binds to a TRK non-ATP binding site;
ii) the degradation tag is a molecule that binds to an ubiquitin ligase (preferably, the ubiquitin ligase is an E3 ligase; more preferably, the E3 ligase is selected from the group consisting of a cereblon E3 ligase, a VHL E3 ligase, an IAP ligase, a MDM2 ligase, a TRIM24 ligase, a TRIM21 ligase, a KEAP1 ligase, DCAF16 ligase, RNF4 ligase, RNF114 ligase, and AhR ligase; more preferably, the E3 ligase is selected from a cereblon E3 ligase or a VHL E3 ligase) or is a hydrophobic group or a tag that leads to misfolding of the TRK proteins; and
iii) the linker moiety is a molecule that covalently connect the TRK ligand moiety and the degradation tag moiety.

According to one aspect of the present disclosure, a heterobifunctional compound disclosed herein comprises a compound of FORMULA I

M_{TRK}-M_{L}-M_{DT} (FORMULA I);
or a pharmaceutically acceptable salt, hydrate, solvate, prodrug, stereoisomer, tautomer, or analog thereof,
wherein M_{TRK} is a TRK ligand; M_{L} is a linker moiety; and M_{DT} is a degradation tag;
and wherein
a) the TRK ligand is a moiety of FORMULA 1:

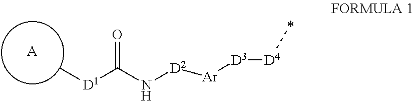

wherein
* indicates the connection to the linker moiety of the heterobifunctional compound;
D¹ is selected from null, —NH—, —O—, optionally substituted C₁-C₃ alkylene, optionally substituted C₁-C₃ alkylene-O—, or optionally substituted C₁-C₃ alkylene-NH—, and optionally substituted C₁-C₃ alkylene-N (optionally substituted C₁-C₈ alkyl)-;
D² is selected from null or optionally substituted C₁-C₃ alkylene;
D³ and D⁴ are independently selected from the group consisting of null, —O—, —S—, —NR¹—, —CO—, —CO₂—, —CONR¹—, —SO—, —SO₂—, —SO₂NR¹—, —NR¹CO—, —NR¹CO₂—, —NR¹C(O)NR²—, —NR¹SO—, —NR¹SO₂—, —NR¹SO₂NR²—, —OCO₂—, —OCONR¹—, optionally substituted C₁-C₈ alkylene, optionally substituted 1 to 8 membered heteroalkylene, optionally substituted C₁-C₈alkylene-O—C₁-C₈alkylene, optionally substituted C₁-C₈ haloalkylene, optionally substituted C₁-C₈ hydroxyalkylene, optionally substituted C₁-C₈alkylene-N(C₁-C₈alkyl)-C₁-C₈alkylene, optionally substituted C₃-C₈ cycloalkylene, optionally substituted C₃-C₈ carbocyclyl, optionally substituted 3-8 membered heterocyclyl, optionally substituted C₃-C₈ carbocyclyl-C₁-C₈alkylene, optionally substituted 3-8 membered heterocyclyl-C₁-C₈alkylene, optionally substituted C₃-C₈ carbocyclyl-O—, optionally substituted 3-8 membered heterocyclyl-O, optionally substituted C₃-C₈ carbocyclyl-N(C₁-C₈alkyl)-, and optionally substituted 3-8 membered heterocyclyl-N(C₁-C₈alkyl)-, optionally substituted C₂-C₈ alkenylene, optionally substituted C₂-C₈ alkynylene, optionally substituted 3-8 membered heteroalkenylene, optionally substituted 3-8 membered heteroalkynylene, optionally substituted aryl, and optionally substituted heteroaryl, wherein
R¹ and R² are independently selected from the group consisting of H, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₃-C₁₀ carbocyclyl, optionally substituted 3-10 membered heterocyclyl, optionally substituted aryl, and optionally substituted heteroaryl, or R¹ and R² together with the atom or atoms to which they are connected form a C₃-C₂₀ carbocyclyl ring or 3-20 membered heterocyclyl ring;
Ring A is Ring A′ or Ar², wherein: Ring A′ is selected from the group consisting of optionally substituted C₃-C₁₃ carbocyclyl and optionally substituted 3-13 membered heterocyclyl, and Ring A′ is optionally further substituted with Ar³; and Ar² is selected from the group consisting of optionally substituted aryl, and optionally substituted heteroaryl;
Ar³ is selected from optionally substituted aryl, and optionally substituted heteroaryl; and
Ar is selected from the group consisting of optionally substituted aryl, and optionally substituted heteroaryl, and Ar is optionally further substituted with Ar¹;
Ar¹ is selected from optionally substituted aryl and optionally substituted heteroaryl;
b) the degradation tag is a moiety of FORMULA 5:

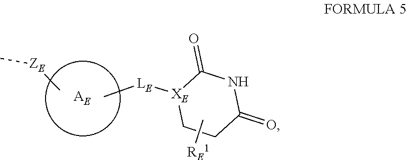

wherein
X_{E} is selected from CR_{E}¹³ or N, wherein R_{E}¹³ is selected from the group consisting of hydrogen, halogen, cyano, nitro, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₃-C₈ carbocyclyl, and optionally substituted 3-8 membered heterocyclyl;
Z_{E} is the attachment to M_{L}, and is a divalent group of formula —(R_{E}^{Z})_{nE}—, wherein subscript n^{E}=0, 1, 2, 3, 4, 5 or 6 (preferably, 0, 1 or 2; more preferably, 0 or 1); and R_{E}^{Z}, at each occurrence, is independently R_{E}^{r}, or R_{E}^{w}, wherein R_{E}^{w}, at each occurrence, is a bond or selected from the group consisting of —CO—, —CR_{E}⁵R_{E}⁶-, —NR_{E}⁵—, —O—, optionally substituted C₁-C₁₀ alkylene, optionally substituted C₂-C₁₀ alkenylene, optionally substituted C₂-C₁₀ alkynylene, optionally substituted C₁-C₁₀ heteroalkylene, optionally substituted C₂-C₁₀ heteroalkenylene, optionally substituted C₂-C₁₀ heteroalkynylene, optionally substituted C₁-C₁₀ haloalkylene; and R_{E}^{r}, at each occurrence, is a bond, or selected from the group consisting of optionally substituted C₃-C₁₀ carbocyclyl (preferably, C₃-C₈ carbocyclyl), optionally substituted 3-10 membered heterocyclyl (preferably, 3-8 membered heterocyclyl), optionally substituted C₄-C₁₃ fused cycloalkyl, optionally substituted 4-13 membered fused heterocyclyl, optionally substituted C₅-C₁₃ bridged cycloalkyl, optionally substituted 5-13 membered bridged heterocyclyl, optionally substituted C₅-C₁₃ spiro cycloalkyl, optionally substituted 5-13 membered spiro heterocyclyl, optionally substituted aryl, and optionally substituted heteroaryl; with the proviso that —R_{E}^{z}—R_{E}^{z}— is not —O—O—; R_{E}⁵ and R_{E}⁶ at each occurrence are independently selected from the group consisting of hydrogen, halogen, oxo, hydroxy, amino, cyano, nitro, optionally substituted C₁-C₈ alkyl, optionally substituted C₂-C₈ alkenyl, optionally substituted C₂-C₈ alkynyl, optionally substituted 1-8 membered heteroalkyl1-8 membered heteroalkyl, optionally substituted 3-8 membered heteroalkenyl, optionally substituted 3-8 membered heteroalkynyl, optionally substituted C₁-C₈ haloalkyl, optionally substituted C₃-C₈ carbocyclyl, and optionally substituted 3 to 8 membered heterocyclyl; or R_{E}⁵ and R_{E}⁶ together with the atom(s) to which they are connected form an optionally substituted C₃-C₈ cycloalkyl or optionally substituted 3 to 8 membered heterocyclyl ring;
R_{E}¹ indicates 1, 2, 3, 4, or 5 substituents (preferably 1 or 2 substituents), and the substituents, at each occurrence, are independently selected from the group consisting of hydrogen, halogen, cyano, nitro, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₃-C₈ carbocyclyl, and optionally substituted 3-8 membered heterocyclyl; or two R_{E}¹ together with the atom(s) to which they are connected form an optionally substituted C₃-C₈ cycloalkyl or optionally substituted 3-8 membered heterocyclyl ring;
L_{E} is a divalent group selected from the group consisting of null, -L_{E}¹-, and -L_{E}¹-L_{E}²- wherein L_{E}¹ and L_{E}² are independently selected from the group consisting of —CO—, —O—, —CR_{E}¹⁰R_{E}¹¹— and —NR_{E}¹⁰-, with the proviso that -L_{E}¹-L_{E}²- is not —O—O—, wherein R_{E}¹⁰ and R_{E}¹¹ are independently selected from the group consisting of hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈ alkoxy, and optionally substituted C₁-C₈ alkylamino;
Ring A_{E} is a divalent group selected from the group consisting of FORMULA A_{E}1, A_{E}2, A_{E}3, A_{E}4, A_{E}5 and A_{E}6:

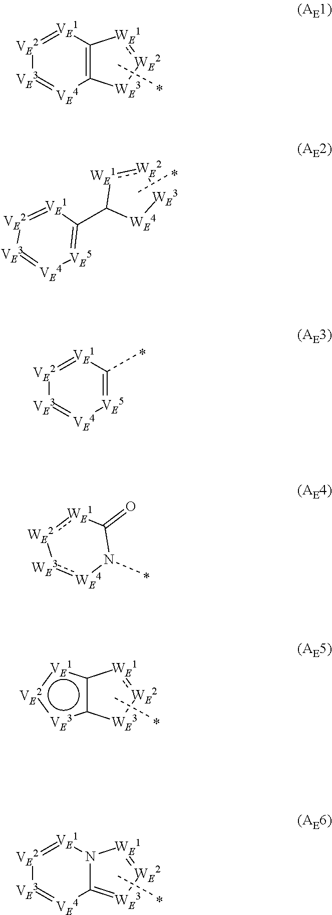

wherein
* indicates the attachment to L_{E}; and Z_{E} is attached to any possible position on the Ring A_{E};
[symbol] indicates a single bond or a double bond;
V_{E}¹, V_{E}², V_{E}³, V_{E}⁴ and V_{E}⁵, at each occurrence, are each independently selected from the group consisting of a bond, C, CR_{E}², N and NR_{E}²; or V_{E}¹ and V_{E}², V_{E}² and V_{E}³, V_{E}³ and V_{E}⁴, or V_{E}⁴ and V_{E}⁵ are combined together to optionally form 6 membered aryl ring or a 5, or 6 membered heteroaryl ring;
R_{E}², at each occurrence, is independently selected from the group consisting of absent, hydrogen, halogen, cyano, nitro, hydroxyl, amino, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈ alkoxy, optionally substituted C₁-C₈ alkylamino, optionally substituted C₃-C₈ carbocyclyl, and optionally substituted 3-8 membered heterocyclyl; or R_{E}² and another R_{E}² together with the atom(s) to which they are connected form optionally substituted C₃-C₈ cycloalkyl, optionally substituted 3-8 membered heterocyclyl ring, optionally substituted aryl, and optionally substituted heteroaryl;
W_{E}¹, W_{E}², W_{E}³ and W_{E}⁴ are each independently selected from the group consisting of —N═, —C≡, —CR_{E}³=, —CO—, —O—, —CR_{E}³R_{E}⁴-, —NR_{E}³—, —CR_{E}³=CR_{E}⁴-, —N═CR_{E}³-, and —N═N—; or W_{E}¹ and W_{E}², W_{E}² and W_{E}³, or W_{E}³ and W_{E}⁴ are combined together to optionally form 6 membered aryl ring or a 5, or 6 membered heteroaryl ring; and
R_{E}³ and R_{E}⁴, at each occurrence, are independently selected from the group consisting of absent, hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₃-C₈ carbocyclyl, and optionally substituted 3 to 8 membered heterocyclyl; or R_{E}³ and R_{E}⁴, on the same atom or on the adjacent atoms, together with the atom(s) to which they are connected form an optionally substituted C₃-C₈ cycloalkyl or 3-8 membered heterocyclyl ring; or
the degradation tag is a moiety of FORMULA 6A, 6B, or 6C:

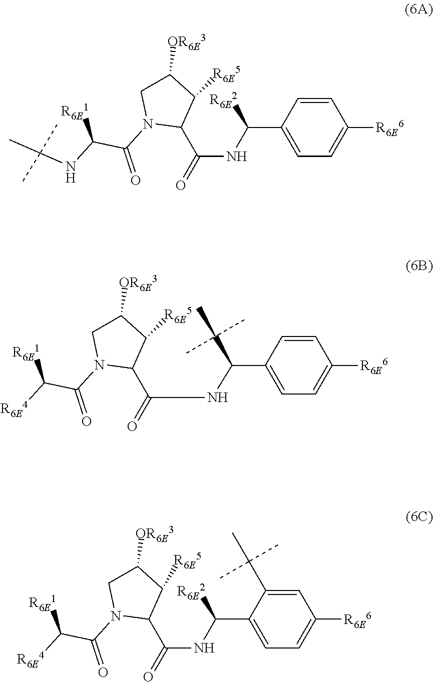

wherein
R_{6E}¹ and R_{6E}² are independently selected from the group consisting of hydrogen, hydroxyl, amino, cyano, nitro, optionally substituted C₁-C₈ alkyl, optionally substituted C₂-C₈ alkenyl, optionally substituted C₂-C₈ alkynyl; optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₁₀ carbocyclyl, and optionally substituted 3-10 membered heterocyclyl;
R_{6E}³ is selected from the group consisting of hydrogen, optionally substituted C(O)C₁-C₈ alkyl, optionally substituted C(O)1-8 membered heteroalkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(C₃-C₁₀ carbocyclyl), optionally substituted C(O)(3-10 membered heterocyclyl), optionally substituted C(O)C₂-C₈ alkenyl, optionally substituted C(O)C₂-C₈ alkynyl, optionally substituted C(O)-3-8 membered heteroalkenyl, optionally substituted C(O)-3-8 membered heteroalkynyl, optionally substituted C(O)OC₁-C₈ alkyl, optionally substituted C(O)O1-8 membered heteroalkyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(C₃-C₁₀ carbocyclyl), optionally substituted C(O)O(3-10 membered heterocyclyl), optionally substituted C(O)OC₂-C₈ alkenyl, optionally substituted C(O)OC₂-C₈ alkynyl, optionally substituted C(O)O-3-8 membered heteroalkenyl, optionally substituted C(O)O-3-8 membered heteroalkynyl, optionally substituted C(O)NHC₁-C₈ alkyl, optionally substituted C(O)NH₁₋₈ membered heteroalkyl, optionally substituted C(O)NHC₁-C₈alkoxyC₁-C₈alkyl, optionally substituted C(O)NHC₁-C₈ haloalkyl, optionally substituted C(O)NHC₁-C₈ hydroxyalkyl, optionally substituted C(O)NHC₁-C₈ aminoalkyl, optionally substituted C(O)NHC₁-C₈alkylaminoC₁-C₈alkyl, optionally substituted C(O)NH(C₃-C₁₀ carbocyclyl), optionally substituted C(O)NH(3-10 membered heterocyclyl), optionally substituted C(O)NHC₂-C₈ alkenyl, optionally substituted C(O)NHC₂-C₈ alkynyl, optionally substituted C(O)NH-3-8 membered heteroalkenyl, optionally substituted C(O)NH-3-8 membered heteroalkynyl, optionally substituted P(O)(OH)₂, optionally substituted P(O)(OC₁-C₈ alkyl)₂, and optionally substituted P(O)(OC₁-C₈ aryl)₂;
R_{6E}⁴ is selected from the group consisting of NR_{6E}⁷R_{6E}⁸,

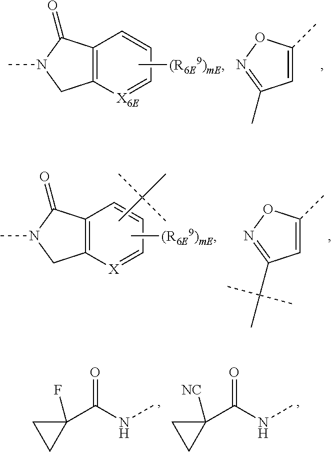

optionally substituted C₁-C₈alkoxy, optionally substituted C₃-C₁₀ carbocyclyl, optionally substituted 3-10 membered heterocyclyl, optionally substituted aryl, and optionally substituted heteraryl, wherein
R_{6E}⁷ is selected from hydrogen, optionally substituted C₁-C₈alkyl, optionally substituted 1-8 membered heteroalkyl, optionally substituted C₁-C₈ haloalkyl, optionally substituted C₃-C₈cycloalkyl, optionally substituted C₁-C₈alkyl-CO, optionally substituted 1-8 membered heteroalkyl-CO, optionally substituted C₁-C₈ haloalkyl-CO, optionally substituted C₃-C₈cycloalkyl-CO, optionally substituted C₃-C₈cycloalkyl-C₁-C₈alkyl-CO, optionally substituted 3-10 membered heterocyclyl-CO, optionally substituted 3-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;
R_{6E}⁸ is selected from hydrogen, optionally substituted C₁-C₈alkyl, optionally substituted 1-8 membered heteroalkyl, optionally substituted C₁-C₈ haloalkyl, and optionally substituted C₃-C₈cycloalkyl;
R_{6E}⁹, at each occurrence, is independently selected from the group consisting of hydrogen, halogen, cyano, optionally substituted C₁-C₈alkyl, optionally substituted 1-8 membered heteroalkyl, optionally substituted C₁-C₈ haloalkyl, optionally substituted C₃-C₈cycloalkyl, optionally substituted 1-8 membered heterocycloalkyl, optionally substituted C₁-C₈alkoxy, optionally substituted C₁-C₈cycloalkoxy, halo substituted C₁-C₈alkyl, halo substituted C₃-C₈cycloalkyl, halo substituted C₁-C₈alkoxyl, halo substituted C₁-C₈cycloalkoxy, and halo substituted 1-8 membered heterocycloalkyl; X_{6E} is selected from CH and N;
m_{E} is 0, 1, 2, 3, or 4;
R_{6E}⁵ is selected from the group consisting of hydrogen and halogen (preferably, H and F); and
R_{6E}⁶ is selected from hydrogen, halogen, hydroxyl, amino, cyano, nitro, optionally substituted C₁-C₈alkyl, optionally substituted 1-8 membered heteroalkyl, optionally substituted C₁-C₈ haloalkyl, optionally substituted C₃-C₈cycloalkyl, optionally substituted C₁-C₈alkoxy, and optionally substituted C₁-C₈cycloalkoxy, optionally substituted 1-8 membered heterocycloalkyl, optionally substituted aryl, and optionally substituted heteroaryl (preferably, halogen, cyano, optionally substituted imidazole, optionally substituted pyrazole, optionally substituted oxadiazole, optionally substituted triazole, 4-methylthiazol-5-yl, or oxazol-5-yl group);
and
c) the linker moiety is of FORMULA 9:

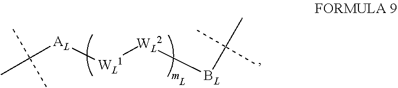

wherein
A_{L}, W_{L}¹, W_{L}², and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}, R_{L}^{d}N(R_{L}¹)R_{L}^{e}, R_{L}^{d}N(R_{L}¹)COR_{L}¹, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted 2-8 membered heteroalkylene, optionally substituted 3-8 membered heteroalkenylene, optionally substituted 3-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl, wherein
R_{L}^{d} and R_{L}^{e}, at each occurrence, are independently selected from the group consisting of null, R_{L}^{r}, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted 2-8 membered heteroalkylene, optionally substituted 3-8 membered heteroalkenylene, optionally substituted 3-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted C₃-C₁₃ carbocyclyl, optionally substituted 3-13 membered heterocyclyl, optionally substituted aryl, and optionally substituted heteroaryl;
R_{L}^{r}, at each occurrence, is selected from optionally substituted C₃-C₁₀ carbocyclyl, optionally substituted 3-10 membered heterocyclyl, optionally substituted aryl, and optionally substituted heteroaryl;
R_{L}¹ and R_{L}², at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted 2-8 membered heteroalkylene, optionally substituted 3-8 membered heteroalkenylene, optionally substituted 3-8 membered heteroalkynylene, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; or
R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are connected optionally form a C₃-C₂₀ carbocyclyl or 3-20 membered heterocyclyl ring; and
m_{L} is 1, 2, 3, 4, 5, 6, 7, 8, 9, 10, 11, 12, 13, 14, or 15.

In some embodiments, the cycloalkyl includes monocyclic cycloalkyl, fused cycloalkyl, bridged cycloalkyl, or spiro cycloalkyl.

In some embodiments, the carbocyclyl includes monocyclic carbocyclyl, fused carbocyclyl, bridged carbocyclyl, or spiro carbocyclyl.

In some embodiments, the heterocyclyl includes monocyclic heterocyclyl, fused heterocyclyl, bridged heterocyclyl, or spiro heterocyclyl.

In some embodiments, the aryl includes monocyclic aryl, bicyclic fused aryl, or tricyclic fused aryl.

In some embodiments, the heteroaryl includes monocyclic heteroaryl, bicyclic fused heteroaryl, or tricyclic fused heteroaryl.

In some embodiments, each C₃-C₁₃ cycloalkyl, at each occurrence, is independently selected from the group consisting of C₃-C₁₀ monocyclic cycloalkyl, C₄-C₁₃ fused cycloalkyl, C₅-C₁₃ bridged cycloalkyl, or C₅-C₁₃ spiro cycloalkyl.

In some embodiments, the C₃-C₁₃ carbocyclyl, at each occurrence, is independently selected from the group consisting of C₃-C₁₀ monocyclic carbocyclyl, C₄-C₁₃ fused carbocyclyl, C₅-C₁₃ bridged carbocyclyl, or C₅-C₁₃ spiro carbocyclyl.

In some embodiments, the 3-13 membered heterocyclyl, at each occurrence, is independently selected from the group consisting of 3-10 membered monocyclic heterocyclyl, 5-13 membered fused heterocyclyl, 5-13 membered bridged heterocyclyl, or 5-13 membered spiro heterocyclyl.

In some embodiments, the aryl, at each occurrence, is independently selected from the group consisting of monocyclic aryl, bicyclic fused aryl, and tricyclic fused aryl.

In some embodiments, the heteroaryl, at each occurrence, is independently selected from the group consisting of monocyclic heteroaryl, bicyclic fused heteroaryl, and tricyclic fused heteroaryl.

In some embodiments, the TRK ligand is a moiety of FORMULA 1-1A, or 1-1B:

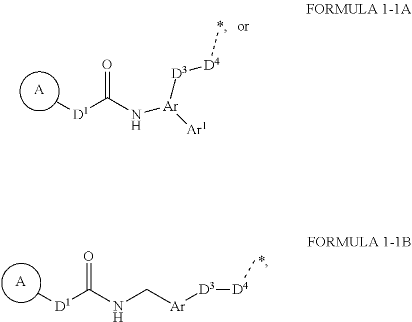

wherein
D¹, D³, D⁴, Ring A, Ar and Ar¹ are defined as in FORMULA 1.

In some embodiments, D¹ is selected from null, —NH—, —CH₂—, and —CH₂NH—.

In some embodiments, D¹ is —NH—.

In some embodiments, the TRK ligand is a moiety of FORMULA 1-2A, 1-2B, 1-2C, 1-2D, 1-2E, or 1-2F:

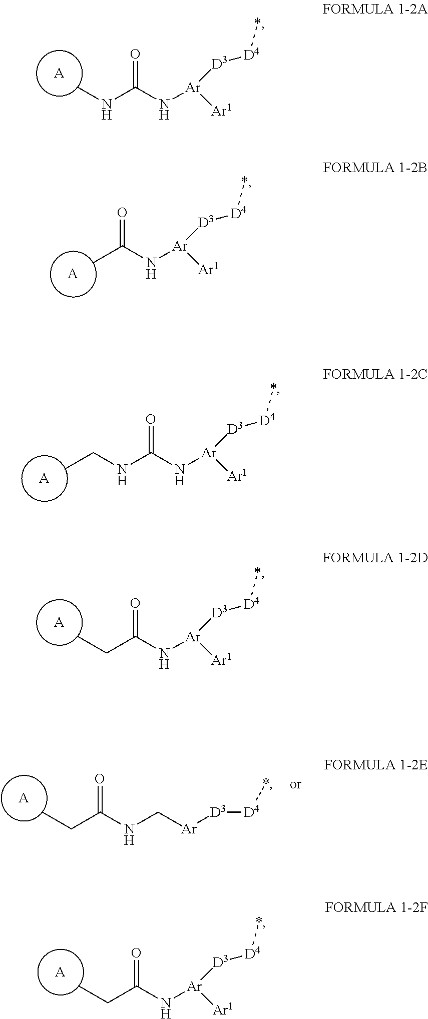

wherein
D³, D⁴, Ring A, Ar, and Ar¹ are defined as in FORMULA 1.

In some embodiments, Ring A is Ring A′, wherein: Ring A′ is selected from the group consisting of optionally substituted C₃-C₁₃ carbocyclyl and optionally substituted 3-13 membered heterocyclyl, and Ring A′ is optionally further substituted with Ar³; and
Ar³ is selected from the group consisting of optionally substituted aryl, and optionally substituted heteroaryl.

In some embodiments, Ring A is Ar²; wherein: Ar² is selected from the group consisting of optionally substituted aryl, and optionally substituted heteroaryl.

In some embodiments, the TRK ligand is a moiety of FORMULA 1-3A, 1-3B, 1-3C, 1-3D, 1-3E, 1-3F, or 1-3G:

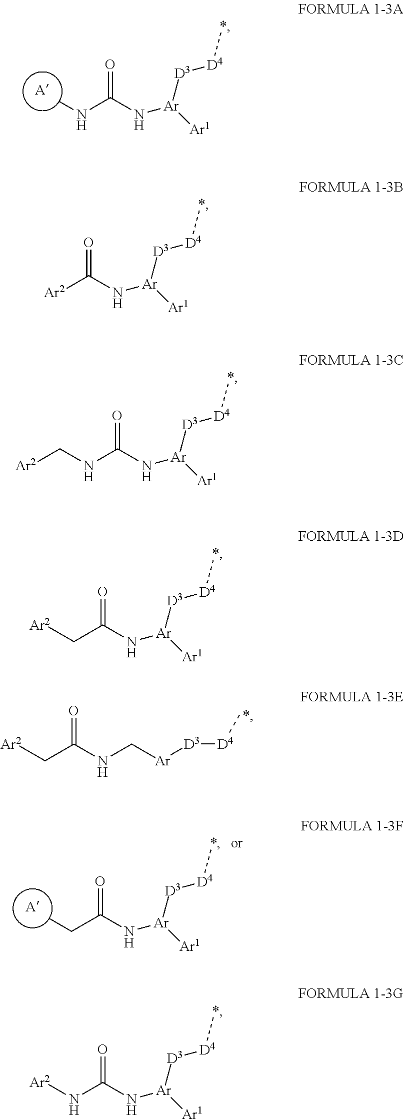

wherein
D³, D⁴, Ar and Ar¹ are defined as in FORMULA 1;
Ring A′ is selected from the group consisting of optionally substituted C₃-C₁₃ carbocyclyl, and optionally substituted 3-13 membered heterocyclyl and Ring A′ is optionally further substituted with Ar³;
Ar³ is selected from the group consisting of optionally substituted aryl, and optionally substituted heteroaryl; and
Ar² is selected from the group consisting of optionally substituted aryl, and optionally substituted heteroaryl.

In some embodiments, Ar is an optionally substituted aromatic ring, wherein the aromatic ring is selected from the group consisting of:

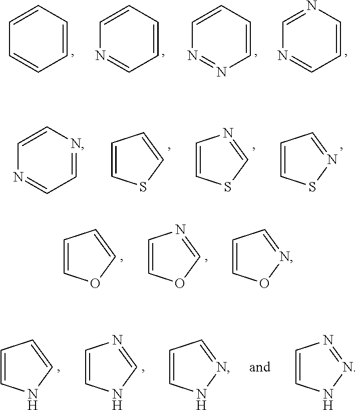

In some embodiments, Ar is an optionally substituted aromatic group, wherein the aromatic group is selected from the group consisting of

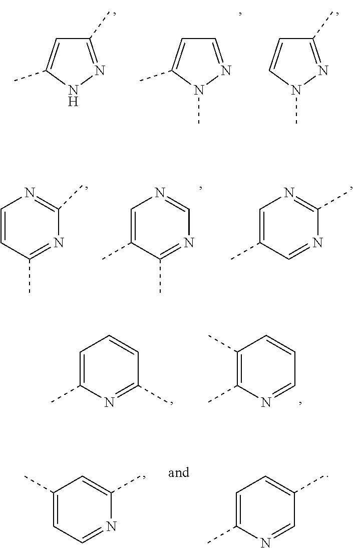

In some embodiments, Ar is substituted with Ar¹, and Ar—Ar¹ is an optionally substituted group, wherein the group is selected from the group consisting of

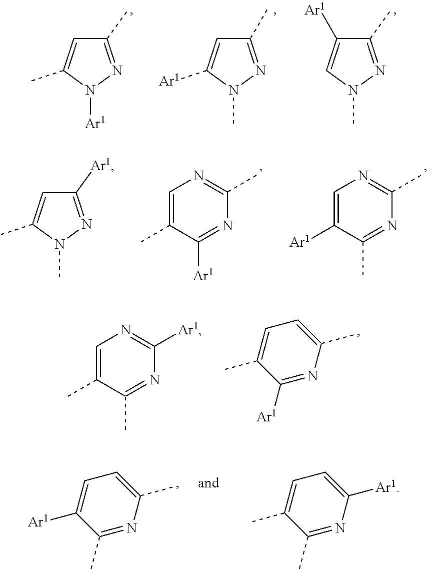

In some embodiments, Ar—Ar¹ is an optionally substituted group, wherein the group is selected from the group consisting of

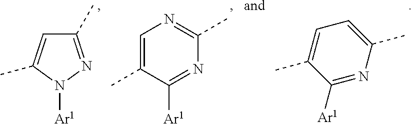

In some embodiments, Ar—Ar¹ is an optionally substituted group of

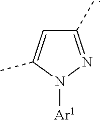

In some embodiments, Ar is optionally substituted with R³, wherein:
R³ is selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₁-C₈alkyl-NHC(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted aryl, and optionally substituted heteroaryl.

In some such embodiments, Ar is substituted with Ar¹, and optionally further substituted with R³.

In some embodiments, the TRK ligand is a moiety of FORMULA 1-4A, 1-4B, 1-4C, 1-4D, 1-4E, 1-4F or 1-4G:

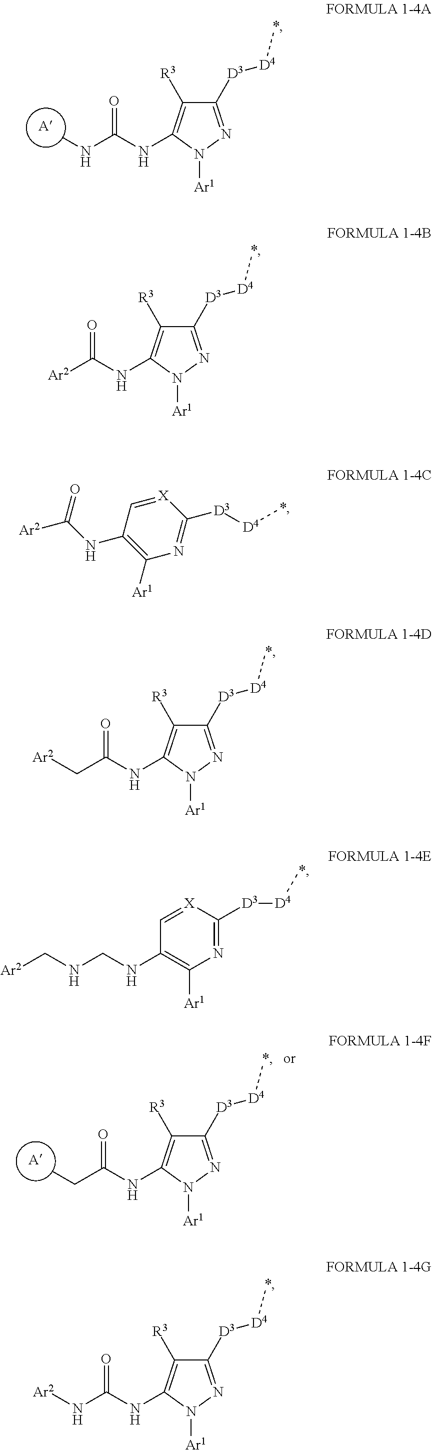

wherein
D³, D⁴, and Ar¹ are defined as in FORMULA 1;
Ring A′ and Ar² are defined as in FORMULA 1-3A, 1-3B, 1-3C, 1-3D, 1-3E, 1-3F, or 1-3G;
X is selected from CH and N; and
R³ is selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₁-C₈alkyl-NHC(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted aryl, and optionally substituted heteroaryl.

In some embodiments, W is selected from the group consisting of hydrogen, optionally substituted C₁-C₈ alkyl.

In some embodiments, R³ is selected from H, CH₃, CF₃, CHF₂, CH(CH₃)₂, and cyclopropyl.

In some embodiments, Ring A′ is optionally substituted with (R⁴)ₙ, where n is an integer from 0-7, and/or R⁵; wherein R⁴ and R⁵ are defined hereinbelow. In some such embodiments, Ring A′ is further substituted with Ar³.

In some embodiments, Ring A′ is a moiety of formula:

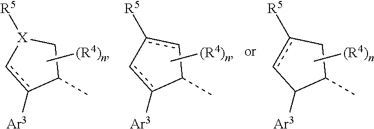

wherein
[symbol] indicates a single bond or a double bond;
X is selected from N and CH;
R⁴, at each occurrence, is independently selected from the group consisting of null, H, F, OH, optionally substituted C₁-C₄ alkyl, optionally substituted C₂-C₄ alkenyl, optionally substituted C₂-C₄ alkynyl, optionally substituted 1-4 membered heteroalkyl, optionally substituted 3-4 membered heteroalkenyl, and optionally substituted 3-4 membered heteroalkynyl;
n is selected from 0, 1, 2, 3, 4, 5, 6 and 7;
R⁵ is selected from null, hydrogen, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkyl-C(O)—, optionally substituted C₁-C₈ alkyl-NH—C(O)—, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, optionally substituted 3-8 membered heterocyclyl-C₁-C₈alkyl, optionally substituted aryl, optionally substituted heteroaryl, optionally substituted aryl-C₁-C₈alkyl, and optionally substituted heteroaryl-C₁-C₈alkyl; or
two R⁴, or R⁴ and R⁵, or R⁴ and Ar³ together with the atom or atoms to which they are connected optionally form a C₃-C₂₀ carbocyclyl, 3-20 membered heterocyclyl, aryl ring, or heteroaryl ring (sometimes referred to as Ring D and/or Ring E herein); and
Ar³ is selected from optionally substituted aryl, and optionally substituted heteroaryl.

In some embodiments, Ring A′ is optionally substituted with (R^{4′})ₙ, where n is an integer from 0-7, and/or R^{5′}, and/or (R⁶)ₘ; wherein m is an integer from 0-8, where R^{4′}, R^{5′} and R⁶ are defined hereinbelow. In some such embodiments, Ring A′ is further substituted with Ar³.

In some embodiments, the TRK ligand is a moiety of FORMULA 1-5A, 1-5B, 1-5C, 1-5D, 1-5E, 1-5F, 1-5G, or 1-5H:

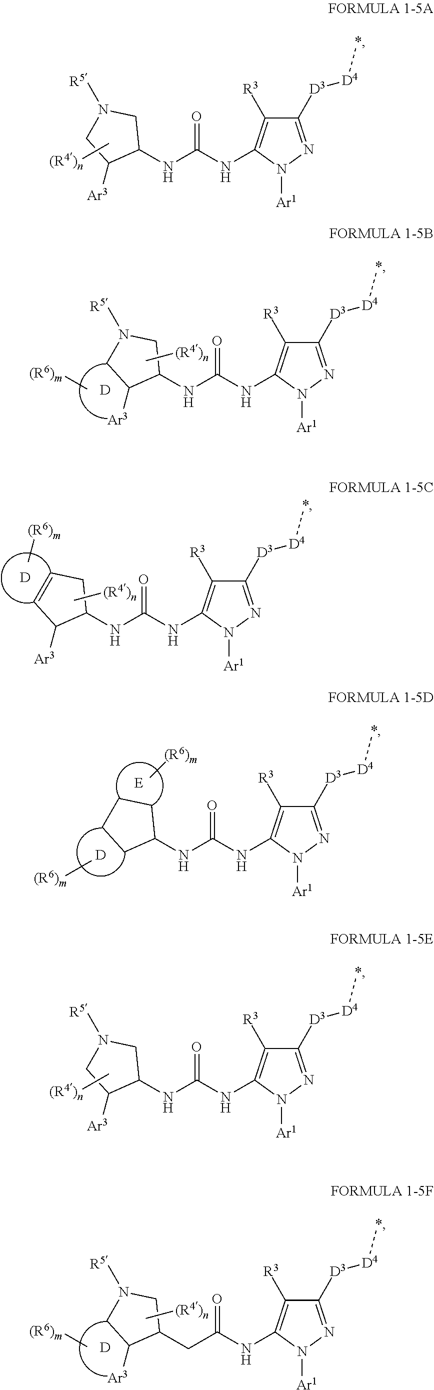

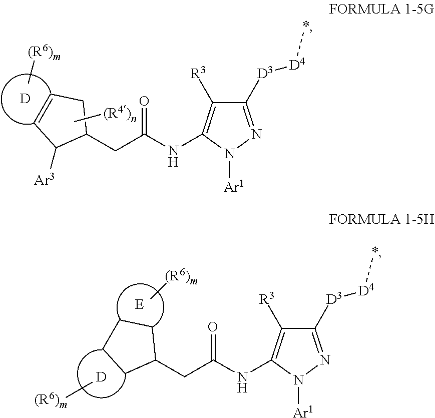

wherein
D³, D⁴, and Ar¹ are defined as in FORMULA 1;
R³ is defined as in FORMULA 1-4A, 1-4B, 1-4C, 1-4D, 1-4E, 1-4F, or 1-4G;
R^{4′}, at each occurrence, is independently selected from the group consisting of null, H, F, OH, optionally substituted C₁-C₃ alkyl, and optionally substituted 1-3 membered heteroalkyl; n is selected from 0, 1, 2, 3, 4, 5, 6 and 7;
R^{5′} is selected from the group consisting of hydrogen, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkyl-C(O)—, optionally substituted C₁-C₈ alkyl-NHC(O)—, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted C₂-C₈ alkenyl, optionally substituted aryl, optionally substituted heteroaryl, optionally substituted aryl-C₁-C₈alkyl, and optionally substituted heteroaryl-C₁-C₈alkyl;
Ring D and Ring E are independently selected from the group consisting of optionally substituted C₃-C₁₃ carbocyclyl, optionally substituted 3-13 membered heterocyclyl, optionally substituted aryl, and optionally substituted heteroaryl;
R⁶, at each occurrence, is independently selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₁-C₈alkyl-NH—C(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted C₂-C₈ alkenyl, optionally substituted aryl, optionally substituted heteroaryl, optionally substituted aryl-C₁-C₈alkyl, and optionally substituted heteroaryl-C₁-C₈alkyl;
m is selected from 0, 1, 2, 3, 4, 5, 6, 7 and 8; and
Ar³ is selected from optionally substituted aryl, and optionally substituted heteroaryl.

In some embodiments, Ar³ and —NH—C(O)—NH— group are trans to each other.

In some embodiments,

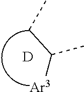

is a fused ring formed by Ring D and Ar³.

In some embodiments, the TRK ligand is a moiety of FORMULA 1-6A, 1-6B, 1-6C, 1-6D, 1-6E, 1-6F, or their corresponding enantiomers:

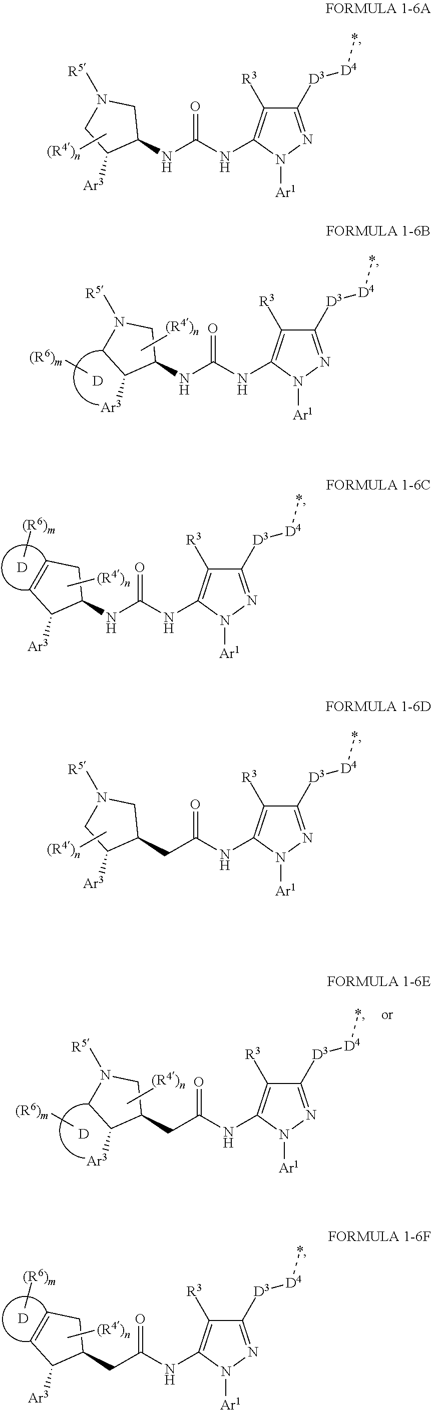

wherein
D³, D⁴, and Ar¹ are defined as in FORMULA 1;
R³ is defined as in FORMULA 1-4A, 1-4B, 1-4C, 1-4D, 1-4E, 1-4F, or 1-4G; and Ring D, Ar³, R^{4′}, R^{5′}, R⁶, m, and n are defined as in 1-5A, 1-5B, 1-5C, 1-5D, 1-5E, 1-5F, 1-5G, or 1-5H.

In some embodiments, Ar³ is an optionally substituted aromatic ring, wherein the aromatic ring is selected from the group consisting of:

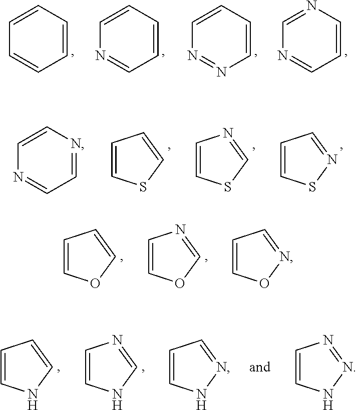

In some embodiments, Ar³ is selected from the group consisting of

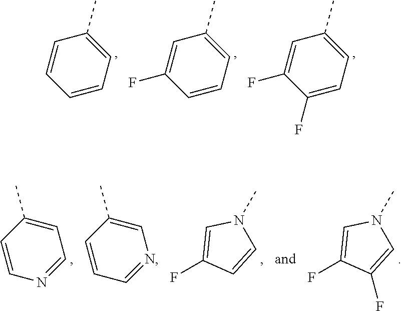

In some embodiments, the TRK ligand is a moiety of FORMULA 1-7A, 1-7B, 1-7C, 1-7D, 1-7E, 1-7F, 1-7G, 1-7H, 1-7I, 1-7J, 1-7K, 1-7L, or their corresponding enantiomers:

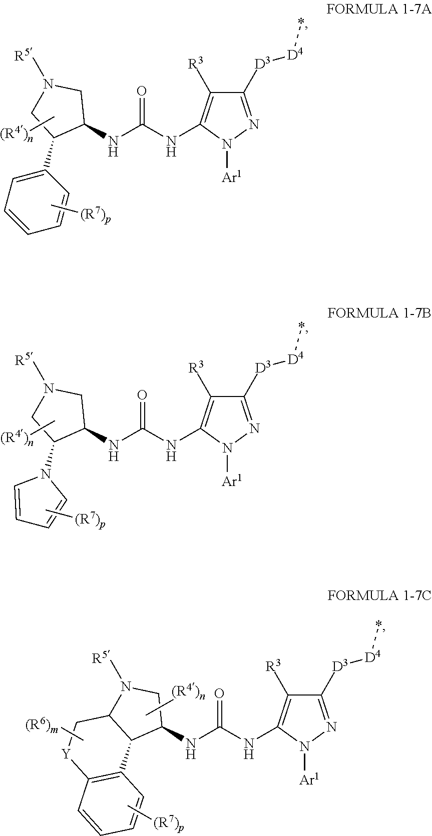

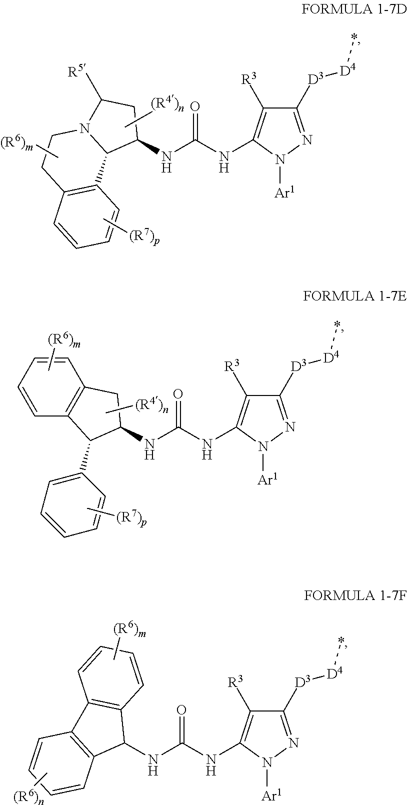

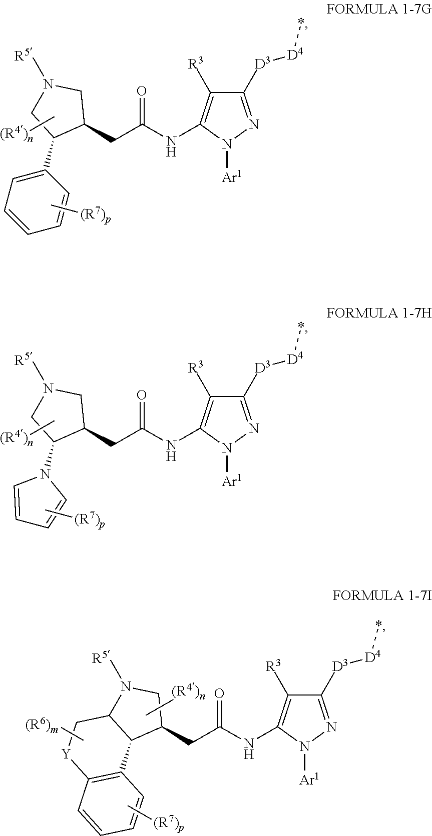

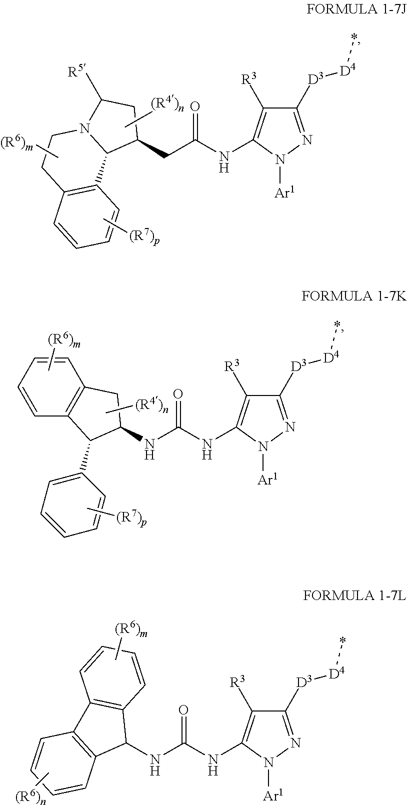

wherein
D³, D⁴, and Ar¹ are defined as in FORMULA 1;
R³ is defined as in FORMULA 1-4A, 1-4B, 1-4D, 1-4F, or 1-4G;
R^{4′}, R^{5′}, R⁶, m, and n are defined as in FORMULA 1-5A, 1-5B, 1-5C, or 1-5D;
Y is selected from CH₂, NH, and O;
R⁷, at each occurrence, is independently selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₁-C₈alkyl-NH—C(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted aryl, optionally substituted heteroaryl, optionally substituted aryl-C₁-C₈alkyl, and optionally substituted heteroaryl-C₁-C₈alkyl; and
p is selected from 1, 2, 3, 4, and 5.

In some embodiments, R^{4′}, at each occurrence, is independently selected from the group consisting of null, H, F, Cl, OH, CH₃, CHF₂, CF₃, isopropyl, and cyclopropyl.

In some embodiments, R^{5′} is selected from the group consisting of methoxyethyl and difluoroethyl.

In some embodiments, R⁶, at each occurrence, is independently selected from the group consisting of H, F, Cl, CN, OH, optionally substituted C₁-C₃ alkyl, optionally substituted C₂-C₃ alkenyl, optionally substituted C₂-C₃ alkynyl, optionally substituted 1-3 membered heteroalkyl, optionally substituted 3 membered heteroalkenyl, optionally substituted 3 membered heteroalkynyl, optionally substituted C₁-C₃alkoxy, optionally substituted C₁-C₃alkyl-S—, C₁-C₃alkylamino, optionally substituted C₁-C₃alkoxyC₁-C₃alkyl, optionally substituted C₁-C₃ haloalkyl, optionally substituted C₁-C₃ hydroxyalkyl, optionally substituted C₁-C₃alkylaminoC₁-C₃alkyl, optionally substituted C₃-C₆ cycloalkyl, optionally substituted C₃-C₆ carbocyclyl, optionally substituted 3-6 membered heterocyclyl, optionally substituted C₃-C₆ carbocyclyl-C₁-C₃alkyl, and optionally substituted 3-6 membered heterocyclyl-C₁-C₃alkyl, optionally substituted aryl, optionally substituted heteroaryl, optionally substituted aryl-C₁-C₃alkyl, and optionally substituted heteroaryl-C₁-C₃alkyl.

In some embodiments, R⁶, at each occurrence, is independently selected from the group consisting of H, F, Cl, CN, OH, CH₃, CHF₂, CF₃, isopropyl, cyclopropyl, CH₃O, CHF₂O, CF₃O, isopropoxy, cyclopropoxy, CH₃OCH₂, CHF₂OCH₂, CF₃OCH₂, isopropoxy-CH₂—, and cyclopropoxy-CH₂—.

In some embodiments, R⁷, at each occurrence, is independently selected from the group consisting of H, F, Cl, CN, OH, optionally substituted C₁-C₃ alkyl, optionally substituted C₂-C₃ alkenyl, optionally substituted C₂-C₃ alkynyl, optionally substituted 1-3 membered heteroalkyl, optionally substituted 3 membered heteroalkenyl, optionally substituted 3 membered heteroalkynyl, optionally substituted C₁-C₃alkoxy, optionally substituted C₁-C₃alkyl-S—, C₁-C₃alkylamino, optionally substituted C₁-C₃alkoxyC₁-C₃alkyl, optionally substituted C₁-C₃ haloalkyl, optionally substituted C₁-C₃ hydroxyalkyl, optionally substituted C₁-C₃alkylaminoC₁-C₃alkyl, optionally substituted C₃-C₆ cycloalkyl, optionally substituted C₃-C₆ carbocyclyl, optionally substituted 3-6 membered heterocyclyl, optionally substituted C₃-C₆ carbocyclyl-C₁-C₃alkyl, and optionally substituted 3-6 membered heterocyclyl-C₁-C₃alkyl, optionally substituted aryl, optionally substituted heteroaryl, optionally substituted aryl-C₁-C₃alkyl, and optionally substituted heteroaryl-C₁-C₃alkyl.

In some embodiments, R⁷, at each occurrence, is independently selected from the group consisting of H, F, Cl, CN, CH₃, CHF₂, CF₃, isopropyl, and cyclopropyl.

In some embodiments, Ar² is an optionally substituted group, wherein the group is selected from the group consisting of

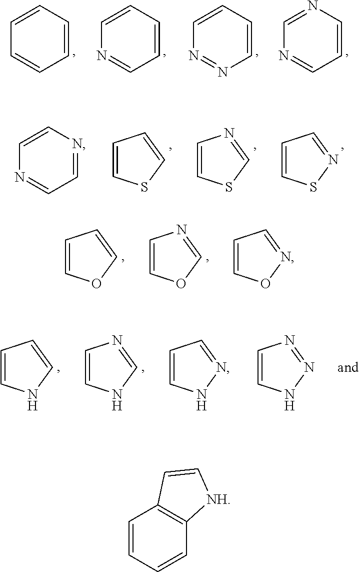

In some embodiments, Ar² is selected from optionally substituted phenyl, optionally substituted pyrrolyl, optionally substituted pyrazolyl, and optionally substituted triazolyl.

In some embodiments, Ring A is Ar², and Ar² is optionally substituted with (R′)_{q}, where q is an integer from 1-5, where R⁸ is defined hereinbelow.

In some embodiments, the TRK ligand is a moiety of FORMULA 1-5I, 1-5J, 1-5K, 1-5L, 1-5M, or 1-5N:

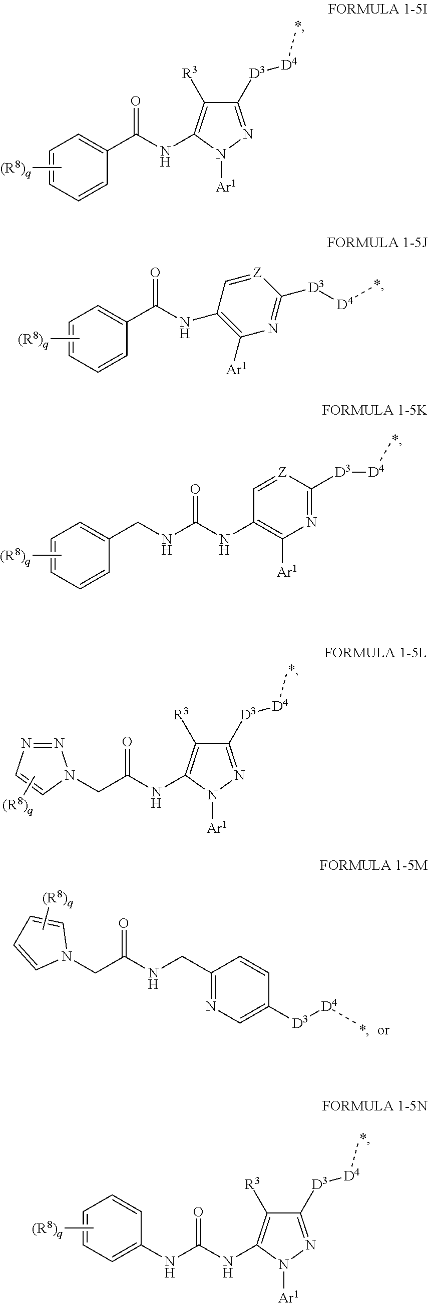

wherein
D³, D⁴, and Ar¹ are defined as in FORMULA 1;
R³ is defined as in FORMULA 1-4A, 1-4B, 1-4C, 1-4D, 1-4E, 14F, or 1-4G;
Ring A is Ar²;
Ar² is substituted with (R⁸)_{q};
Z is selected from CH and N;
R⁸, at each occurrence, is independently selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₂-C₈alkyl-NH—C(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted C₂-C₈ alkenyl, optionally substituted aryl, optionally substituted heteroaryl, optionally substituted aryl-C₁-C₈alkyl, and optionally substituted heteroaryl-C₁-C₈alkyl; or two R⁸ together with the atom or atoms to which they are connected optionally form C₃-C₂₀ carbocyclyl, 3-20 membered heterocyclyl, aryl ring, or heteroaryl ring; and
q is selected from 1, 2, 3, 4, and 5.

In some embodiments, R³ is selected from the group consisting of H, F, Cl, CN, OH, optionally substituted C₁-C₃ alkyl, optionally substituted C₂-C₃ alkenyl, optionally substituted C₂-C₃ alkynyl, optionally substituted 1-3 membered heteroalkyl, optionally substituted 3 membered heteroalkenyl, optionally substituted 2-3 membered heteroalkynyl, optionally substituted C₁-C₃alkoxy, optionally substituted C₁-C₃alkyl-S—, C₁-C₃alkylamino, optionally substituted C₁-C₃alkoxyC₁-C₃alkyl, optionally substituted C₁-C₃ haloalkyl, optionally substituted C₁-C₃ hydroxyalkyl, optionally substituted C₁-C₃alkylaminoC₁-C₃alkyl, optionally substituted C₃-C₆ cycloalkyl, optionally substituted C₃-C₆ carbocyclyl, optionally substituted 3-6 membered heterocyclyl, optionally substituted C₃-C₆ carbocyclyl-C₁-C₃alkyl, and optionally substituted 3-6 membered heterocyclyl-C₁-C₃alkyl, optionally substituted aryl, optionally substituted heteroaryl, optionally substituted aryl-C₁-C₃alkyl, and optionally substituted heteroaryl-C₁-C₃alkyl.

In some embodiments, R³ is selected from the group consisting of H, F, Cl, CN, CH₃, CHF₂, CF₃, isopropyl, and cyclopropyl.

In some embodiments, R³ is H or CH₃.

In some embodiments, Ar¹ is an optionally substituted group selected from the group consisting of

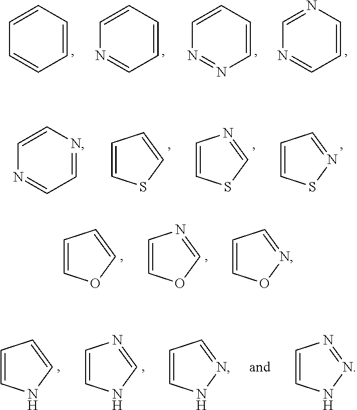

In some embodiments, Ar¹ is selected from optionally substituted phenyl and optionally substituted pyridinyl.

In some embodiments, Ar¹ is selected from

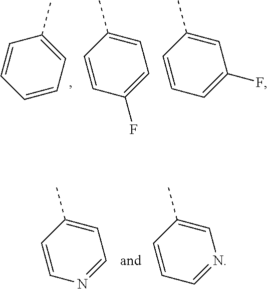

In some embodiments, D³ and D⁴ are independently selected from the group consisting of null, —O—, —NR¹—, —CO—, —CONR¹—, —SO₂—, —SO₂NR¹—, —NR¹CO—, —NR¹C(O)NR²—, —NR¹SO₂—, —NR¹SO₂NR²—, —OCONR¹—, optionally substituted C₁-C₄ alkylene, optionally substituted C₂-C₄ alkenylene, optionally substituted C₂-C₄ alkynylene, optionally substituted 1-4 membered heteroalkylene, optionally substituted 3-4 membered heteroalkenylene, optionally substituted 2-4 membered heteroalkynylene, optionally substituted C₁-C₄alkylene-O—C₁-C₄alkylene, optionally substituted C₁-C₄ haloalkylene, optionally substituted C₁-C₄ hydroxyalkylene, optionally substituted C₁-C₄alkylene-N(C₁-C₄alkyl)-C₁-C₄alkylene, optionally substituted C₃-C₆ cycloalkylene, optionally substituted C₃-C₆ carbocyclyl, optionally substituted 3-6 membered heterocyclyl, optionally substituted C₃-C₆ carbocyclyl-C₁-C₄alkylene, optionally substituted 3-6 membered heterocyclyl-C₁-C₄alkylene, optionally substituted C₃-C₆ carbocyclyl-O—, optionally substituted 3-6 membered heterocyclyl-O, optionally substituted C₃-C₆ carbocyclyl-N(C₁-C₄alkyl)-, and optionally substituted 3-6 membered heterocyclyl-N(C₁-C₄alkyl)-, optionally substituted aryl, and optionally substituted heteroaryl, wherein
R¹ and R² are independently selected from the group consisting of H, optionally substituted C₁-C₄ alkyl, optionally substituted C₂-C₄ alkenyl, optionally substituted C₂-C₄ alkynyl, optionally substituted 1-4 membered heteroalkyl, optionally substituted 3-4 membered heteroalkenyl, optionally substituted 2-4 membered heteroalkynyl, optionally substituted C₃-C₁₀ carbocyclyl, optionally substituted 3-10 membered heterocyclyl, optionally substituted aryl, and optionally substituted heteroaryl; or R¹ and R² together with the atom or atoms to which they are connected optionally form a C₃-C₂₀ carbocyclic ring or 3-20 membered heterocyclic ring;

In some embodiments, D³ and D⁴ are independently selected from the group consisting of null, —O—, —NR¹—, —CO—, —CONR¹—, —SO₂—, —SO₂NR¹—, —NR¹CO—, —NR¹SO₂—, optionally substituted C₁-C₄ alkylene, optionally substituted C₃-C₆ carbocyclyl, optionally substituted 3-6 membered heterocyclyl, optionally substituted aryl, and optionally substituted heteroaryl.

In some embodiments, D³ and D⁴ are independently selected from the group consisting of null, —O—, —NH—, —CO—, —CONH—, —SO₂—, —SO₂NH—, —NHCO—, —NHSO₂—, optionally substituted C₁-C₄ alkylene, and an optionally substituted group selected from the group consisting of

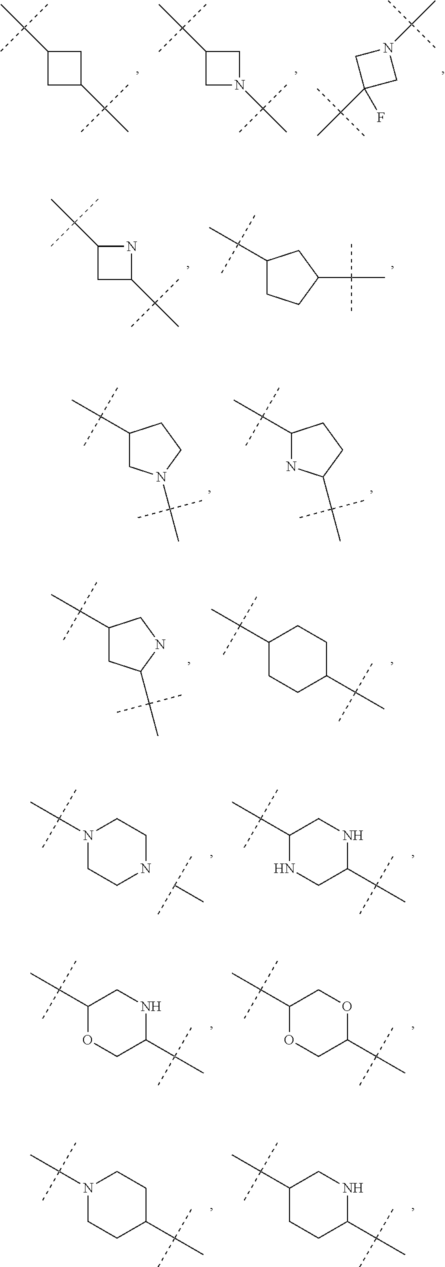

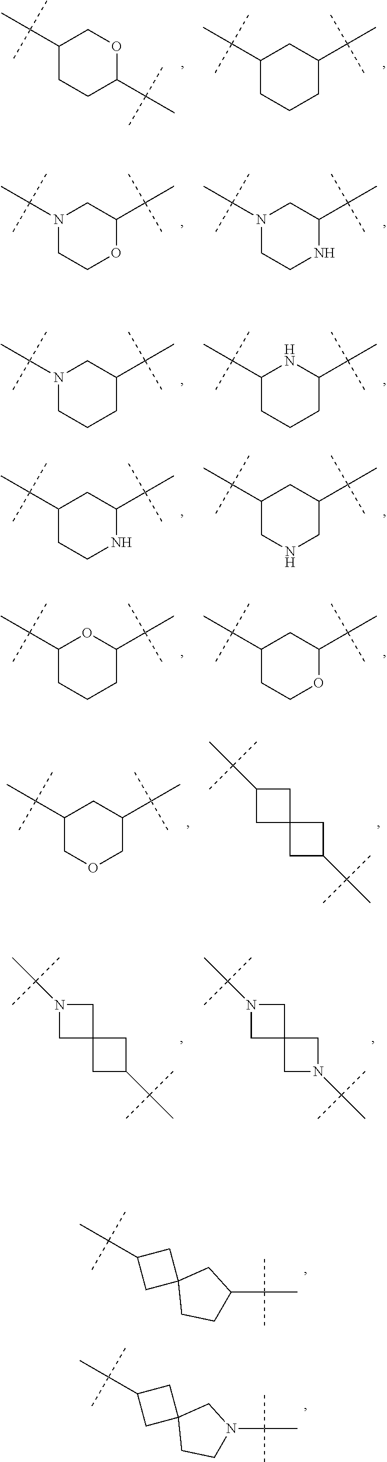

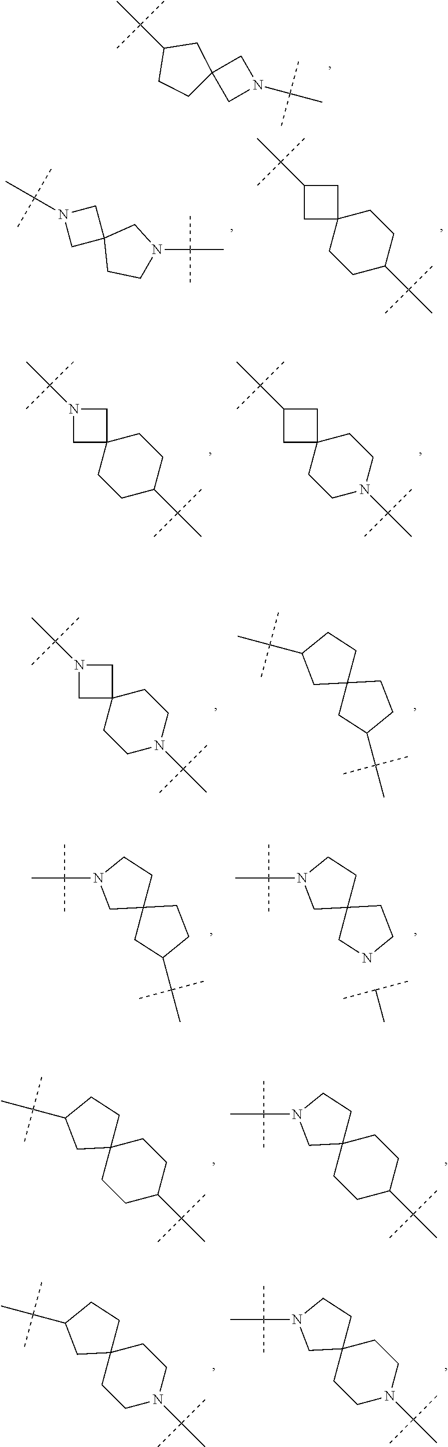

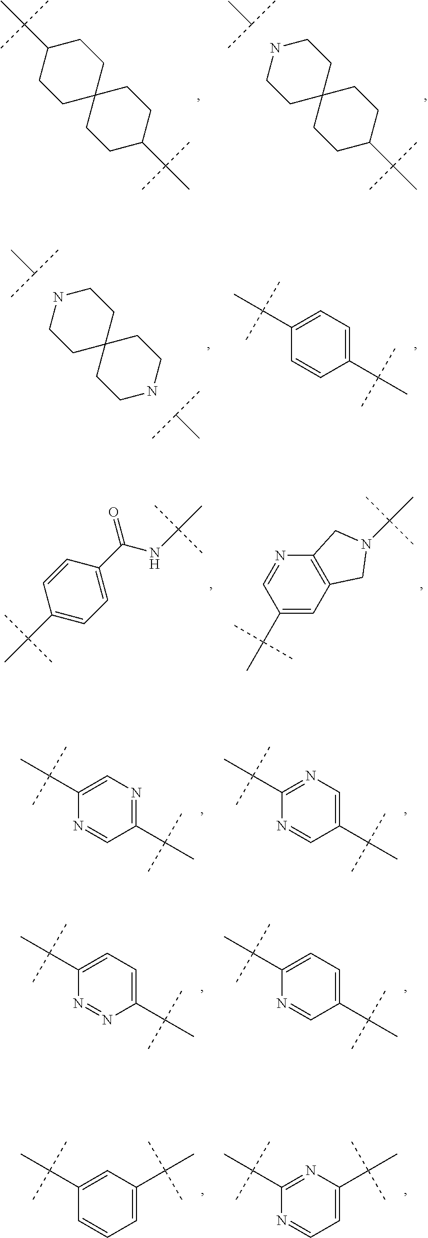

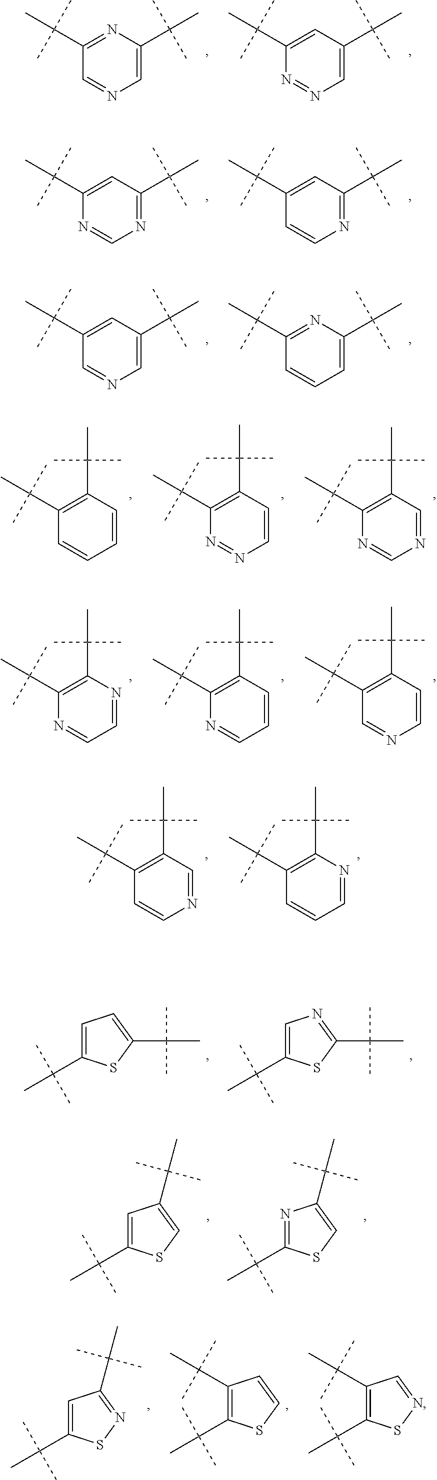

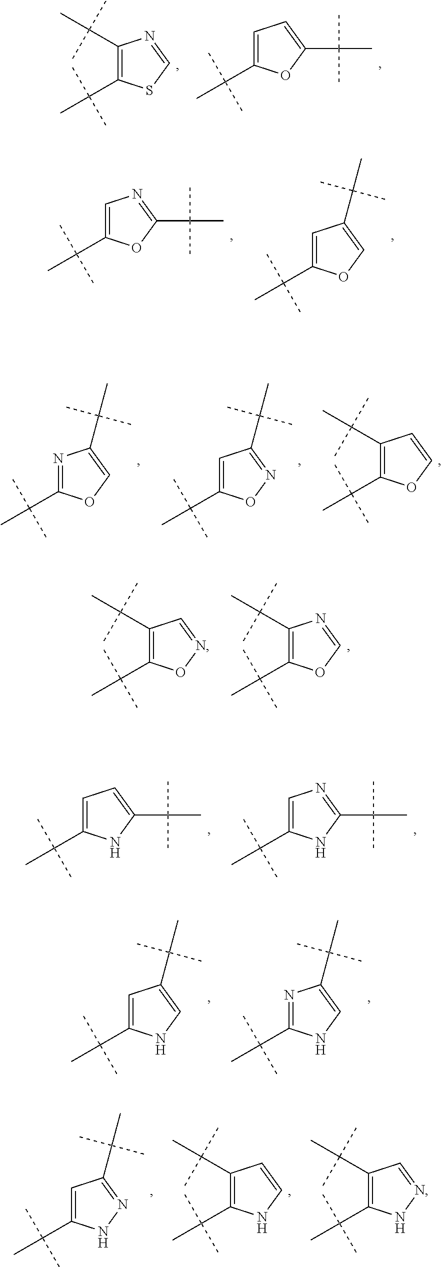

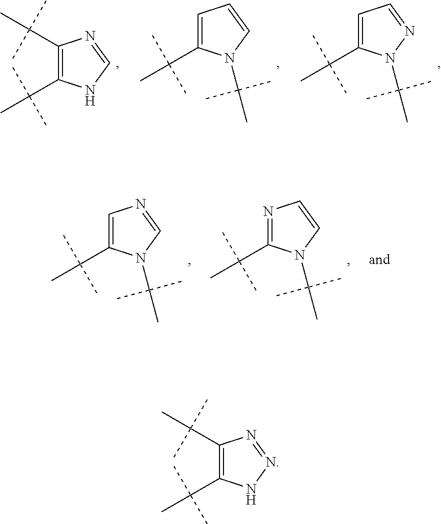

In some embodiments, D³ and D⁴ are independently selected from the group consisting of null, —O—, —NH—, —CO—, —CONH—, —SO₂—, —SO₂NH—, —NHCO—, —NHSO₂—, optionally substituted C₁-C₄ alkylene, and a optionally substituted group selected from the group consisting of

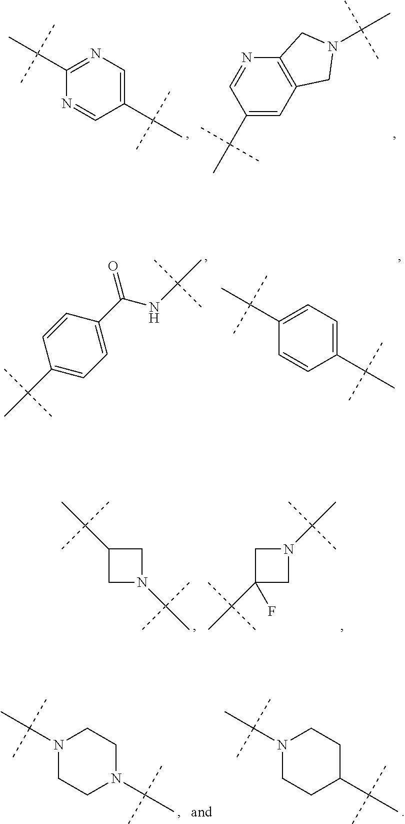

In some embodiments, -D³-D⁴- is selected from the group consisting of

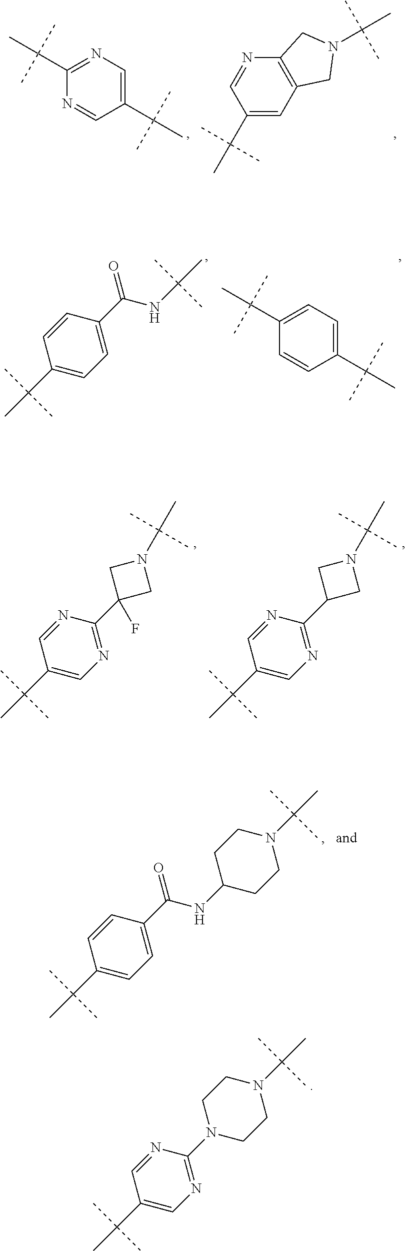

In some embodiments, the TRK ligand is a moiety of any of FORMULAE 1-8A to 1-8BB, or their corresponding enantiomers:

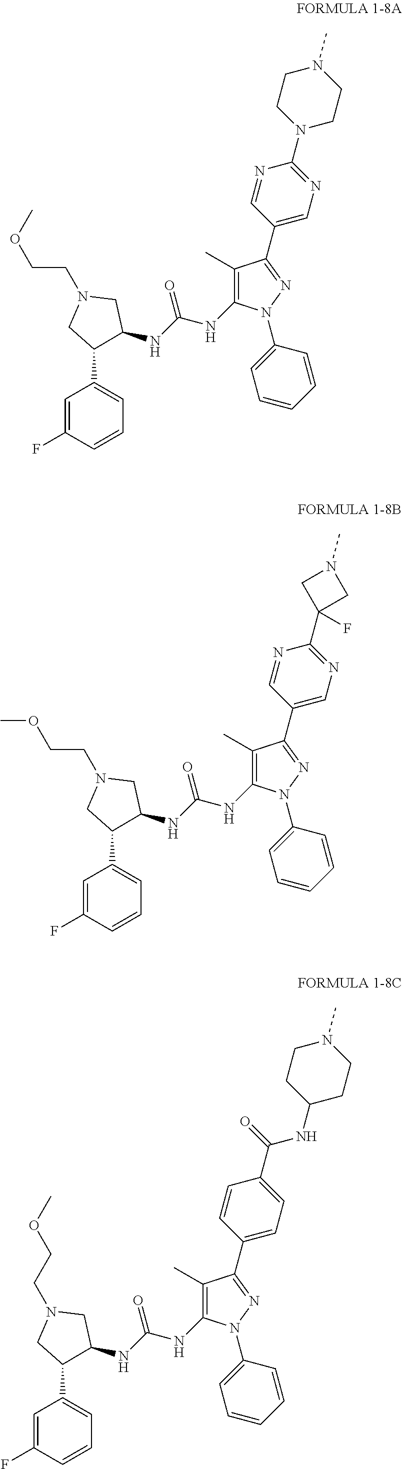

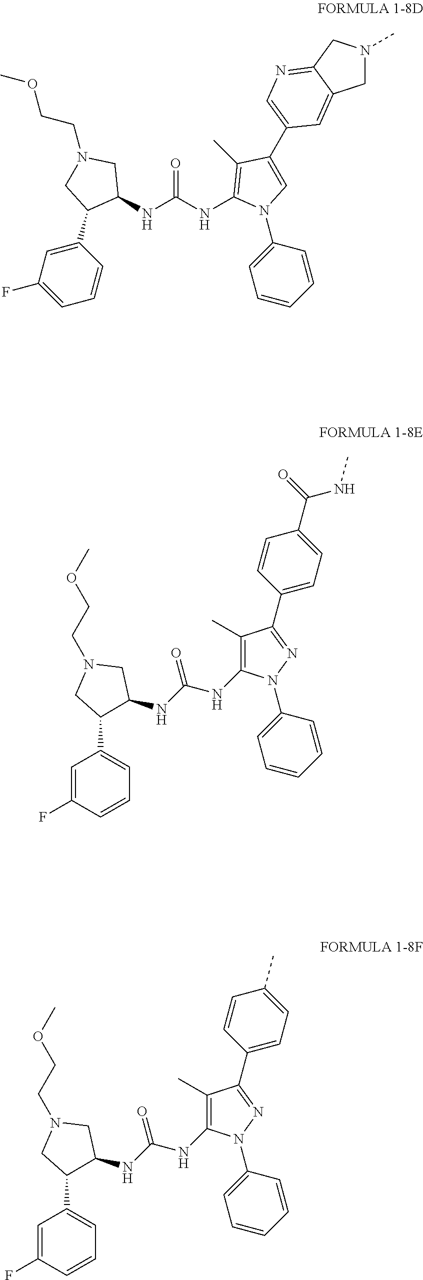

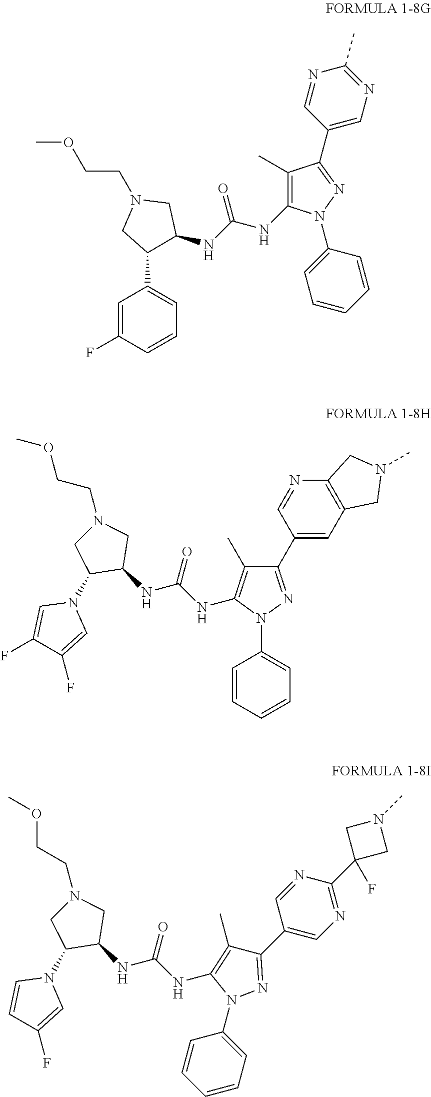

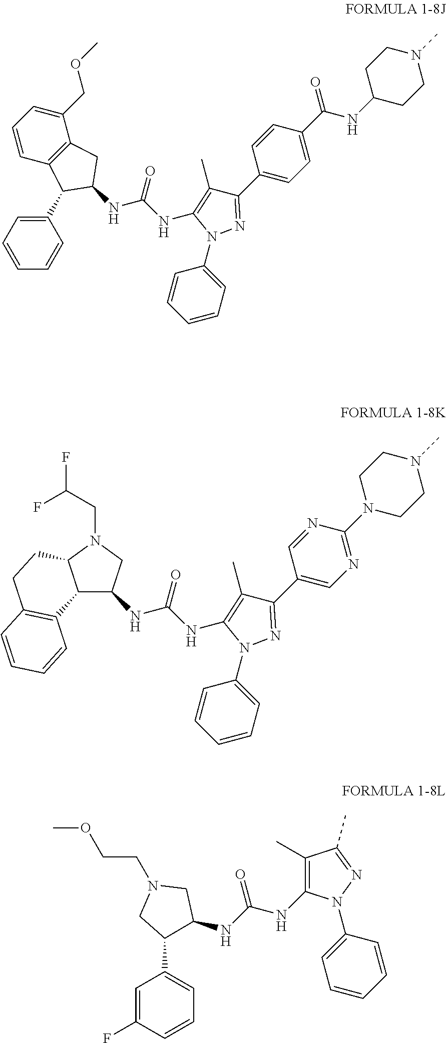

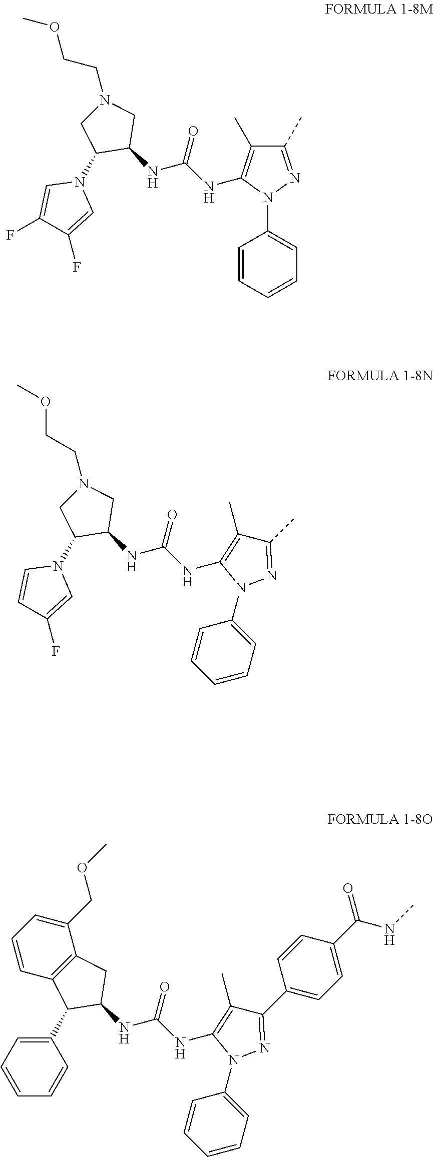

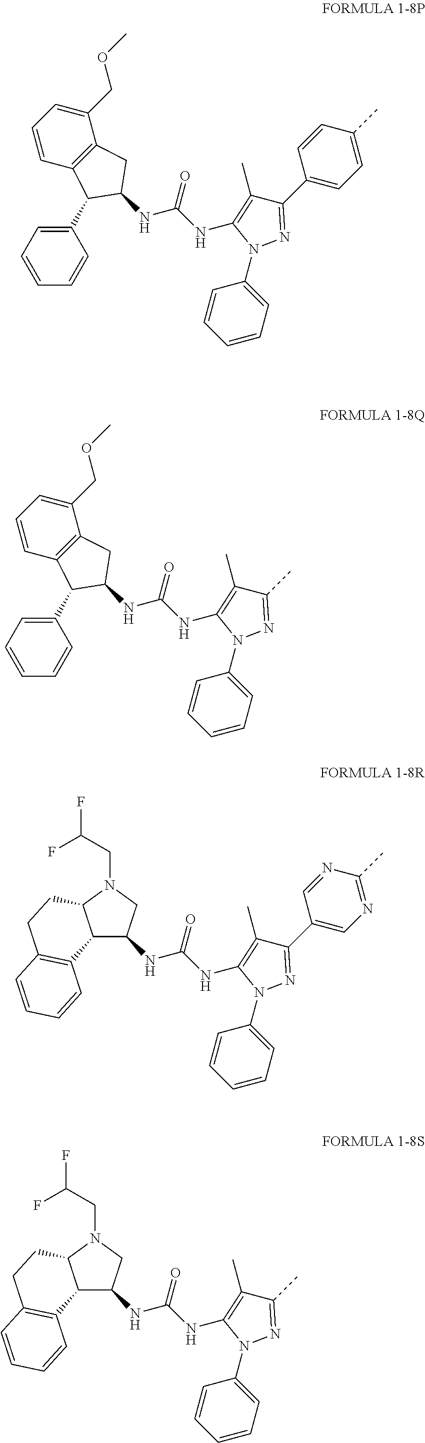

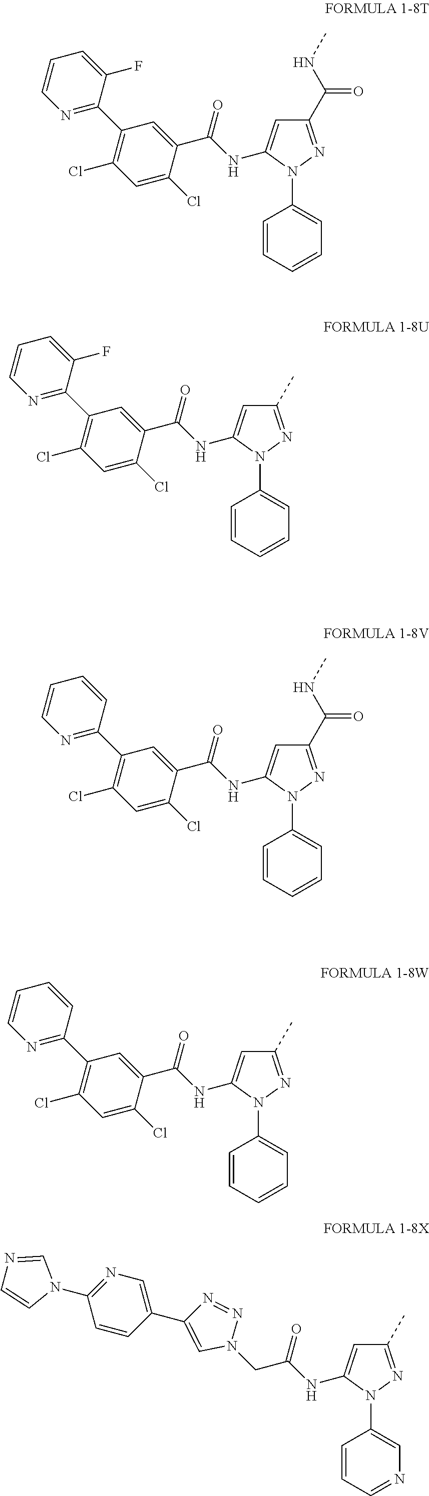

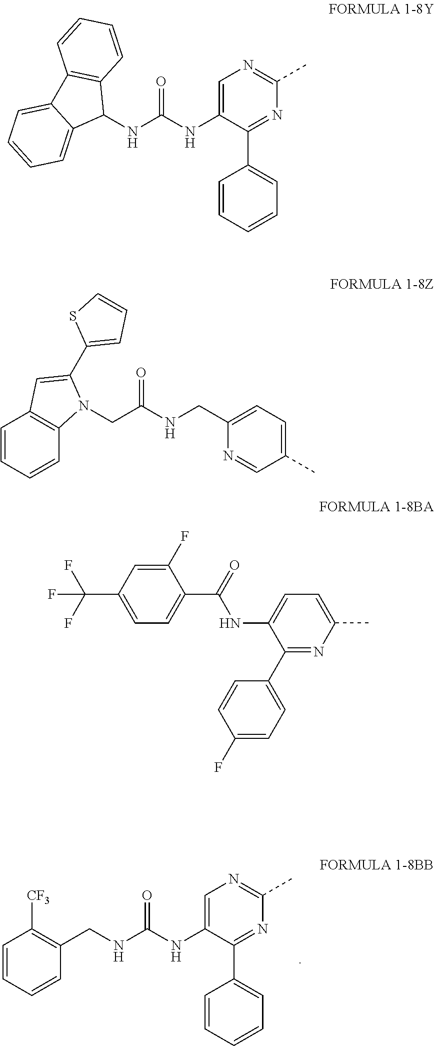

In some embodiments, the TRK ligand is a moiety of any of FORMULAE 1-6A, 1-7A, 1-8A, 1-8B, 1-8C, 1-8D, 1-8E, 1-8F, 1-8G, 1-8H, 1-81, 1-8J, 1-8K, 1-8L, 1-8M, 1-8N, 1-80, 1-8P, 1-8Q, 1-8R, 1-8S, 1-8T, 1-8U, 1-8V, 1-8W, 1-8X, 1-8Y, 1-8Z, 1-8BA, or 1-8BB, or their corresponding enantiomers.

In another embodiment, Ring A_{E} is a divalent group selected from the group consisting of FORMULA A_{E}1, A_{E}2, A_{E}3, and A_{E}4; V_{E}¹, V_{E}², V_{E}³, V_{E}⁴ and V_{E}⁵, at each occurrence, are each independently selected from the group consisting of a bond, C, CR_{E}², and N; or V_{E}¹ and V_{E}², V_{E}² and V_{E}³, V_{E}³ and V_{E}⁴, or V_{E}⁴ and V_{E}⁵ are combined together to optionally form 6 membered aryl ring or a 5, or 6 membered heteroaryl ring.

In another embodiment, R_{E}² at each occurrence, is independently selected from the group consisting of absent, hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈ alkoxy, optionally substituted C₁-C₈ alkylamino, optionally substituted C₃-C₈ carbocyclyl, and optionally substituted 3-8 membered heterocyclyl.

In another embodiment, the degradation tag is a moiety of FORMULA 5, and wherein V_{E}¹, V_{E}², V_{E}³, V_{E}⁴ and V_{E}⁵, at each occurrence, are each independently selected from the group consisting of C, CR_{E}² and N; or V_{E}¹ and V_{E}², V_{E}² and V_{E}³, V_{E}³ and V_{E}⁴, or V_{E}⁴ and V_{E}⁵ are combined together to optionally form C₆ aryl ring or a 5, or 6 membered heteroaryl ring.

In another embodiment, the degradation tag is a moiety of FORMULA 5, and wherein Ring A_{E} is a group consisting of FORMULA A_{E}1, and wherein V_{E}¹, V_{E}², V_{E}³, and V_{E}⁴ are each independently selected from the group consisting of C, CR_{E}² and N.

In another embodiment, the degradation tag is a moiety of FORMULA 5, and wherein Ring A_{E} is a group consisting of FORMULA A_{E}2, and wherein V_{E}¹, V_{E}², V_{E}³, V_{E}⁴ and V_{E}⁵, at each occurrence, are each independently selected from the group consisting of C, CR_{E}² and N.

In another embodiment, the degradation tag is a moiety of FORMULA 5, and wherein Ring A_{E} is a group consisting of FORMULA A_{E}3, and wherein V_{E}¹, V_{E}², V_{E}³, V_{E}⁴ and V_{E}⁵ are each independently selected from the group consisting of CR_{E}² and N; or V_{E}¹ and V_{E}², V_{E}² and V_{E}³, V_{E}³ and V_{E}⁴, or V_{E}⁴ and V_{E}⁵ are combined together to optionally form 6 membered aryl ring or a 5, or 6 membered heteroaryl ring.

In another embodiment, the degradation tag is a moiety of FORMULA 5, and wherein Ring A_{E} is of FORMULA A_{E}3, and L_{E} is not null.

In another embodiment, the degradation tag is a moiety of FORMULA 5, and wherein Ring A_{E} is of FORMULA A_{E}3 and L_{E} is -L_{E}¹-, where -L_{E}¹-, is selected from the group consisting of —NH—, —N(C₁-C₄ alkyl)-, —CO—, or L_{E} is -L_{E}¹-L_{E}²-, wherein -L_{E}¹-L_{E}²- is selected from the group consisting of —NH—CO—, —N(C₁-C₄ alkyl)-CO—, —CO—NH—, and —CO—N(C₁-C₄ alkyl)-.

In another embodiment, the degradation tag is a moiety of FORMULA 5 and wherein Ring A_{E} is a group consisting of FORMULA A_{E}4, and wherein [symbol] is a single bond and W_{E}¹, W_{E}², W_{E}³ and W_{E}⁴ are each independently selected from the group consisting of —N═, —CR_{E}³=, —CO—, —O—, —CR_{E}³R_{E}⁴—, and —NR_{E}³—.

In another embodiment, the degradation tag is a moiety of FORMULA 5, and wherein Ring A_{E} is a group consisting of FORMULA A_{E}5, and wherein V_{E}¹, V_{E}², and V_{E}³ are each independently selected from the group consisting of CR_{E}², N, and NR_{E}², with the proviso that at least one of V_{E}¹, V_{E}², and V_{E}³ is N or NR_{E}².

In another embodiment, the degradation tag is a moiety of FORMULA 5, and wherein Ring A_{E} is a group consisting of FORMULA A_{E}1, A_{E}2, and A_{E}5, and W_{E}¹, W_{E}², W_{E}³ and W_{E}⁴ are each independently selected from the group consisting of —N═, —CR_{E}³=, —CO—, —O—, —CR_{E}³R_{E}⁴—, and —NR_{E}³—.

In another embodiment, the degradation tag is a moiety of FORMULA 5, and wherein R_{E}¹ is selected from hydrogen, halogen, cyano, nitro, optionally substituted C₁-C₆ alkyl, optionally substituted C₁-C₆ heteroalkyl, optionally substituted C₁-C₆ haloalkyl, optionally substituted C₃-C₈ carbocyclyl, and optionally substituted 3-8 membered heterocyclyl; preferably, R_{E}¹ is selected from hydrogen, halogen, cyano, nitro, and C₁-C₈ alkyl; more preferably, R_{E}¹ is selected from H, CH₃, or F.

In another embodiment, the degradation tag is a moiety of FORMULA 5, and wherein R_{E}² is selected from hydrogen, halogen, cyano, nitro, hydroxy, amino, optionally substituted C₁-C₆ alkyl, optionally substituted C₁-C₆ alkyl, optionally substituted C₁-C₆ heteroalkyl, optionally substituted C₁-C₆ haloalkyl, optionally substituted C₁-C₆ alkoxyl, optionally substituted C₁-C₆ alkylamino, optionally substituted C₃-C₈ carbocyclyl, and optionally substituted 3 to 8 membered heterocyclyl; preferably, R_{E}² is selected from hydrogen, halogen, cyano, nitro, optionally substituted C₁-C₆ alkyl, optionally substituted C₁-C₆ heteroalkyl, optionally substituted C₁-C₆ haloalkyl, optionally substituted C₁-C₆ alkoxyl, optionally substituted C₃-C₈ carbocyclyl, and optionally substituted 3 to 8 membered heterocyclyl; more preferably, R_{E}² is selected from H, F, OMe, O-iPr, or O-cPr.

In another embodiment, the degradation tag is a moiety of FORMULA 5, and wherein R_{E}³ and R_{E}⁴ are independently selected from the group consisting of hydrogen, halogen, cyano, nitro, optionally substituted C₁-C₆ alkyl, optionally substituted C₁-C₆ heteroalkyl, optionally substituted C₁-C₆ haloalkyl, optionally substituted C₃-C₈ carbocyclyl, and optionally substituted 3 to 8 membered heterocyclyl; or R_{E}³ and R_{E}⁴ together with the atom(s) to which they are connected form a C₃-C₈ carbocyclyl, or 3-8 membered heterocyclyl.

In another embodiment, R_{E}^{r} is selected from Group R_{E} and Group R_{E}′.

Group R_{E} consists of the following optionally substituted groups:

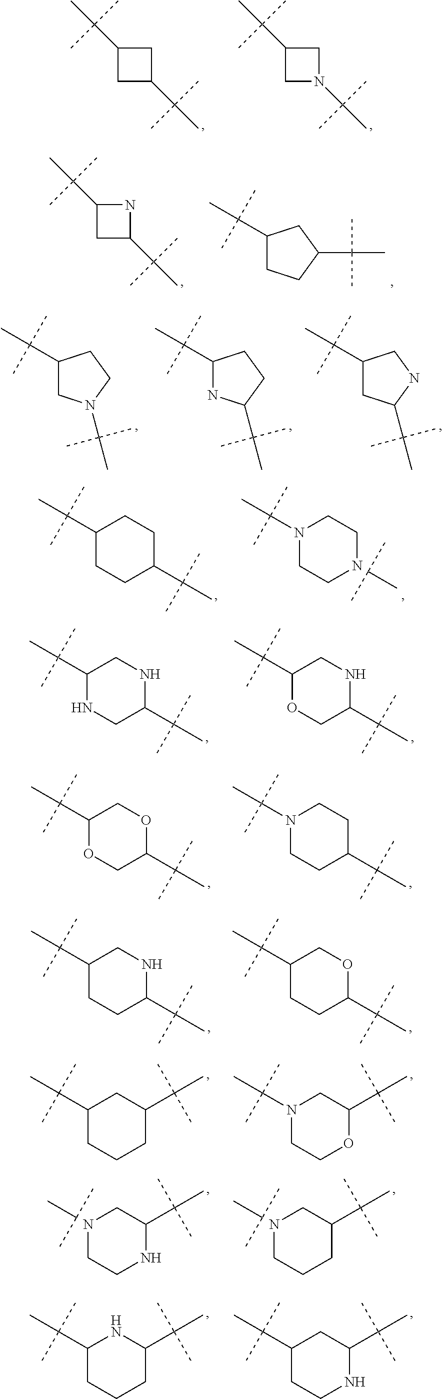

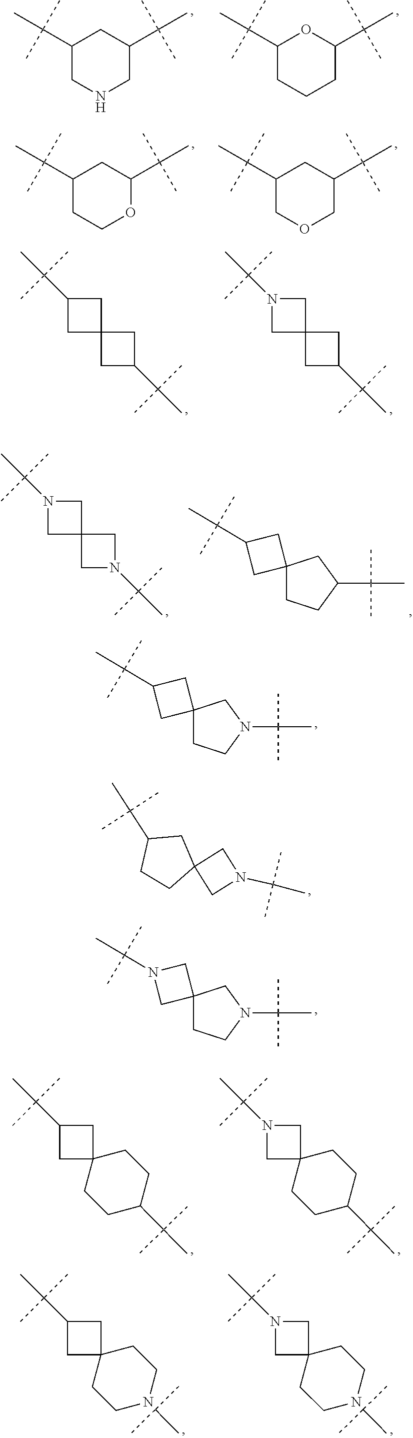

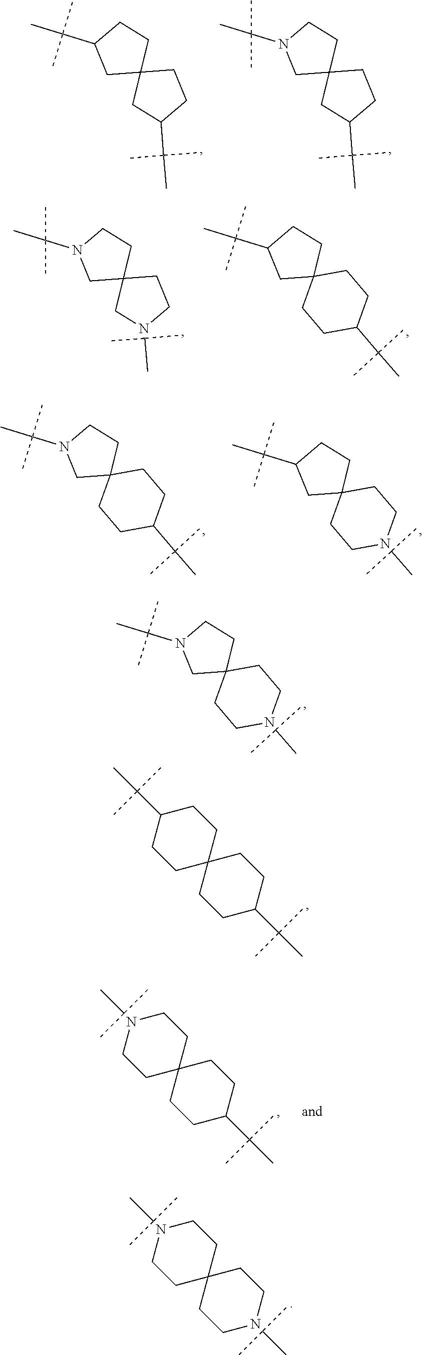

Group R_{E}′ consists of the following optionally substituted groups:

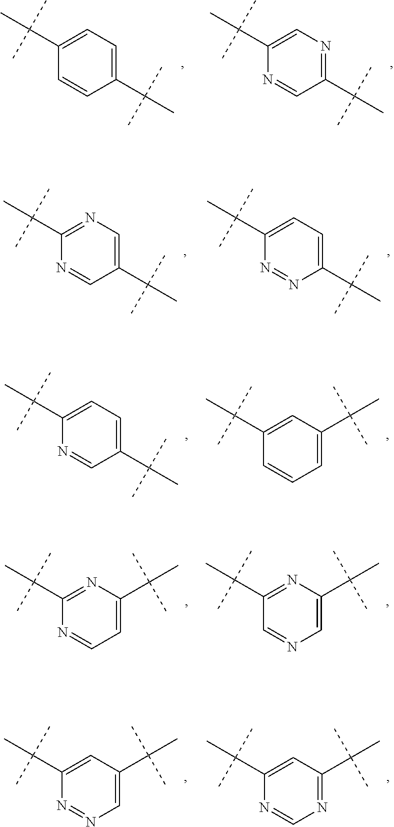

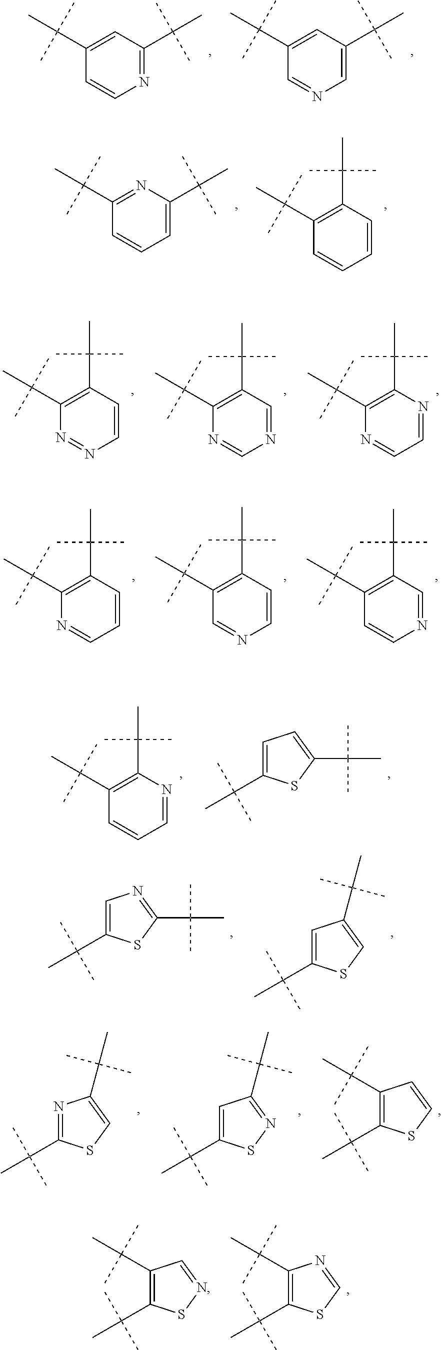

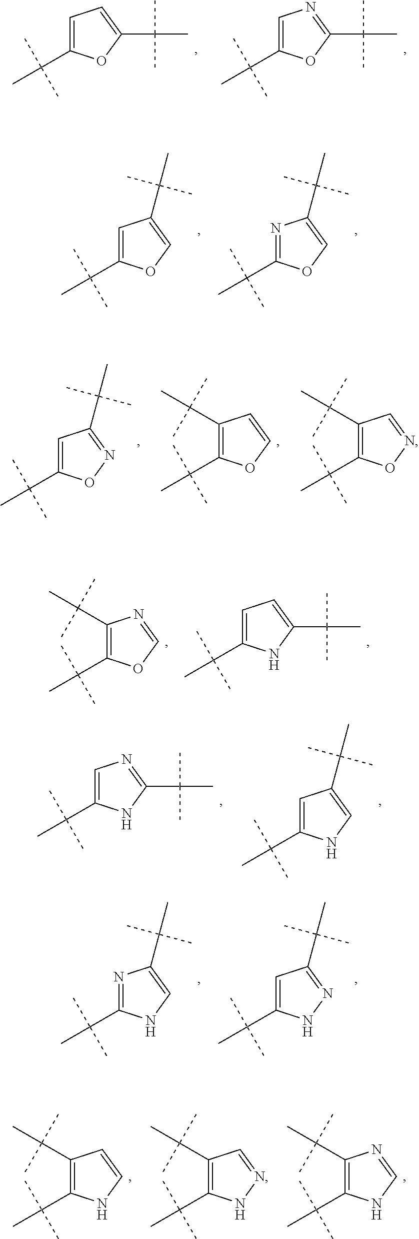

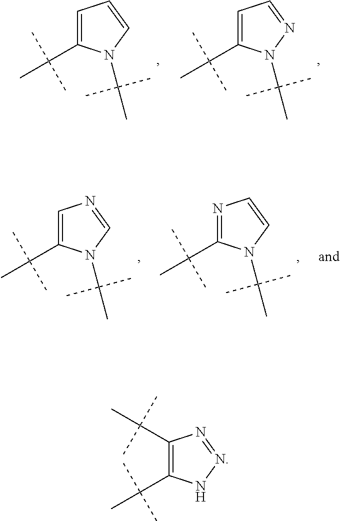

In another embodiment, in FORMULA 5, in the group of Z_{E}, at most one R_{E}^{z} is R_{E}^{r}.

In another embodiment, in FORMULA 5, R_{E}⁵ and R_{E}⁶ at each occurrence are independently selected from the group consisting of hydrogen, halogen, oxo, hydroxyl, amino, cyano, nitro, optionally substituted C₁-C₆ alkyl, optionally substituted C₁-C₆ heteroalkyl, optionally substituted C₁-C₆ haloalkyl, optionally substituted C₃-C₈ carbocyclyl, and optionally substituted 3 to 8 membered heterocyclyl; or R_{E}⁵ and R_{E}⁶ together with the atom(s) to which they are connected form a C₃-C₈ cycloalkyl or 3-8 membered heterocyclyl ring.

In another embodiment, in FORMULA 5, R_{E}^{z} is selected from —CO—, —CR_{E}⁵R_{E}⁶—, —NR_{E}⁵—, —O—, optionally substituted C₁-C₁₀ alkylene, optionally substituted C₂-C₁₀ alkenylene, optionally substituted C₂-C₁₀ alkynylene, optionally substituted C₁-C₁₀ heteroalkylene, optionally substituted C₂-C₁₀ heteroalkenylene, optionally substituted C₂-C₁₀ heteroalkynylene, optionally substituted C₁-C₁₀ haloalkylene, optionally substituted C₃-C₈ carbocyclyl, optionally substituted 3-8 membered heterocyclyl.

In another embodiment, in FORMULA 5, R_{E}^{z} is null, —CR_{E}⁵R_{E}⁶—, or —NR_{E}⁵—, wherein R_{E}⁵ and R_{E}⁶ at each occurrence are independently hydrogen or C₁-C₆ alkyl.

In another embodiment, in FORMULA 5, Z_{E} is selected from a bond, —CH₂—, —CH═CH—, —C≡C—, NH, and O; and preferably, Z_{E} is null or NH.

In another embodiment, in FORMULA 5, and wherein

Ring A_{E} is of FORMULA A_{E}1; L_{E} is selected from the group consisting of —NH—, —N(C₁-C₄ alkyl)-, —CO—, —NH—CO—, —N(C₁-C₄ alkyl)-CO—, —CO—NH—, and —CO—N(C₁-C₄ alkyl)-(preferably, L_{E} is —NH—, or —N(C₁-C₄ alkyl)-); or

Ring A_{E} is of A_{E}1; and L_{E} is null.

In another embodiment, in FORMULA 5, Ring A_{E} is of FORMULA A_{E}1, wherein L_{E} is null.

In another embodiment, the moiety of FORMULA 5 has a structure selected from the group consisting of FORMULA 5-1, 5-2, 5-3, 5-4, 5-5 and 5-6:

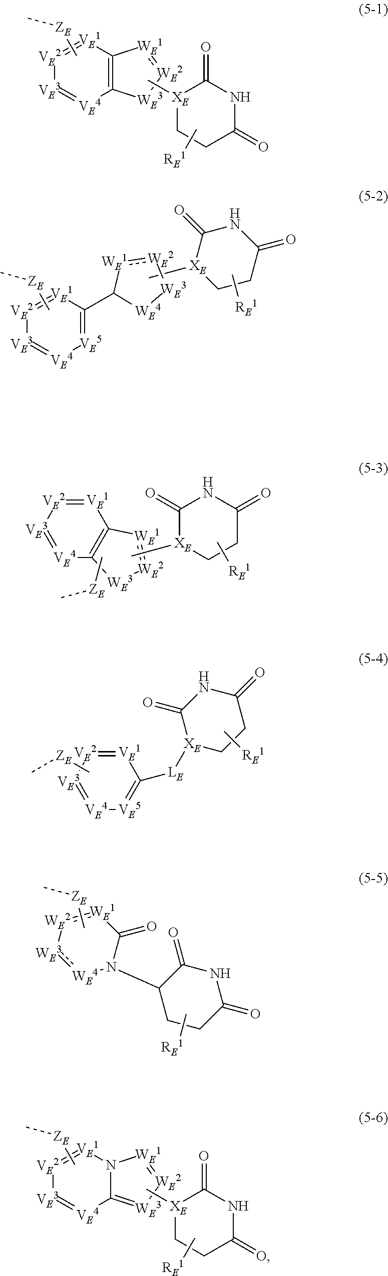

wherein
X_{E}, Z_{E}, R_{E}¹, L_{E}, [symbol] , V_{E}¹, V_{E}², V_{E}³, V_{E}⁴, V_{E}⁵, W_{E}¹, W_{E}², W_{E}³ and W_{E}⁴ are defined as in FORMULA 5.

In another embodiment, the moiety of FORMULA 5 is a moiety selected from the group consisting of FORMULA 5A, 513, 5C, 5D, 5E, 5F, 5G, 5H, 5I, 5J, 5K, 5L, 5M, 5N, 5O, 5P, 5Q, 5R, 5S, 5T, 5U, 5V, 5W, and 5X:

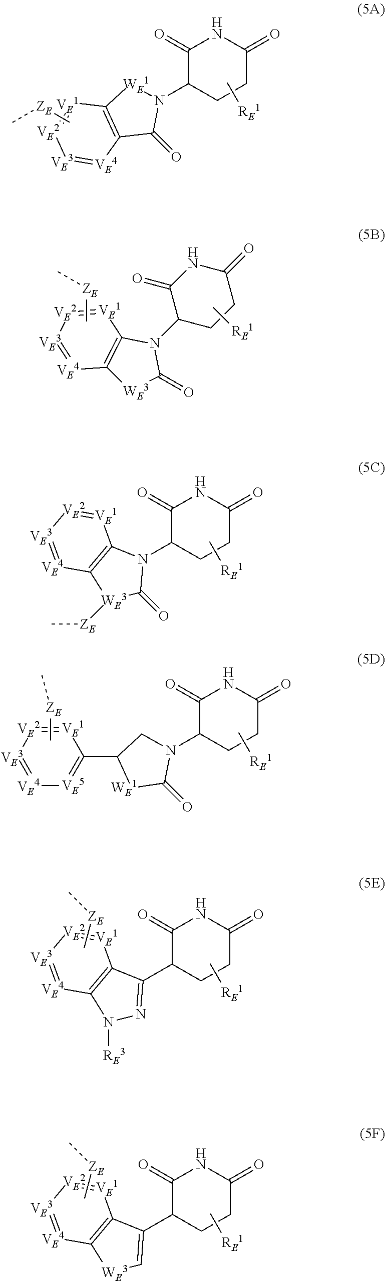

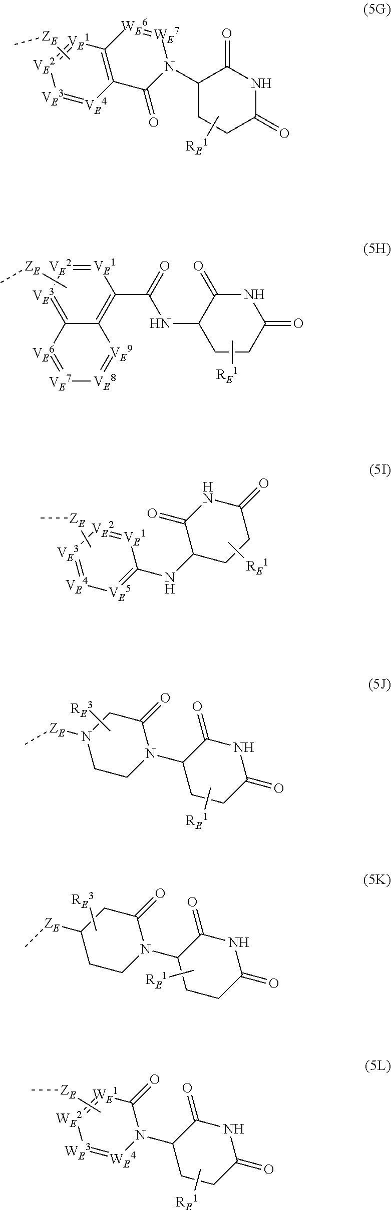

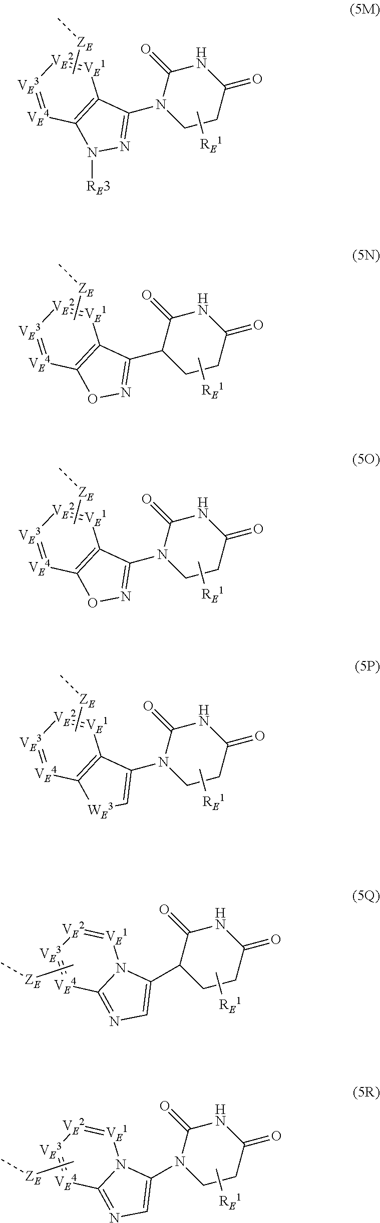

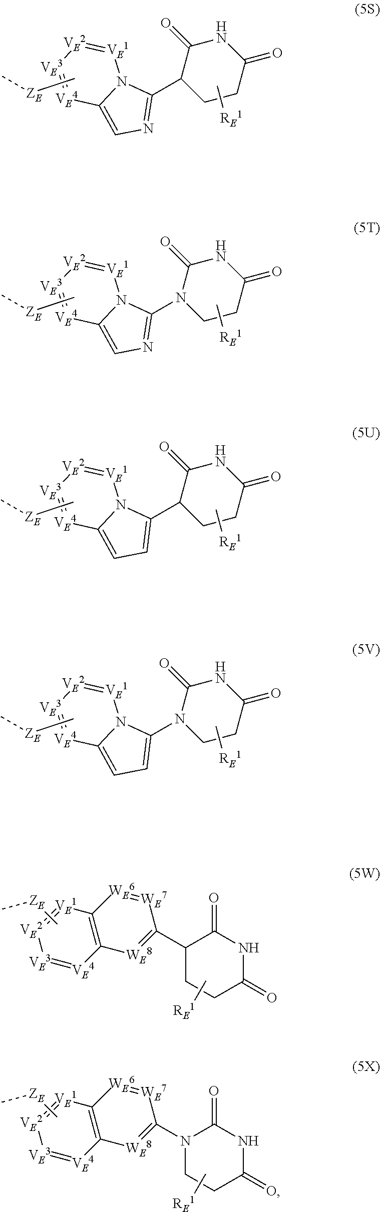

wherein
V_{E}⁶, V_{E}⁷, V_{E}⁸, and V_{E}⁹ are each independently selected from the group consisting of a bond C, CR_{E}¹² and N; or V_{E}¹ and V_{E}², V_{E}² and V_{E}³, V_{E}³ and V_{E}⁴, or V_{E}⁴ and V_{E}⁵ are combined together to optionally form 6 membered aryl ring or a 5, or 6 membered heteroaryl ring;
R_{E}¹², at each occurrence, is independently selected from the group consisting of hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈ alkoxy, optionally substituted C₁-C₈ alkylamino, optionally substituted C₃-C₈ carbocyclyl, and optionally substituted 3-8 membered heterocyclyl;
W_{E}⁶, W_{E}⁷ and W_{E}⁸ are each independently selected from the group consisting of —CR_{E}²═ and —N═;
W_{E}¹, W_{E}², W_{E}³, W_{E}⁴, V_{E}¹, V_{E}², V_{E}³, V_{E}⁴, V_{E}⁵, R_{E}¹, R_{E}³, and Z_{E} are defined as in FORMULA 5.

In another embodiment, W_{E}¹ is selected from —CO—, —O—, —CR_{E}³R_{E}⁴—, —NR_{E}³—, —CR_{E}³=CR_{E}⁴-, —N═CR_{E}³-, and —N═N—.

In another embodiment, the moiety of FORMULA 5 is a moiety of FORMULA 5-1, or FORMULA 5-3:

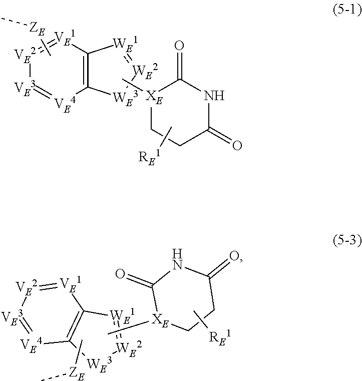

wherein
V_{E}¹, V_{E}², V_{E}³, and V_{E}⁴ are each independently selected from the group consisting of a bond, C, CR_{E}², and N; or V_{E}¹ and V_{E}², V_{E}² and V_{E}³, or V_{E}³ and V_{E}⁴ are combined together to optionally form 6 membered aryl ring or 5, or 6 membered heteroaryl ring; [symbol]
[symbol] indicates a single bond or a double bond, wherein: (i) when there is a single bond between W_{E}¹ and W_{E}² (i.e. the [symbol] between W_{E}¹ and W_{E}² indicates single bond), W_{E}¹, W_{E}² and W_{E}³ are each independently selected from the group consisting of —N═, —CR_{E}³=, —CO—, —O—, —CR_{E}³R_{E}⁴—, —NR_{E}³—, —CR_{E}³═CR_{E}⁴—, —N═CR_{E}³, and —N═N—; or (ii) when there is a double bond between W_{E}¹ and W_{E}² (i.e. the [symbol] between W_{E}¹ and W_{E}² indicates a double bond), W_{E}¹ and W_{E}² are each independently selected from the group consisting of —N═, —C≡ and —CR_{E}³=; W_{E}³ is selected from the group consisting of —O—, —N═, —NR_{E}³—, —C(O)NR_{E}³—, —CR_{E}³=CR_{E}⁴-, and —CR_{E}³═N—;
Z_{E}, R_{E}², R_{E}³, R_{E}⁴ and R_{E}¹ are defined as in FORMULA 5.

In another embodiment, in FORMULA 5-1 or 5-3, V_{E}¹, V_{E}², V_{E}³, and V_{E}⁴ are each independently selected from C, N, and CR_{E}².

In another embodiment, the moiety of FORMULA 5-1 has a structure of FORMULA 5A, 5B, 5E, 5F or 5G:

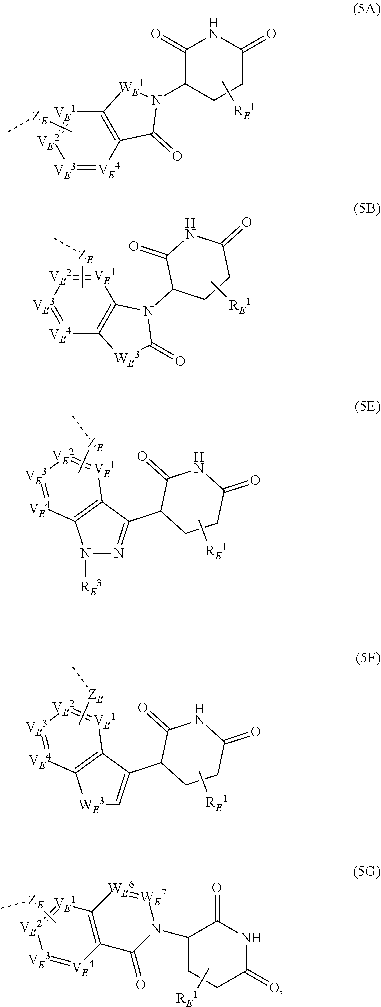

wherein W_{E}⁶ and W_{E}⁷ are each independently selected from the group consisting of —CR_{E}²═ and —N═; and V_{E}¹, V_{E}², V_{E}³, V_{E}⁴, W_{E}¹, W_{E}³, Z_{E}, R_{E}³ and R_{E}⁴ are defined as in FORMULA 5-1.

In another embodiment, in FORMULA 5A, 5B, 5E, 5F or 5G, V_{E}¹, V_{E}², V_{E}³, and V_{E}⁴ are each independently selected from a bond, C, CR_{E}² and N (preferably, C, CR_{E}² and N).

In another embodiment, in FORMULA 5A, 5B, 5E, 5F or 5G, W_{E}¹ and W_{E}³ are each independently selected from —CO—, —O—, —CR_{E}³R_{E}⁴—, —NR_{E}³—, —CR_{E}³=CR_{E}⁴-, —N═CR_{E}³-, and —N═N—; preferably, W_{E}¹ and W_{E}³ are each independently selected from —CO—, —O—, —CR_{E}³R_{E}⁴—, and —NR_{E}³—

In another embodiment, the moiety of FORMULA 5-3 has a structure of FORMULA 5C:

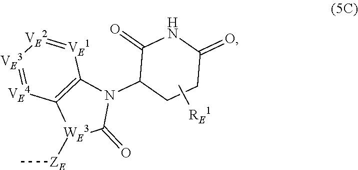

wherein W_{E}³ is N or CR_{E}³; and V_{E}¹, V_{E}², V_{E}³, V_{E}⁴, Z_{E}, and R_{E}¹ are defined as in FORMULA 5-3.

In another embodiment, in FORMULA 5C, V_{E}¹, V_{E}², V_{E}³, and V_{E}⁴ are each independently selected from a bond, CR_{E}² and N.

In another embodiment, the moiety of FORMULA 5 is a moiety of FORMULA 5-2:

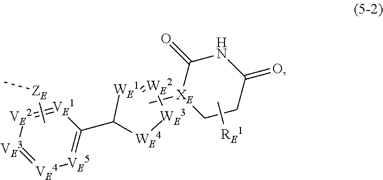

V_{E}¹, V_{E}², V_{E}³, V_{E}⁴ and V_{E}⁵ are each independently selected from a bond, C, CR_{E}², and N; or V_{E}¹ and V_{E}², V_{E}² and V_{E}³, V_{E}³ and V_{E}⁴, or V_{E}⁴ and V_{E}⁵ are combined together to optionally form 6 membered aryl ring or 5, or 6 membered heteroaryl ring;
[symbol] indicates a single bond or a double bond; (i) when there is a single bond between W_{E}¹ and W_{E}² (i.e. the [symbol] between W_{E}¹ and W_{E}² indicates single bond), W_{E}¹ and W_{E}⁴ are each independently selected from —N═, —CR_{E}³=, —CO—, —O—, —CR_{E}³R_{E}⁴—, —NR_{E}³—, —CR_{E}³=CR_{E}⁴—, —N═CR_{E}³-, and —N═N—, and W_{E}² and W_{E}³ are each independently selected from —N═, —CR_{E}³═—CO—, —O—, —CR_{E}³R_{E}⁴—, and —NR_{E}³—; or (ii) when there is a double bond between W_{E}¹ and W_{E}² (i.e. the [symbol] between W_{E}¹ and W_{E}² indicates a double bond), W_{E}¹ and W_{E}² are each independently selected from —N═, C and —CR_{E}²=; W_{E}³ is selected from —N═, —CR_{E}³=, —CO—, —O—, —CR_{E}³R_{E}⁴—, and —NR_{E}³—; and W_{E}⁴ is selected from —N═, —CR_{E}³=, —CO—, —O—, —CR_{E}³R_{E}⁴—, —NR_{E}³—, —CR_{E}³=CR_{E}⁴-, —N═CR_{E}³-, and —N═N—;
Z_{E}, R_{E}², R_{E}³, R_{E}⁴ and R_{E}¹ are defined as in FORMULA 5.

In another embodiment, in FORMULA 5-2, wherein V_{E}¹, V_{E}², V_{E}³, V_{E}⁴ and V_{E}⁵ are each independently selected from a bond, C, CR_{E}², and N.

In another embodiment, in FORMULA 5-2, wherein [symbol] indicates a single bond.

In another embodiment, in FORMULA 5-2, wherein [symbol] indicates a single bond, W_{E}¹ and W_{E}⁴ are each independently selected from —CO—, —O—, —CR_{E}³R_{E}⁴—, and —NR_{E}³—, and W_{E}² and W_{E}³ are each independently selected from —N═, —CR_{E}³=, —CO—, —O—, —CR_{E}³R_{E}⁴—, and —NR_{E}³-.

In another embodiment, the moiety of FORMULA 5-2 is moiety of FORMULA 5D:

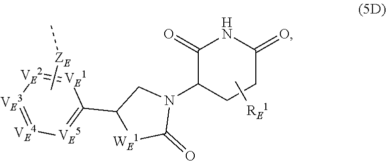

wherein V_{E}¹, V_{E}², V_{E}³, V_{E}⁴, V_{E}⁵, W_{E}¹, Z_{E}, and R_{E}¹ are defined as in FORMULA 5-2.

In another embodiment, in FORMULA 5D, W_{E}¹ is selected from —CO—, —O—, —CR_{E}³R_{E}⁴—, —NR_{E}³—, —CR_{E}³=CR_{E}⁴-, —N═CR_{E}³-, and —N═N—; preferably, W_{E}¹ is selected from —CO—, —O—, —CR_{E}³R_{E}⁴—, and —NR_{E}³—.

In another embodiment, in FORMULA 5D, V_{E}¹, V_{E}², V_{E}³, V_{E}⁴, and V_{E}⁵ are each independently selected from a bond, C, CR_{E}² and N; or V_{E}¹ and V_{E}², V_{E}² and V_{E}³, V_{E}³ and V_{E}⁴, or V_{E}⁴ and V_{E}⁵ are combined together to optionally form a 6 membered aryl ring or 5, or 6 membered heteroaryl ring; preferably, V_{E}¹, V_{E}², V_{E}³, V_{E}⁴, and V_{E}⁵ are each independently selected from a bond, C, CR_{E}² and N.

In another embodiment, the moiety of FORMULA 5 is a moiety of FORMULA 5-4:

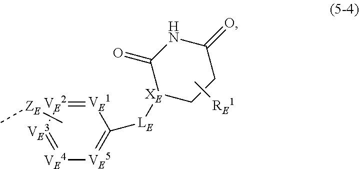

wherein V_{E}¹, V_{E}², V_{E}³, V_{E}⁴, V_{E}⁵, L_{E}, Z_{E}, and R_{E}¹ are defined as in FORMULA 5.

In another embodiment, the degradation tag is a moiety of FORMULA 5-4, wherein L_{E} is not null.

In another embodiment, the degradation tag is a moiety of FORMULA 5-4, wherein L_{E} is selected from the group consisting of —NH—, —N(C₁-C₄ alkyl)-, —CO—, —NH—CO—, —N(C₁-C₄ alkyl)-CO—, —CO—NH—, and —CO—N(C₁-C₄ alkyl)-; and preferably L_{E} is —NH—, or —N(C₁-C₄ alkyl)-.

In another embodiment, the degradation tag is a moiety of FORMULA 5-4, wherein
V_{E}¹, V_{E}², V_{E}³, V_{E}⁴ and V_{E}⁵, at each occurrence, are each independently selected from the group consisting of C, CR_{E}² and N; or
V_{E}¹ and E², V_{E}² and V_{E}³, V_{E}³ and V_{E}⁴, or V_{E}⁴ and V_{E}⁵ are combined together to optionally form a ring of

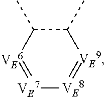

wherein V_{E}⁶, V_{E}⁷, V_{E}⁸, and V_{E}⁹ are each independently selected from the group consisting of C, CR_{E}¹² and N; and
R_{E}¹², at each occurrence, is independently selected from the group consisting of hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈ haloalkyl optionally substituted C₁-C₈ alkoxy, optionally substituted C₁-C₈ alkylamino, optionally substituted C₃-C₈ carbocyclyl, and optionally substituted 3-8 membered heterocyclyl.

In another embodiment, in FORMULA 5-4, V_{E}⁶, V_{E}⁷, V_{E}⁸, and V_{E}⁹ are each independently selected from the group consisting of CR_{E}¹² and N.

In another embodiment, in FORMULA 5-4, R_{E}¹², at each occurrence, is independently selected from the group consisting of hydrogen, halogen, cyano, nitro, hydroxy, amino, optionally substituted C₁-C₆ alkyl, optionally substituted C₁-C₆ heteroalkyl, or optionally substituted C₁-C₆ haloalkyl.

In another embodiment, in FORMULA 5-4,

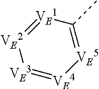

is selected from the group consisting of

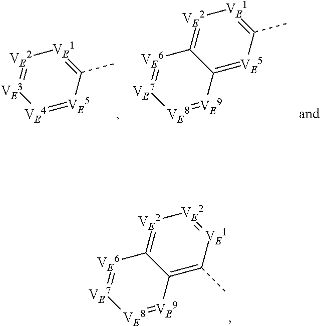

wherein
V_{E}¹, V_{E}², V_{E}³, V_{E}⁴ and V_{E}⁵ are each independently selected from the group consisting of C, CR_{E}² and N; and V_{E}⁶, V_{E}⁷, V_{E}⁸, and V_{E}⁹ are each independently selected from the group consisting of CR_{E}¹² and N.

In another embodiment, in FORMULA 5-4, and wherein Z_{E} is null, —CH₂—, —O—, or —NH—.

In another embodiment, the moiety of FORMULA 5-4 is a moiety of FORMULA 5H, or 5I:

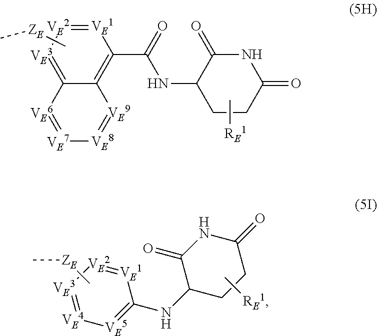

wherein V_{E}¹, V_{E}², V_{E}³, V_{E}⁴, V_{E}⁵, V_{E}⁶, V_{E}⁷, V_{E}⁸, and V_{E}⁹ are each independently selected from the group consisting of a bond, C, CR_{E}² and N; and Z_{E} and R_{E}¹ are defined as in FORMULA 5-4.

In another embodiment, the moiety of FORMULA 5 is a moiety of FORMULA 5-5:

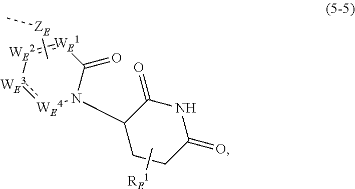

wherein [symbol] W_{E}¹, W_{E}², W_{E}³, W_{E}⁴, Z_{E} and R_{E}¹ are defined as in FORMULA 5.

In another embodiment, in FORMULA 5-5, W_{E}¹, W_{E}², W_{E}³ and W_{E}⁴ are each independently selected from the group consisting of —N═, —C≡, —CR_{E}³=, —CO—, —O—, —CR_{E}³R_{E}⁴—, and —NR_{E}³-.

In another embodiment, in FORMULA 5-5, W_{E}¹, W_{E}², W_{E}³ and W_{E}⁴ are each independently selected from the group consisting of —N═, —C≡, —CH═, —CO—, —O—, —N—, —CH₂—, and —NH—.

In another embodiment, the moiety of FORMULA 5-5 is a moiety of FORMULA 5J, 5K or 5L:

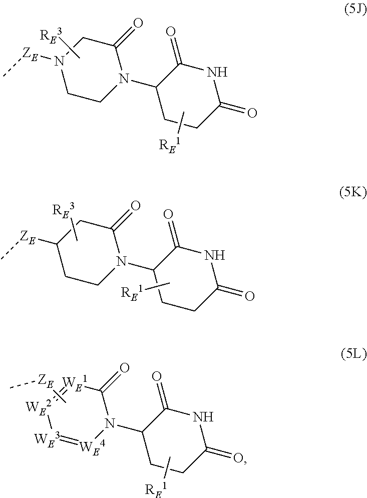

wherein W_{E}¹, W_{E}², W_{E}³, W_{E}⁴, Z_{E}, R_{E}³ and R_{E}¹ are defined as in FORMULA 5-5.

In another embodiment, the degradation tag is a moiety of FORMULA 5-6,

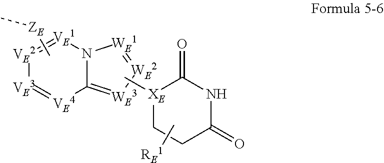

wherein X_{E}, Z_{E}, R_{E}¹, V_{E}¹, V_{E}², V_{E}³, V_{E}⁴, W_{E}¹, W_{E}², and W_{E}³ are defined as in FORMULA 5.

In another embodiment, the degradation tag is a moiety of any of FORMULAE 8A to 8HM:

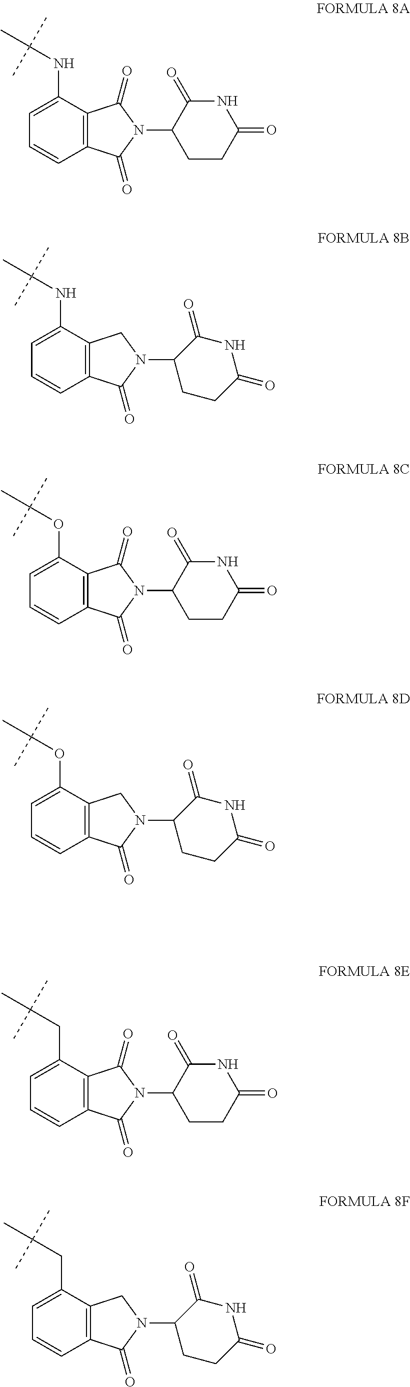

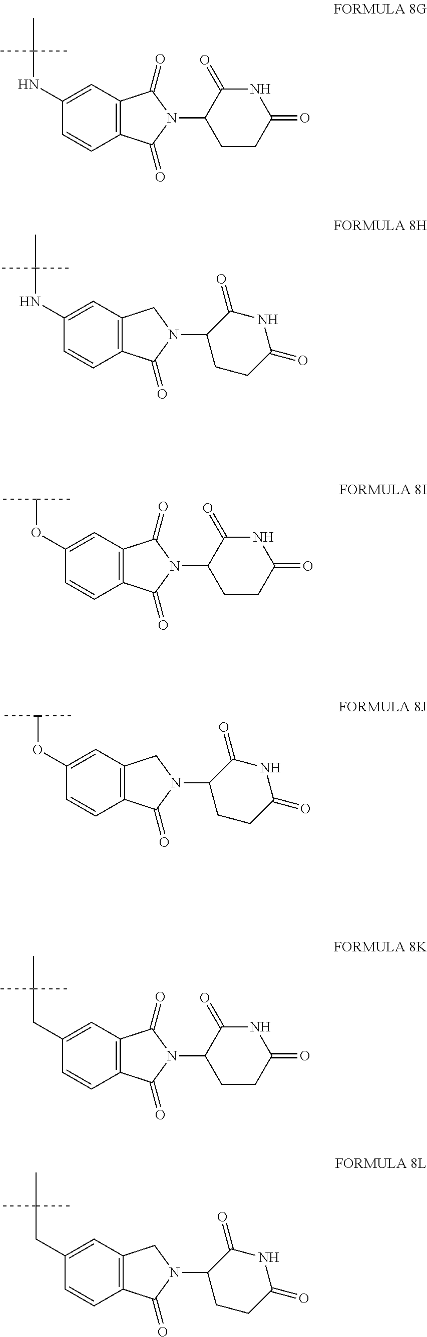

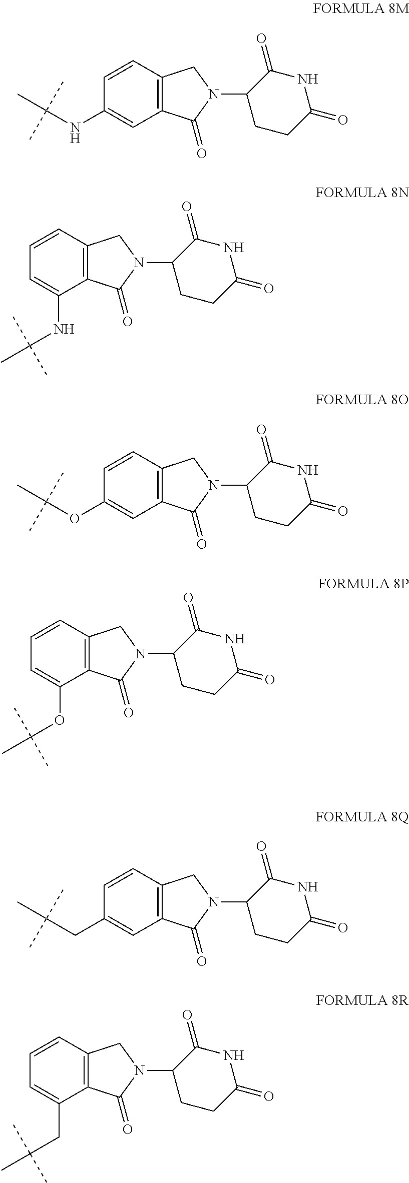

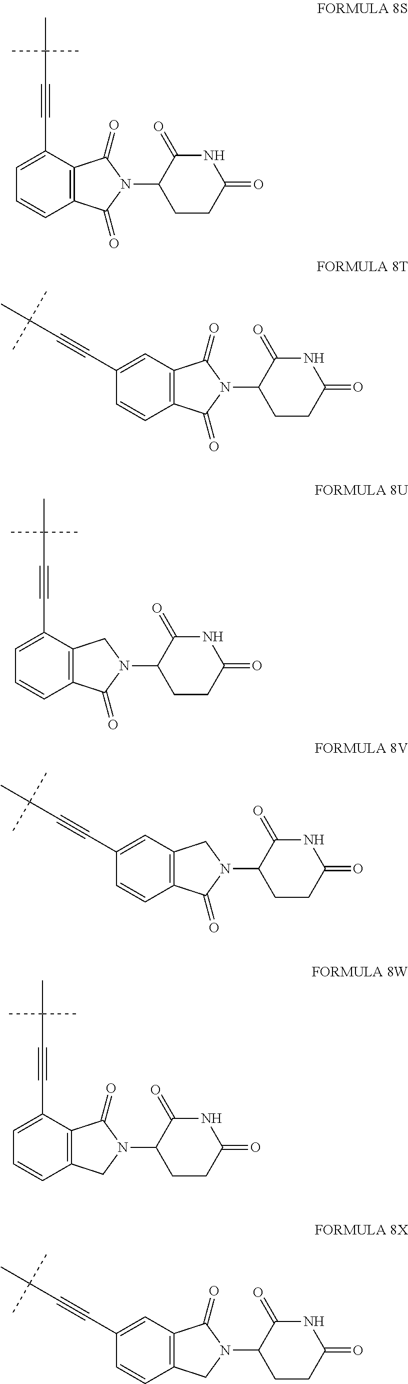

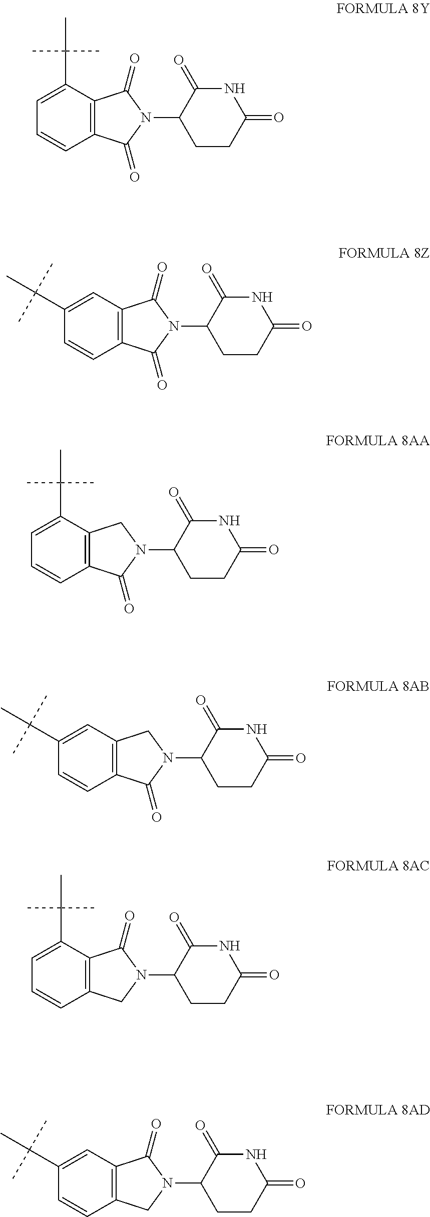

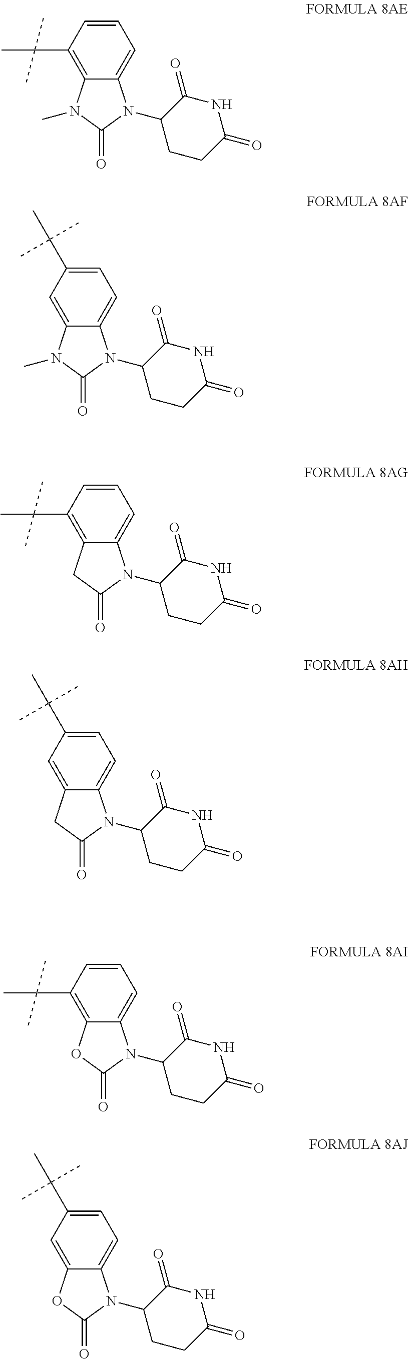

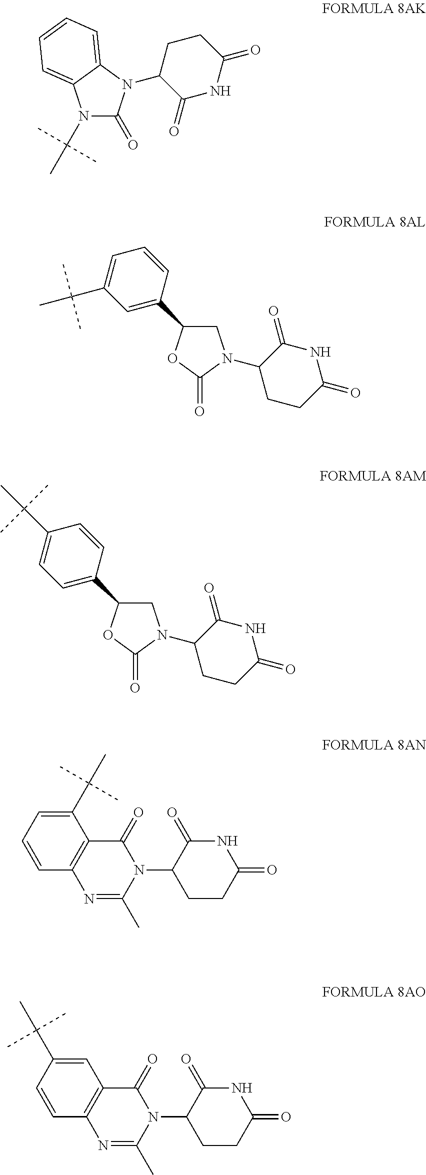

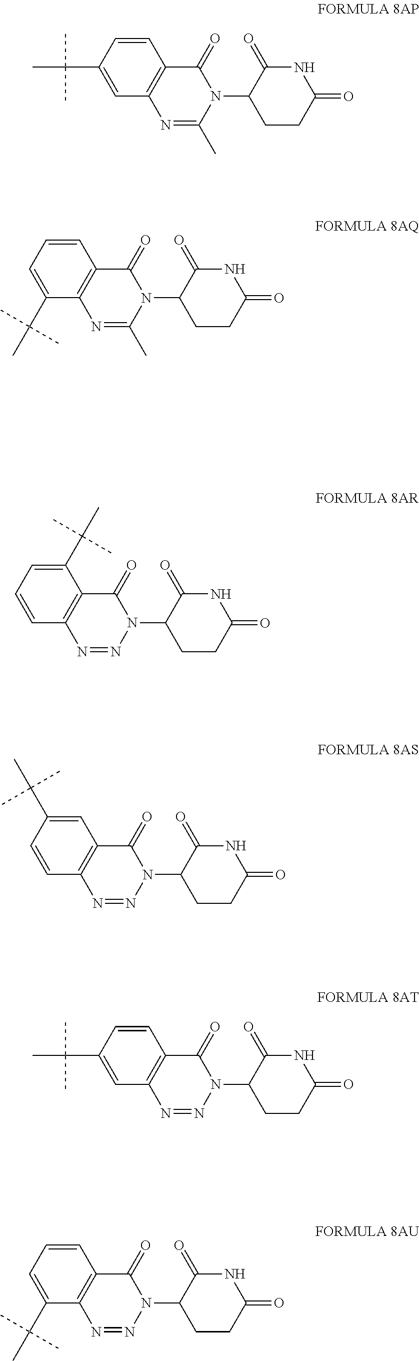

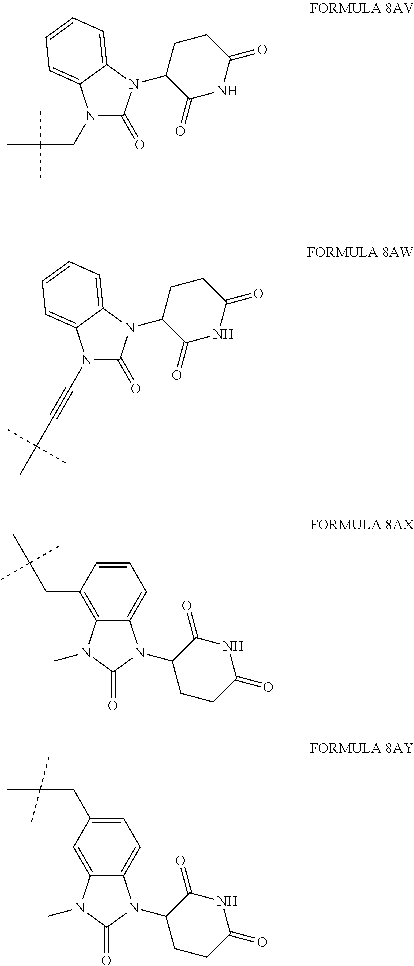

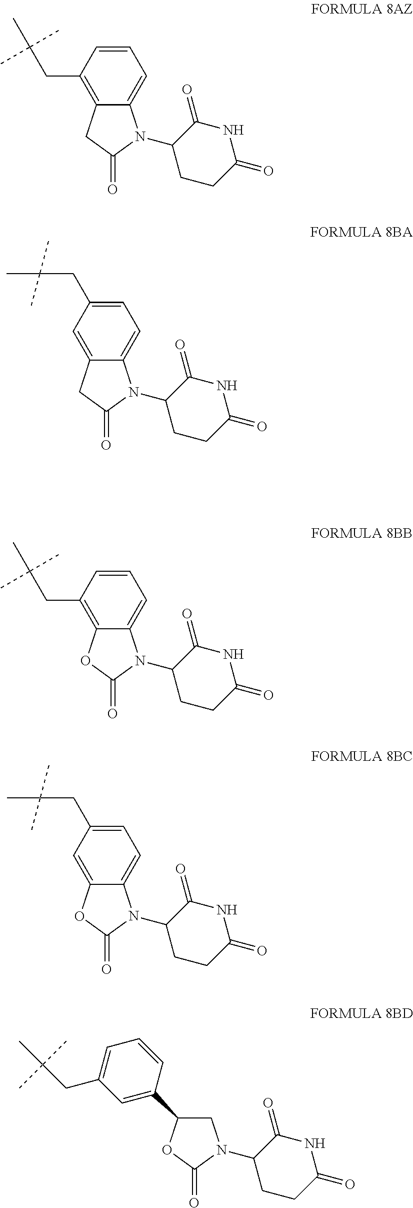

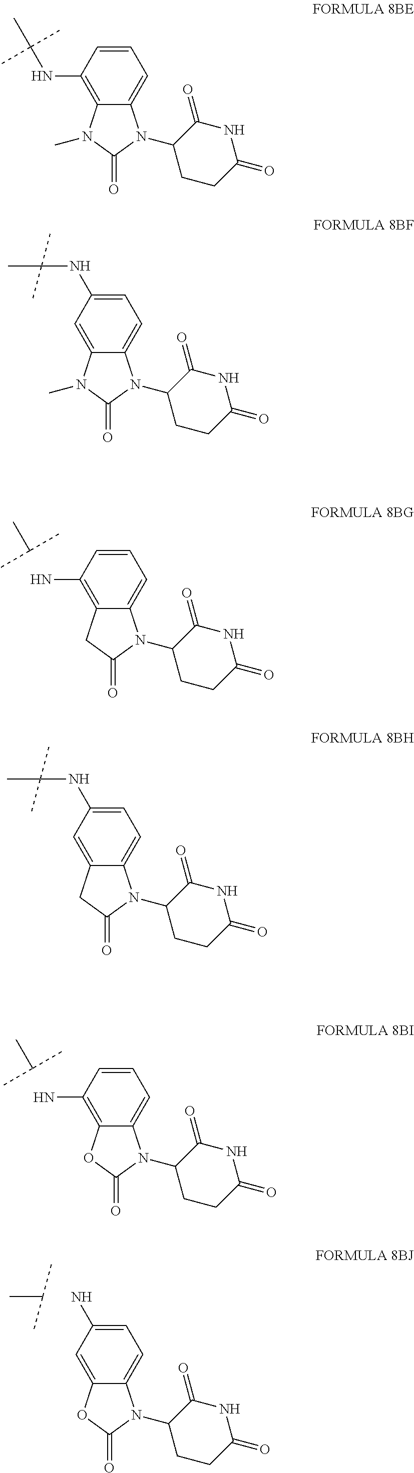

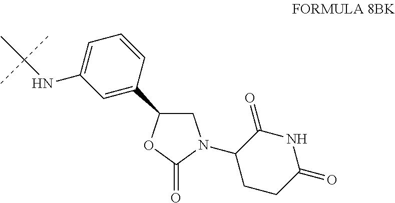

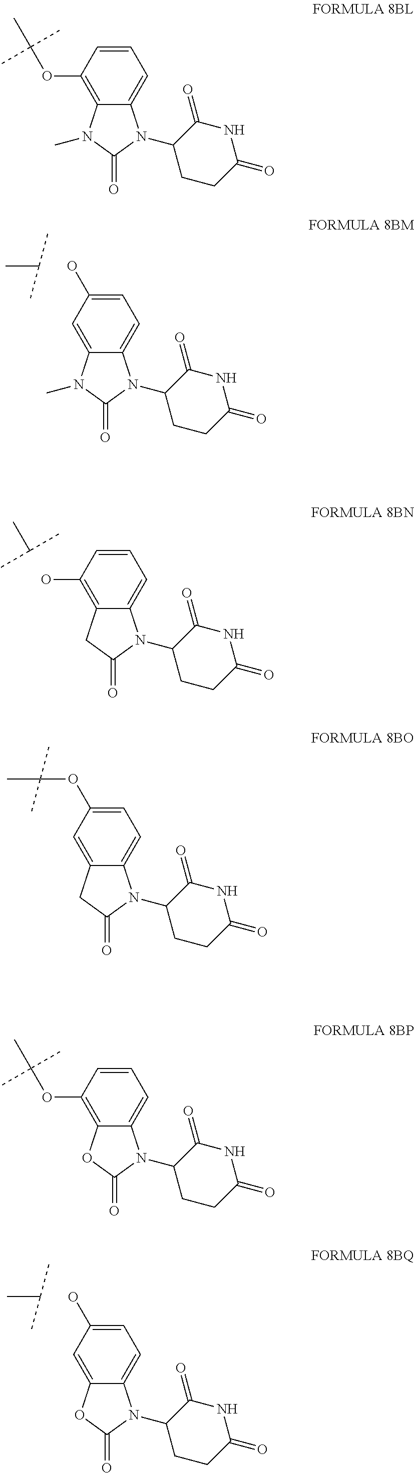

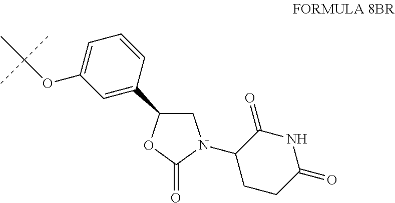

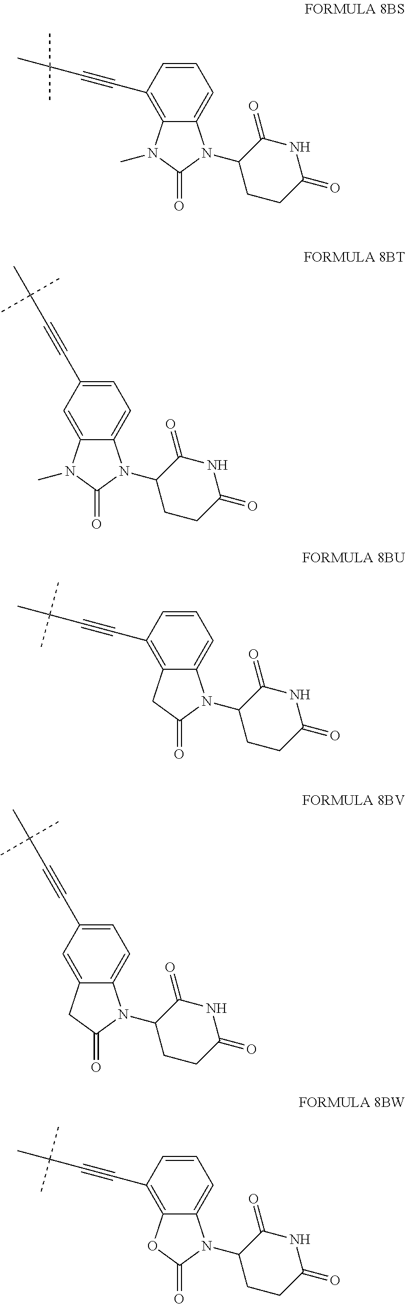

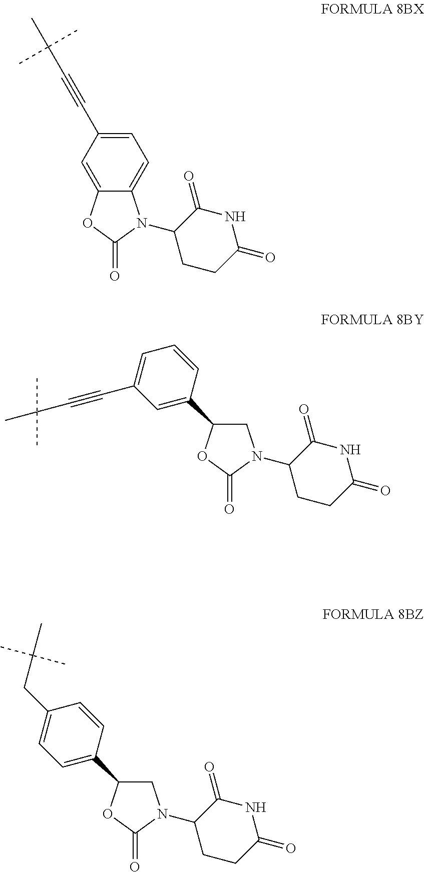

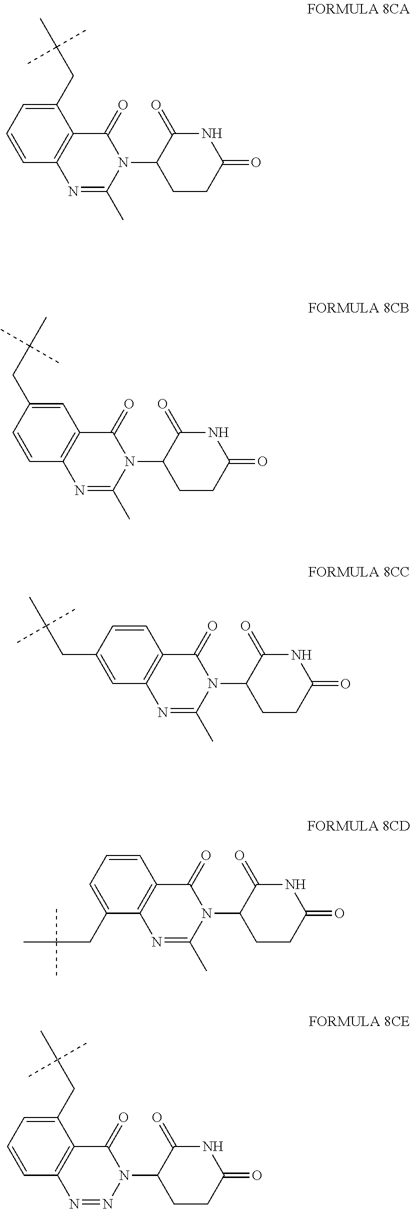

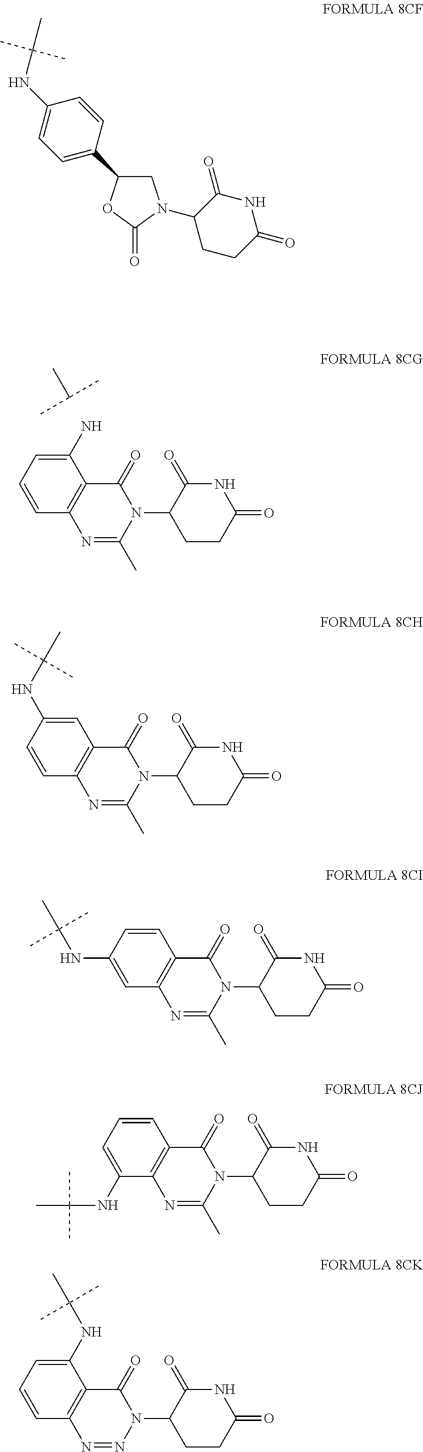

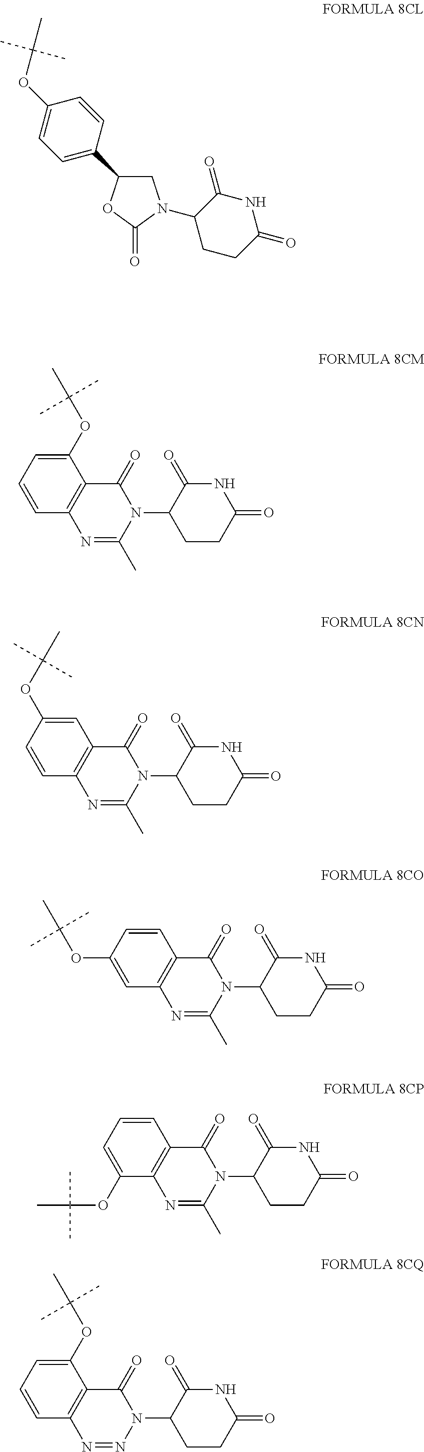

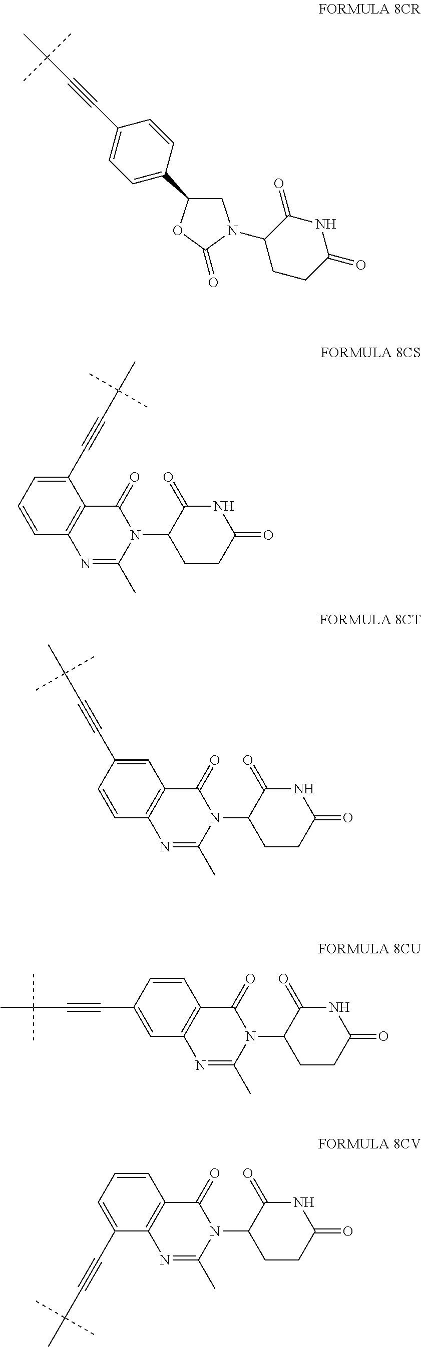

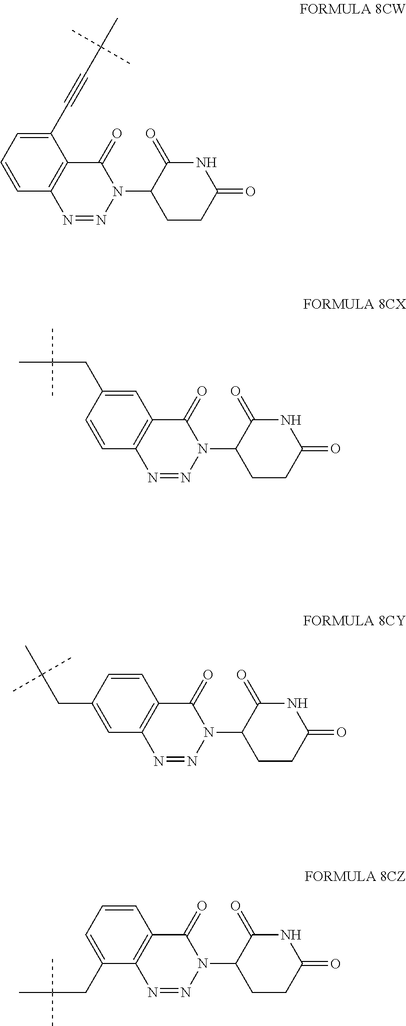

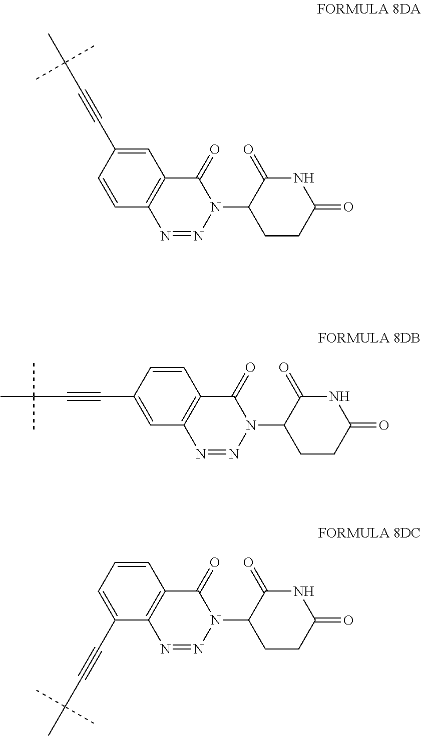

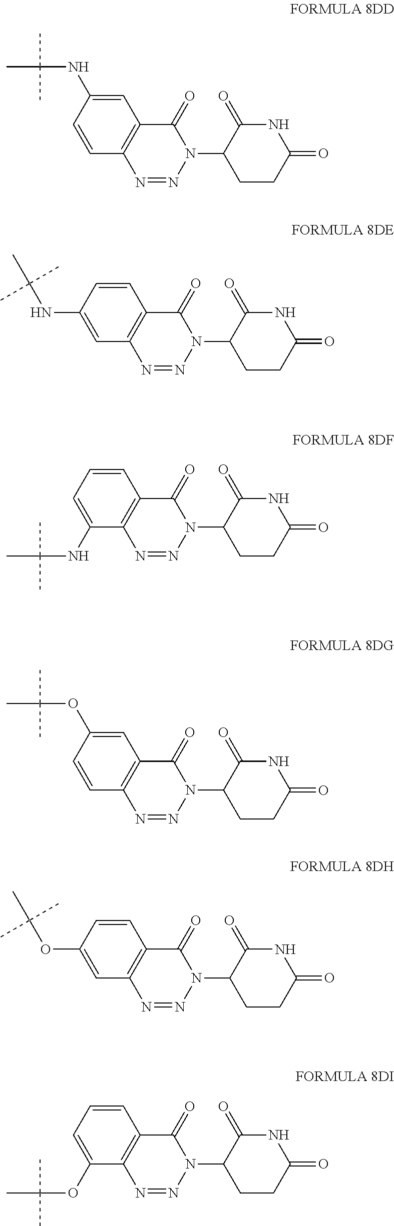

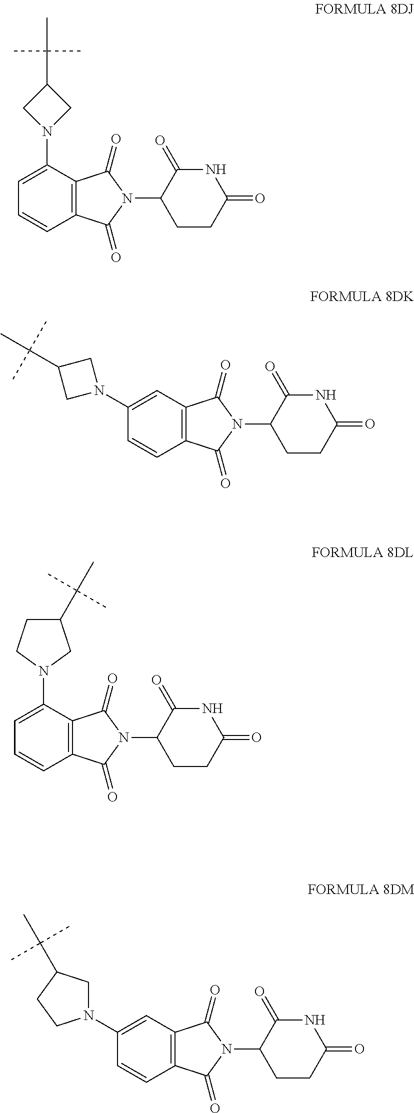

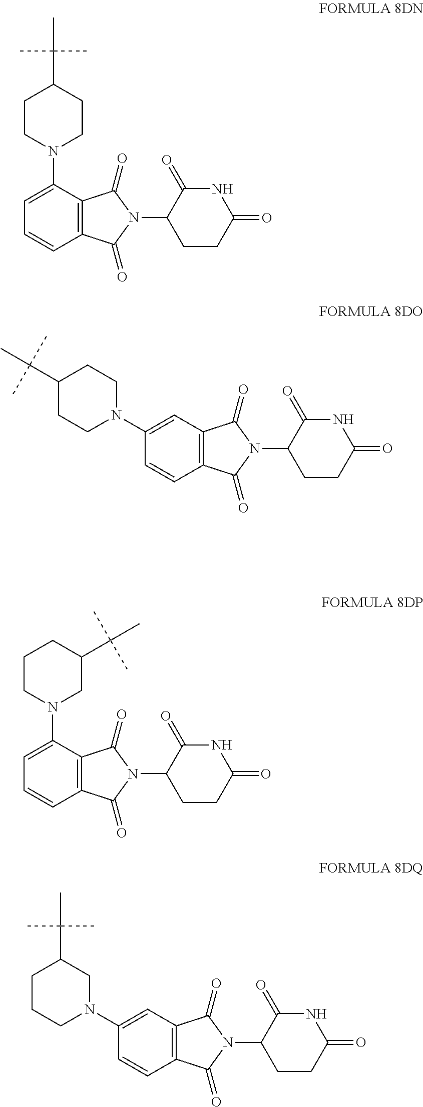

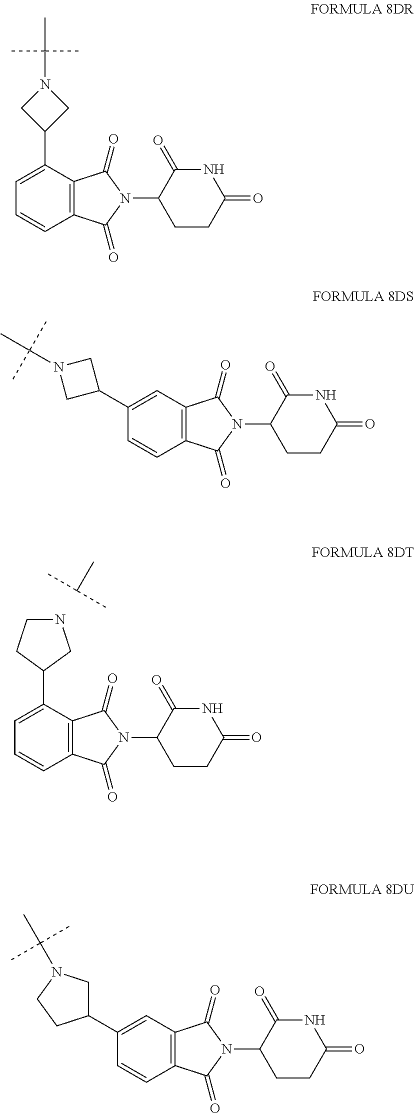

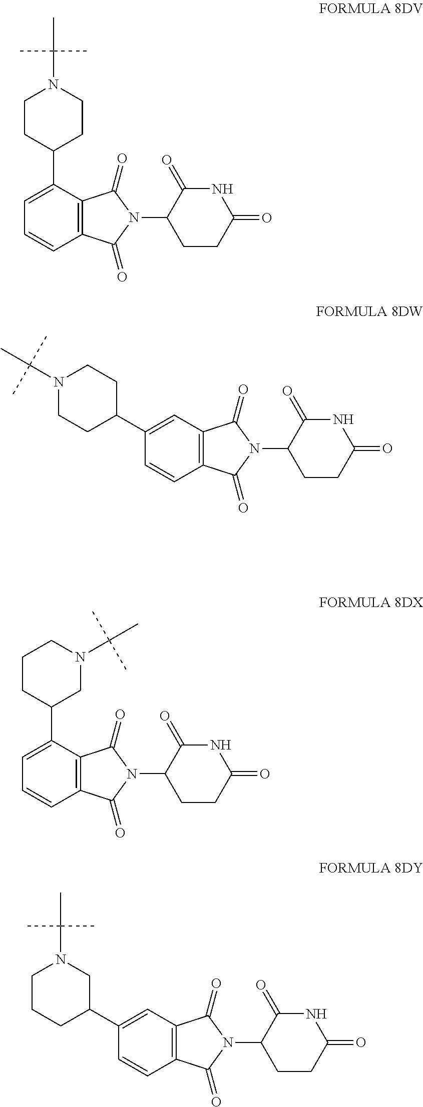

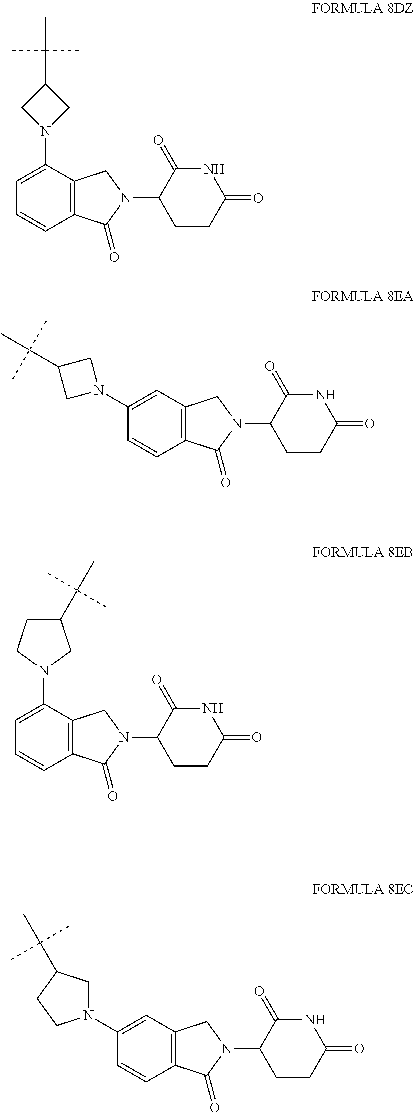

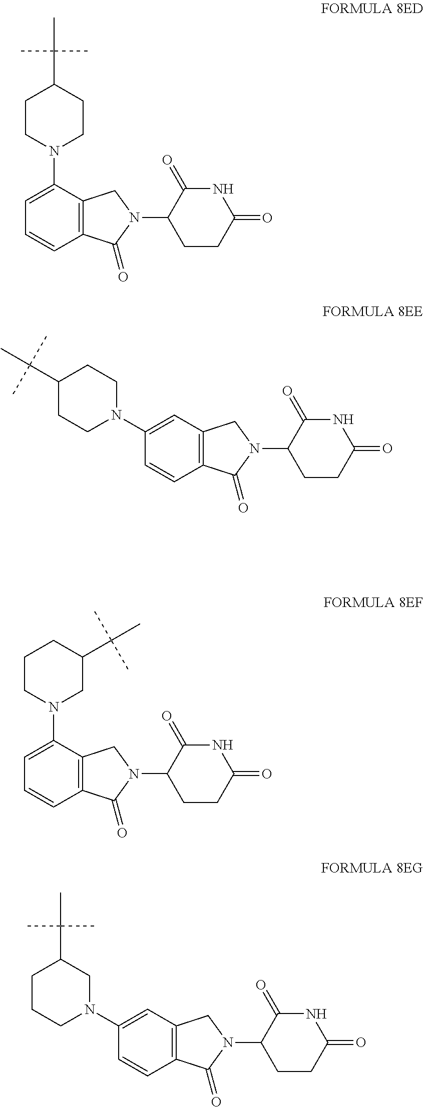

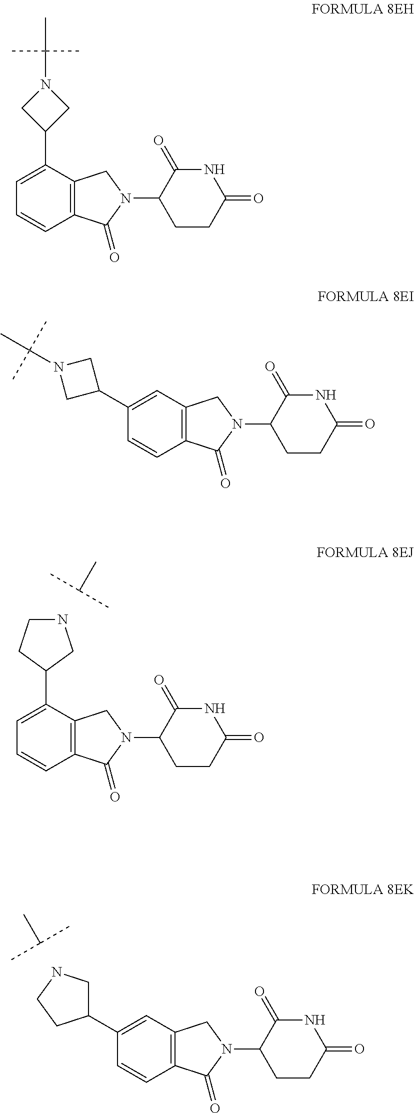

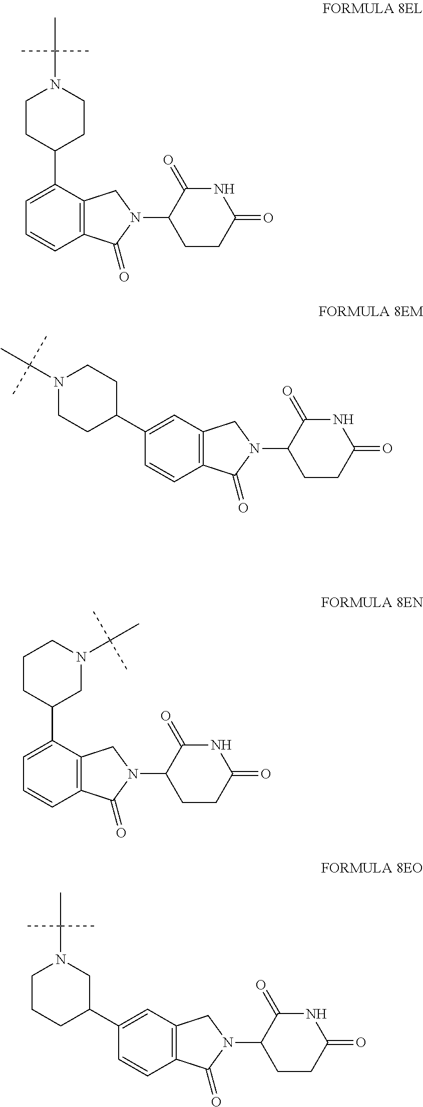

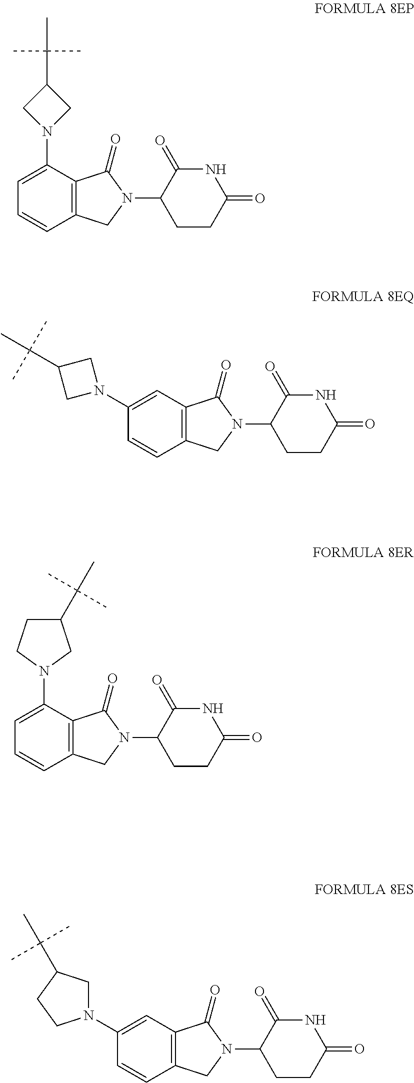

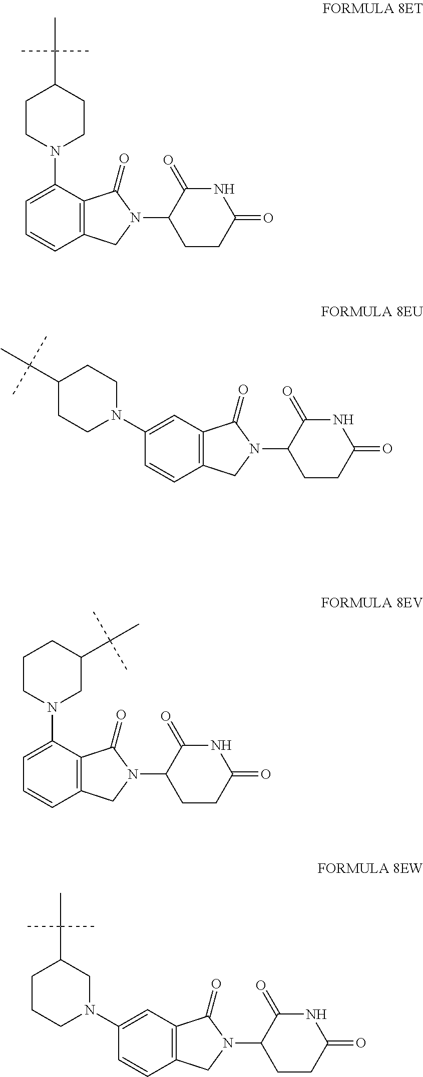

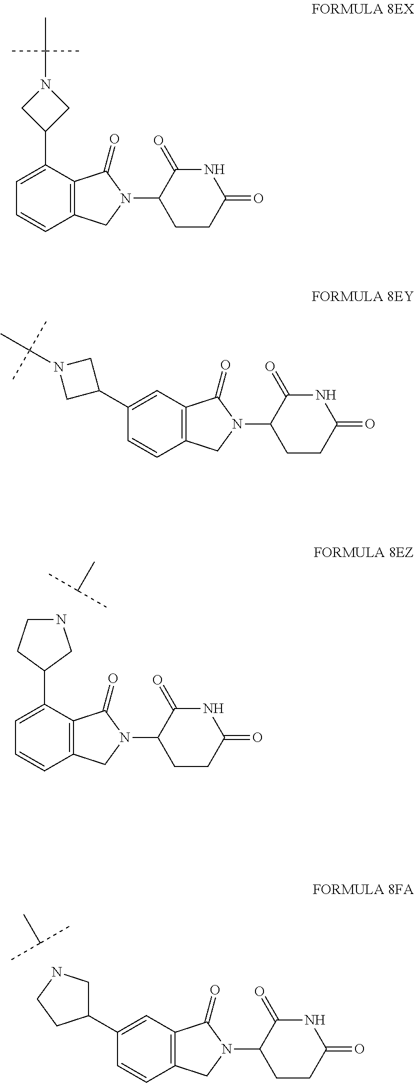

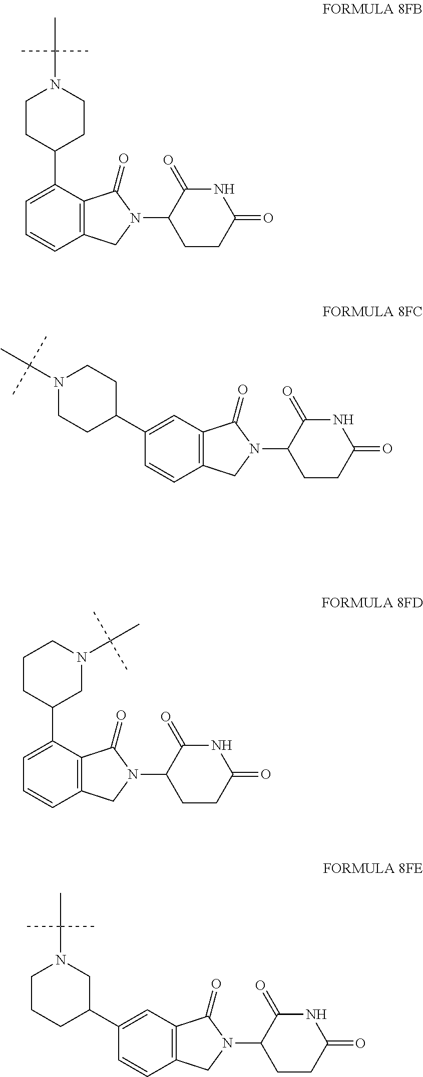

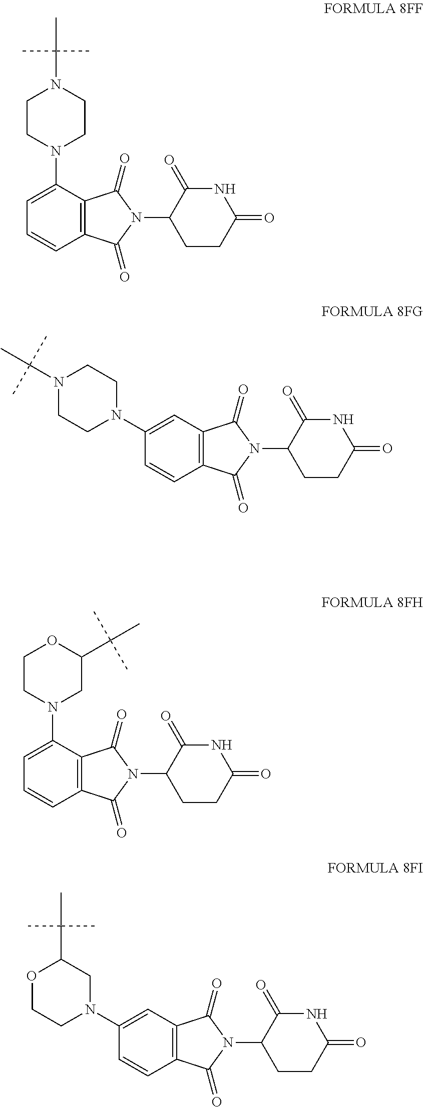

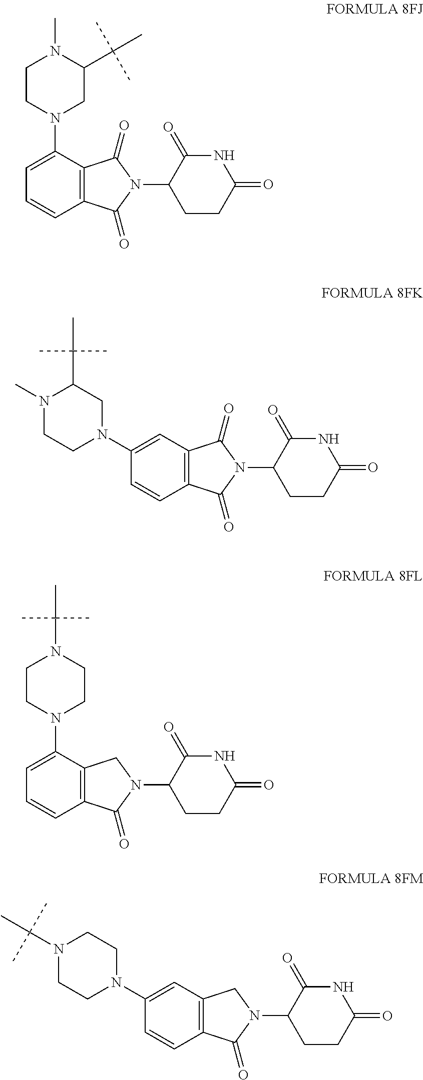

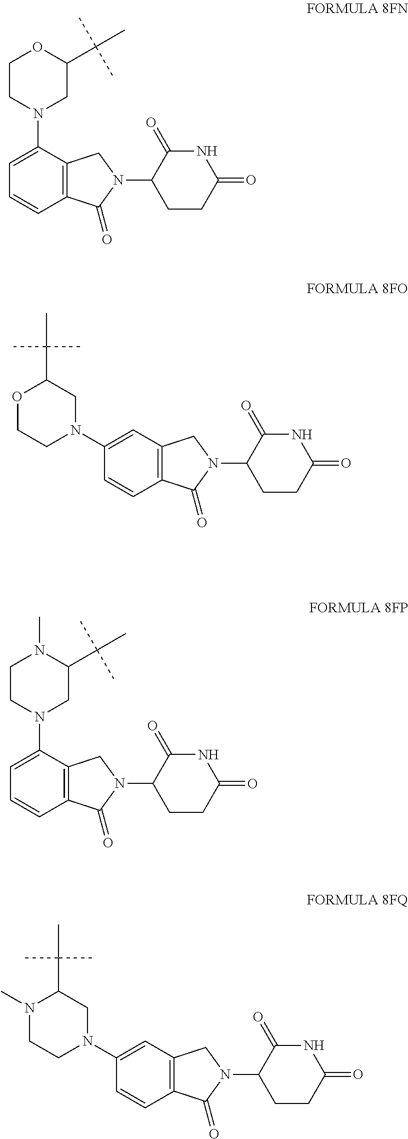

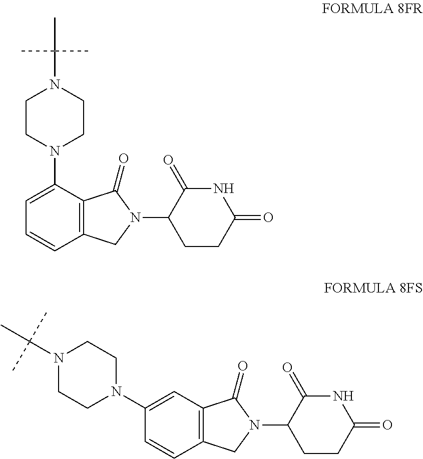

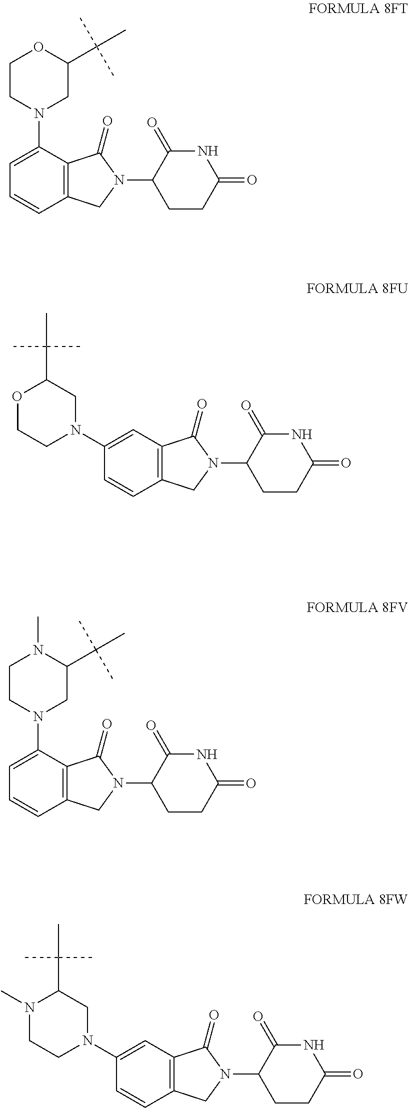

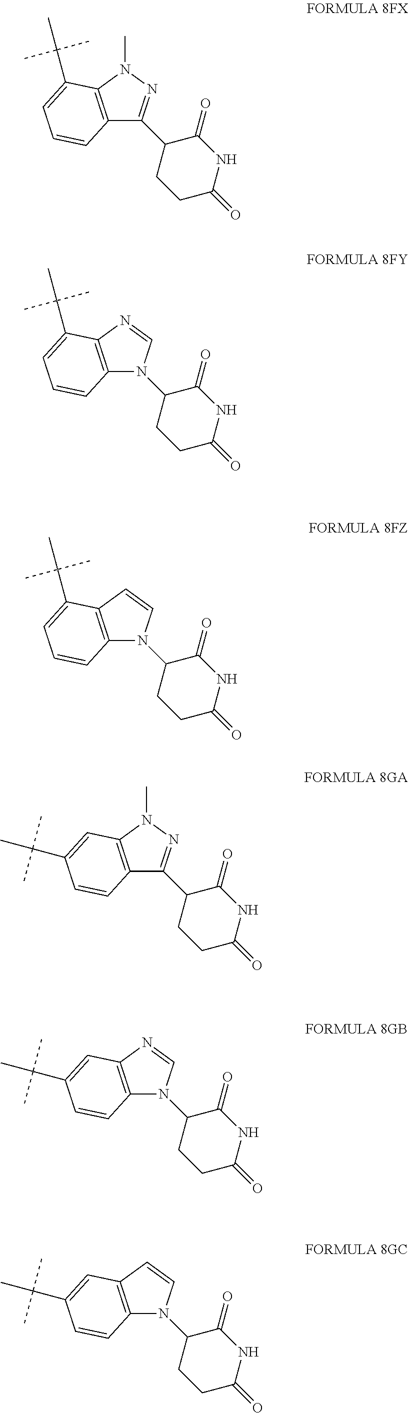

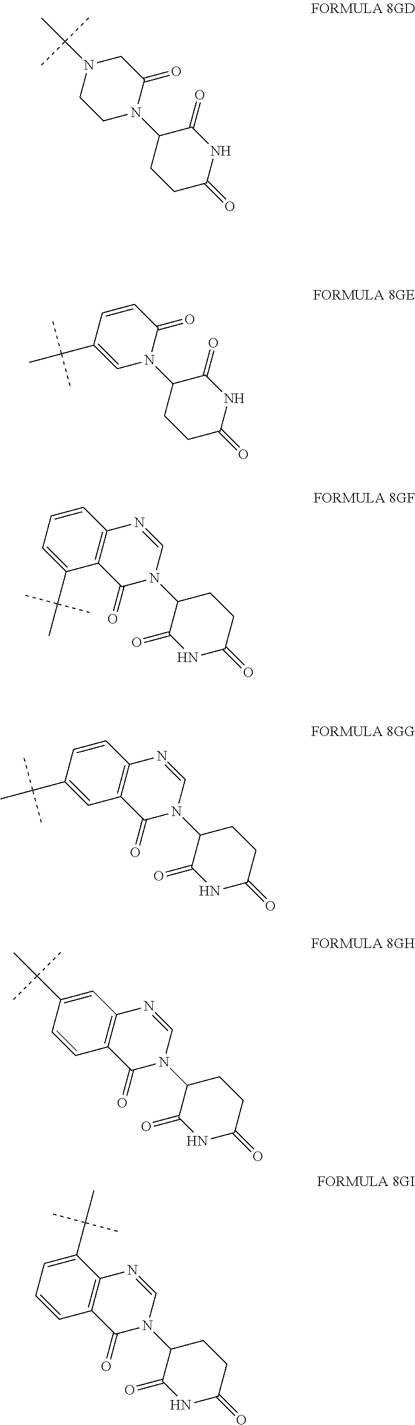

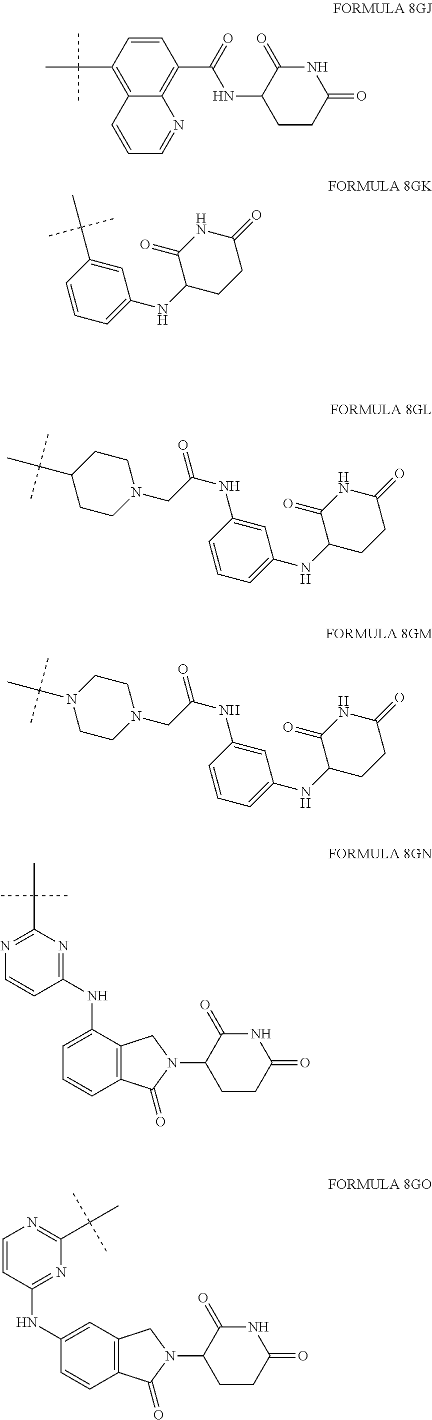

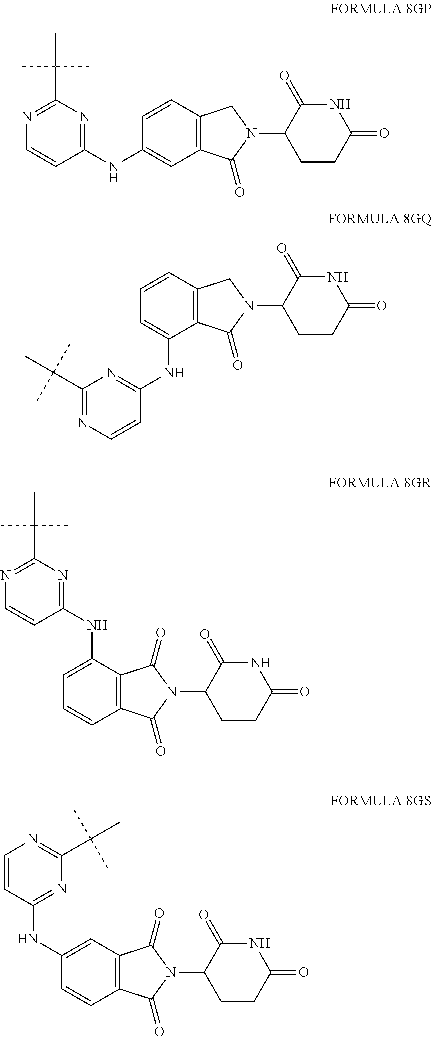

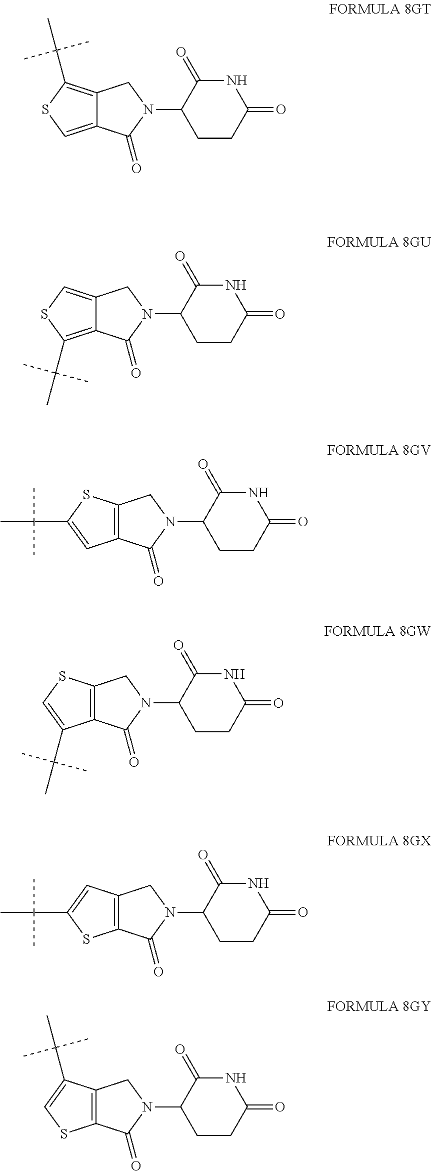

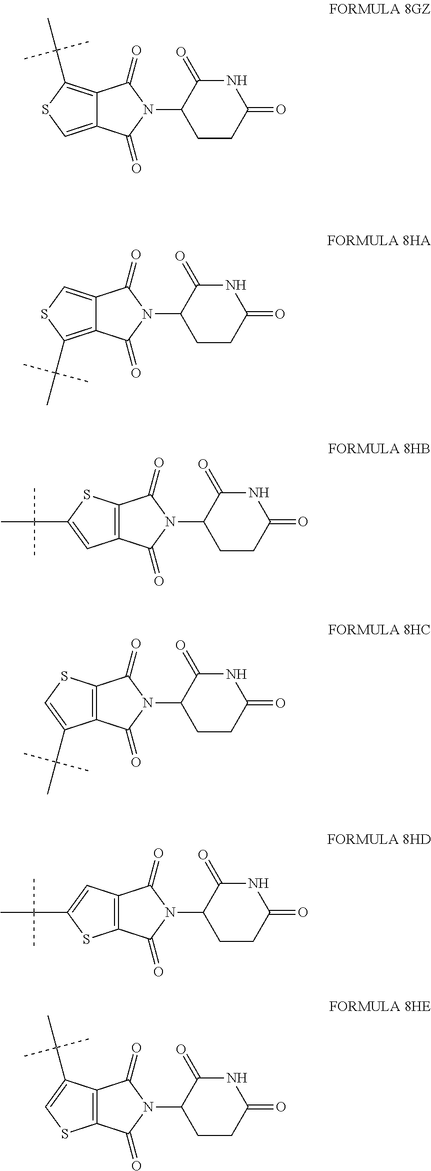

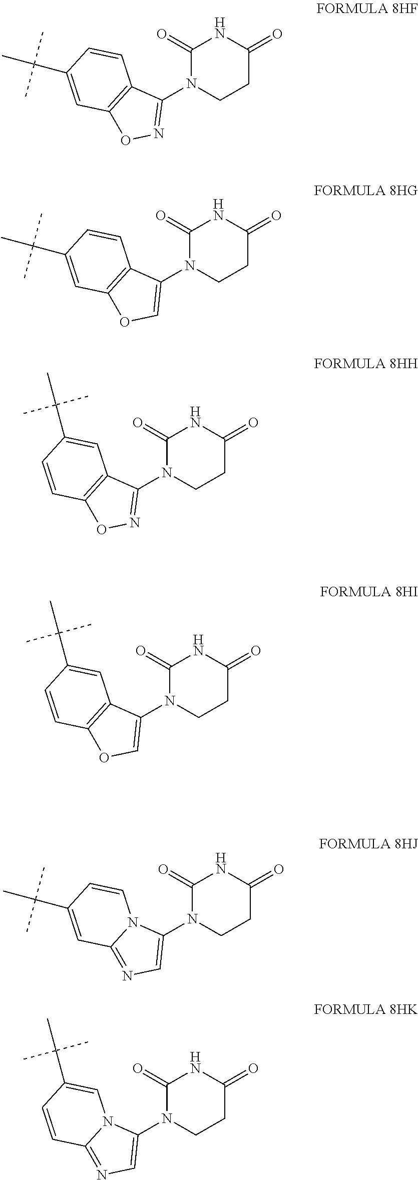

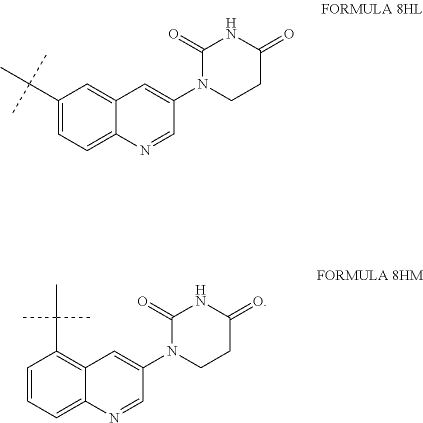

In some embodiments, the degradation tag is a moiety of FORMULA 8A, 8B, 8C, 8D, 8E, 8F, 8G, 8H, 8I, 8J, 8K, 8L, 8M, 8N, 80, 8P, 8Q, 8R, 8S, 8T, 8U, 8V, 8W, 8X, 8Y, 8Z, 8AA, 8AB, 8AC, or 8AD.

In some embodiments, the degradation tag is a moiety of FORMULA 6A, 6B, or 6C, wherein R_{6E}¹ is C₁-C₆ alkyl.

In some embodiments, the degradation tag is a moiety of FORMULA 6A, 6B, or 6C, and wherein R_{6E}¹ is selected from isopropyl and tert-butyl.

In some embodiments, the degradation tag is a moiety of FORMULA 6A-1, 6B-1, 6C-1, 6A-2, 6B-2, or 6C-2.

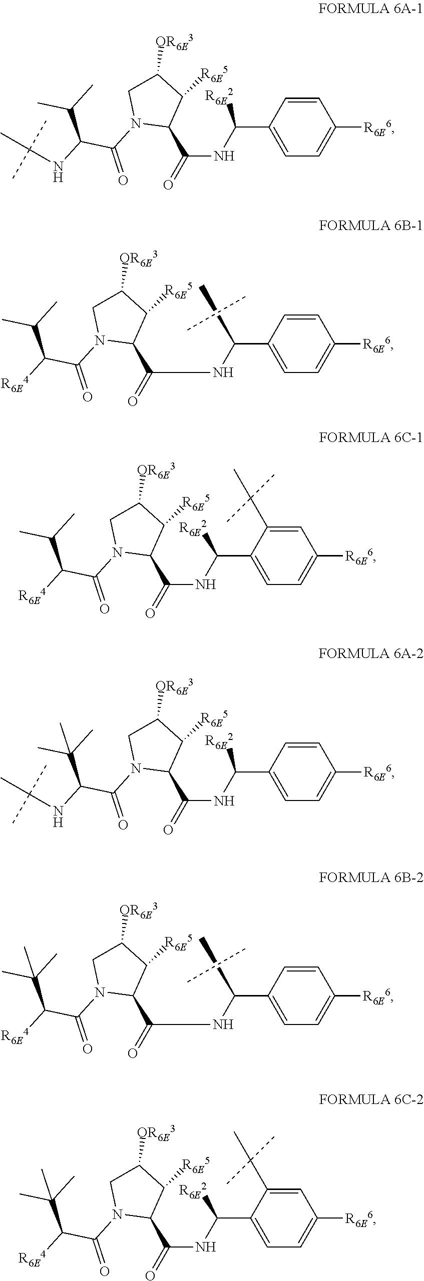

wherein R_{6E}², R_{6E}³, R_{6E}⁴, R_{6E}⁵, and R_{6E}⁶ are defined as in FORMULA 6A, 6B, or 6C.

In some embodiments, R_{6E}² is H or C₁-C₄ alkyl.

In some embodiments, R_{6E}² is H or Me.

In some embodiments, the degradation tag is a moiety of FORMULA 6A-3, 6B-3, 6C-3, 6A-4, 6B-4, or 6C-4:

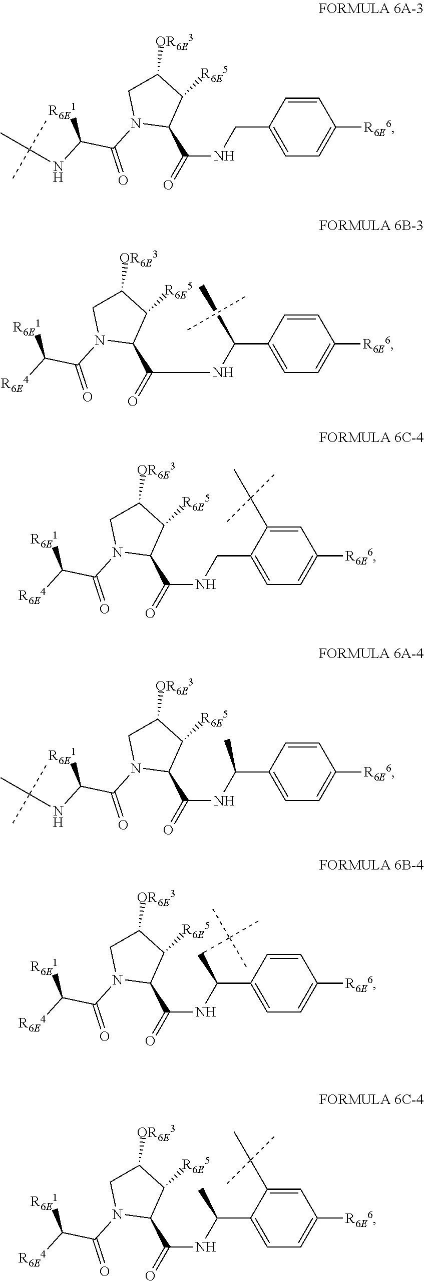

wherein R_{6E}¹, R_{6E}³, R_{6E}⁴, R_{6E}⁵, and R_{6E}⁶ are defined as in FORMULA 6A, 6B, or 6C.

In some embodiments, R_{6E}³ is H.

In some embodiments, the degradation tag is a moiety of FORMULA 6A-5, 6B-5, or 6C-5:

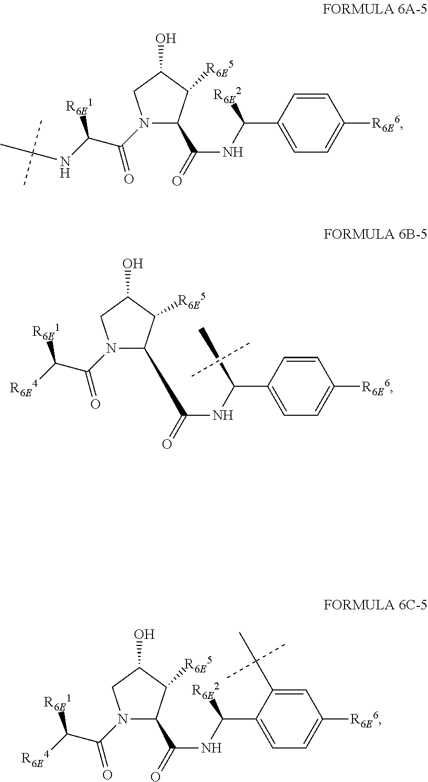

wherein
R_{6E}¹, R_{6E}², R_{6E}⁴, R_{6E}⁵, and R_{6E}⁶ are defined as in FORMULA 6A, 6B, or 6C.

In some embodiments, R_{6E}⁵ is H or F.

In some embodiments, the degradation tag is a moiety of FORMULA 6A-6, 6B-6, 6C-6, 6A-7, 6B-7, or 6C-7:

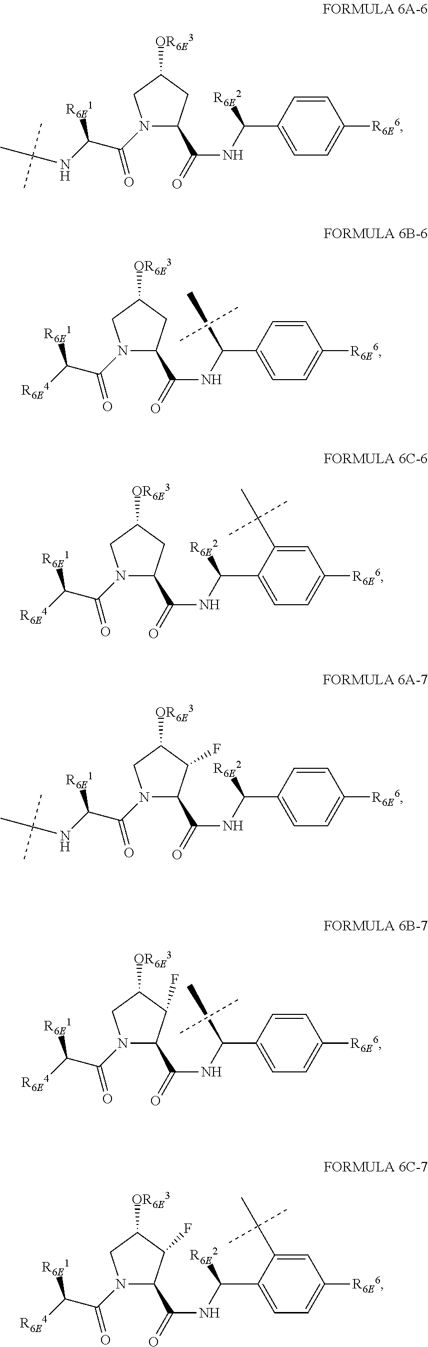

wherein
R_{6E}¹, R_{6E}², R_{6E}³, R_{6E}⁴, and R_{6E}⁶ are defined as in FORMULA 6A, 6B, or 6C.

In some embodiments, R_{6E}⁶ is selected from hydrogen, halogen, cyano, optionally substituted aryl, and optionally substituted heteroaryl.

In some embodiments, R_{6E}⁶ is selected from the group consisting of halogen, cyano, optionally substituted thiazole, optionally substituted oxazole, optionally substituted imidazole, optionally substituted pyrazole, optionally substituted oxadiazole, optionally substituted triazole, and optionally substituted isoxazole. In some preferred embodiments, R_{6E}⁶ is 4-methylthiazol-5-yl, or oxazol-5-yl.

In some embodiments, R_{6E}⁶ is methyl thiazole (preferably 2-methyl thiazole or 4-methyl thiazole).

In some embodiments, the degradation tag is a moiety of FORMULA 6A-8, 6B-8, or 6C-8:

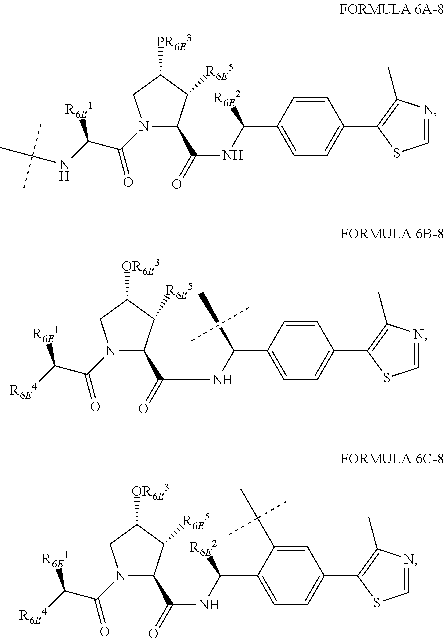

wherein
R_{6E}¹, R_{6E}², R_{6E}³, R_{6E}⁴, and R_{6E}⁵ are defined as in FORMULA 6A, 6B, or 6C.

In some embodiments R_{6E}⁴ is selected from NR_{6E}⁷R_{6E}⁸,

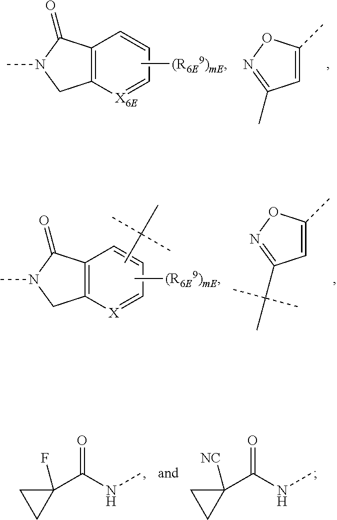

wherein
R_{6E}⁹, X_{6E}, and mE are defined as in FORMULA 6A, 6B, or 6C.

In some embodiments, R_{6E}⁷ is selected from hydrogen, optionally substituted C₁-C₈alkyl, optionally substituted 1-8 membered heteroalkyl, optionally substituted C₁-C₈ haloalkyl, optionally substituted C₃-C₈cycloalkyl, optionally substituted C₁-C₈alkyl-CO, optionally substituted 1-8 membered heteroalkyl-CO, optionally substituted C₁-C₈ haloalkyl-CO, optionally substituted C₃-C₈cycloalkyl-CO, optionally substituted C₃-C₈cycloalkyl-C₁-C₈alkyl-CO, optionally substituted 3-10 membered heterocyclyl-CO, optionally substituted 3-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;
R_{6E}⁸ is selected from hydrogen, optionally substituted C₁-C₈alkyl, optionally substituted 1-8 membered heteroalkyl, optionally substituted C₁-C₈ haloalkyl, and optionally substituted C₃-C₈cycloalkyl;
R_{6E}⁹, at each occurrence, is independently selected from the group consisting of hydrogen, halogen, cyano, optionally substituted C₁-C₈alkyl, optionally substituted 1-8 membered heteroalkyl, optionally substituted C₃-C₈cycloalkyl, optionally substituted C₃-C₈ heterocycloalkyl, optionally substituted C₁-C₈alkoxy, optionally substituted C₁-C₈cycloalkoxy; X_{6E} is selected from CH and N; and
m_{E} is 0, 1, 2, 3, or 4.

In some embodiments, R_{6E}⁵ is selected from hydrogen and halogen, preferably, H and F.

In some embodiments, R_{6E}⁹, at each occurrence, is independently selected from the group consisting of hydrogen, halogen, cyano, optionally substituted C₁-C₈alkyl, optionally substituted 1-8 membered heteroalkyl, optionally substituted C₃-C₈cycloalkyl, optionally substituted 3-8 membered heterocycloalkyl, optionally substituted C₁-C₈alkoxy, and optionally substituted C₃-C₈cycloalkoxy; and
m_{E} is 0, 1, 2, 3, or 4.

In some embodiments, the substituent(s) for R_{E}¹¹ and R_{E}^{11′} are independently optionally substituted groups selected from C₁-C₄ alkyl, C₁-C₄ haloalkyl, halogen (such as F), and CN.

In some embodiments, R_{6E}⁴ is selected from NH₂, NHC(O)Me,

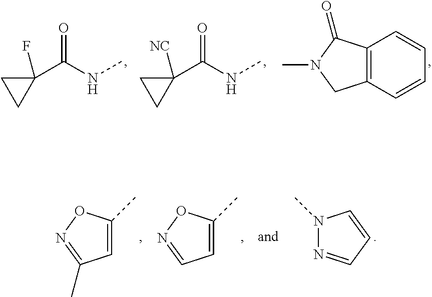

In some embodiments, the degradation tag is a moiety of FORMULA 6A-9, 6A-10, 6A-11, 6A-12, 6A-13, 613-9, 613-10, 613-11, 613-12, 613-13, 613-14, 613-15, 6C-9, 6C-10, 6C-11, 6C-12, 6C-13, 6C-14, or 6C-15:

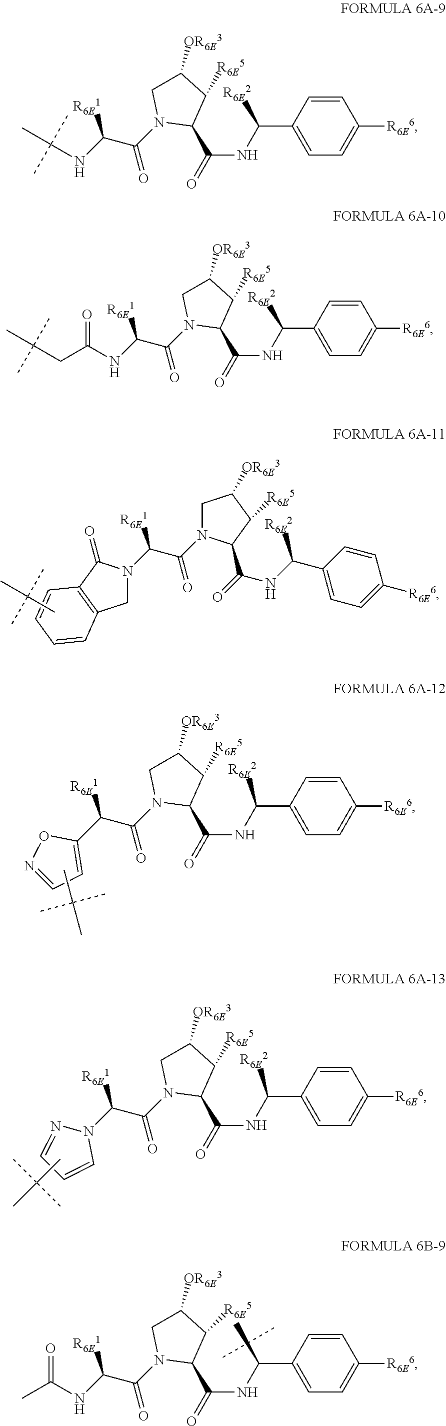

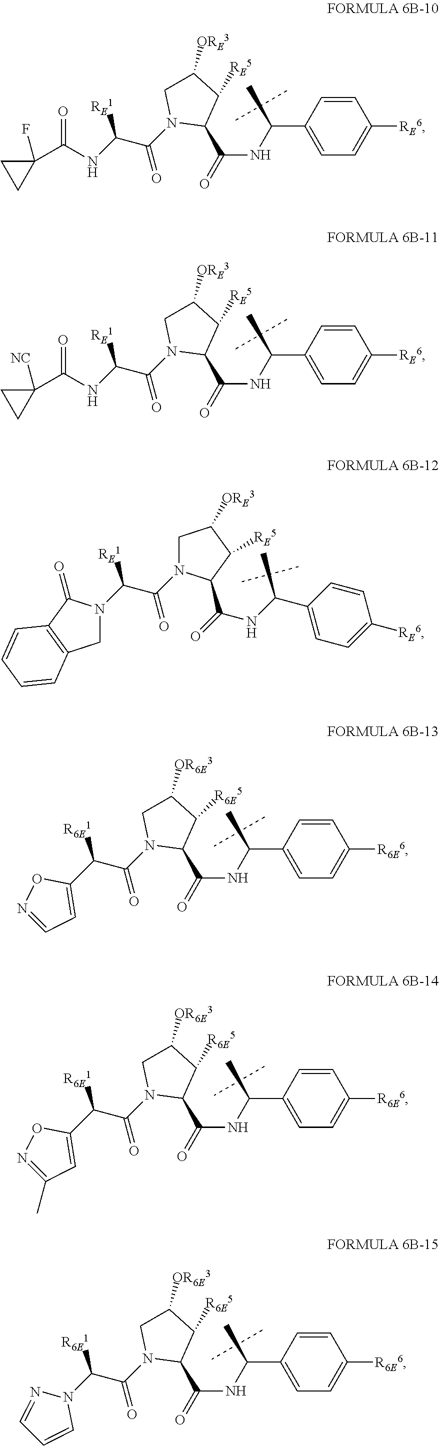

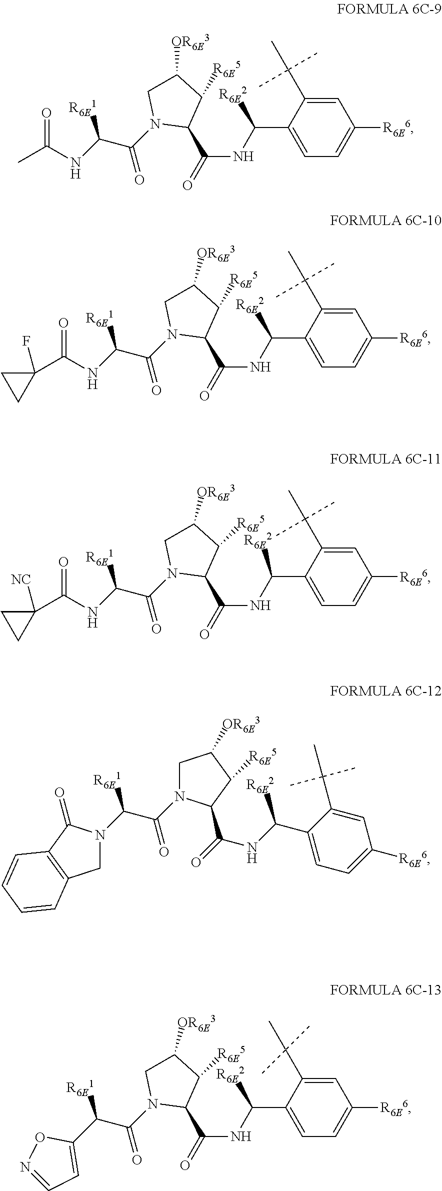

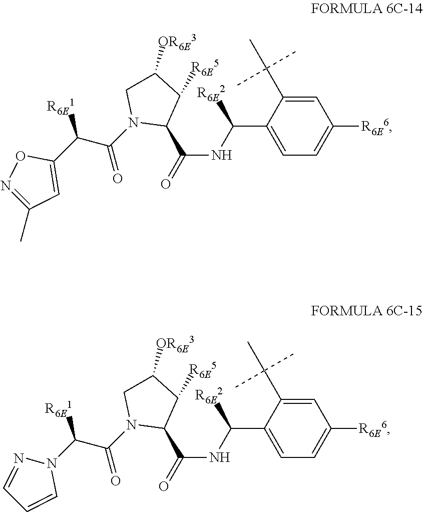

wherein R_{6E}¹, R_{6E}², R_{6E}³, R_{6E}⁵, and R_{6E}⁶ are defined as in FORMULA 6A, 613, or 6C.

In some embodiments, the degradation tag is a moiety of FORMULA 6A, 6B, or 6C, and wherein
R_{6E}¹ is C₁-C₆ alkyl (such as isopropyl or tert-butyl);
R_{6E}² is H or C₁-C₄ alkyl (such as Me);
R_{6E}⁴ is selected from NH₂, NHC(O)Me,

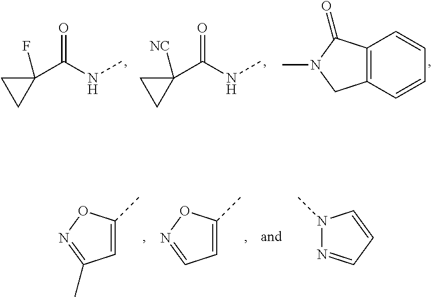

(preferably, is

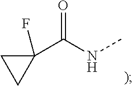

R_{6E}^{2′} is null or C₁-C₄ alkylene (such as CH₂);
R_{6E}^{4′} is selected from NH, C(O)NH, CH₂C(O)NH,

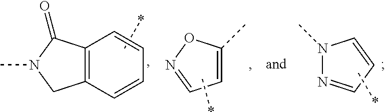

R_{6E}⁵ is selected from the group consisting of hydrogen and halogen (such as F); and
R_{6E}⁶ is selected from the group consisting of halogen, cyano, optionally substituted thiazolyl, optionally substituted oxazolyl, optionally substituted imidazolyl, optionally substituted pyrazolyl, optionally substituted oxadiazolyl, optionally substituted triazolyl, and optionally substituted isoxazolyl (preferably, is optionally substituted thiazolyl, such as 2-methyl thiazolyl).

In some embodiments, the degradation tag is a moiety of any of FORMULAE 7A to 7BJ:

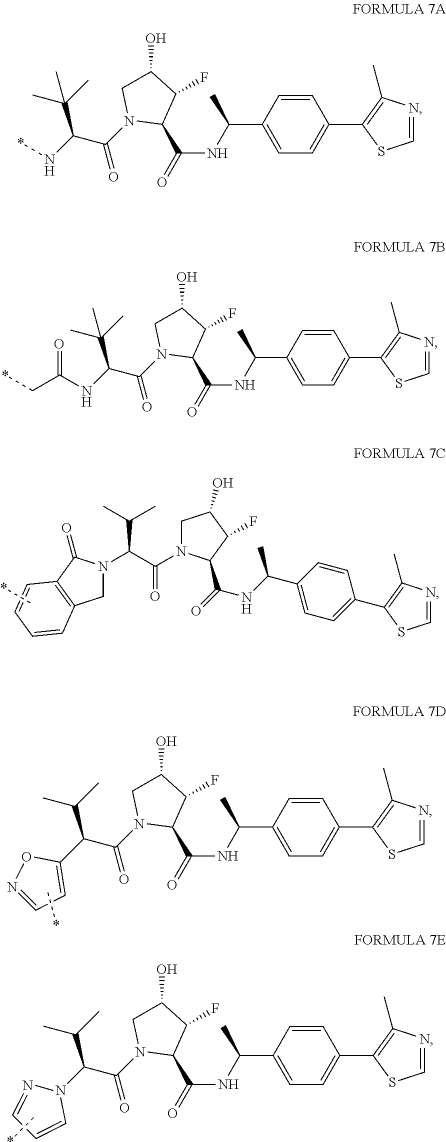

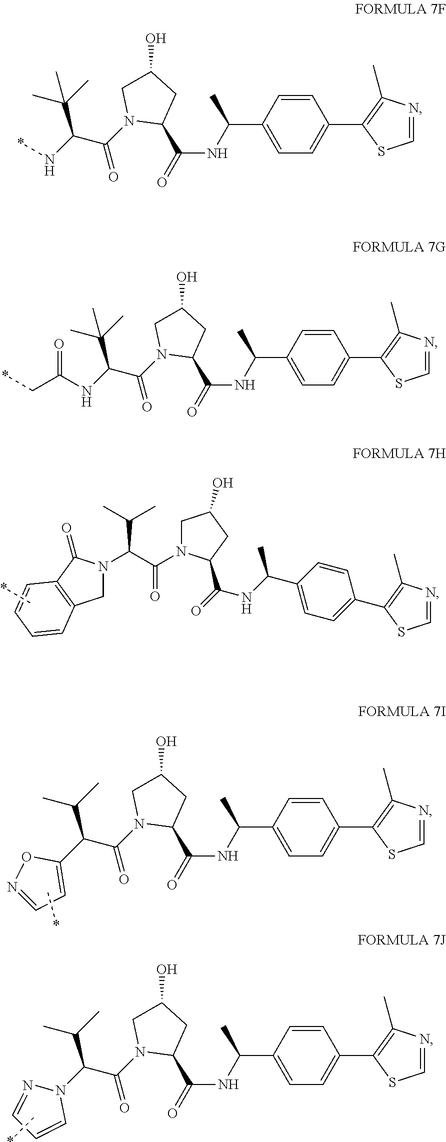

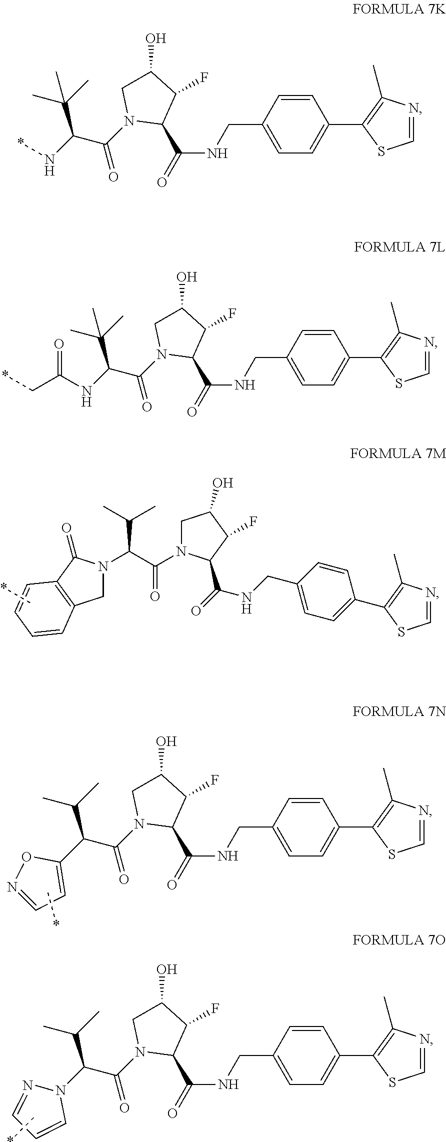

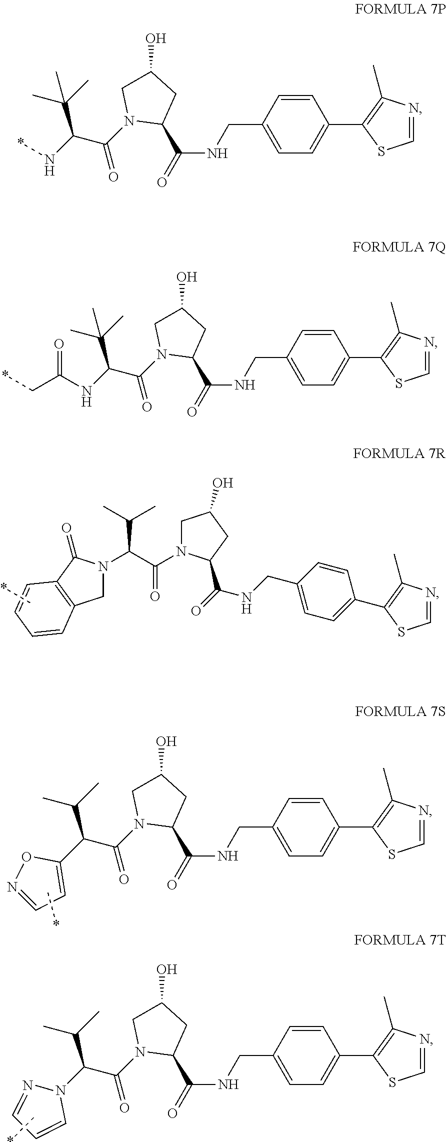

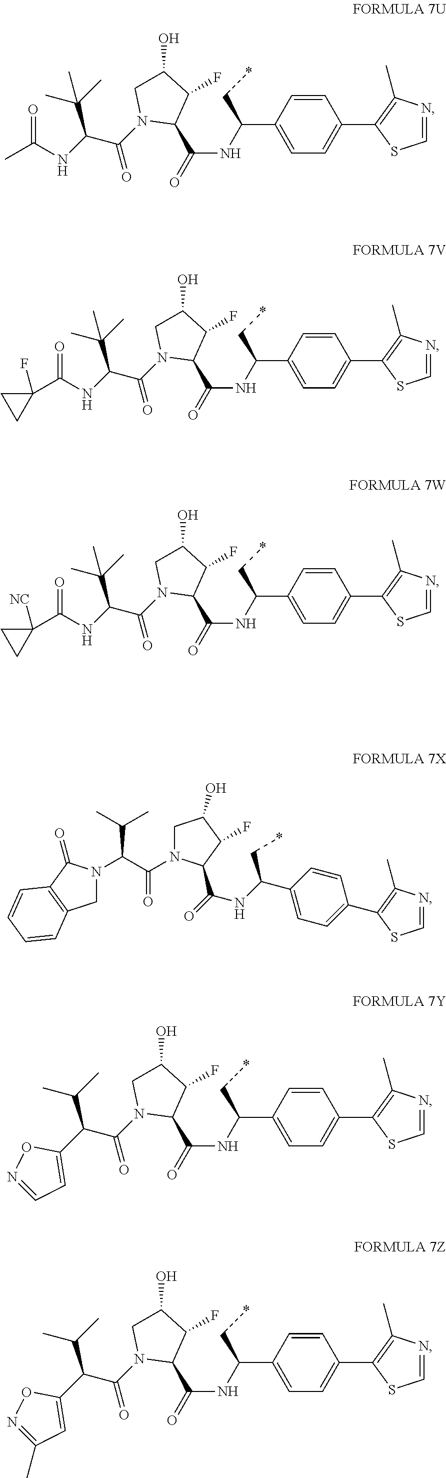

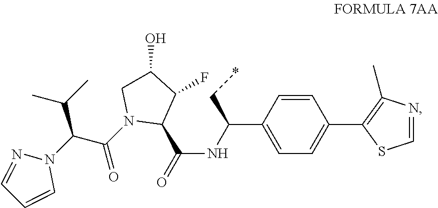

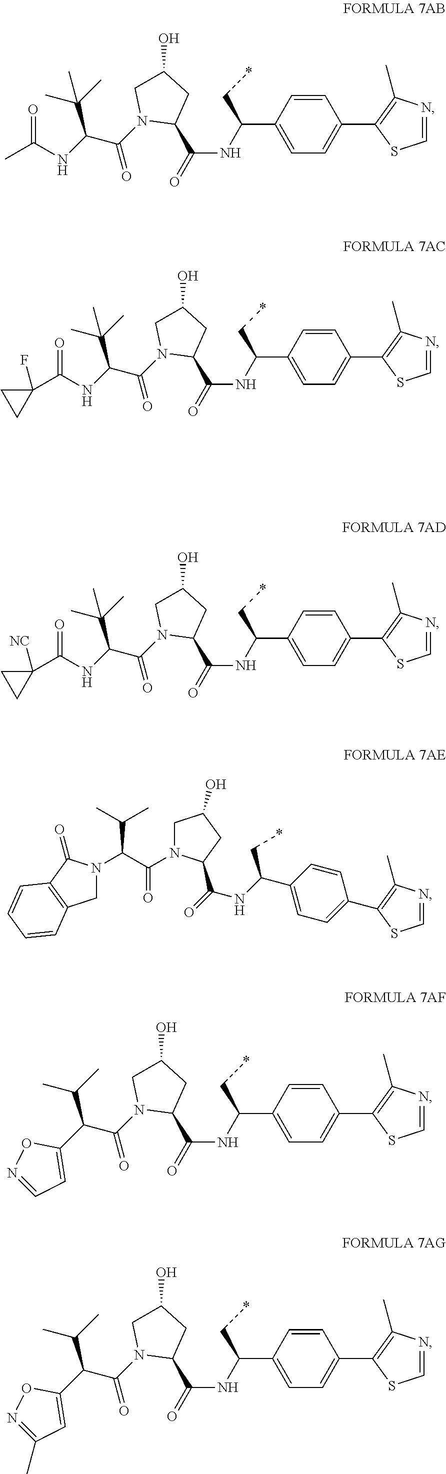

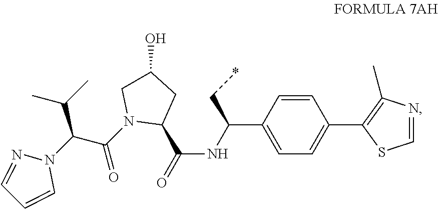

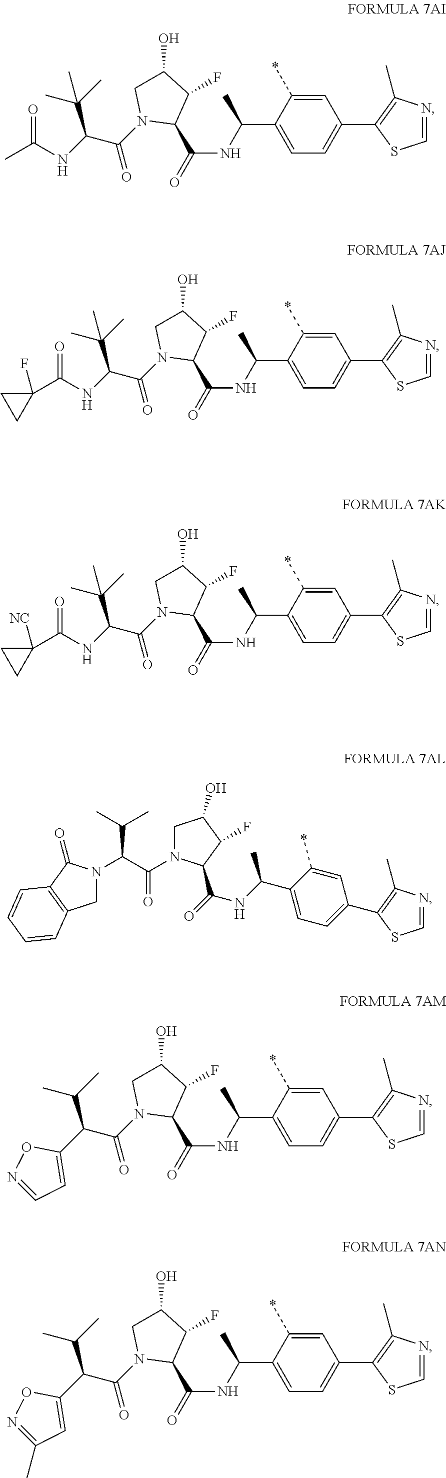

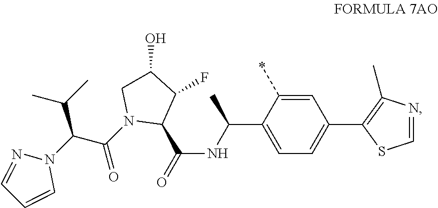

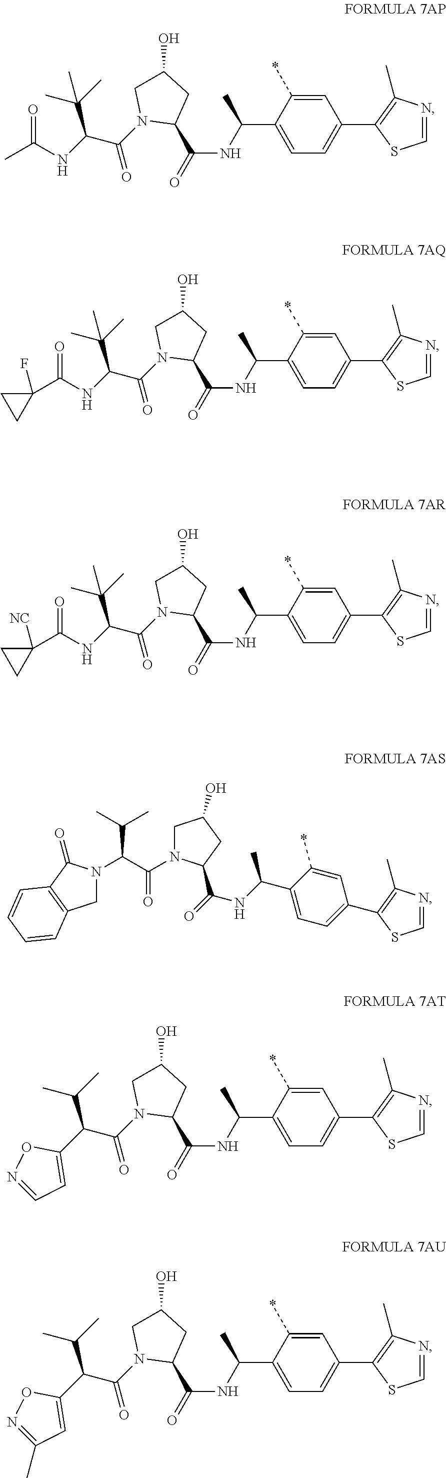

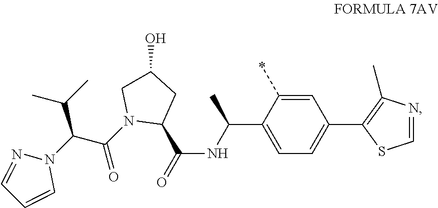

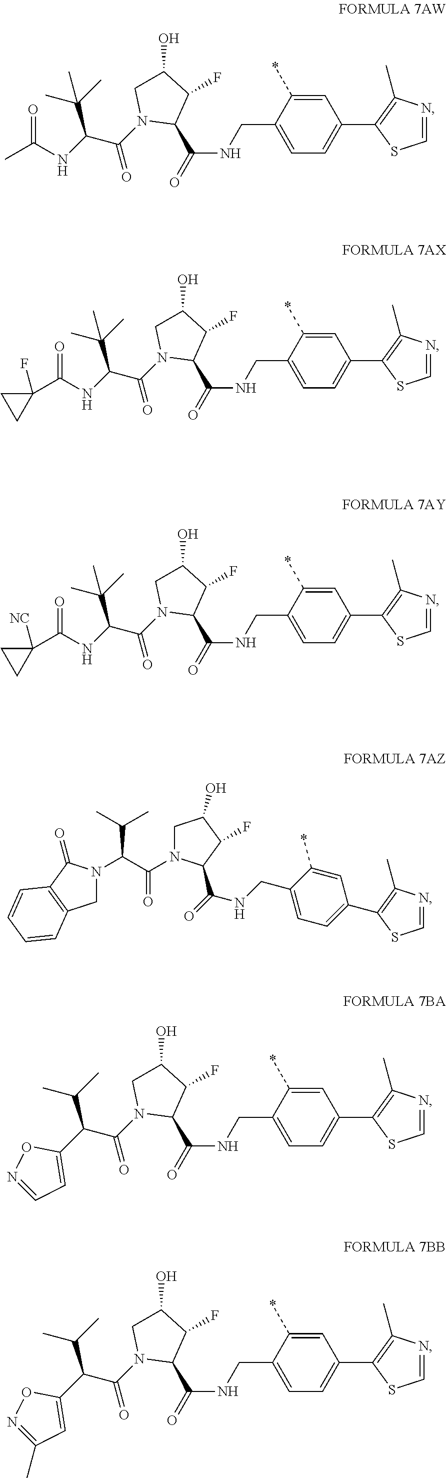

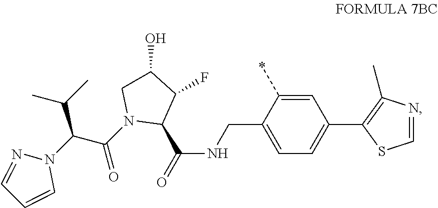

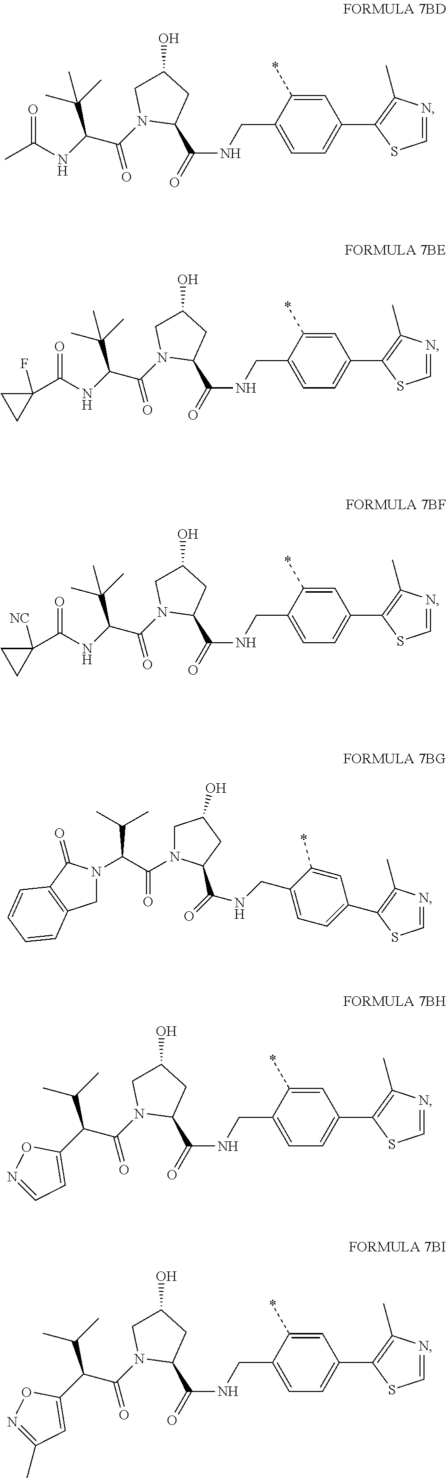

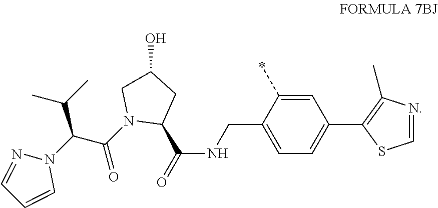

In another embodiment, the degradation tag is a moiety of FORMULA 4A:

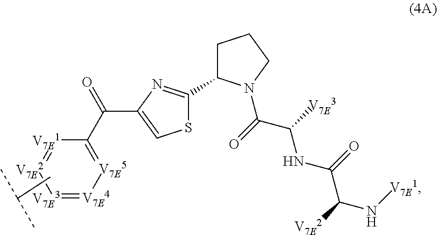

wherein
V_{7E}¹, V_{7E}², V_{7E}³, V_{7E}⁴, and V_{7E}⁵, are independently selected from the group consisting of CR_{7E}⁴ and N; and
R_{7E}¹, R_{7E}², R_{7E}³, and R_{7E}⁴ are independently selected from the group consisting of hydrogen, halogen, cyano, nitro, optionally substituted C₁-C₈ alkyl, optionally substituted C₂-C₈ alkenyl, and optionally substituted C₂-C₈ alkynyl; optionally substituted 1-8 membered heteroalkyl, optionally substituted 3-8 membered heteroalkenyl, optionally substituted 3-8 membered heteroalkynyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkoxy, optionally substituted C₁-C₈alkylamino, optionally substituted C₃-C₁₀ carbocyclyl, and optionally substituted 3-10 membered heterocyclyl.

In another embodiment, the degradation tag is a moiety of FORMULA 4B:

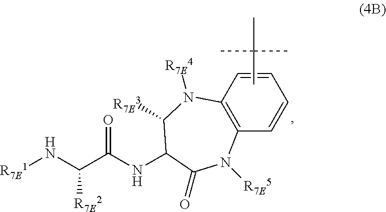

wherein
R_{7E}¹, R_{7E}², and R_{7E}³ are independently selected from the group consisting of hydrogen, halogene, optionally substituted C₁-C₈ alkyl, optionally substituted 1-8 membered heteroalkyl, optionally substituted 3-8 membered heteroalkenyl, optionally substituted 3-8 membered heteroalkynyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl; and
   R_{7E}⁴ and R_{7E}⁵ are independently selected from the group consisting of hydrogen, COR_{7E}⁶, CO₂R_{7E}⁶, CONR_{7E}⁶R_{7E}⁷, SOR_{7E}⁶, SO₂R_{7E}⁶, SO₂NR_{7E}⁶R_{7E}⁷, optionally substituted C₁-C₈ alkyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted C₃-C₈ cycloalkyl, optionally substituted 3-8 membered heterocyclyl, optionally substituted aryl, and optionally substituted heteroaryl, wherein
   R_{7E}⁶ and R_{7E}⁷ are independently selected from the group consisting of hydrogen, optionally substituted C₁-C₈ alkyl, optionally substituted 1-8 membered heteroalkyl, optionally substituted 3-8 membered heteroalkenyl, optionally substituted 3-8 membered heteroalkynyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted C₃-C₈ cycloalkyl, optionally substituted 3-8 membered heterocyclyl, optionally substituted aryl, and optionally substituted heteroaryl; or
   R_{7E}⁶ and R_{7E}⁷ together with the atom(s) to which they are connected form a 3-8 membered C₁-C₁₀ cycloalkyl or heterocyclyl ring.

In another embodiment, the degradation tag is a moiety of FORMULA 5.

In another embodiment, the degradation tag is a moiety of any of FORMULAE 5-1 to 5-6.

In another embodiment, the degradation tag is a moiety of any of FORMULAE 5A to 5X.

In another embodiment, the degradation tag is a moiety of any of FORMULAE 5-1 and 5A.

In another embodiment, the degradation tag is a moiety of any of FORMULAE 8A to 8IM.

In another embodiment, the degradation tag is a moiety of FORMULA 6A, 6B, and 6C.

In another embodiment, the degradation tag is a moiety of FORMULA 6A.

In another embodiment, the degradation tag is a moiety of FORMULA 6B.

In another embodiment, the degradation tag is a moiety of any of FORMULAE 6A-1 to 6A-13.

In another embodiment, the degradation tag is a moiety of any of FORMULAE 6B-1 to 6B-15.

In another embodiment, the degradation tag is a moiety of any of FORMULAE 7A to 7BJ.

In some embodiments, the linker comprises acyclic or cyclic saturated or unsaturated carbon, ethylene glycol, amide, amino, ether, urea, carbamate, aromatic, heteroaromatic, heterocyclic or carbonyl groups.

In certain embodiments, the length of the linker is 0, 1, 2, 3, 4, 5, 6, 7, 8, 9, 10, 11, 12, 13, 14, 15, 16, 17, 18, 19, 20 or more atoms.

In another embodiment, A_{L} and B_{L}, at each occurrence, are independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}OR_{L}^{E}, R_{L}^{d}SR_{L}^{e}, R_{L}^{d}N(R_{L}¹)R_{L}^{e}, R_{L}^{d}N(R_{L}¹)COR_{L}^{e}; wherein R_{L}^{d} and R_{L}^{E}, at each occurrence, are independently selected from the group consisting of null, optionally substituted C₁, C₂ or C₃ alkylene, R_{L}^{r}, R_{L}^{r}—(C₁, C₂ or C₃ alkylene), (C₁, C₂ or C₃ alkylene)-R_{E}^{r}, and (C₁, C₂ or C₃ alkylene) —R_{L}^{r}—(C₁, C₂ or C₃ alkylene).

In another embodiment, A_{L} and B_{L}, at each occurrence, are independently selected from the group consisting of null, R_{L}^{d}—R_{L}^{e}, R_{L}^{d}COR_{L}^{e}, R_{L}^{d}C(O)N(R_{L}¹)R_{L}^{e}, R_{L}^{d}OR_{L}^{e}, R_{L}^{d}N(R_{L}¹)COR_{L}^{e}; wherein R_{L}^{d} and R_{L}^{e}, at each occurrence, are independently selected from the group consisting of null, and optionally substituted C₁, C₂ or C₃ alkylene.

In another embodiment, W_{L}¹ and W_{L}², at each occurrence, are independently selected from the group consisting of null, O, S, NH, R_{L}^{r}, and optionally substituted C₁-C₃ alkylene, with the proviso that at least one of W_{L}¹ and W_{L}² is not null.

In another embodiment, none of W_{L}¹-W_{L}², A_{L}-W_{L}¹ and W_{L}²-B_{L} is a moiety of —O—O—.

In another embodiment, W_{L}², at each occurrence, is independently null, O, or NH; and W_{L}¹, at each occurrence, is independently selected from the group consisting of R_{L}^{r}, and optionally substituted C₁, C₂ or C₃ alkylene.

In another embodiment, W_{L}¹, at each occurrence, is independently null, O, or NH; and W_{L}², at each occurrence, is independently selected from the group consisting of R_{L}^{r}, and optionally substituted C₁, C₂ or C₃ alkylene.

In another embodiment, W_{L}², at each occurrence, is independently null, or O; and W_{L}¹, at each occurrence, is independently optionally substituted C₁, C₂ or C₃ alkylene.

In another embodiment, W_{L}¹, at each occurrence, is independently null, or O; and W_{L}², at each occurrence, is independently optionally substituted C₁, C₂ or C₃ alkylene.

In another embodiment, one of W_{L}² and W_{L}¹ is null, and the other is optionally substituted C₁, C₂ or C₃ alkylene.

In another embodiment, A_{L} is the attachment to the TRK ligand;
A_{L} is selected from R_{L}^{d}—R_{L}^{e}, R_{L}^{d}C(O)R_{L}^{e}, R_{L}^{d}C(O)NHR_{L}^{e}, R_{L}^{d}NHC(O)R_{L}^{e}, R^{d}C(O)NHR_{L}^{e}, and R^{d}NHC(O)R_{L}^{e};
B_{L} is selected from the group consisting of null, R_{L}^{d}C(O)NHR_{L}^{e}, R_{L}^{d}C(O)R_{L}^{e}, R_{L}^{d}OR_{L}^{e}, R_{L}^{d}NHC(O)R_{L}^{e}, R_{L}^{d}NHR_{L}^{e};
R_{L}^{d} and R_{L}^{e}, at each occurrence, are independently selected from the group consisting of null, optionally substituted C₁, C₂ or C₃ alkylene, R_{L}^{r}, R_{L}^{r}-(C₁, C₂ or C₃ alkylene), (C₁, C₂ or C₃ alkylene)-R_{L}^{r}, and (C₁, C₂ or C₃ alkylene)-R_{L}^{r}-(C₁, C₂ or C₃ alkylene);
W_{L}², at each occurrence, is independently selected from the group consisting of null, O, or NH; and W_{L}¹, at each occurrence, is independently selected from the group consisting of R_{L}^{r}, and optionally substituted C₁, C₂ or C₃ alkylene.

In another embodiment, A_{L} is the attachment to the TRK ligand;
A_{L} is R_{L}^{d}C(O)R_{L}^{e};
B_{L} is selected from the group consisting of null, R_{L}^{d}C(O)NHR_{L}^{e}, R_{L}^{d}OR_{L}^{e}, R_{L}^{d}NHC(O)R_{L}^{e}, and R_{L}^{d}NHR_{L}^{e};
R_{L}^{d} and R_{L}^{e}, at each occurrence, are independently selected from the group consisting of null, optionally substituted C₁, C₂ or C₃ alkylene;
W_{L}², at each occurrence, is independently selected from the group consisting of null, or O; and
W_{L}¹, at each occurrence, is independently optionally substituted C₁, C₂ or C₃ alkylene.

In another embodiment, A_{L} is the attachment to the TRK ligand;
A_{L} is R_{L}^{d}C(O)R_{L}^{e};
B_{L} is selected from the group consisting of null, R_{L}^{d}C(O)NHR_{L}^{e}, R_{L}^{d}C(O)R_{L}^{e}, R_{L}^{d}OR_{L}^{e}, R_{L}^{d}NHC(O)R_{L}^{e}, R_{L}^{d}NHR_{L}^{e};
R_{L}^{d} and R_{L}^{e}, at each occurrence, are independently selected from the group consisting of null, optionally substituted C₁, C₂ or C₃ alkylene;
one of W_{L}² and W_{L}¹ is null; and the other is optionally substituted C₁, C₂ or C₃ alkylene (preferably C₁ alkylene);
m_{L}=1, 2, 3, 4, 5, 6, 7, 8, 9 or 10 (preferably 1, 2, 3, 4, 5, 6, 7 or 8).

In another refinement, A_{L} and B_{L}, at each occurrence, are independently selected from the group consisting of null, R_{L}^{d}—R_{L}^{e}, R_{L}^{d}COR_{L}^{e}, R_{L}^{d}C(O)N(R_{L}¹)R_{L}^{e}, R_{L}^{d}OR_{L}^{e}, R_{L}^{d}N(R_{L}¹)COR_{L}^{e};
R_{L}^{d} and R_{L}^{e}, at each occurrence, are independently selected from the group consisting of null, and optionally substituted C₁, C₂ or C₃ alkylene;
one of W_{L}² and W_{L}¹ is null, and the other is optionally substituted C₁, C₂ or C₃ alkylene; and
m_{L} is 1, 2, 3, 4, 5, 6, 7, 8, 9, or 10.

In another embodiment, R_{L}^{r}, at each occurrence, is selected from the group consisting of FORMULA C1, C2, C3, C4, and C5

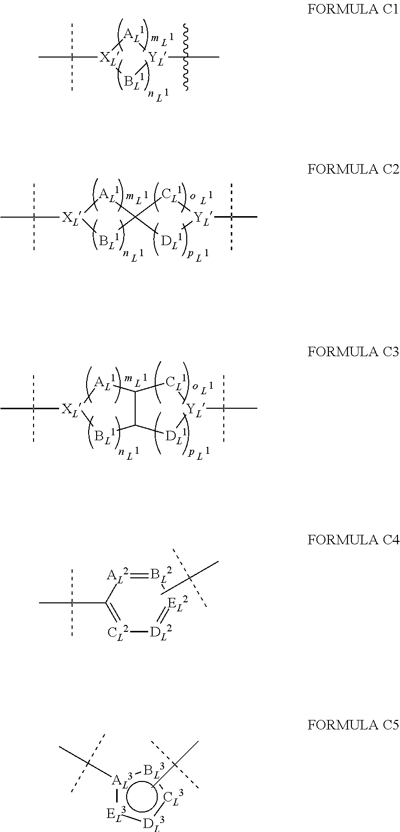

wherein
A_{L}¹, B_{L}¹, C_{L}¹ and D_{L}¹, at each occurrence, are independently selected from the group consisting of null, O, CO, SO, SO₂, NR_{L}^{b}, and CR_{L}^{b}R_{L}^{c};
X_{L}¹, Y_{L}¹, A_{L}², B_{L}², C_{L}², D_{L}² and E_{L}², at each occurrence, are independently selected from the group consisting of N, and CR_{L}^{b};
A_{L}³, B_{L}³, C_{L}³, D_{L}³, and E_{L}³, at each occurrence, are independently selected from the group consisting of N, O, S, NR_{L}^{b}, and CR_{L}^{b};
R_{L}^{b} and R_{L}^{c}, at each occurrence, are independently selected from the group consisting of hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ hetroalkyl, optionally substituted C₂-C₈ hetroalkenyl, optionally substituted C₂-C₈ hetroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁—C₈ alkylamino, and optionally substituted C₁-C₈ alkylaminoC₁-C₈ alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl; and
m_{L}¹, n_{L}¹, o_{L}¹ and p_{L}¹ are independently selected from the group consisting of 0, 1, 2, 3, 4 and 5.

In another embodiment, R_{L}^{r}, at each occurrence, is selected from Group R_{L}^{r1} and Group R_{L}^{r2}, and

Group R_{L}^{r1} consists of optionally substituted following cyclic groups

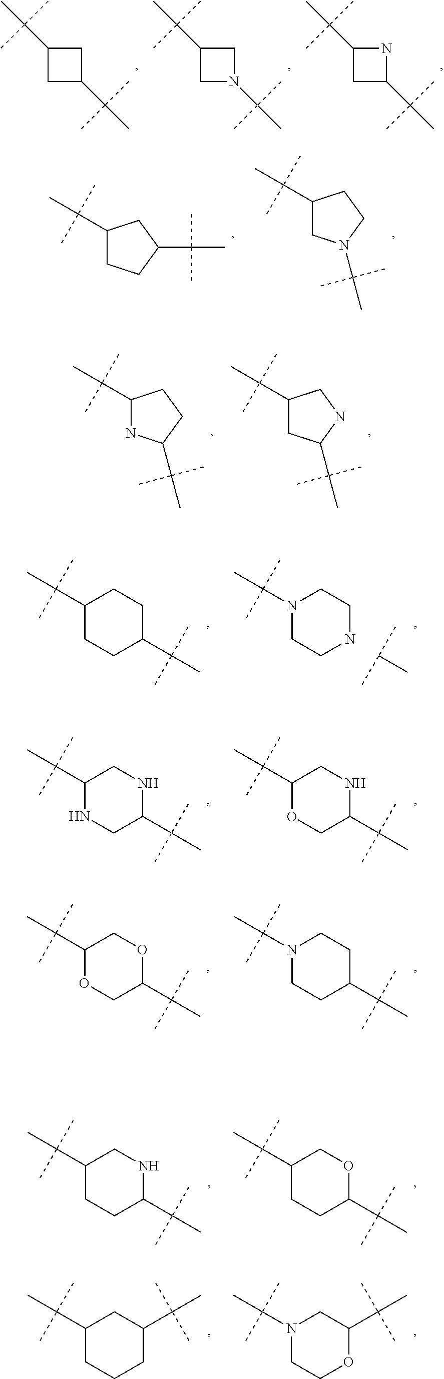

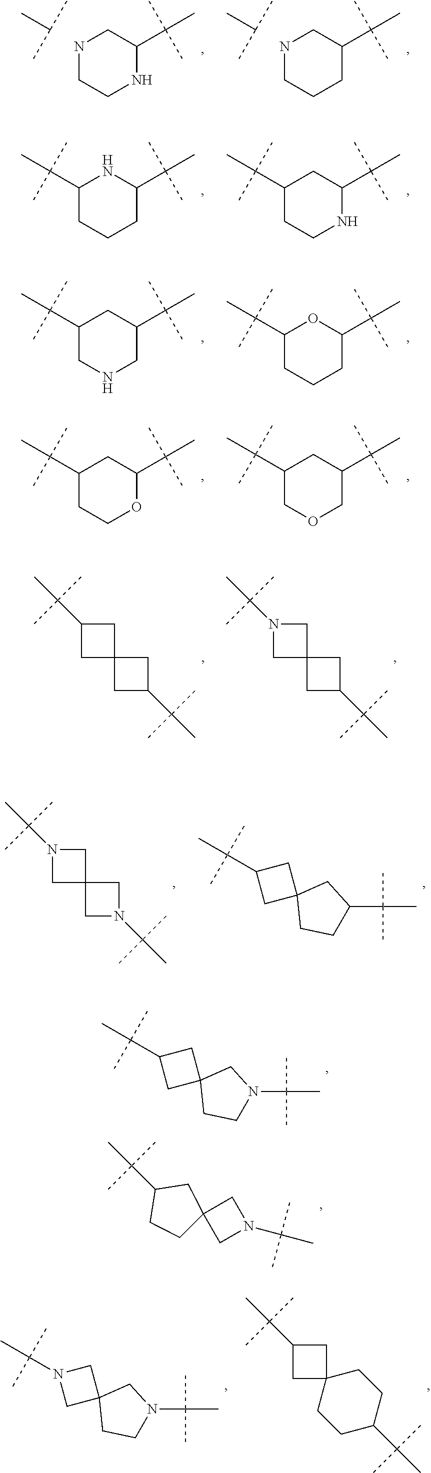

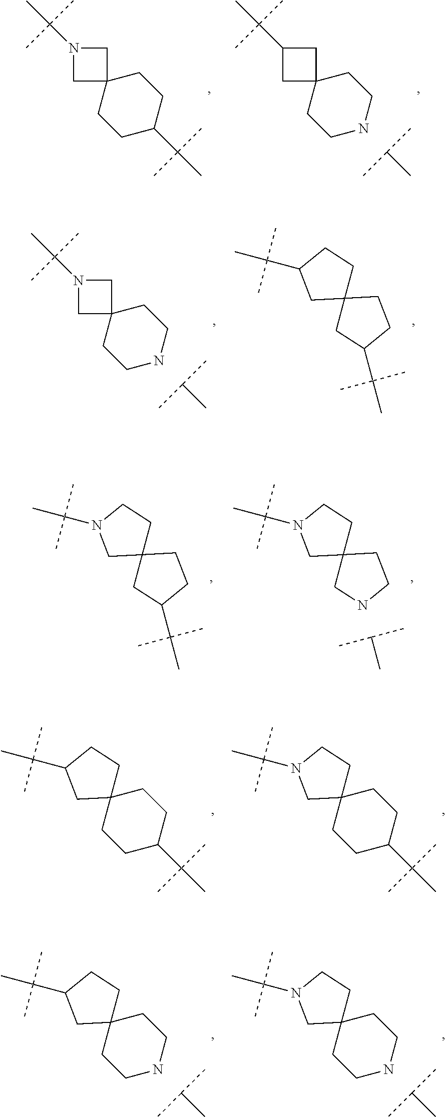

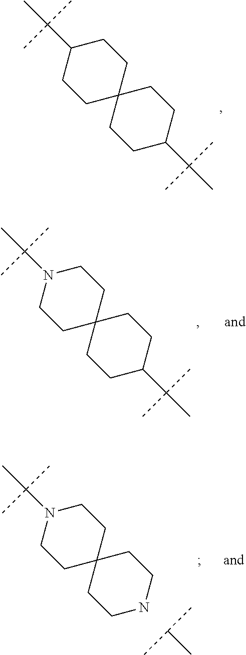

Group R_{L}^{r2} consists of optionally substituted following cyclic groups

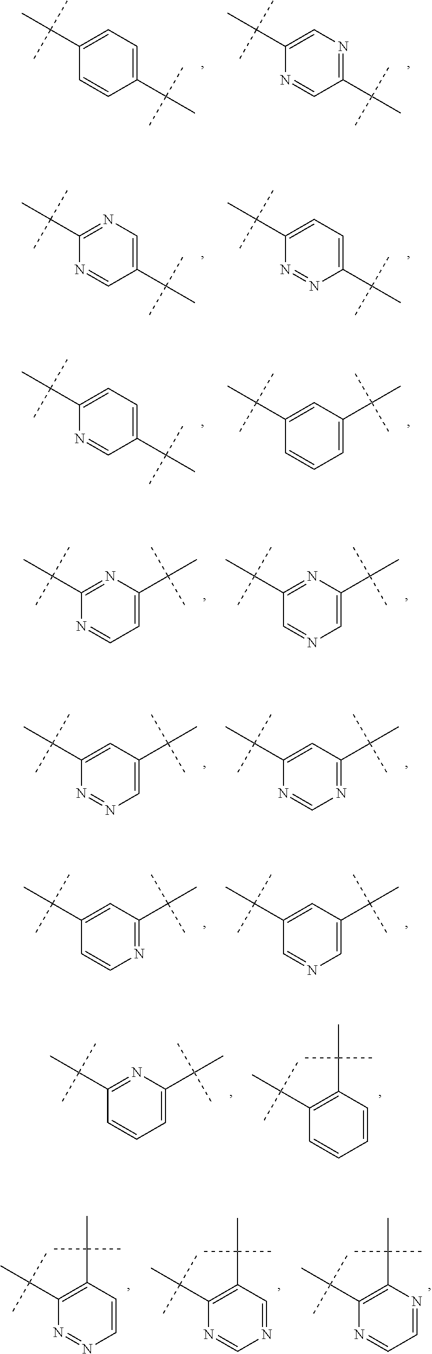

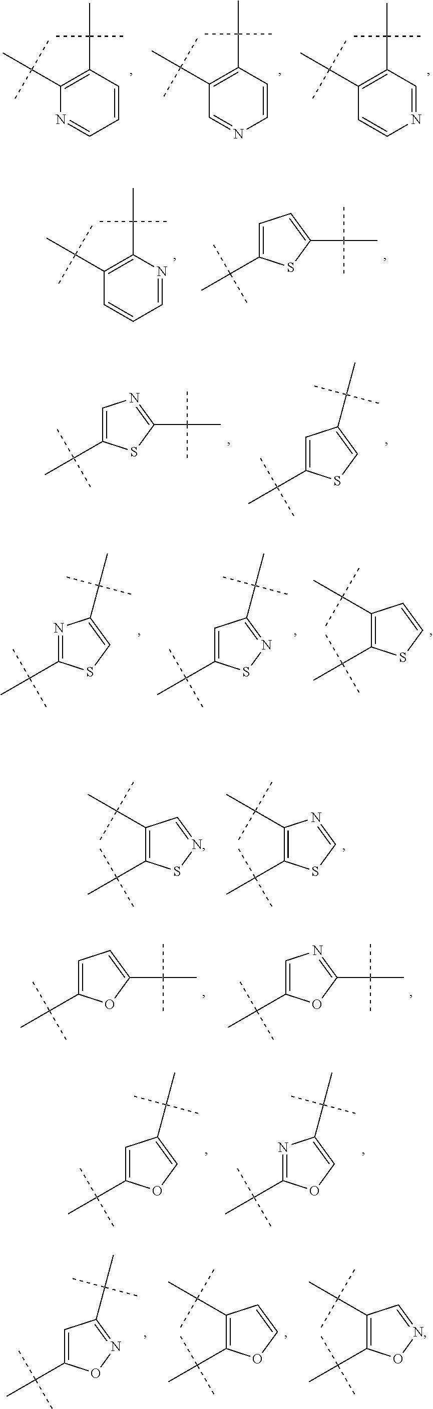

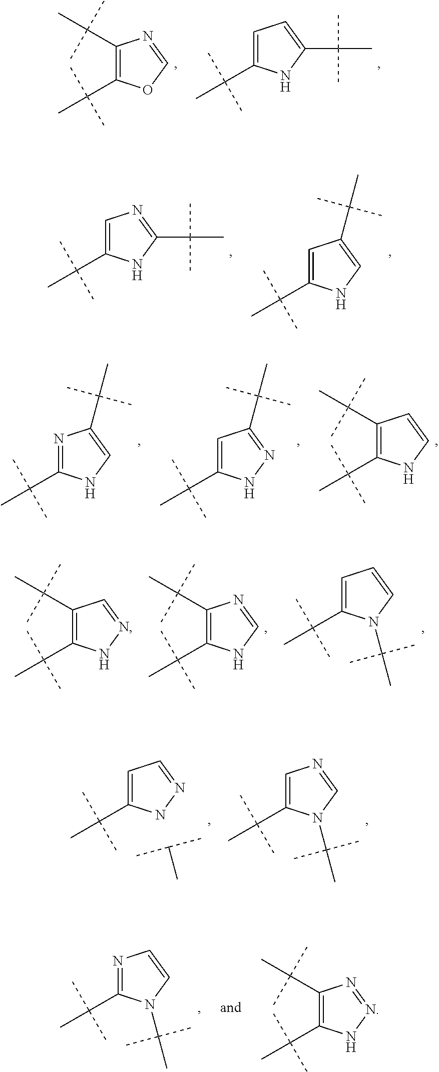

In one embodiment, the linker moiety is of FORMULA 9A.

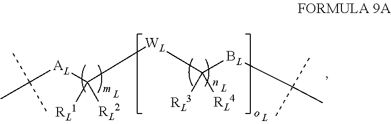

wherein
R_{L}¹, R_{L}², R_{L}³ and R_{L}⁴, at each occurrence, are independently selected from the group consisting of hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl, or
R_{L}¹ and R_{L}², R_{L}³ and R_{L}⁴ together with the atom(s) to which they are connected form a C₃-C₂₀ cycloalkyl or 3-20 membered heterocyclyl ring;
A_{L}, W_{L} and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}⁵)R_{L}^{e}, R_{L}^{d}C(S)N(R_{L}⁵)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁵)R_{L}^{e}, R_{L}^{d}N(R_{L}⁵)R_{L}^{e}, R_{L}^{d}N(R_{L}⁵)COR_{L}^{e}, R_{L}^{d}N(R_{L}⁵)CON(R_{L}⁶)R_{L}^{e}, R_{L}^{d}N(R_{L}⁵)C(S)R_{L}^{e}, optionally substituted C₁-C₈ alkylene, optionally substituted C₂-C₈ alkenylene, optionally substituted C₂-C₈ alkynylene, optionally substituted 2-8 membered heteroalkylene, optionally substituted 3-8 membered heteroalkenylene, optionally substituted 3-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ carbocyclyl, optionally substituted 3-13 membered heterocyclyl, optionally substituted aryl, and optionally substituted heteroaryl, wherein
R_{L}^{d} and R_{L}^{e}, at each occurrence, are independently selected from the group consisting of null, optionally substituted (C₁-C₈ alkyl)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted 2-8 membered heteroalkylene, optionally substituted 3-8 membered heteroalkenylene, optionally substituted 3-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl;
R_{L}^{r} is defined as in FORMULA 9;
R_{L}⁵ and R_{L}⁶, at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; or
R_{L}^{d} and R_{L}^{e}, R_{L}⁵ and R_{L}⁶, R_{L}^{d} and R_{L}⁵, R_{L}^{d} and R_{L}⁶, R_{L}^{e} and R_{L}⁵, R_{L}^{e} and R_{L}⁶ together with the atom(s) to which they are connected optionally form a C₃-C₂₀cycloalkyl or 3-20 membered heterocyclyl ring;
m_{L} is 0 to 15 (such as 0, 1, 2, 3, 4, 5, 6, 7, 8, 9, 10, 11, 12, 13, 14, or 15);
n_{L}, at each occurrence, is 0 to 15 (such as 0, 1, 2, 3, 4, 5, 6, 7, 8, 9, 10, 11, 12, 13, 14, or 15); and
o_{L} is 0 to 15 (such as 0, 1, 2, 3, 4, 5, 6, 7, 8, 9, 10, 11, 12, 13, 14, or 15).

In another embodiment, the linker moiety is of FORMULA 9B:

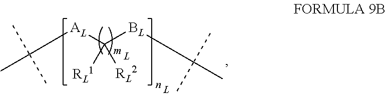

wherein
R_{L}¹ and R_{L}², at each occurrence, are independently selected from the group consisting of hydrogen, halogen, hydroxyl, amino, cyano, nitro, and optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl, or
R_{L}¹ and R_{L}² together with the atom(s) to which they are connected form a C₃-C₂₀cycloalkyl or 3-20 membered heterocyclyl ring;
A_{L} and B_{L}, at each occurrence, are independently null, or a bivalent moiety selected from the group consisting of R_{L}^{d}—R_{L}^{e}, R_{L}^{d}COR_{L}^{e}, R_{L}^{d}CO₂R_{L}^{e}, R_{L}^{d}C(O)N(R_{L}³)R_{L}^{e}, R_{L}^{d}C(S)N(R_{L}³)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}³)R_{L}, R_{L}^{d}N(R_{L}³)R_{L}^{e}, R_{L}^{d}N(R_{L}³)COR_{L}^{e}, R_{L}^{d}N(R_{L}³)CON(R_{L}⁴)R_{L}^{e}, R_{L}^{d}N(R_{L}³)C(S)R_{L}^{e}, optionally substituted C₁-C₈ alkylene, optionally substituted C₂-C₈ alkenylene, optionally substituted C₂—C₈ alkynylene, optionally substituted 2-8 membered heteroalkylene, optionally substituted 3-8 membered heteroalkenylene, optionally substituted 3-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ carbocyclyl, optionally substituted 3-13 membered heterocyclyl, optionally substituted aryl, and optionally substituted heteroaryl, wherein
R_{L}^{d} and R_{L}^{e}, at each occurrence, are independently selected from the group consisting of null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted 2-8 membered heteroalkylene, optionally substituted 3-8 membered heteroalkenylene, optionally substituted 3-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl;
R_{L}^{r} is defined as in FORMULA 9;
R_{L}³ and R_{L}⁴, at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; or
R_{L}^{d} and R_{L}^{e}, R_{L}³ and R_{L}⁴, R_{L}^{d} and R_{L}³, R_{L}^{d} and R_{L}⁴, R_{L}^{e} and R_{L}³, R_{L}^{e} and R_{L}⁴ together with the atom(s) to which they are connected optionally form a C₃-C₂₀ cycloalkyl or 3-20 membered heterocyclyl ring;
each m_{L} is 0 to 15 (such as 0, 1, 2, 3, 4, 5, 6, 7, 8, 9, 10, 11, 12, 13, 14, or 15); and
n_{L} is 0 to 15 (such as 0, 1, 2, 3, 4, 5, 6, 7, 8, 9, 10, 11, 12, 13, 14, or 15).

In another embodiment, the linker moiety is of FORMULA 9C:

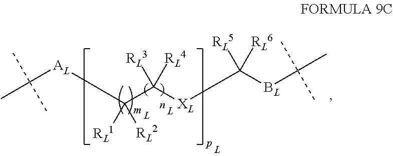

wherein
X_{L}, at each occurrence, is selected from O and NR_{L};
R_{L}¹, R_{L}², R_{L}³, R_{L}⁴, R_{L}⁵, and R_{L}⁶, at each occurrence, are independently selected from the group consisting of hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted C₃-C₁₀ carbocyclyl, optionally substituted C₃-C₁₀ cycloalkoxy, optionally substituted 3-10 membered heterocyclyl, optionally substituted aryl, and optionally substituted heteroaryl; A_{L} and B_{L}, at each occurrence, are independently selected from the group consisting of null, or bivalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}⁸)R_{L}^{e}, R_{L}^{d}C(S)N(R_{L}⁸)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁸)R_{L}^{e}, R_{L}^{d}N(R_{L}⁸)R_{L}^{e}, R_{L}^{d}N(R_{L}⁸)COR_{L}^{e}, R_{L}^{d}N(R_{L}⁸)CON(R_{L}⁹)R_{L}^{e}, R_{L}^{d}N(R_{L}⁸)C(S)R_{L}^{e}, optionally substituted C₁-C₈ alkylene, optionally substituted C₂-C₈ alkenylene, optionally substituted C₂-C₈ alkynylene, optionally substituted 2-8 membered heteroalkylene, optionally substituted 3-8 membered heteroalkenylene, optionally substituted 3-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted 4-13 membered fused cycloalkyl, optionally substituted 5-13 membered fused heterocyclyl, optionally substituted 5-13 membered bridged cycloalkyl, optionally substituted 5-13 membered bridged heterocyclyl, optionally substituted 5-13 membered spiro cycloalkyl, optionally substituted 5-13 membered spiro heterocyclyl, optionally substituted C₃-C₁₀ carbocyclyl, optionally substituted 3-10 membered heterocyclyl, optionally substituted aryl, and optionally substituted heteroaryl, wherein
R_{L}^{d} and R_{L}^{e}, at each occurrence, are independently selected from the group consisting of null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 3-8 membered heteroalkenylene, optionally substituted 3-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl;
R_{L}^{r} is defined as in FORMULA 9;
R_{L}⁷, R_{L}⁸ and R_{L}⁹, at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; or
R_{L}^{d} and R_{L}^{e}, R_{L}⁸ and R_{L}⁹, R_{L}^{d} and R_{L}⁸, R_{L}^{d} and R_{L}⁹, R_{L}^{e} and R_{L}⁸, R_{L}^{e} and R_{L}⁹ together with the atom(s) to which they are connected optionally form a C₃-C₂₀ cycloalkyl or 3-20 membered heterocyclyl ring;
m_{L}, at each occurrence, is 0 to 15 (such as 0, 1, 2, 3, 4, 5, 6, 7, 8, 9, 10, 11, 12, 13, 14, or 15);
n_{L}, at each occurrence, is 0 to 15 (such as 0, 1, 2, 3, 4, 5, 6, 7, 8, 9, 10, 11, 12, 13, 14, or 15);
o_{L} is 0 to 15 (such as 0, 1, 2, 3, 4, 5, 6, 7, 8, 9, 10, 11, 12, 13, 14, or 15); and
p_{L} is 0 to 15 (such as 0, 1, 2, 3, 4, 5, 6, 7, 8, 9, 10, 11, 12, 13, 14, or 15).

In another refinement, the length of the linker is 3 to 20 chain atoms.

In another refinement, 1) the TRK ligand is defined as any one of the embodiments above; 2) the degradation tag is a moiety of FORMULA 5; and 3) the linker moiety is of FORMULA 9.

In another refinement, the TRK ligand is defined as any one of the embodiments above; 2) the degradation tag is a moiety of FORMULA 5-1; and 3) the linker moiety is of FORMULA 9.

In another refinement, 1) the TRK ligand is defined as any one of the embodiments above; 2) the degradation tag is a moiety of FORMULA 5A; and 3) the linker moiety is of FORMULA 9.

In another refinement, 1) the TRK ligand is defined as any one of the embodiments above; 2) the degradation tag is a moiety selected from the group consisting of FORMULA 8A, 8B, 8C, 8D, 8E, 8F, 8G, 8H, 8I, 8J, 8K, 8L, 8M, 8N, 80, 8P, 8Q, 8R, 8S, 8T, 8U, 8V, 8W, 8X, 8Y, 8Z, 8AA, 8AB, 8AC, and 8AD; and 3) the linker moiety is of FORMULA 9.

In another refinement, 1) the TRK ligand is defined as any one of the embodiments above; 2) the degradation tag is a moiety of FORMULA 5 or a moiety of FORMULA 6A, 6B, or 6C; and 3) the linker moiety is of FORMULA 9, wherein A_{L} and B_{L}, at each occurrence, are independently selected from the group consisting of null, R_{L}^{d}—R_{L}^{e}, R_{L}^{d}COR_{L}^{e}, R_{L}^{d}C(O)N(R_{L}¹)R_{L}^{e}, R_{L}^{d}OR_{L}^{e}, R_{L}^{d}N(R_{L}¹)COR_{L}^{e}; R_{L}^{d} and R_{L}^{e}, at each occurrence, are independently selected from the group consisting of null, and optionally substituted C₁, C₂ or C₃ alkylene; one of W_{L}² and W_{L}¹ is null; and the other is optionally substituted C₁, C₂, or C₃ alkylene; and m_{L} is 1, 2, 3, 4, 5, 6, 7, 8, 9, or 10.

In another refinement, 1) the TRK ligand is defined as any one of the embodiments above; 2) the degradation tag is a moiety selected from the group consisting of FORMULA 8A, 8B, 8C, 8D, 8E, 8F, 8G, 8H, 8I, 8J, 8K, 8L, 8M, 8N, 80, 8P, 8Q, 8R, 8S, 8T, 8U, 8V, 8W, 8X, 8Y, 8Z, 8AA, 8AB, 8AC, and 8AD; and 3) the linker moiety is of FORMULA 9, A_{L} and B_{L}, at each occurrence, are independently selected from the group consisting of null, R_{L}^{d}—R_{L}^{e}, R_{L}^{d}COR_{L}^{e}, R_{L}^{d}C(O)N(R_{L}¹)R_{L}^{e}, R_{L}^{d}OR_{L}^{e}, R_{L}^{d}N(R_{L}¹)COR_{L}^{e}; R_{L}^{d} and R_{L}^{e}, at each occurrence, are independently selected from the group consisting of null, and optionally substituted C₁, C₂ or C₃ alkylene; one of W_{L}² and W_{L}¹ is null; and the other is optionally substituted C₁, alkylene; and m_{L} is 1, 2, 3, 4, 5, 6, 7, 8, 9, or 10.

In another embodiment, the heterobifunctional compound disclosed herein comprises a moiety of FORMULA I″:

M_{TRK}-M_{L}-M_{DT} (FORMULA I″),
or a pharmaceutically acceptable salt, hydrate, solvate, prodrug, stereoisomer, tautomer, or analog thereof, wherein M_{TRK} refers to the TRK allosteric ligand moiety; M_{L} refers to the linker moiety; and M_{DT} refers to the degradation tag moiety;
and wherein
i) the TRK allosteric ligand comprises a moiety of FORMULA 1″:

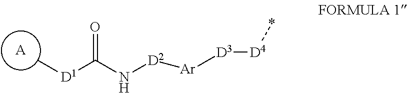

wherein
* indicates the connection to the linker moiety of the heterobifunctional compound;
D¹ is selected from null, —NH—, —O—, optionally substituted C₁-C₃ alkylene, optionally substituted C₁-C₃ alkylene-O—, or optionally substituted C₁-C₃ alkylene-NH—, and optionally substituted C₁-C₃ alkylene-N (optionally substituted C₁-C₈ alkyl)-;
D² is selected from null or optionally substituted C₁-C₃ alkylene;
D³ and D⁴ are independently selected from null, —O—, —S—, —NR¹—, —CO—, —CO₂—, —CONR¹—, —SO—, —SO₂—, —SO₂NR¹—, —NR¹CO—, —NR¹CO₂—, —NR¹C(O)NR²—, —NR¹SO—, —NR¹SO₂—, —NR¹SO₂NR²—, —OCO₂—, —OCONR¹—, optionally substituted C₁-C₈ alkylene, optionally substituted 1 to 8 membered heteroalkylene, optionally substituted C₁-C₈alkylene-O—C₁-C₈alkylene, optionally substituted C₁-C₈ haloalkylene, optionally substituted C₁-C₈ hydroxyalkylene, optionally substituted C₁-C₈alkylene-N(C₁-C₈alkyl)-C₁-C₈alkylene, optionally substituted C₃-C₈ cycloalkylene, optionally substituted 3-8 membered carbocyclyl, optionally substituted 3-8 membered heterocyclyl, optionally substituted 3-8 membered carbocyclyl-C₁-C₈alkylene, optionally substituted 3-8 membered heterocyclyl-C₁-C₈alkylene, optionally substituted 3-8 membered carbocyclyl-O—, optionally substituted 3-8 membered heterocyclyl-O, optionally substituted 3-8 membered carbocyclyl-N(C₁-C₈alkyl)-, and optionally substituted 3-8 membered heterocyclyl-N(C₁-C₈alkyl)-, optionally substituted C₂-C₈ alkenylene, optionally substituted C₂-C₈ alkynylene, optionally substituted 3-8 membered heteroalkenylene, optionally substituted 3-8 membered heteroalkynylene, optionally substituted aryl, and optionally substituted heteroaryl, wherein
R¹ and R² are independently selected from the group consisting of H, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₃-C₁₀ carbocyclyl, optionally substituted 3-10 membered heterocyclyl, optionally substituted aryl, and optionally substituted heteroaryl, or R¹ and R² together with the atom or atoms to which they are connected form a C₃-C₂₀ carbocyclyl ring or 3-20 membered heterocyclyl ring;
Ring A is selected from the group consisting of optionally substituted C₃-C₁₃ carbocyclyl, optionally substituted 3-13 membered heterocyclyl, optionally substituted aryl, and optionally substituted heteroaryl; and Ring A is optionally further substituted with Ar³;
Ar³ is selected from optionally substituted aryl, and optionally substituted heteroaryl; and
Ar is selected from the group consisting of optionally substituted aryl, and optionally substituted heteroaryl, and Ar is optionally further substituted with Ar¹;
Ar¹ is selected from optionally substituted aryl and optionally substituted heteroaryl; and/or
c) the degradation tag is a moiety of FORMULA 5:

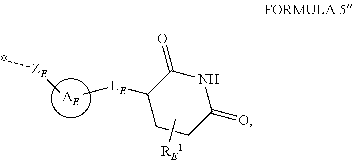

wherein
* indicates the connection to the linker moiety of the heterobifunctional compound;
Z_{E} is a divalent group of —(R_{E}^{z})_{nE}—; wherein subscript n_{E}=0, 1, 2, 3, 4, 5 or 6; wherein R_{E}^{z} at each occurrence, is independently R_{E}^{r}, or R_{E}^{w}; wherein R_{E}^{w}, at each occurrence, is a bond or selected from the group consisting of —CO—, —CR_{E}⁵R_{E}⁶—, —NR_{E}⁵—, —O—, optionally substituted C₁-C₁₀ alkylene, optionally substituted C₂-C₁₀ alkenylene, optionally substituted C₂-C₁₀ alkynylene, optionally substituted 2-10 membered heteroalkylene, optionally substituted 3-10 membered heteroalkenylene, optionally substituted 2-10 membered heteroalkynylene; and R_{E}^{r} at each occurrence, is a bond, or selected from the group consisting of optionally substituted C₃-C₁₃ carbocyclyl, optionally substituted 3-13 membered heterocyclyl, optionally substituted aryl, and optionally substituted heteroaryl; with the proviso that —R_{E}^{z}—R_{E}^{z}— is not —O—O—;
R_{E}⁵ and R_{E}⁶, at each occurrence, are independently selected from the group consisting of hydrogen, halogen, oxo, hydroxy, amino, cyano, nitro, optionally substituted C₁-C₆ alkyl, optionally substituted 1-6 membered heteroalkyl, optionally substituted 3 to 8 membered carbocyclyl, and optionally substituted 3 to 8 membered heterocyclyl; or R_{E}⁵ and R_{E}⁶ together with the atom(s) to which they are connected form an optionally substituted 3-8 membered cycloalkyl or heterocyclyl ring;
R_{E}¹ is selected from the group consisting of hydrogen, halogen, cyano, nitro, optionally substituted C₁-C₆ alkyl, optionally substituted 1-6 membered heteroalkyl, optionally substituted 3-8 membered carbocyclyl, and optionally substituted 3-8 membered heterocyclyl;
L_{E} is a divalent group selected from the group consisting of null, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰-, with the proviso that -L_{E}¹-L_{E}²- is not —O—O—; wherein R_{E}¹⁰ and R_{E}¹¹ are independently selected from the group consisting of hydrogen, halogen, cyano, nitro, hydroxy, amino, optionally substituted C₁-C₆ alkyl, optionally substituted 1-6 membered heteroalkyl, optionally substituted C₁-C₆ alkoxy, and optionally substituted C₁-C₆ alkylamino;
Ring A_{E} is a divalent group selected from the group consisting of FORMULA A_{E}1, A_{E}2, A_{E}3, A_{E}4 and A_{E}5:

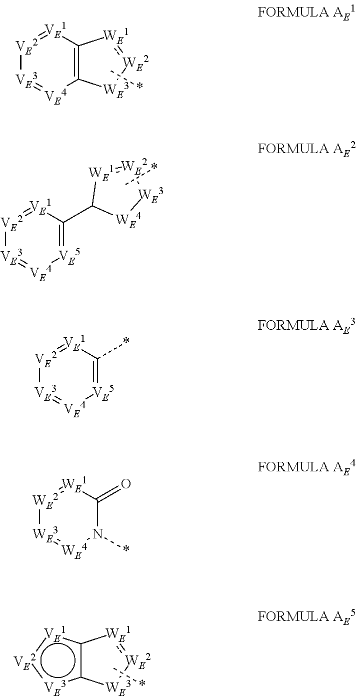

wherein
* indicates the attachment to L_{E}, and Z_{E} is attached to any possible position on the Ring A_{E};
[symbol] indicates a single bond or a double bond;
V_{E}¹, V_{E}², V_{E}³, V_{E}⁴ and V_{E}⁵, at each occurrence, are each independently selected from the group consisting of a bond, C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined together to optionally form C₆ aryl ring or a 5, 6 or 7 membered heteroaryl ring;
R_{E}², at each occurrence, is independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted 1-6 membered heteroalkyl, optionally substituted 3-6 membered heteroalkenyl, optionally substituted 3-6 membered heteroalkynyl, optionally substituted C₁-C₆ alkoxy, optionally substituted C₁-C₆ alkylamino, optionally substituted 3-8 membered cycloalkyl, and optionally substituted 3-8 membered heterocyclyl; or R_{E}² and another R_{E}² together with the atom(s) to which they are connected form optionally substituted 3-8 membered cycloalkyl, optionally substituted 3-8 membered heterocyclyl ring, optionally substituted aryl, and optionally substituted heteroaryl;
W_{E}¹, W_{E}², W_{E}³ and W_{E}⁴ are each independently selected from the group consisting of —N═, —C≡, —CR_{E}³=, —CO—, —O—, —CR_{E}³R_{E}⁴—, —NR_{E}³—, —CR_{E}³=CR_{E}⁴-, —N═CR_{E}³-, and —N═N—; or W_{E}¹ and W_{E}², W_{E}² and W_{E}³, or W_{E}³ and W_{E}⁴ are combined together to optionally form C₆ aryl ring or a 5, 6 or 7 membered heteroaryl ring;
R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted 1-6 membered heteroalkyl, optionally substituted 3-6 membered heteroalkenyl, optionally substituted 3-6 membered heteroalkynyl, optionally substituted 3 to 8 membered carbocyclyl, and optionally substituted 3 to 8 membered heterocyclyl; or R_{E}³ and R_{E}⁴, on the same atom or on the adjacent atoms, together with the atom(s) to which they are connected form an optionally substituted 3-8 membered cycloalkyl or heterocyclyl ring; or, the degradation tag is a moiety of FORMULAE 6A″, 6B″, or 6C″:

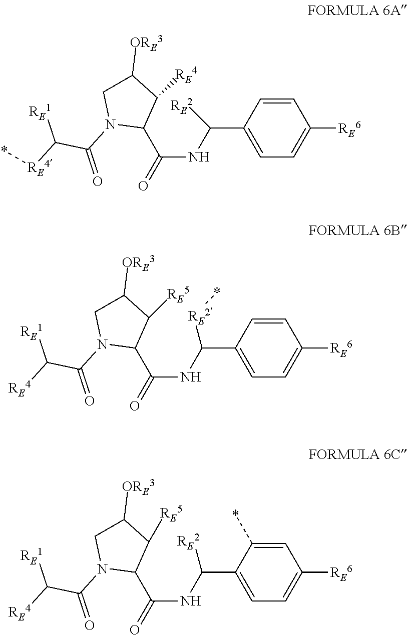

wherein
* indicates the connection to the linker moiety of the heterobifunctional compound; R_{E}¹ and R_{E}² are independently selected from the group consisting of hydrogen, hydroxyl, amino, cyano, nitro, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₁₀alkylaminoC₁-C₈alkyl, optionally substituted C₃-C₁₀ carbocyclyl, optionally substituted 3-10 membered heterocyclyl;
R_{E}²′ is a divalent group selected from the group consisting of null, O, NH, optionally substituted C₁-C₈ alkylene, optionally substituted C₂-C₈ alkenylene, optionally substituted C₂-C₈ alkynylene, optionally substituted 1-8 membered heteroalkylene, optionally substituted 3-8 membered heteroalkenylene, optionally substituted 3-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₁-C₈ aminoalkylene, optionally substituted C₁-C₈alkylaminoC₁-C₈alkylene, optionally substituted C₃-C₁₀ carbocyclyl, and optionally substituted 3-10 membered heterocyclyl;
R_{E}³ is selected from the group consisting of hydrogen, optionally substituted —C(O)R_{E}⁷, —C(O)OR_{E}⁷, —C(O)NR_{E}⁷R_{E}⁸, —P(O)(OR_{E}⁷)², and —CR_{E}⁷R_{E}⁸—OP(O)(OR_{E}⁹)², wherein
R_{E}⁷, R_{E}⁸ and R_{E}⁹ are independently selected from the group consisting of hydrogen, optionally substituted C₁-C₈ alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl and optionally substituted heteroaryl;
R_{E}⁴ is selected from the group consisting of —N(R_{E}¹⁰)R_{E}¹, —OR_{E}¹⁰, —N(R_{E}¹⁰)C(O)R_{E}¹¹, optionally substituted C₃-C₁₀ carbocyclyl, optionally substituted 3-10 membered heterocyclyl, optionally substituted aryl, and optionally substituted heteroaryl;
R_{E}⁴′ is a divalent group selected from the group consisting of —N(R_{E}¹⁰)—, —O—, —N(R_{E}¹⁰)C(O)R_{E}¹¹-, optionally substituted C₃-C₁₀ carbocyclyl, optionally substituted 3-10 membered heterocyclyl, optionally substituted aryl, and optionally substituted heteroaryl;
R_{E}¹⁰ is selected from the group consisting of hydrogen, optionally substituted C₁-C₈alkyl, optionally substituted 1-8 membered heteroalkyl, optionally substituted C₃-C₈cycloalkyl, optionally substituted C₁-C₈alkyl-CO, optionally substituted C₁-C₈cycloalkyl-CO, optionally substituted C₃-C₈cycloalkyl-C₁-C₈alkyl-CO, optionally substituted 3-10 membered heterocyclyl-CO, optionally substituted 3-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted C₃-C₁₀ carbocyclyl, optionally substituted 3-10 membered heterocyclyl, optionally substituted aryl, and optionally substituted heteroaryl;
R_{E}¹¹ is selected from the group consisting of hydrogen, optionally substituted C₁-C₈alkyl, optionally substituted 1-8 membered heteroalkyl, optionally substituted C₃-C₈cycloalkyl, optionally substituted 3-8 membered heterocycloalkyl, optionally substituted C₃-C₈ carbocyclyl, and optionally substituted 3-8 membered heterocyclyl;
R_{E}^{11′}, at each occurrence, is a divalent group independently selected from the group consisting of null, O, optionally substituted C₁-C₈alkylene, optionally substituted 1-8 membered heteroalkylene, optionally substituted C₃-C₈ cycloalkylene, optionally substituted 3-8 membered heterocycloalkylene, optionally substituted C₃-C₈ carbocyclyl, and optionally substituted 3-8 membered heterocyclyl;
R_{E}⁵ is selected from the group consisting of hydrogen and halogen; and
R_{E}⁶ is selected from hydrogen, halogen, hydroxyl, amino, cyano, nitro, optionally substituted C₁-C₈alkyl, optionally substituted C₂-C₈ alkenyl, optionally substituted C₂-C₈ alkynyl, optionally substituted 1-8 membered heteroalkyl, optionally substituted 3-8 membered heteroalkenyl, optionally substituted 3-8 membered heteroalkynyl, optionally substituted C₃-C₈cycloalkyl, optionally substituted C₁-C₈alkoxy, and optionally substituted C₃-C₈cycloalkoxy, optionally substituted 3-8 membered heterocycloalkyl, optionally substituted C₃-C₈ carbocyclyl, optionally substituted 3-8 membered heterocyclyl, optionally substituted aryl, and optionally substituted heteroaryl;
and/or
3) the linker moiety is of FORMULA 9:

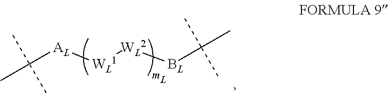

wherein
A_{L}, W_{L}¹, W_{L}², and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted 1-8 membered heteroalkylene, optionally substituted 3-8 membered heteroalkenylene, optionally substituted 3-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl, wherein
R_{L}^{d} and R_{L}^{e}, at each occurrence, are independently selected from null, R_{L}^{r}, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 3-8 membered heteroalkenylene, optionally substituted 3-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted C₃-C₁₃ carbocyclyl, optionally substituted 3-13 membered heterocyclyl, optionally substituted aryl, and optionally substituted heteroaryl;
R_{L}^{r}, at each occurrence, is selected from optionally substituted C₃-C₁₀ carbocyclyl, optionally substituted 3-10 membered heterocyclyl, optionally substituted aryl, and optionally substituted heteroaryl;
R_{L}¹ and R_{L}², at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted 1-8 membered heteroalkylene, optionally substituted 3-8 membered heteroalkenylene, optionally substituted 3-8 membered heteroalkynylene, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; or
R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are connected optionally form a C₃-C₂₀ carbocyclyl or 3-20 membered heterocyclyl ring; and
m_{L} is 1, 2, 3, 4, 5, 6, 7, 8, 9, 10, 11, 12, 13, 14, or 15.

In another embodiment, in FORMULA 1″, D¹, D², D³, D⁴, D¹, Ar, Ar¹, Ring A is defined as in FORMULA 1

In another embodiment, in FORMULA 5″, Z_{E}, A_{E}, L_{E}, and R_{E}¹ are as in FORMULA 5;

In another embodiment, in FORMULA 6A″, 6B″, and 6C″, R_{E}¹ is defined as R_{6E}¹, R_{E}² is defined as R_{6E}², R_{E}³ is defined as R_{6E}³, R_{E}⁴ is defined as R_{6E}⁴, R_{E}⁵ is defined as R_{6E}⁵, R_{E}⁶ is defined as R_{6E}⁶, R_{E}²′ is null, and R_{E}⁴′ is NH;

In another embodiment, in FORMULA 9″, A_{L}, W_{L}¹, W_{L}² and B_{L} are defined as in FORMULA 9.

Without wishing to be bound by any particular theory, it is contemplated herein that, in some embodiments, attaching VHL-1 or pomalidomide to either portion of the molecule can recruit the VHL E3 ligase or cereblon E3 ligase to TRK.

The heterobifunctional compounds disclosed herein can selectively affect TRK-mediated disease cells compared to WT (wild type) cells (i.e., an heterobifunctional compound able to kill or inhibit the growth of an TRK-mediated disease cell while also having a relatively low ability to lyse or inhibit the growth of a WT cell), e.g., possess a GI₅₀ for one or more TRK-mediated disease cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the TRK-mediated disease cells.

In some aspects, provided herein is a method for identifying a heterobifunctional compound which mediates degradation or reduction of TRK, the method comprising: providing a heterobifunctional test compound comprising an TRK ligand conjugated to a degradation tag through a linker; contacting the heterobifunctional test compound with a cell comprising a ubiquitin ligase and TRK; determining whether TRK level is decreased in the cell; and identifying the heterobifunctional test compound as a heterobifunctional compound which mediates degradation or reduction of TRK. In certain embodiments, the cell is a cancer cell. In certain embodiments, the cancer cell is a TRK-mediated cancer cell.

### Synthesis and Testing of Heterobifunctional Compounds

The binding affinity of novel synthesized heterobifunctional compounds can be assessed using standard biophysical assays known in the art (e.g., isothermal titration calorimetry (ITC), surface plasmon resonance (SPR)). Cellular assays can then be used to assess the heterobifunctional compound's ability to induce TRK degradation and inhibit cancer cell proliferation. Besides evaluating a heterobifunctional compound's induced changes in the protein levels of TRK, TRK mutants, TRK deletions, or TRK fusion proteins, protein-protein interaction or kinase enzymatic activity can also be assessed. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable cell lines for use in any or all of these steps are known in the art and include KM12 cells. Suitable mouse models for use in any or all of these steps are known in the art and include subcutaneous xenograft models, orthotopic models, patient-derived xenograft models, and patient-derived orthotopic models.

By way of non-limiting example, detailed synthesis protocols are described in the Examples for specific exemplary heterobifunctional compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (3H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂₀ in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

### Characterization of Exemplary Heterobifunctional Compounds

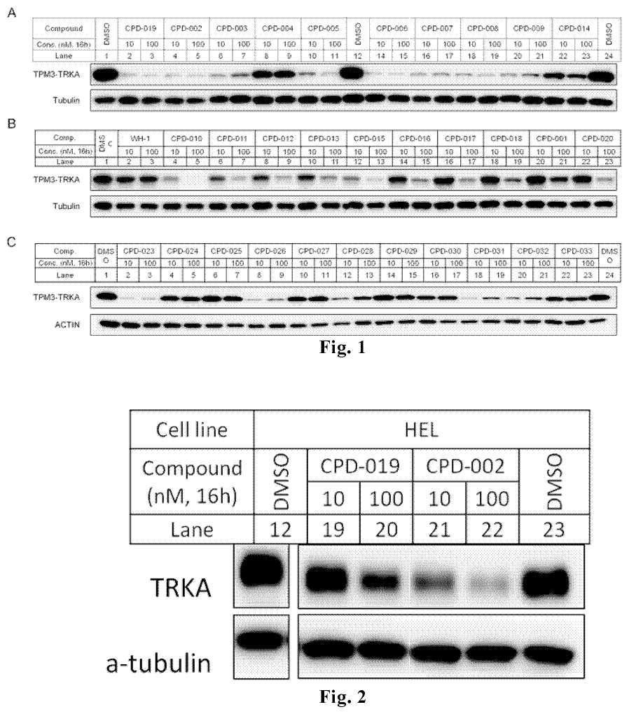

Specific exemplary heterobifunctional compounds were characterized in KM12 cells. KM12 cells that express TPM3-TRKA fusion protein were treated with 10 nM or 100 nM heterobifunctional compounds disclosed herein or their corresponding warheads/inhibitors for 16 hours. Cells were collected, lysed and subject to immunoblotting using an antibody specific to TRK proteins. Following a 16-hour treatment of various heterobifunctional compounds, TPM3-TRKA levels in KM12 cells were significantly decreased (FIG. 1). Furthermore, heterobifunctional compounds, exemplified in FIG. 2, also showed ability to degrade wildtype TRKA in HEL cells (FIG. 2).

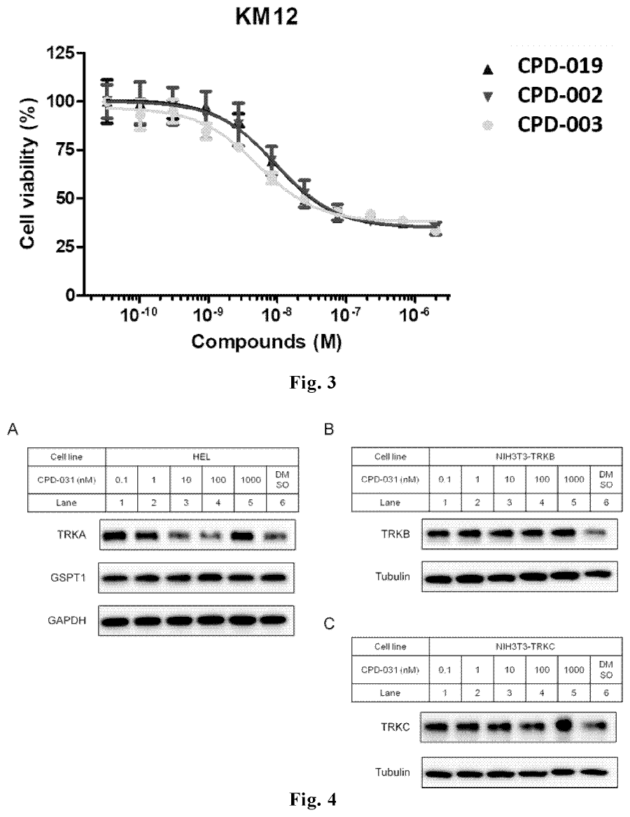

The kinase activity of TRK is known to play important roles in tumors expressing TRK-fusion proteins, as TRK kinase inhibitors compromise cell proliferation, survival, and induce marked clinical responses (Amatu et al., 2016; Drilon et al., 2018; Drilon et al., 2017; Khotskaya et al., 2017). KM12 cells seeded in 96-well plates were treated with heterobifunctional compounds following a 11-point 3-fold serial dilution. Three days after treatment, cell viability was determined using the CellTiter-Glo kit, and normalized to the mean values of 3 replicates of untreated cells. Dose-dependent response was analyzed following the least-squares non-linear regression method using the GraphPad Prism software. Each data point in the figure represents the mean values of three technical replicates ±standard deviation. Heterobifunctional compounds dose-dependently suppressed viability of TPM3-TRKA-expressing KM12 cells, as exemplified in FIG. 3. The vast majority of tested heterobifunctional compounds showed IC₅₀ values smaller than 1000 nM in KM12 cells (Table 2).

### DEFINITION OF TERMS

As used herein, the terms “comprising” and “including” are used in their open, non-limiting sense.

As used herein, the term “heterobifunctional compound(s)” and “bivalent compound(s)” can be used interchangeably.

As used herein, the terms “Tropomyosin Receptor Kinase ligand” and “TRK ligand”, or “TRK targeting moiety” are to be construed to encompass any molecules ranging from small molecules to large proteins that associate with or bind to TRK proteins. The TRK ligand is capable of binding to a TRK protein comprising TRK, a TRK mutant, a TRK deletion, or a TRK fusion protein. The TRK ligand can be, for example but not limited to, a small molecule 5 compound (i.e., a molecule of molecular weight less than about 1.5 kilodaltons (kDa)), a peptide or polypeptide, a nucleic acid or oligonucleotide, a carbohydrate such as an oligosaccharide, or an antibody or fragment thereof.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In certain embodiments, an alkyl comprises 1 to 6 carbon atoms (e.g., C₁-C₆ alkyl) or an alkyl comprises 1 to 4 carbon atoms (e.g., C₁-C₄ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond. For example, the alkyl includes methyl (Me), ethyl (Et), n-propyl (nPr), 1-methylethyl (iso-propyl, iPr), n-butyl, n-pentyl, 1,1-dimethylethyl (t-butyl), pentyl, 3-methylhexyl, 2-methylhexyl, and the like. Unless stated otherwise specifically in the specification, an alkyl group is optionally substituted by one or more of the following substituents: halo, cyano, nitro, oxo, thioxo, imino, oximo, trimethylsilanyl, R^{m}, —OR^{m}, —SR^{m}, —OC(O)—R^{m}, —N(R^{m})₂, —C(O)R^{m}, —C(O)OR^{m}, —C(O)N(R^{m})₂, —N(R^{m})C(O)OR^{o}, —OC(O)—N(R^{m})₂, —N(R^{m})C(O)R^{o}, —N(R^{m})S(O)₂R^{o} (where t is 1 or 2), —S(O)₂OR^{m} (where t is 1 or 2), —S(O)₂R^{m} (where t is 1 or 2) and —S(O)₂N(R^{m})₂ (where t is 1 or 2) where each R^{m} and each R^{o} are independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond. For example, the alkenyl includes ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, R^{m}, —OR^{m}, —SR^{m}, —OC(O)—R^{m}, —N(R^{m})₂, —C(O)R^{m}, —C(O)OR^{m}, —C(O)N(R^{m})₂, —N(R^{m})C(O)OR^{o}, —OC(O)—N(R^{m})₂, —N(R^{m})C(O)R^{o}, —N(R^{m})S(O)₂R^{o} (where t is 1 or 2), —S(O)₂OR^{m} (where t is 1 or 2), —S(O)₂R^{m} (where t is 1 or 2) and —S(O)₂N(R^{m})₂ (where t is 1 or 2) where each R^{m} and each R^{o} are independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like. Unless stated otherwise specifically in the specification, an alkynyl group is optionally substituted by one or more of the following substituents: halo, cyano, nitro, oxo, thioxo, imino, oximo, trimethylsilanyl, R^{m}, —OR^{m}, —SR^{m}, —OC(O)—R^{m}, —N(R^{m})₂, —C(O)R^{m}, —C(O)OR^{m}, —C(O)N(R^{m})₂, —N(R′)C(O)OR^{o}, —OC(O)—N(R^{m})₂, —N(R^{m})C(O)R^{o}, —N(R^{m})S(O)₂R^{o} (where t is 1 or 2), —S(O)₂OR^{m} (where t is 1 or 2), —S(O)₂R^{m}(where t is 1 or 2) and —S(O)₂N(R^{m})₂ (where t is 1 or 2) where each R^{m} and each R^{o} are independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “haloalkyl”, as used herein, means an alkyl group that is substituted by one or more halogens. Exemplary haloalkyl groups include trifluoromethyl, difluoromethyl, trichloromethyl, 2,2,2 trifluoroethyl, 1,2 difluoroethyl, 3 bromo 2 fluoropropyl, and 1,2 dibromoethyl.

The term “heteroalkyl”, “heteroalkenyl” or “heteroalkynyl”, as used herein, respectively means an alkyl, alkenyl or alkynyl group as defined above in which one or more carbon atoms have been independently replaced with heteroatoms, respectively. Exemplary heteroatoms include, e.g., O, N, P, Si, S, or combinations thereof, wherein the nitrogen, phosphorus, and sulfur atoms may optionally be oxidized and the nitrogen heteroatom may optionally be quaternized. If given, a numerical range refers to the total number of atoms, including carbon atoms not being replaced and those being replaced. For example, a 3- to 8-membered heteroalkyl means C₃₋₈ alkyl (containing 3 to 8 carbon atoms) in which one or more carbon atoms being replaced with atoms other than carbon. Connection of heteroalkyl, heteroalkenyl or heteroalkynyl to the rest of the molecule may be through either a heteroatom or a carbon in the heteroalkyl, heteroalkenyl or heteroalkynyl group. Unless stated otherwise specifically in the specification, the heteroalkyl, heteroalkenyl, or heteroalkynyl group is optionally substituted by one or more substituents such as those substituents described herein (such as those substituents for alkyl).

The term “aryl”, as used herein, refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl or 6-14 membered aryl). In certain embodiments, an aryl comprises six to ten carbon atoms (C₆-C₁₀ aryl or 6-10 membered aryl). Examples of such groups include, but are not limited to, phenyl, fluorenyl and naphthyl. The terms “Ph” and “phenyl,” as used herein, mean a —C₆H₅ group. Unless stated otherwise specifically in the specification, the term “aryl” or the prefix “ar-” (such as in “aralkyl”) is meant to include aryl radicals optionally substituted by one or more substituents independently selected from the group consisting of alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{m}, —R^{o}—OR^{m}, —R^{o}—OC(O)—R^{m}, —R^{o}—OC(O)—OR^{m}, —R^{o}—OC(O)—N(R^{m})₂, —R^{o}—N(R^{m})₂, —R^{o}—C(O)R^{m}, —R^{o}—C(O)OR^{m}, —R^{o}—C(O)N(R^{m})₂, —R^{o}—O—R^{p}—C(O)N(R^{m})₂, —R^{o}—N(R^{m})C(O)OR^{m}, —R^{o}—N(R^{m})C(O)R^{m}, —R^{o}—N(R^{m})S(O)₂R^{m}(where t is 1 or 2), —R^{o}—S(O)₂R^{m}(where t is 1 or 2), —R^{o}—S(O)₂OR^{m} (where t is 1 or 2) and —R^{o}—S(O)₂N(R^{m})₂ (where t is 1 or 2), where each R^{m} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{o} is independently a direct bond or a straight or branched alkylene or alkenylene chain, and R^{p} is a straight or branched alkylene or alkenylene chain, and where each of the above substituents is unsubstituted unless otherwise indicated.

The term “heteroaryl”, refers to a radical derived from a 3- to 18-membered aromatic ring radical (i.e. 3-18 membered heteroaryl) that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In certain embodiments, a heteroaryl refers to a radical derived from a 3- to 10-membered aromatic ring radical (3-10 membered heteroaryl). In certain embodiments, a heteroaryl refers to a radical derived from 5-, or 6-membered aromatic ring (5, or 6 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, a heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{m}, —R^{o}—OR^{m}, —R^{o}—OC(O)—R^{m}, —R^{o}—OC(O)—OR^{m}, —R^{o}—OC(O)—N(R′)₂, —R^{o}—N(R′)₂, —R^{o}—C(O)R^{m}, —R^{o}—C(O)OR^{m}, —R^{o}—C(O)N(R′)₂, —R^{o}—O—R^{p}—C(O)N(R′)₂, —R^{o}—N(R^{m})C(O)OR^{m}, —R^{o}—N(R^{m})C(O)R^{m}, —R^{o}—N(R^{m})S(O)₂R^{m} (where t is 1 or 2), —R^{o}—S(O)₂R^{m} (where t is 1 or 2), —R^{o}—S(O)₂OR^{m} (where t is 1 or 2) and —R^{o}—S(O)₂N(R^{m})₂ (where t is 1 or 2), where each R^{m} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{o} is independently a direct bond or a straight or branched alkylene or alkenylene chain, and R^{p} is a straight or branched alkylene or alkenylene chain, and where each of the above substituents is unsubstituted unless otherwise indicated.

The term “heterocyclyl”, as used herein, means a non-aromatic, monocyclic, bicyclic, tricyclic, or tetracyclic radical having a total of 4, 5, 6, 7, 8, 9, 10, 11, 12, 13, 14, 15, or 16 atoms in its ring system, and containing from 3 to 12 (such as 3, 4, 5, 6, 7, 8, 9, 10, 11, or 12)carbon atoms and from 1 to 4 (such as 1, 2, 3 or 4) heteroatoms each independently selected from the group consisting of O, S and N, and with the proviso that the ring of said group does not contain two adjacent 0 atoms or two adjacent S atoms. A heterocyclyl group may include fused, bridged or spirocyclic ring systems. In certain embodiments, a heterocyclyl group comprises 3 to 10 ring atoms (3-10 membered heterocyclyl). In certain embodiments, a heterocyclyl group comprises 3 to 8 ring atoms (3-8 membered heterocyclyl). In certain embodiments, a heterocyclyl group comprises 3 to 10 ring atoms (3-10 membered heterocyclyl). In certain embodiments, a heterocyclyl group comprises 3 to 8 ring atoms (3-8 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached). Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{m}, —R^{o}—OR^{m}, —R^{o}—OC(O)—R^{m}, —R^{o}—OC(O)—OR^{m}, —R^{o}—OC(O)—N(R^{m})₂, —R^{o}—N(R^{m})₂, —R^{o}—C(O)R^{m}, —R^{o}—C(O)OR^{m}, —R^{o}—C(O)N(R^{m})₂, —R^{o}—O—R^{p}—C(O)N(R^{m})₂, —R^{o}—N(R^{m})C(O)OR^{m}, —R^{o}—N(R^{m})C(O)R^{m}, —R^{o}—N(R^{m})S(O)₂R^{m}(where t is 1 or 2), —R^{o}—S(O)₂R^{m}(where t is 1 or 2), —R^{o}—S(O)₂OR^{m} (where t is 1 or 2) and —R^{o}—S(O)₂N(R^{m})₂ (where t is 1 or 2), where each R^{m} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{o} is independently a direct bond or a straight or branched alkylene or alkenylene chain, and R^{p} is a straight or branched alkylene or alkenylene chain, and where each of the above substituents is unsubstituted unless otherwise indicated.

The term “cycloalkyl” or “carbocyclyl” means a saturated, monocyclic, bicyclic, tricyclic, or tetracyclic radical having a total of from 3, 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 10 carbon ring atoms (3-10 membered or C₃-C₁₀ cycloalkyl). In certain embodiments, a cycloalkyl comprises 3 to 8 carbon ring atoms (3-8 membered or C₃-C₈carbocyclyl). In certain embodiments, a cycloalkyl comprises 3, 4, 5, 6, or 7 carbon ring atoms (i.e. C₁, C₂, C₃, C₄, C₅, C₆ or C₇ carbocyclyl). Examples of such groups include, but are not limited to, cyclopropyl(cPr), cyclobutyl, cyclopentyl, cyclopentenyl, cyclohexyl, cycloheptyl, adamantyl, and the like. Unless otherwise stated specifically in the specification, the term “carbocyclyl” is meant to include carbocyclyl radicals that are optionally substituted by one or more substituents independently selected from the group consisting of alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{m}, —R^{o}—OR^{m}, —R^{o}—OC(O)—R^{m}, —R^{o}—OC(O)—OR^{m}, —R^{o}—OC(O)—N(R^{m})₂, —R^{o}—N(R^{m})₂, —R^{o}—C(O)R^{m}, —R^{o}—C(O)OR^{m}, —R^{o}—C(O)N(R^{m})₂, —R^{o}—O—R^{p}—C(O)N(R^{m})₂, —R^{o}—N(R^{m})C(O)OR^{m}, —R^{o}—N(R^{m})C(O)R^{m}, —R^{o}—N(R^{m})S(O)₂R^{m} (where t is 1 or 2), —R^{o}—S(O)₂R^{m} (where t is 1 or 2), —R^{o}—S(O)₂OR^{m} (where t is 1 or 2) and —R^{o}—S(O)₂N(R^{m})₂ (where t is 1 or 2), where each R^{m} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{o} is independently a direct bond or a straight or branched alkylene or alkenylene chain, and R^{p} is a straight or branched alkylene or alkenylene chain, and where each of the above substituents is unsubstituted unless otherwise indicated.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alkyl-amino group (such as —CH₂(NH₂)), as defined herein.

An “alkylamino” group refers to an -amino-alkyl group (such as —NH(CH₃)), as defined herein.

An “cycloalkylamino” group refers to an -amino-cycloalkyl group (such as

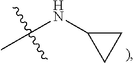 [SMILES: *NC1CC1]

as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “alkylene” is a bidentate radical obtained by removing a hydrogen atom from an alkyl group as defined above. Examples of such groups include, but are not limited to, —CH₂—, —CH₂CH₂—, etc. The term “cycloalkylene” or “carbocyclylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like. Similarly, the terms “alkenylene”, “alkynylene”, “alkoxyalkylene”, “haloalkylene”, “hydroxyalkylene”, “aminoalkylene”, “alkylaminoalkylene”, and “heterocyclylene”, “heteroalkylene”, “heteroalkenylene” or “heteroalkynylene” are bidentate radicals obtained by removing a hydrogen atom from an alkenyl radical, an alkynyl radical, an alkoxyalkyl radical, a haloalkyl radical, an hydroxyalkyl radical, aminoalkyl radical, and an alkylaminoalkyl radical, heteroalkyl radical, heteroalkenyl radical and heteroalkynyl radical, respectively. Unless stated otherwise specifically in the specification, an alkylene chain is optionally substituted by one or more of the following substituents: halo, cyano, nitro, oxo, thioxo, imino, oximo, trimethylsilanyl, R^{m}, —OR^{m}, —SR^{m}, —OC(O)—R^{m}, —N(R^{m})₂, —C(O)R^{m}, —C(O)OR^{m}, —C(O)N(R^{m})₂, —N(R^{m})C(O)OR^{m}, —OC(O)—, N(R^{m})₂, —N(R′)C(O)R^{m}, —N(R^{m})S(O)₂R^{m}(where t is 1 or 2), —S(O)₂OR^{m} (where t is 1 or 2), —S(O)₂R^{m} (where t is 1 or 2) and —S(O)₂N(R^{m})₂ (where t is 1 or 2) where each R^{m} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

The term “length” when refers to a moiety means the smallest number of carbon and/or hetero atoms from one end to the other end of the moiety. When it refers to the linker, it means the smallest number of atoms from the end connects to the TRK ligand and the end connects to the degradation tag. It applies to both situations where the linker is linear or branched, and where the linker comprises a ring system.

The term “substituted”, unless otherwise stated, means that the specified group or moiety bears one or more substituents independently selected from the group consisting of C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO2NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies. Unless otherwise specified, an optionally substituted radical may be a radical unsubstituted or substituted with one or more substituents selected from halogen, CN, NO₂, OR^{m}, SR^{m}, NRⁿR^{o}, COR^{m}, CO₂R^{m}, CONRⁿR^{o}, SOR^{m}, SO₂R^{m}, SO₂NRⁿR^{o}, NRⁿCOR^{o}, NR^{m}C(O)NRⁿR^{o}, NRⁿSOR^{o}, NRⁿSO₂R^{o}, C₁-C₈ alkyl, C₁-C₈alkoxyC₁-C₈alkyl, C₁-C₈ haloalkyl, C₁-C₈ hydroxyalkyl, C₁-C₈alkylaminoC₁-C₈ alkyl, C₃-C₇ cycloalkyl, 3-7 membered heterocyclyl, C₂-C₈ alkenyl, C₂-C₈ alkynyl, aryl, and heteroaryl, wherein R^{m}, Rⁿ, and R^{o} are independently selected from the group consisting of null, hydrogen, C₁-C₈ alkyl, C₂-C₈ alkenyl, C₂-C₈ alkynyl, C₃-C₇ cycloalkyl, 3-7 membered heterocyclyl, aryl, and heteroaryl, or Rⁿ and R^{o} together with the atom to which they are connected form a C₃-C₈ cycloalkyl or 3-8 membered heterocyclyl ring.

As used herein, the same symbol in different FORMULA means different definition, for example, the definition of R1 in FORMULA 1 is as defined with respect to FORMULA 1 and the definition of R1 in FORMULA 6 is as defined with respect to FORMULA 6.

As used herein each unit in the linker moiety (e.g., —(W_{L}¹—W_{L}²)-,

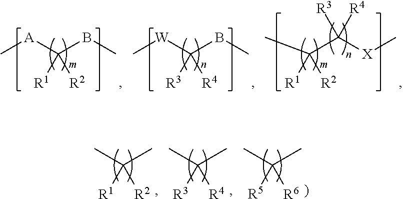

can be the same as or different from each other. In certain embodiments, each unit in the linker moiety is the same as each other.

As used herein, when m (or n or o or p) is defined by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1, 2, or 3) or is any integer in the defined range.

“Pharmaceutically acceptable salt” includes both acid and base addition salts. A pharmaceutically acceptable salt of any one of the heterobifunctional compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more heterobifunctional compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents or any other cancer treatment known in the art. When co-administered, heterobifunctional compounds disclosed herein can operate in conjunction with conventional chemotherapeutic agents or any other cancer treatment known in the art to produce mechanistically additive or synergistic therapeutic effects.

In some aspects, the pH of the compositions disclosed herein can be adjusted with pharmaceutically acceptable acids, bases, or buffers to enhance the stability of the heterobifunctional compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a patient, together with a compound of the invention, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifungal agents, isotonic and absorption delaying agents, and the like, compatible with pharmaceutical administration.

In particular, pharmaceutically acceptable excipients, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the heterobifunctional compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

As used herein, the heterobifunctional compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The heterobifunctional compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In some aspects, the pharmaceutical compositions disclosed herein can include an effective amount of one or more heterobifunctional compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringers solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of the heterobifunctional compounds described herein and one or more additional compounds (e.g., one or more additional compounds, drugs, or agents used for the treatment of cancer or any other condition or disease, including conditions or diseases known to be associated with or caused by cancer, inflammation, and/or autoimmune diseases), both the heterobifunctional compounds and the additional compounds may be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, provided herein are a heterobifunctional compound described herein for preventing or treating a disease or condition.

In some aspects, provided herein are a heterobifunctional compound described herein for treating or preventing one or more diseases or conditions disclosed herein in a subject in need thereof. In certain embodiments, the disease or condition is a TRK-mediated disease or condition. In certain embodiments, the disease or condition is resulted from TRK expression, mutation, deletion, or fusion. In certain embodiments, the diseases or conditions are cancer, pain, inflammation, and immunological diseases

In some aspects, provided herein are use of a heterobifunctional compound in manufacture of a medicament for preventing or treating one or more diseases or conditions disclosed herein.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had, having an elevated risk to have, or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition disclosed herein (e.g., an TRK-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the heterobifunctional compounds, compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. In certain embodiments, the subject has an elevated risk of developing one or more TRK-mediated diseases. Exemplary TRK-mediated diseases that can be treated with heterobifunctional compounds include, for example, cancer, pain, inflammation, and immune diseases.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Example 1: (2S,4R)-1-((S)-2-(1-Fluorocyclopropane-1-carboxamido)-3,3-dimethylbutanoyl)-N—((S)-22-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-1-(4-(4-methylthiazol-5-yl)phenyl)-3,22-dioxo-7,10,13,16,19-pentaoxa-4-azadocosyl)-4-hydroxypyrrolidine-2-carboxamide (CPD-001)

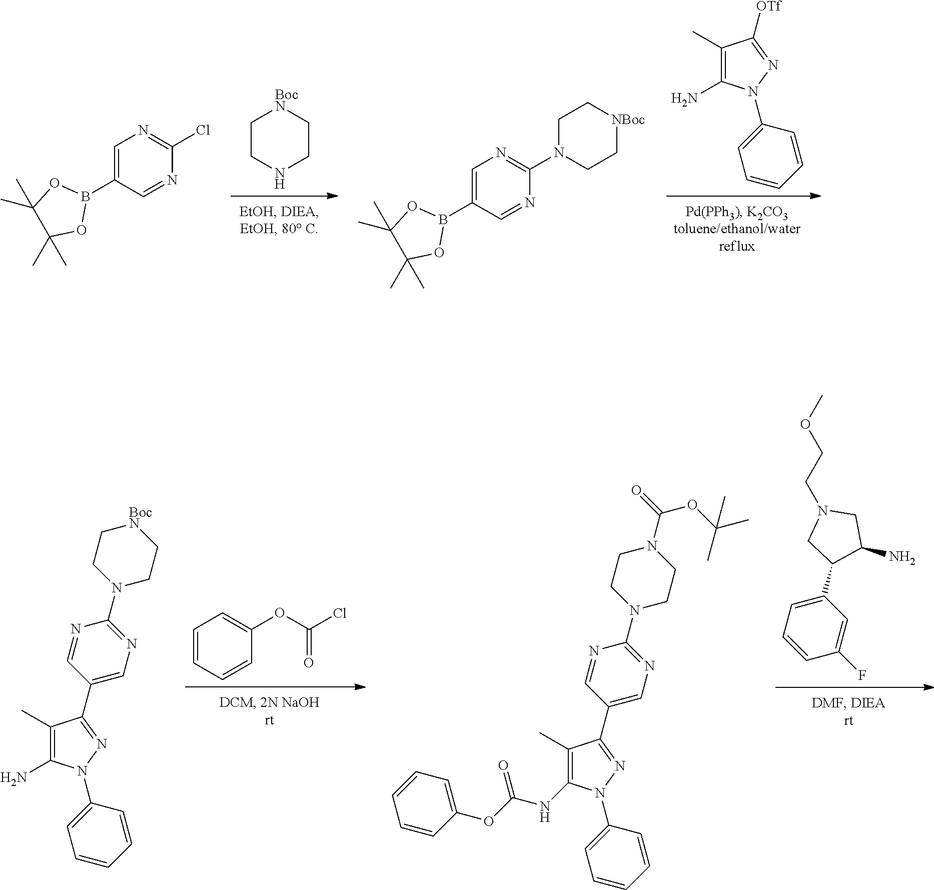

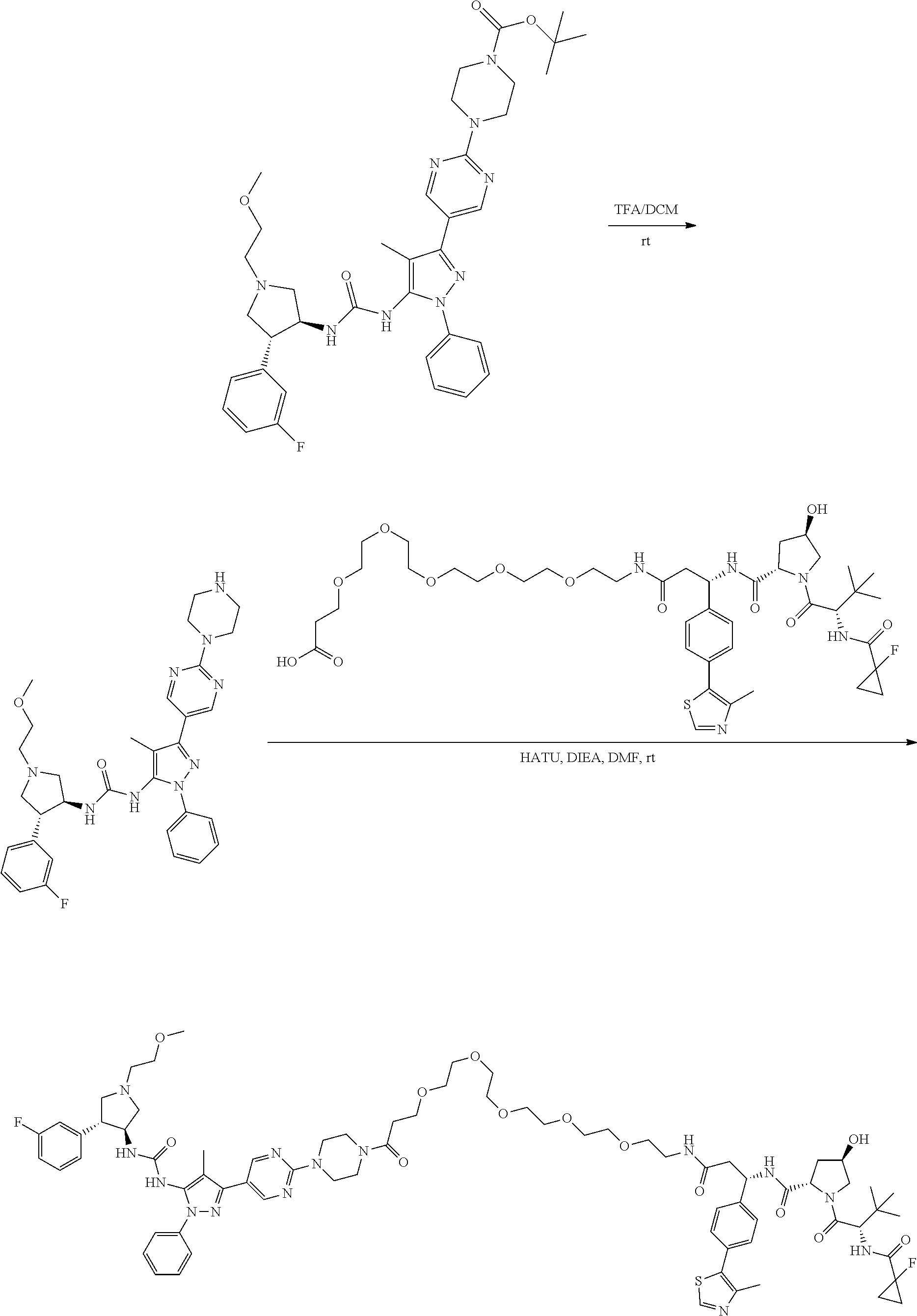

### Step 1. Synthesis of tert-butyl 4-(5-(4,4,5,5-tetramethyl-1,3,2-dioxaborolan-2-yl)pyrimidin-2-yl)piperazine-1-carboxylate

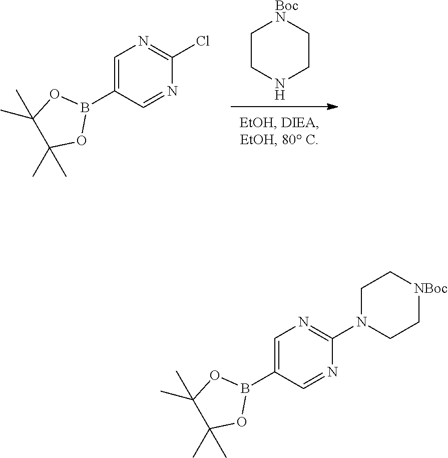 [SMILES: CC1(C)OB(c2cnc(Cl)nc2)OC1(C)C ; CC(C)(C)OC(=O)N1CCNCC1 ; CC(C)(C)OC(=O)N1CCN(c2ncc(B3OC(C)(C)C(C)(C)O3)cn2)CC1]

A mixture of 2-chloro-5-(4,4,5,5-tetramethyl-1,3,2-dioxaborolan-2-yl)pyrimidine (1.5 g, 6.24 mmol), tert-butyl piperazine-1-carboxylate (1.16 g, 6.24 mmol) and DIEA (806 mg, 6.24 mmol) in ethanol (15 mL) was heated at 80° C. for 3 h. The mixture was cooled to rt and filtered to afford the title compound (2.1 g, yield: 72%) as white solid. MS (ESI) m/z=391.4 [M+H]⁺.

### Step 2. Synthesis of tert-butyl 4-(5-(5-amino-4-methyl-1-phenyl-1H-pyrazol-3-yl)pyrimidin-2-yl)piperazine-1-carboxylate

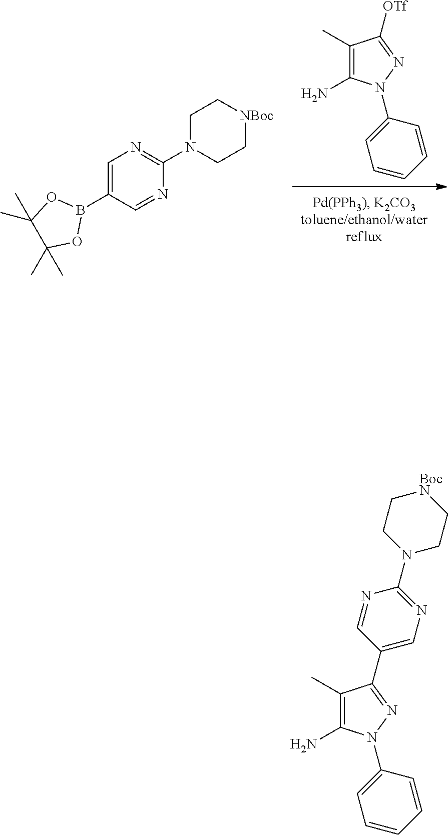 [SMILES: CC(C)(C)OC(=O)N1CCN(c2ncc(B3OC(C)(C)C(C)(C)O3)cn2)CC1 ; Cc1c(OS(=O)(=O)C(F)(F)F)nn(-c2ccccc2)c1N ; Cc1c(-c2cnc(N3CCN(C(=O)OC(C)(C)C)CC3)nc2)nn(-c2ccccc2)c1N]

A mixture of (5-amino-4-methyl-1-phenyl-pyrazol-3-yl) trifluoromethanesulfonate (1.07 g, 3.33 mmol), tert-butyl 4-(5-(4,4,5,5-tetramethyl-1,3,2-dioxaborolan-2-yl)pyrimidin-2-yl)piperazine-1-carboxylate (1.0 g, 2.56 mmol), K₂CO₃ (1.03 g, 7.45 mmol) and Pd(PPh₃)₄ (150 mg, 2.56 mmol) in toluene (10 mL), water (5 mL) and ethanol (2.5 mL) was heated at reflux under N₂ overnight. The mixture was cooled to rt and concentrated. The residue was purified by silica gel chromatography to afford the title compound (500 mg, yield: 40%) as white solid. MS (ESI) m/z=436.7 [M+H]⁺.

### Step 3. Synthesis of tert-butyl 4-(5-(4-methyl-5-((phenoxycarbonyl)amino)-1-phenyl-1H-pyrazol-3-yl)pyrimidin-2-yl)piperazine-1-carboxylate

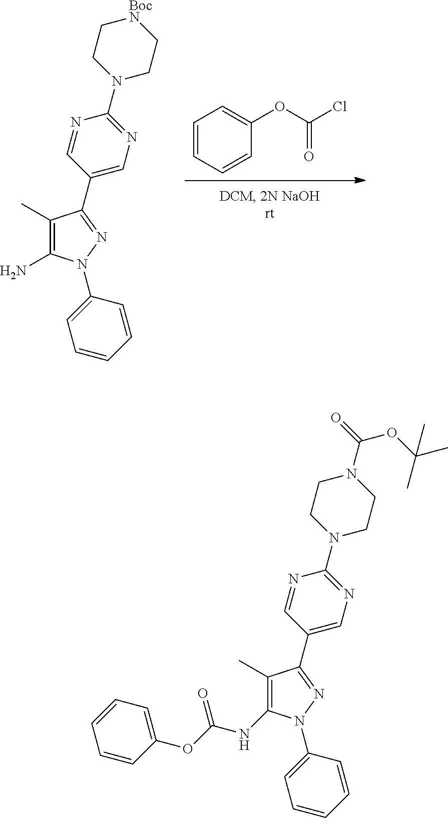 [SMILES: Cc1c(-c2cnc(N3CCN(C(=O)OC(C)(C)C)CC3)nc2)nn(-c2ccccc2)c1N ; O=C(Cl)Oc1ccccc1 ; Cc1c(-c2cnc(N3CCN(C(=O)OC(C)(C)C)CC3)nc2)nn(-c2ccccc2)c1NC(=O)Oc1ccccc1]

To a solution of tert-butyl 4-(5-(5-amino-4-methyl-1-phenyl-1H-pyrazol-3-yl)pyrimidin-2-yl)piperazine-1-carboxylate (300 mg, 619.95 umol) in DCM (8 mL) was added aq. NaOH solution (2 N, 4 mL) at rt. After the reaction mixture was stirred for 3 min, phenyl carbonochloridate (350 mg, 2.24 mmol) was added. The mixture was stirred at rt for 3 h, before it was extracted with DCM (10 mL). The organic layer was dried over Na₂SO₄, filtered and concentrated. The residue was purified by silica gel chromatography to afford the title compound (200 mg, yield: 58%) as colorless oil. MS (ESI) m/z=556.7 [M+H]⁺.

### Step 4. Synthesis of tert-butyl 4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazine-1-carboxylate

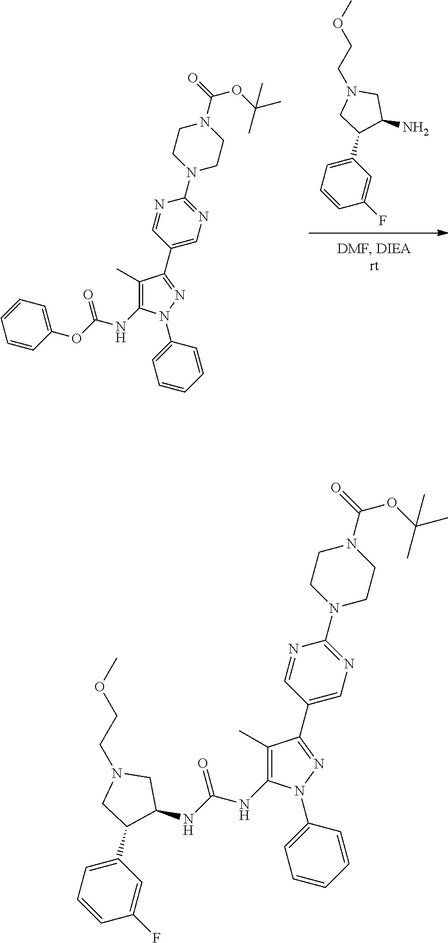 [SMILES: Cc1c(-c2cnc(N3CCN(C(=O)OC(C)(C)C)CC3)nc2)nn(-c2ccccc2)c1NC(=O)Oc1ccccc1 ; COCCN1C[C@@H](N)[C@H](c2cccc(F)c2)C1 ; COCCN1C[C@@H](NC(=O)Nc2c(C)c(-c3cnc(N4CCN(C(=O)OC(C)(C)C)CC4)nc3)nn2-c2ccccc2)[C@H](c2cccc(F)c2)C1]

To a solution of tert-butyl 4-(5-(4-methyl-5-((phenoxycarbonyl)amino)-1-phenyl-1H-pyrazol-3-yl)pyrimidin-2-yl)piperazine-1-carboxylate (200 mg, 359.95 umol) in DMF (3 mL) was added (3S,4R)-4-(3-fluorophenyl)-1-(2-methoxyethyl)pyrrolidin-3-amine (120 mg, 453.21 umol) and DIEA (200 mg, 1.55 mmol) at rt. After the mixture was stirred overnight, the reaction was purified by reverse phase chromatography to afford the title compound (190 mg, yield: 62%) as colorless oil. MS (ESI) m/z=700.9 [M+H]⁺.

### Step 5. Synthesis of 1-((3S,4R)-4-(3-fluorophenyl)-1-(2-methoxyethyl)pyrrolidin-3-yl)-3-(4-methyl-1-phenyl-3-(2-(piperazin-1-yl)pyrimidin-5-yl)-1H-pyrazol-5-yl)urea

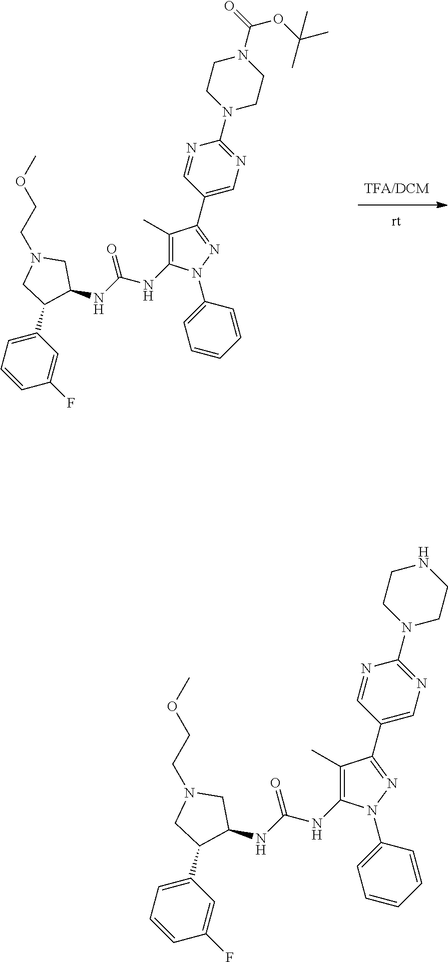 [SMILES: COCCN1C[C@@H](NC(=O)Nc2c(C)c(-c3cnc(N4CCN(C(=O)OC(C)(C)C)CC4)nc3)nn2-c2ccccc2)[C@H](c2cccc(F)c2)C1 ; COCCN1C[C@@H](NC(=O)Nc2c(C)c(-c3cnc(N4CCNCC4)nc3)nn2-c2ccccc2)[C@H](c2cccc(F)c2)C1]

To a solution of tert-butyl 4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazine-1-carboxylate (190 mg, 225.35 umol) in DCM (3 mL) was added TFA (1.5 mL) at rt. The mixture was stirred for 1 h, before it was concentrated. The residue was dissolved in DCM (20 mL), washed with saturated Na₂CO₃ solution (20 mL), brine (20 mL), dried over Na₂SO₄, filtered and concentrated to afford the title compound (100 mg, yield: 73%) as white solid. MS (ESI) m/z=600.5 [M+H]⁺.

### Step 6. Synthesis of (2S,4R)-1-((S)-2-(1-fluorocyclopropane-1-carboxamido)-3,3-dimethylbutanoyl)-N—((S)-22-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-1-(4-(4-methylthiazol-5-yl)phenyl)-3,22-dioxo-7,10,13,16,19-pentaoxa-4-azadocosyl)-4-hydroxypyrrolidine-2-carboxamide (CPD-001)

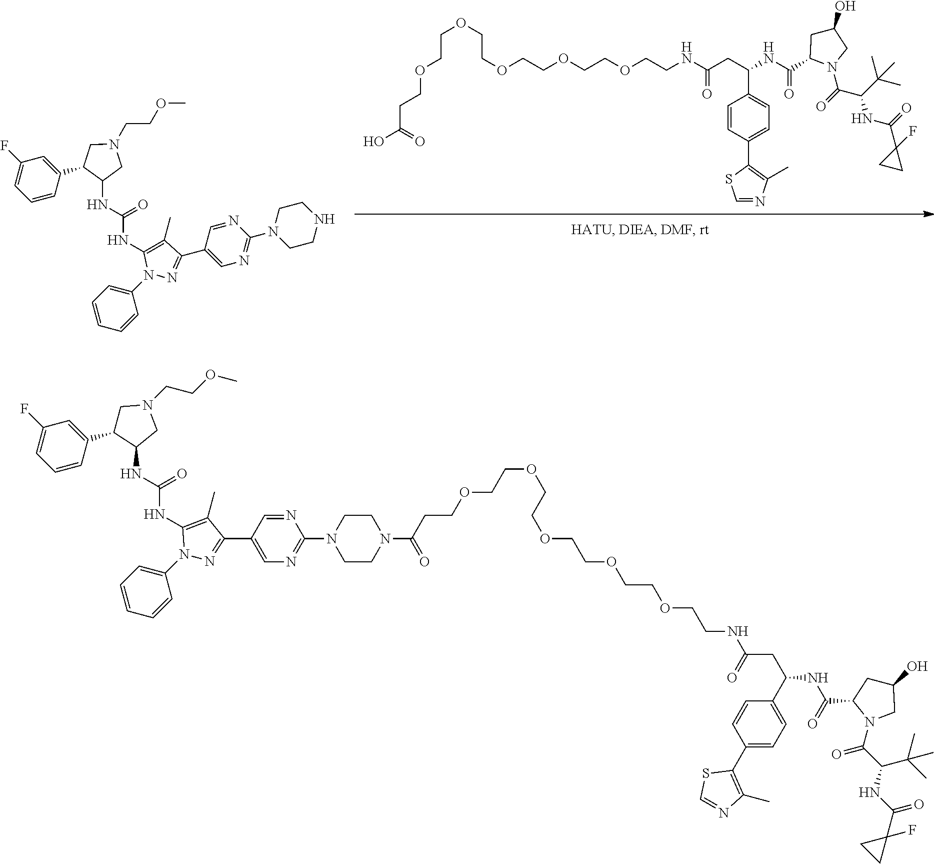 [SMILES: COCCN1CC(NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCOCCOCCOCCOCCOCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCOCCOCCOCCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

A mixture of 1-[5-(6,7-dihydro-5H-pyrrolo[3,4-b]pyridin-3-yl)-4-methyl-2-phenyl-pyrazol-3-yl]-3-[(3S,4R)-4-(3-fluorophenyl)-1-(2-methoxyethyl)pyrrolidin-3-yl]urea (6 mg, 10.69 μmol), (S)-1-((2S,4R)-1-((S)-2-(1-fluorocyclopropane-1-carboxamido)-3,3-dimethylbutanoyl)-4-hydroxypyrrolidin-2-yl)-3-(4-(4-methylthiazol-5-yl)phenyl)-1,5-dioxo-9,12,15,18,21-pentaoxa-2,6-diazatetracosan-24-oic acid (10 mg, 11.55 μmol), HATU (15 mg, 39.47 μmol) and DIEA (20 mg, 155.04 μmol) in DMF (1 mL) was stirred at rt overnight. The mixture was purified by reverse phase chromatography and prep-TLC to afford the title product (1.9 mg, yield: 12%) as white solid. MS (ESI) m/z=1448.5 [M+H]⁺.

### Example 2: 1-(3-(2-(4-(4-((2-(2,6-Dioxopiperidin-3-yl)-1,3-dioxoisoindolin-5-yl)amino)butanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-002)

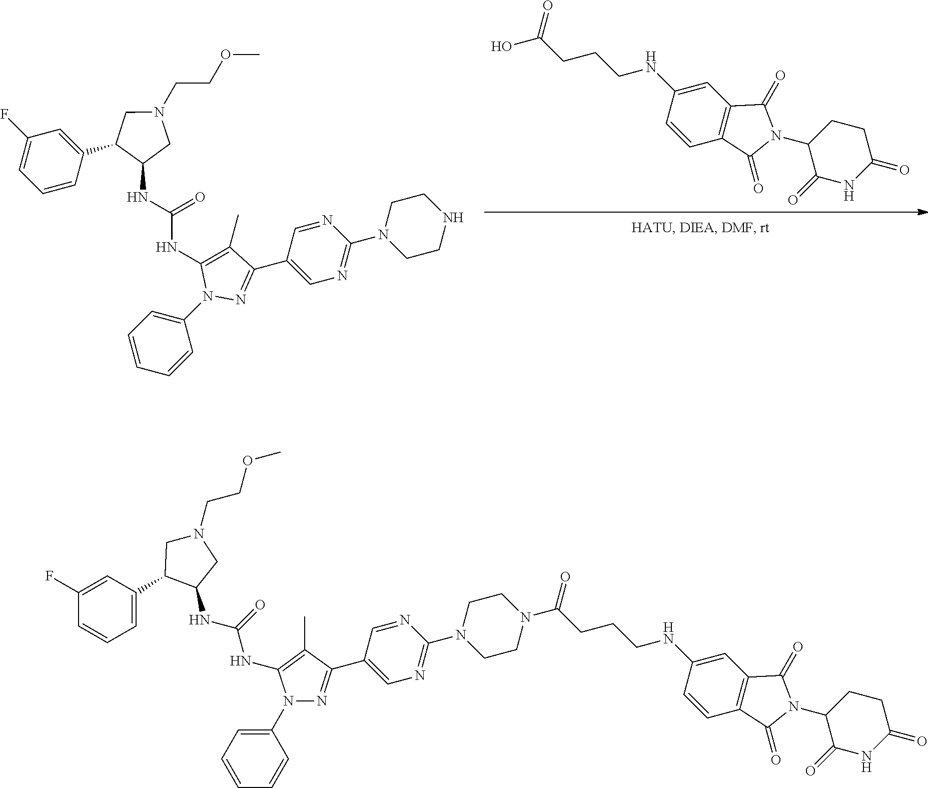 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCCNc1ccc2c(c1)C(=O)N(C1CCC(=O)NC1=O)C2=O ; COCCN1C[C@@H](NC(=O)Nc2c(C)c(-c3cnc(N4CCN(C(=O)CCCNc5ccc6c(c5)C(=O)N(C5CCC(=O)NC5=O)C6=O)CC4)nc3)nn2-c2ccccc2)[C@H](c2cccc(F)c2)C1]

CPD-002 was synthesized following the same procedure as CPD-001 (3 mg, yield: 36%). MS (ESI) m/z=941.8 [M+H]⁺.

### Example 3: 1-(3-(2-(4-((2-(2,6-Dioxopiperidin-3-yl)-1,3-dioxoisoindolin-4-yl)glycyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-003)

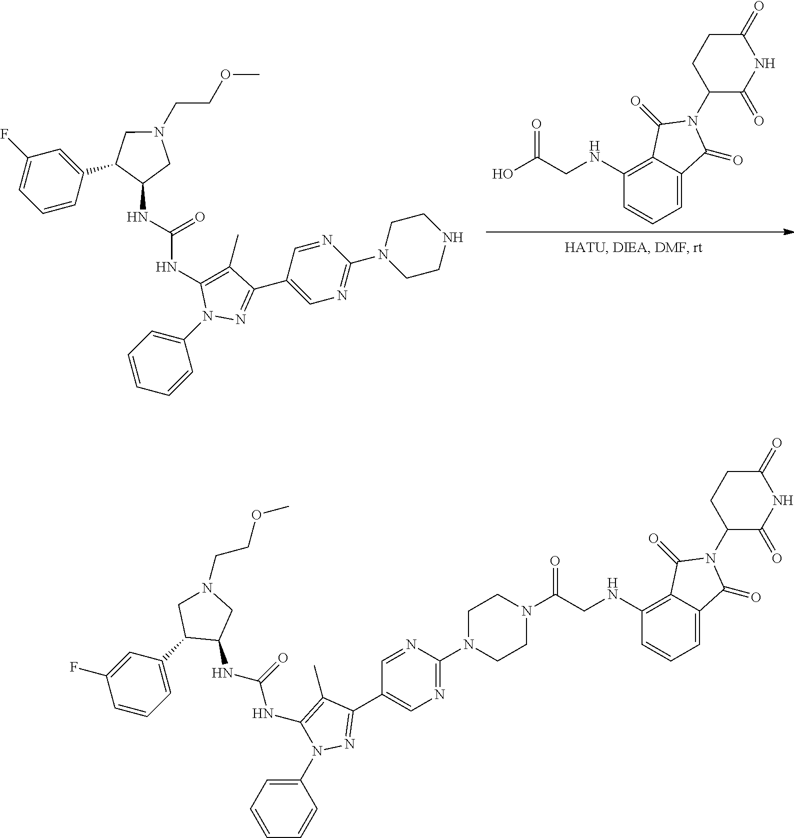 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CNc1cccc2c1C(=O)N(C1CCC(=O)NC1=O)C2=O ; COCCN1C[C@@H](NC(=O)Nc2c(C)c(-c3cnc(N4CCN(C(=O)CNc5cccc6c5C(=O)N(C5CCC(=O)NC5=O)C6=O)CC4)nc3)nn2-c2ccccc2)[C@H](c2cccc(F)c2)C1]

CPD-003 was synthesized following the same procedure as CPD-001 (2 mg, yield: 26%). MS (ESI) m/z=913.8 [M+H]⁺.

### Example 4: 1-(3-(2-(4-(4-((2-(2,6-Dioxopiperidin-3-yl)-1,3-dioxoisoindolin-4-yl)amino)butanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-004)

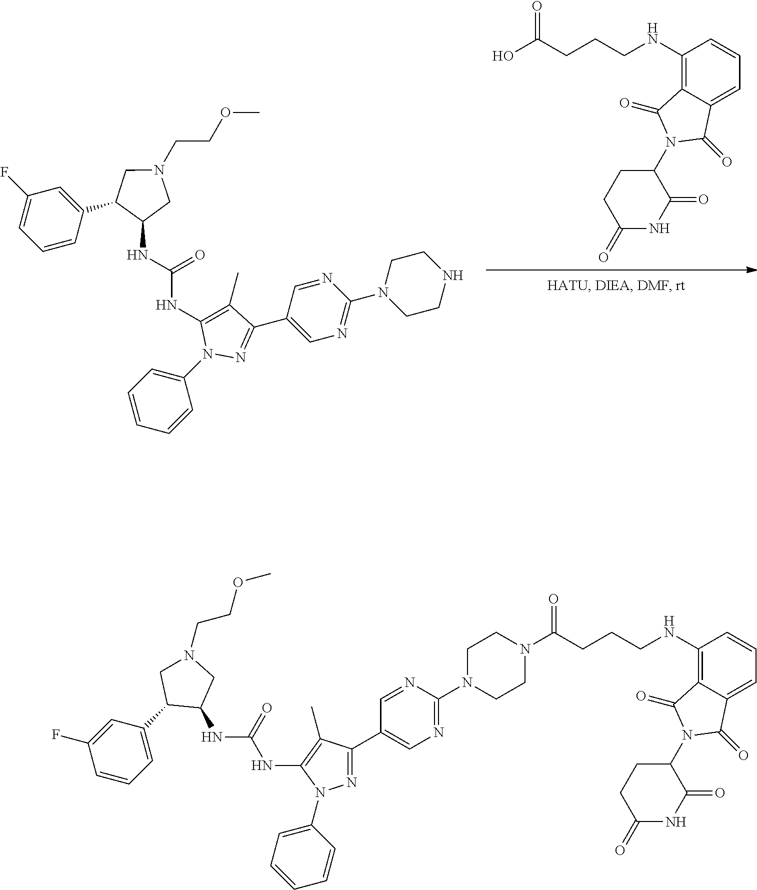 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCCNc1cccc2c1C(=O)N(C1CCC(=O)NC1=O)C2=O ; COCCN1C[C@@H](NC(=O)Nc2c(C)c(-c3cnc(N4CCN(C(=O)CCCNc5cccc6c5C(=O)N(C5CCC(=O)NC5=O)C6=O)CC4)nc3)nn2-c2ccccc2)[C@H](c2cccc(F)c2)C1]

CPD-004 was synthesized following the same procedure as CPD-001 (3 mg, yield: 37%). MS (ESI) m/z=941.9 [M+H]⁺.

### Example 5: 1-(3-(2-(4-(3-(2-(2-(2-((2-(2,6-Dioxopiperidin-3-yl)-1,3-dioxoisoindolin-4-yl)amino)ethoxy)ethoxy)ethoxy)propanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-005)

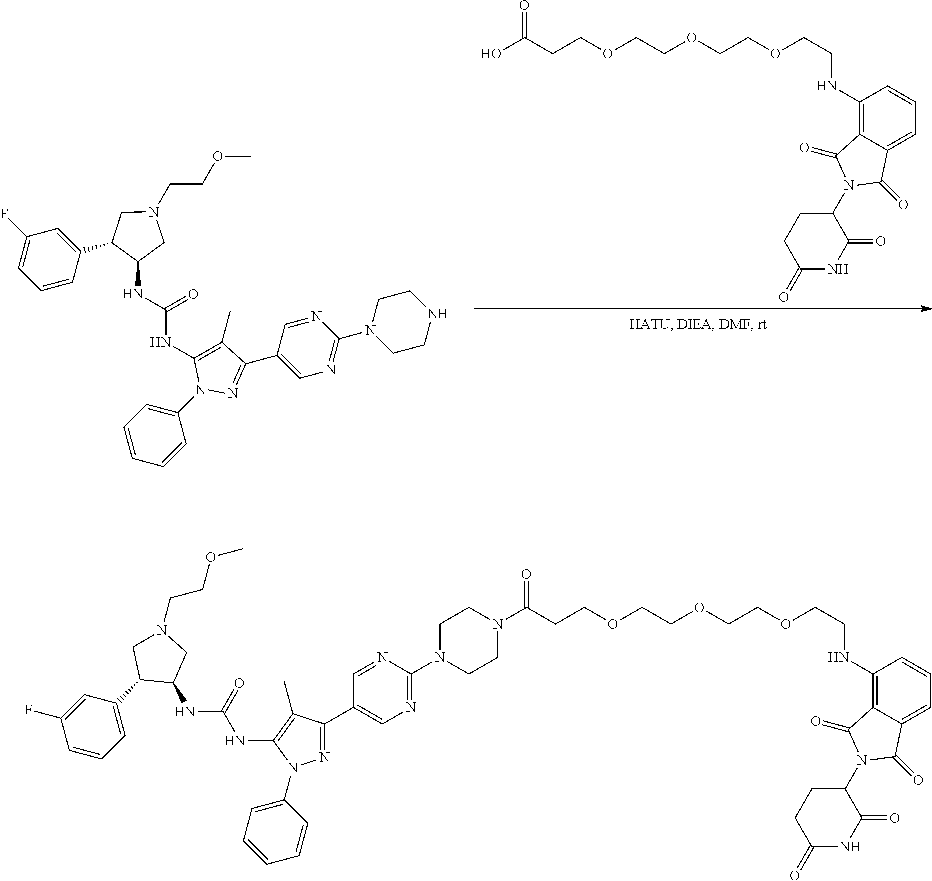 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCOCCOCCOCCNc1cccc2c1C(=O)N(C1CCC(=O)NC1=O)C2=O ; COCCN1C[C@@H](NC(=O)Nc2c(C)c(-c3cnc(N4CCN(C(=O)CCOCCOCCOCCNc5cccc6c5C(=O)N(C5CCC(=O)NC5=O)C6=O)CC4)nc3)nn2-c2ccccc2)[C@H](c2cccc(F)c2)C1]

CPD-005 was synthesized following the same procedure as CPD-001 (3 mg, yield: 32%). MS (ESI) m/z=1061.2 [M+H]⁺.

### Example 6: 1-(3-(2-(4-(8-((2-(2,6-Dioxopiperidin-3-yl)-1,3-dioxoisoindolin-5-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-006)

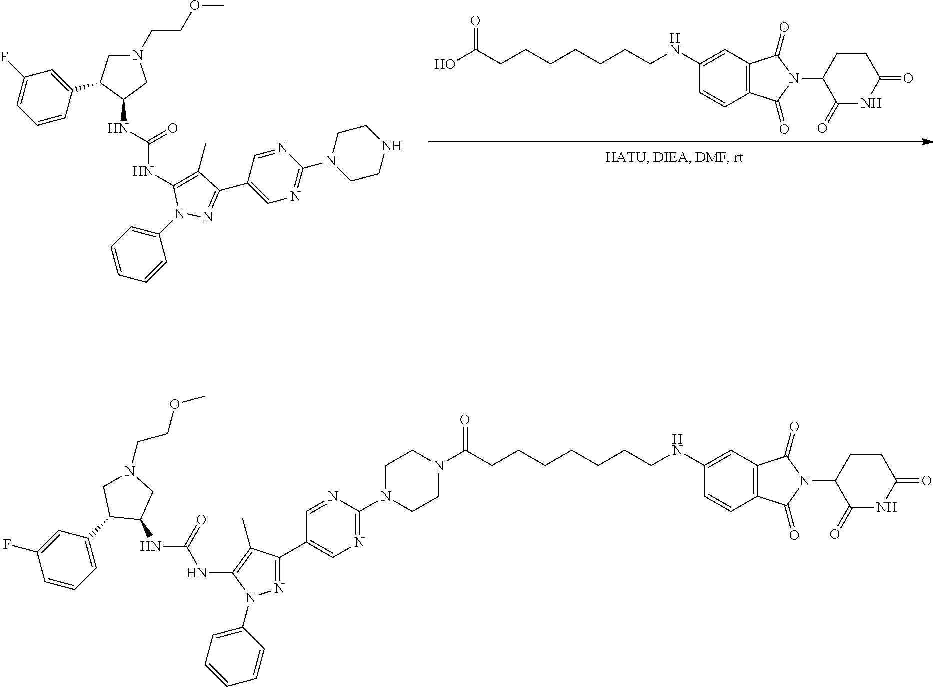 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCCCCCCNc1ccc2c(c1)C(=O)N(C1CCC(=O)NC1=O)C2=O ; COCCN1C[C@@H](NC(=O)Nc2c(C)c(-c3cnc(N4CCN(C(=O)CCCCCCCNc5ccc6c(c5)C(=O)N(C5CCC(=O)NC5=O)C6=O)CC4)nc3)nn2-c2ccccc2)[C@H](c2cccc(F)c2)C1]

CPD-006 was synthesized following the same procedure as CPD-001 (4 mg, yield: 48%). MS (ESI) m/z=997.9 [M+H]⁺.

### Example 7: 1-(3-(2-(4-(6-((2-(2,6-Dioxopiperidin-3-yl)-1,3-dioxoisoindolin-4-yl)amino)hexanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-007)

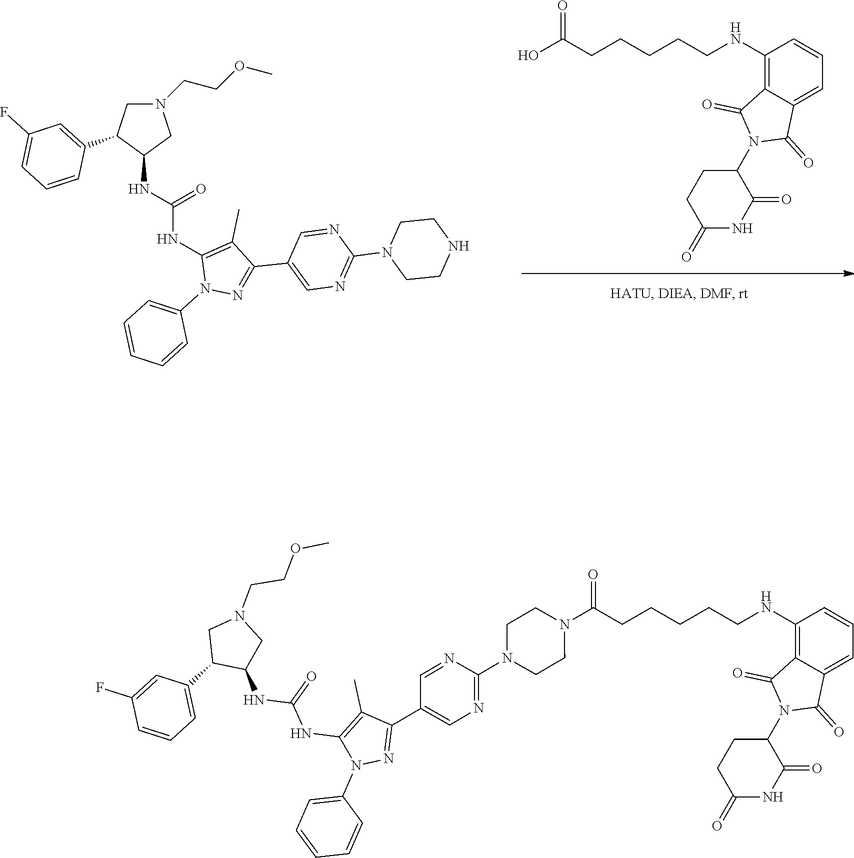 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCCCCNc1cccc2c1C(=O)N(C1CCC(=O)NC1=O)C2=O ; COCCN1C[C@@H](NC(=O)Nc2c(C)c(-c3cnc(N4CCN(C(=O)CCCCCNc5cccc6c5C(=O)N(C5CCC(=O)NC5=O)C6=O)CC4)nc3)nn2-c2ccccc2)[C@H](c2cccc(F)c2)C1]

CPD-007 was synthesized following the same procedure as CPD-001 (4 mg, yield: 48%). MS (ESI) m/z=970.0 [M+H]⁺.

### Example 8: 1-(3-(2-(4-(6-((2-(2,6-Dioxopiperidin-3-yl)-1,3-dioxoisoindolin-5-yl)amino)hexanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-008)

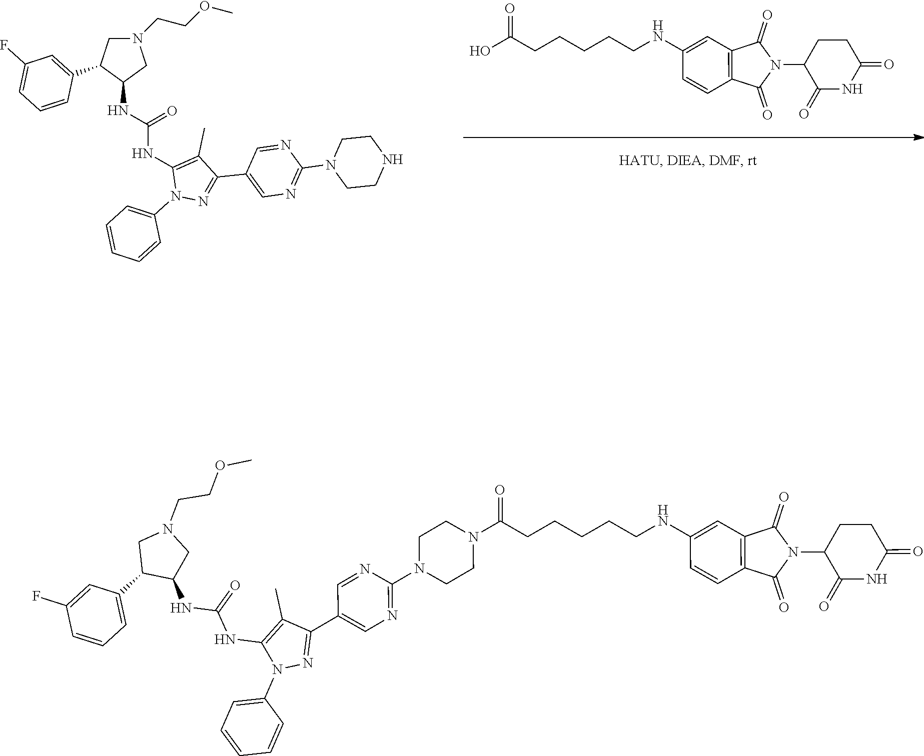 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCCCCNc1ccc2c(c1)C(=O)N(C1CCC(=O)NC1=O)C2=O ; COCCN1C[C@@H](NC(=O)Nc2c(C)c(-c3cnc(N4CCN(C(=O)CCCCCNc5ccc6c(c5)C(=O)N(C5CCC(=O)NC5=O)C6=O)CC4)nc3)nn2-c2ccccc2)[C@H](c2cccc(F)c2)C1]

CPD-008 was synthesized following the same procedure as CPD-001 (4 mg, yield: 50%). MS (ESI) m/z=970.0 [M+H]⁺.

### Example 9: 1-(3-(2-(4-(8-((2-(2,6-Dioxopiperidin-3-yl)-1,3-dioxoisoindolin-4-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-009)

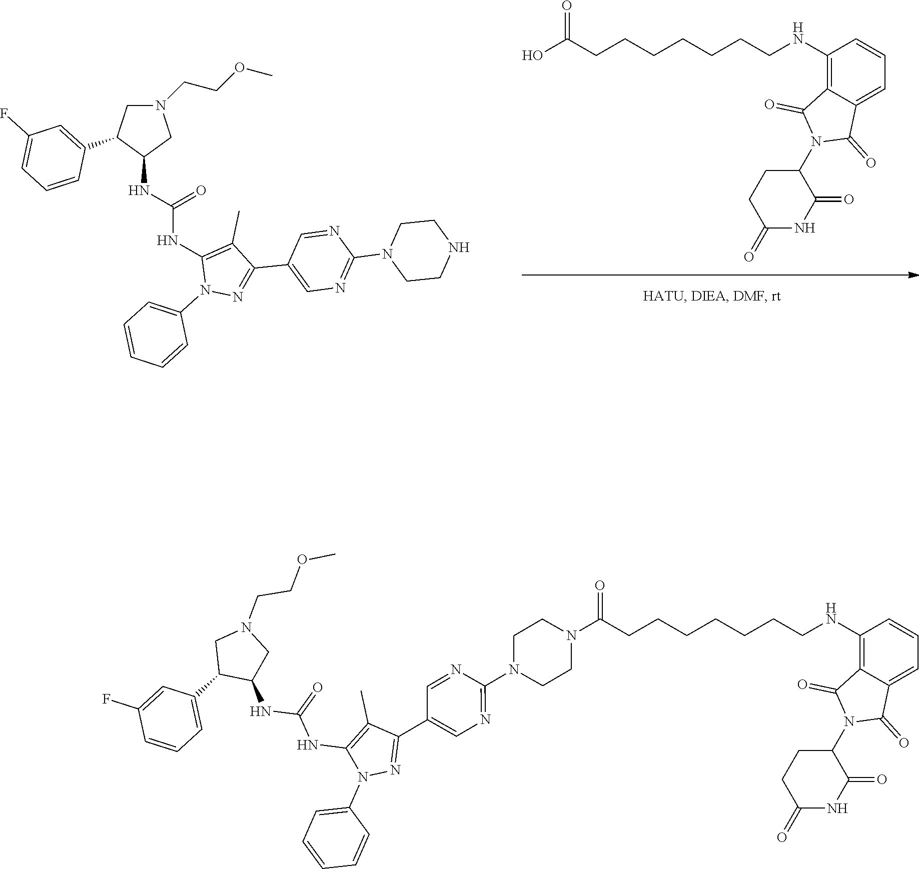 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCCCCCCNc1cccc2c1C(=O)N(C1CCC(=O)NC1=O)C2=O ; COCCN1C[C@@H](NC(=O)Nc2c(C)c(-c3cnc(N4CCN(C(=O)CCCCCCCNc5cccc6c5C(=O)N(C5CCC(=O)NC5=O)C6=O)CC4)nc3)nn2-c2ccccc2)[C@H](c2cccc(F)c2)C1]

CPD-009 was synthesized following the same procedure as CPD-001 (4 mg, yield: 47%). MS (ESI) m/z=998.0 [M+H]⁺.

### Example 10: 1-(3-(2-(4-(8-((2-(2,6-Dioxopiperidin-3-yl)-1,3-dioxoisoindolin-4-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-010)

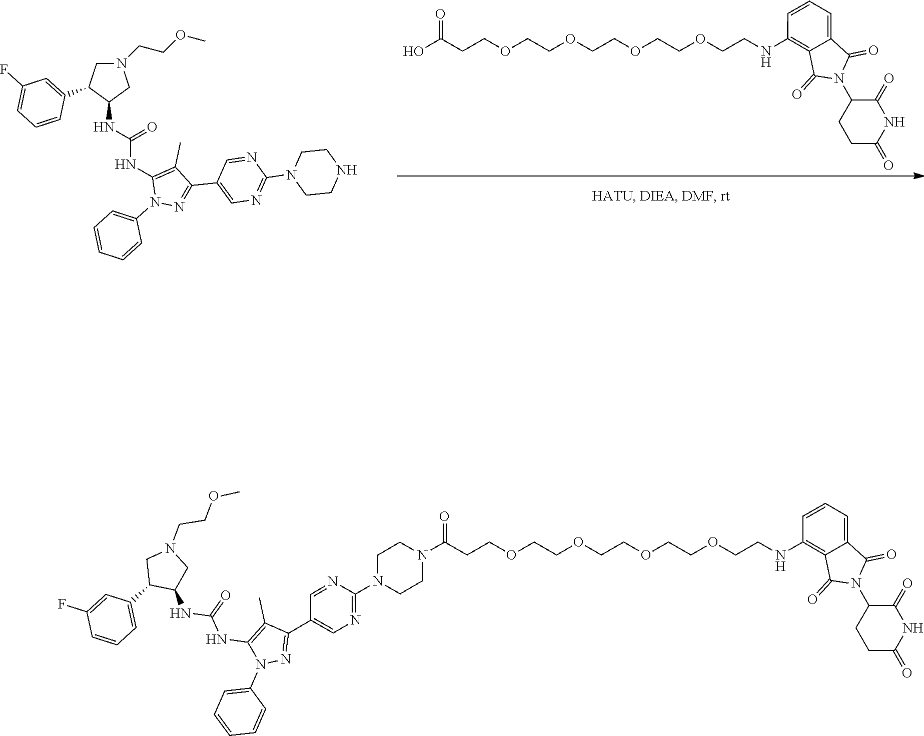 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCOCCOCCOCCOCCNc1cccc2c1C(=O)N(C1CCC(=O)NC1=O)C2=O ; COCCN1C[C@@H](NC(=O)Nc2c(C)c(-c3cnc(N4CCN(C(=O)CCOCCOCCOCCOCCNc5cccc6c5C(=O)N(C5CCC(=O)NC5=O)C6=O)CC4)nc3)nn2-c2ccccc2)[C@H](c2cccc(F)c2)C1]

CPD-010 was synthesized following the same procedure as CPD-001 (4 mg, yield: 43%). MS (ESI) m/z=1104.2 [M+H]⁺.

### Example 11: (2S,4R)-1-((S)-2-(1-Fluorocyclopropane-1-carboxamido)-3,3-dimethylbutanoyl)-N—((S)-3-((2-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-2-oxoethyl)amino)-1-(4-(4-methylthiazol-5-yl)phenyl)-3-oxopropyl)-4-hydroxypyrrolidine-2-carboxamide (CPD-011)

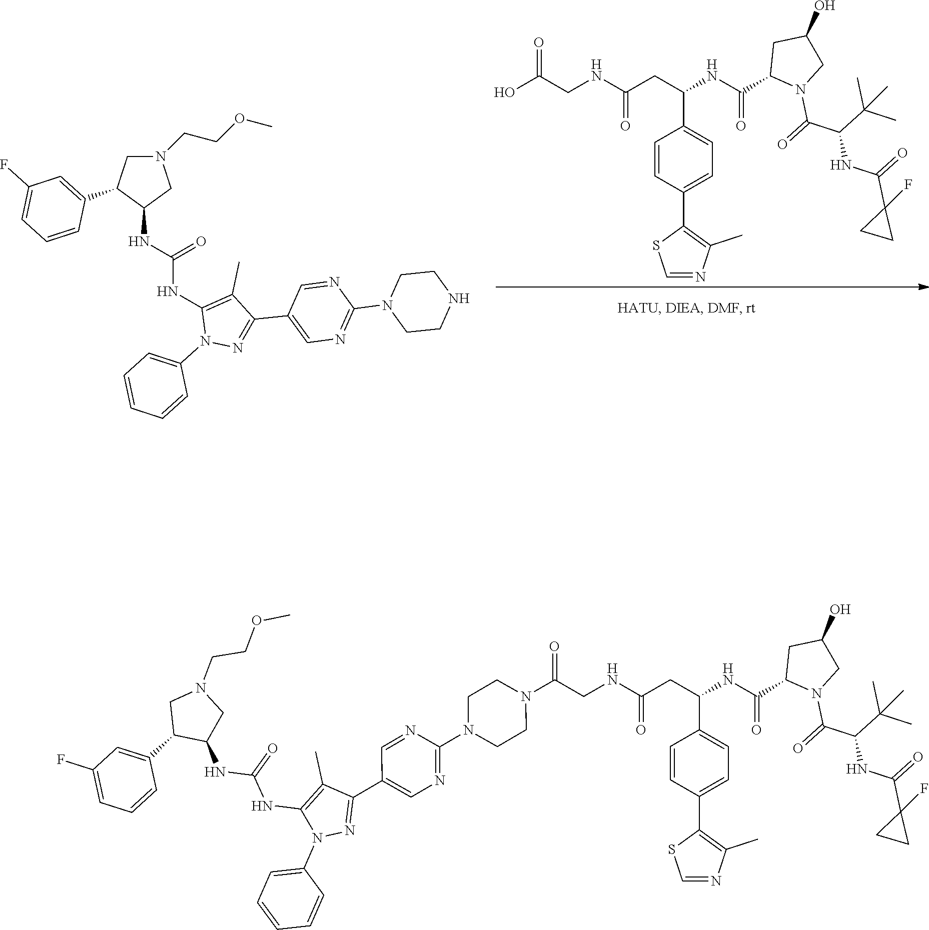 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-011 was synthesized following the same procedure as CPD-001 (4 mg, yield: 390%). MS (ESI) m/z=1214.1 [M+H]⁺.

### Example 12: (2S,4R)-1-((S)-2-(1-Fluorocyclopropane-1-carboxamido)-3,3-dimethylbutanoyl)-N—((S)-3-((6-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-6-oxohexyl)amino)-1-(4-(4-methylthiazol-5-yl)phenyl)-3-oxopropyl)-4-hydroxypyrrolidine-2-carboxamide (CPD-012)

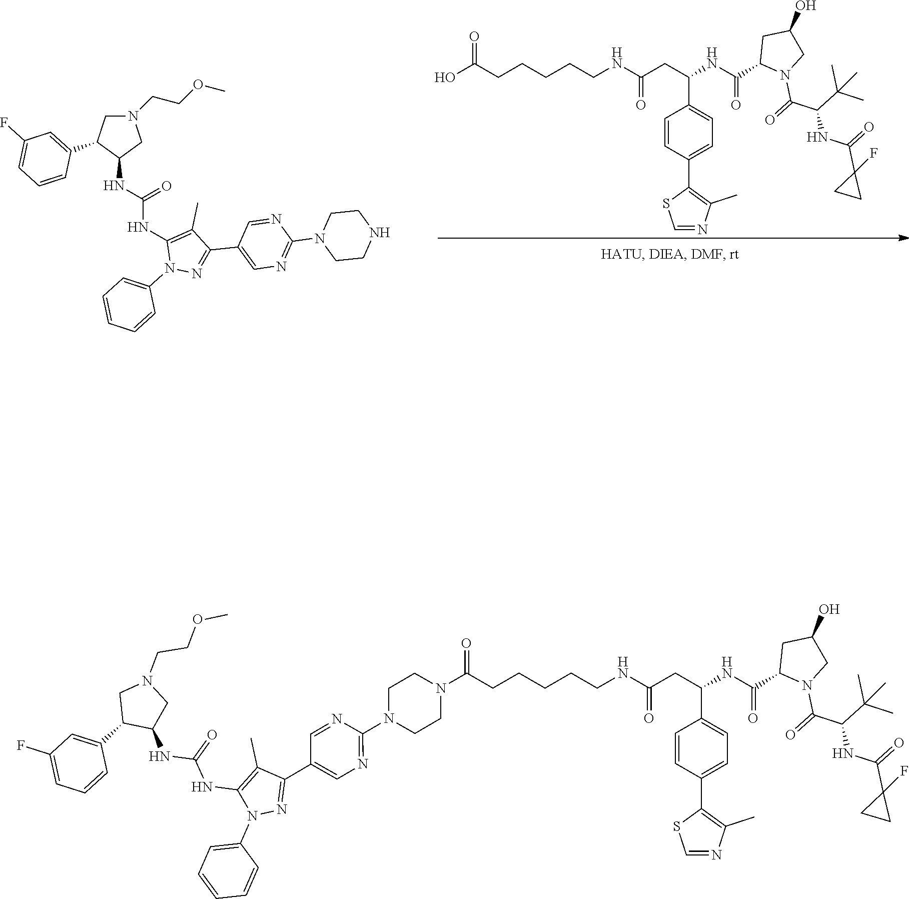 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCCCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCCCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-012 was synthesized following the same procedure as CPD-001 (4 mg, yield: 38%). MS (ESI) m/z=1270.4 [M+H]⁺.

### Example 13: (2S,4R)-1-((S)-2-(1-Fluorocyclopropane-1-carboxamido)-3,3-dimethylbutanoyl)-N—((S)-3-((4-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-4-oxobutyl)amino)-1-(4-(4-methylthiazol-5-yl)phenyl)-3-oxopropyl)-4-hydroxypyrrolidine-2-carboxamide (CPD-013)

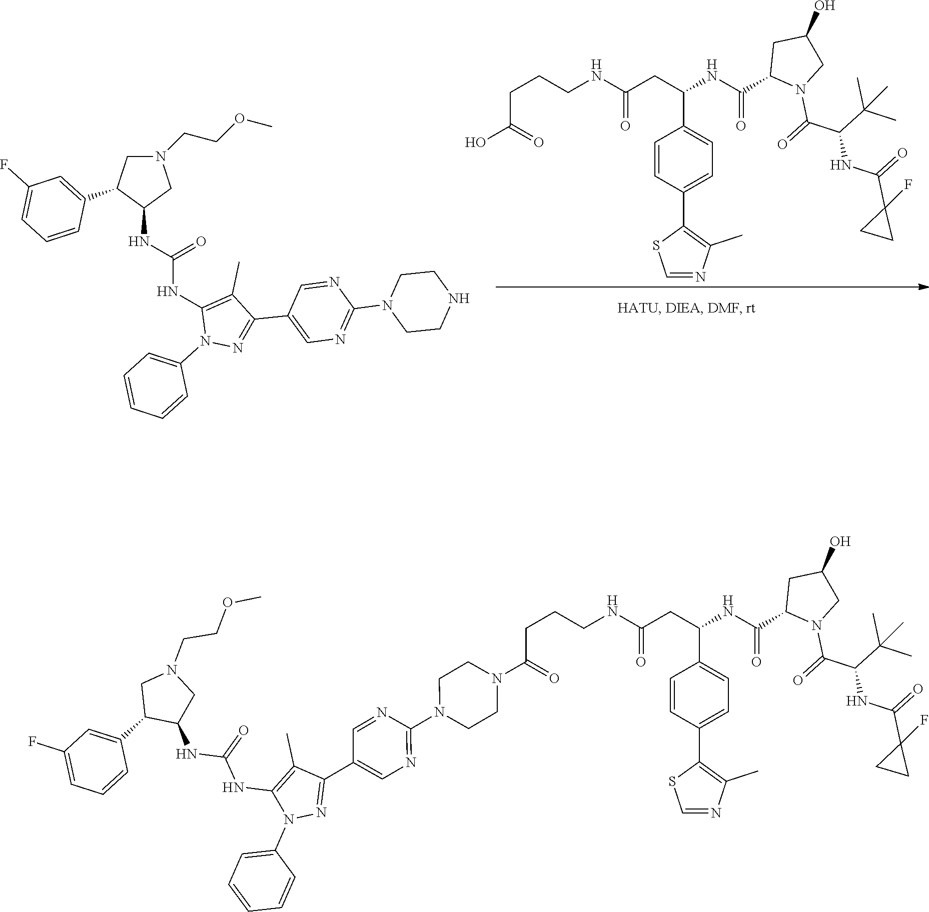 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-013 was synthesized following the same procedure as CPD-001 (4 mg, yield: 39%). MS (ESI) m/z=1242.2 [M+H]⁺.

### Example 14: (2S,4R)-1-((S)-2-(1-Fluorocyclopropane-1-carboxamido)-3,3-dimethylbutanoyl)-N—((S)-3-((8-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-8-oxooctyl)amino)-1-(4-(4-methylthiazol-5-yl)phenyl)-3-oxopropyl)-4-hydroxypyrrolidine-2-carboxamide (CPD-014)

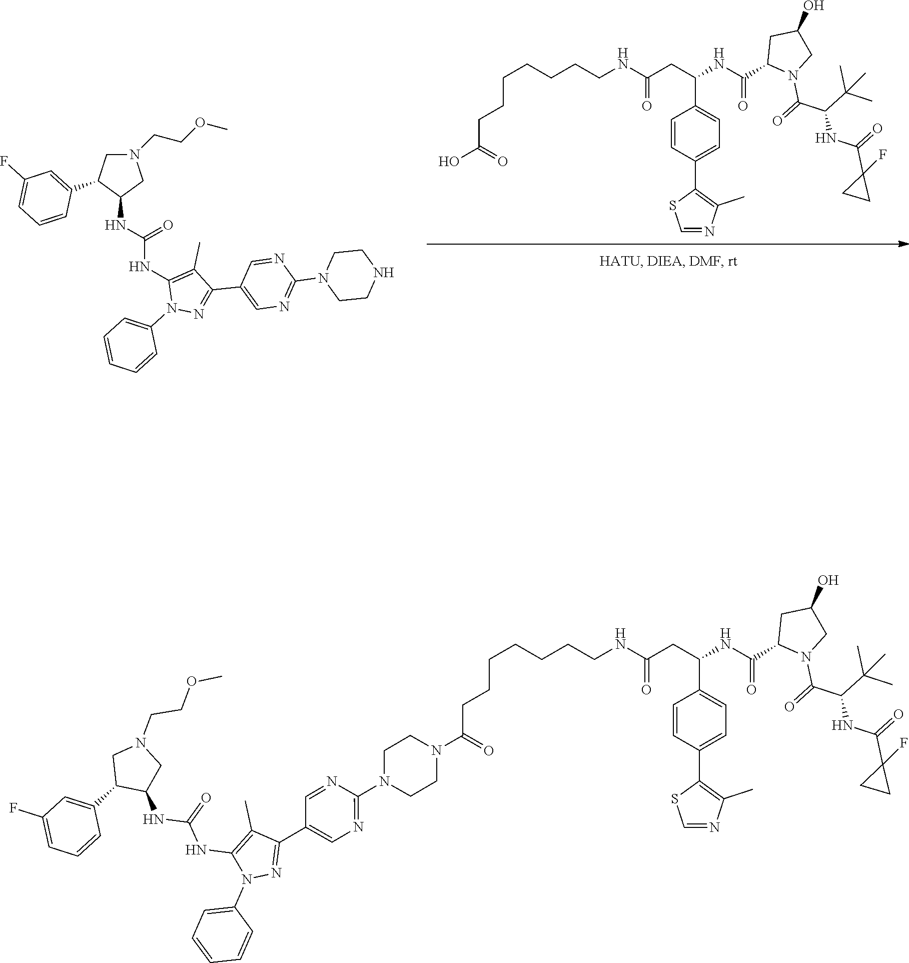 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCCCCCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCCCCCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-014 was synthesized following the same procedure as CPD-001 (4 mg, yield: 37%). MS (ESI) m/z=1298.3 [M+H]⁺.

### Example 15: (2S,4R)-1-((S)-2-(1-Fluorocyclopropane-1-carboxamido)-3,3-dimethylbutanoyl)-N—((S)-3-((10-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-10-oxodecyl)amino)-1-(4-(4-methylthiazol-5-yl)phenyl)-3-oxopropyl)-4-hydroxypyrrolidine-2-carboxamide (CPD-015)

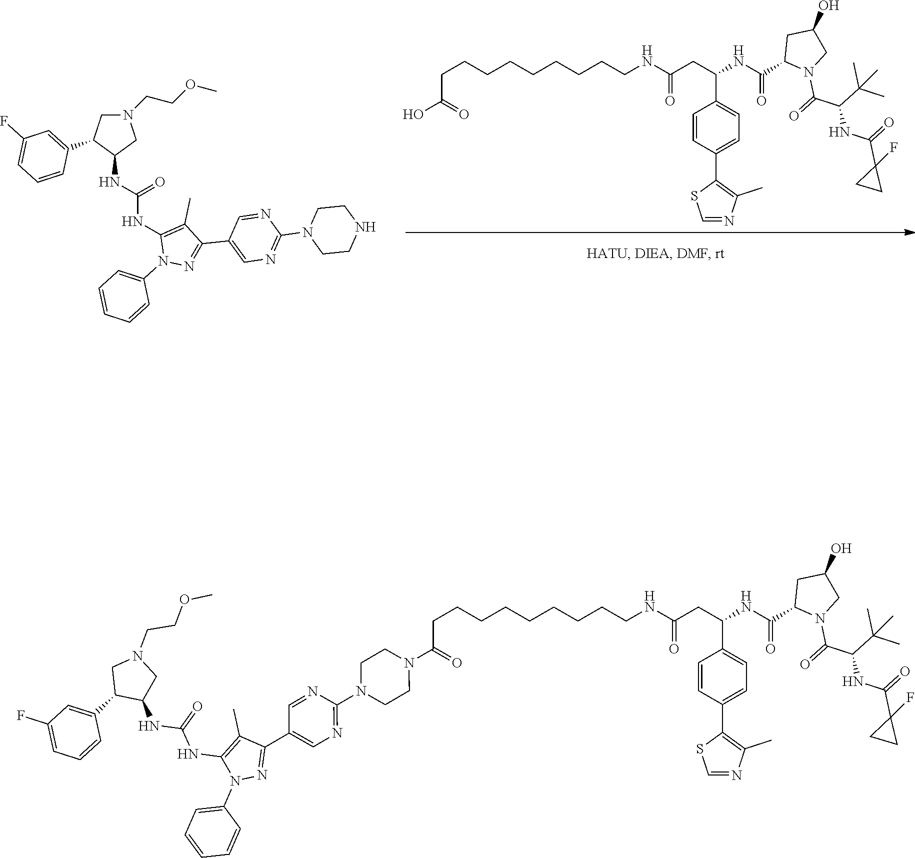 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCCCCCCCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCCCCCCCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-015 was synthesized following the same procedure as CPD-001 (3 mg, yield: 27%). MS (ESI) m/z=1326.4 [M+H]⁺.

### Example 16: (2S,4R)-1-((S)-2-(1-Fluorocyclopropane-1-carboxamido)-3,3-dimethylbutanoyl)-N—((S)-3-((2-(2-(3-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-3-oxopropoxy)ethoxy)ethyl)amino)-1-(4-(4-methylthiazol-5-yl)phenyl)-3-oxopropyl)-4-hydroxypyrrolidine-2-carboxamide (CPD-016)

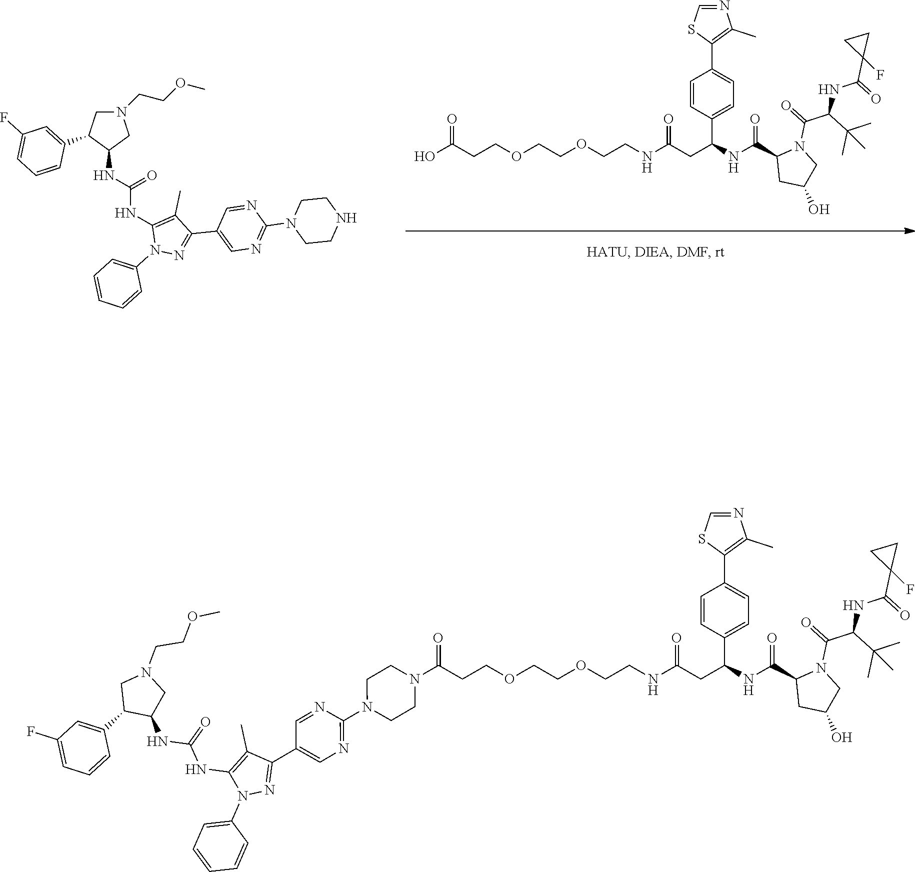 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCOCCOCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-016 was synthesized following the same procedure as CPD-001 (4.3 mg, yield: 30% H). MS(ESI) m/z=1316.3 [M+H]⁺.

### Example 17: (2S,4R)-1-((S)-2-(1-Fluorocyclopropane-1-carboxamido)-3,3-dimethylbutanoyl)-N—((S)-16-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-1-(4-(4-methylthiazol-5-yl)phenyl)-3,16-dioxo-7,10,13-trioxa-4-azahexadecyl)-4-hydroxypyrrolidine-2-carboxamide (CPD-017)

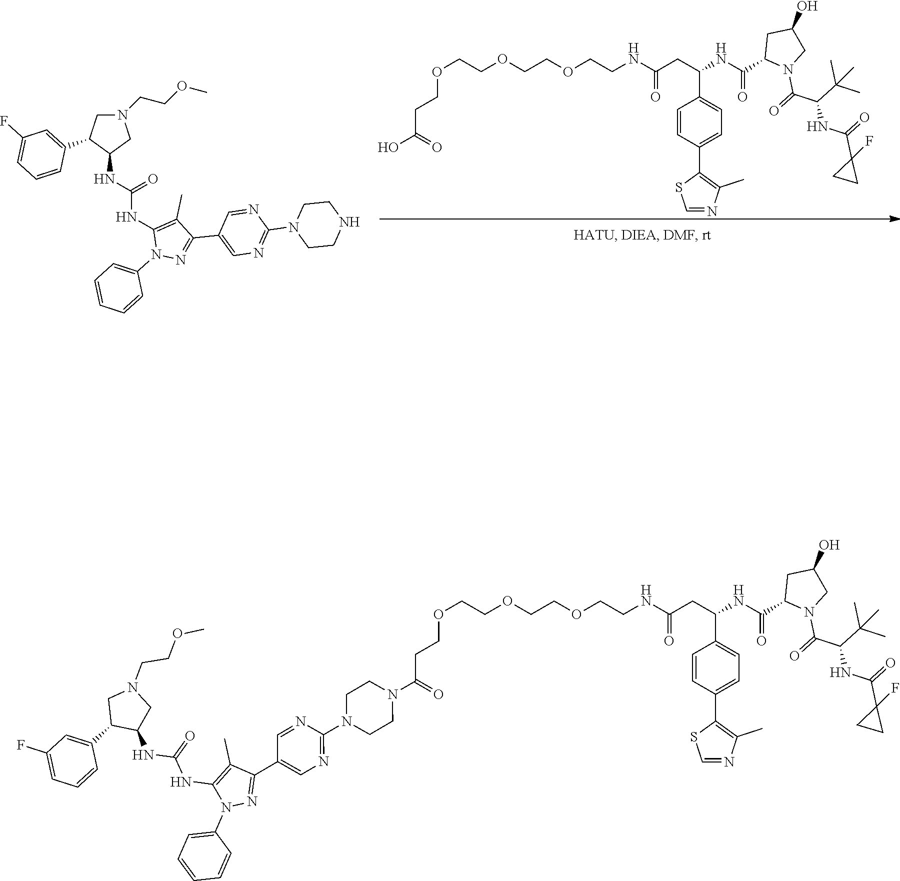 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCOCCOCCOCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCOCCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-017 was synthesized following the same procedure as CPD-001 (4.5 mg, yield: 30%). MS (ESI) m/z=1360.4 [M+H]⁺.

### Example 18: (2S,4R)-1-((S)-2-(1-Fluorocyclopropane-1-carboxamido)-3,3-dimethylbutanoyl)-N—((S)-19-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-1-(4-(4-methylthiazol-5-yl)phenyl)-3,19-dioxo-7,10,13,16-tetraoxa-4-azanonadecyl)-4-hydroxypyrrolidine-2-carboxamide (CPD-018)

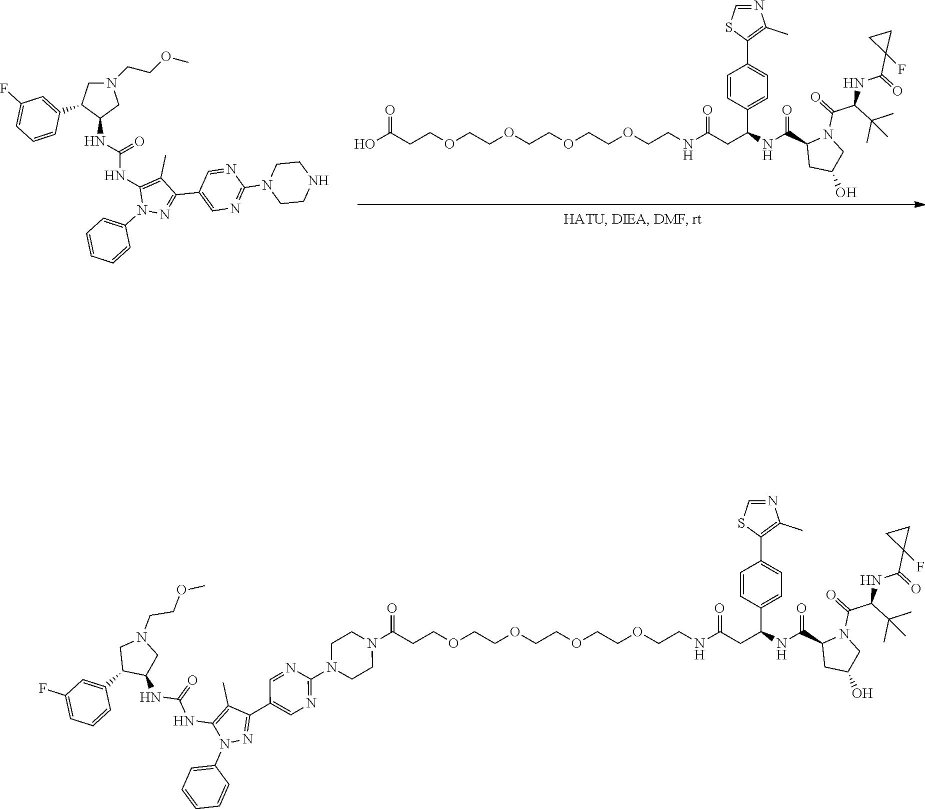 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc([C@H](CC(=O)NCCOCCOCCOCCOCCC(=O)O)NC(=O)[C@@H]2C[C@@H](O)CN2C(=O)[C@@H](NC(=O)C2(F)CC2)C(C)(C)C)cc1 ; COCCN1C[C@@H](NC(=O)Nc2c(C)c(-c3cnc(N4CCN(C(=O)CCOCCOCCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1]

CPD-018 was synthesized following the same procedure as CPD-001 (3.3 mg, yield: 260%). MS (ESI) m/z=1404.2 [M+H]⁺.

### Example 19: 1-(3-(2-(4-((2-(2,6-Dioxopiperidin-3-yl)-1,3-dioxoisoindolin-5-yl)glycyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-019)

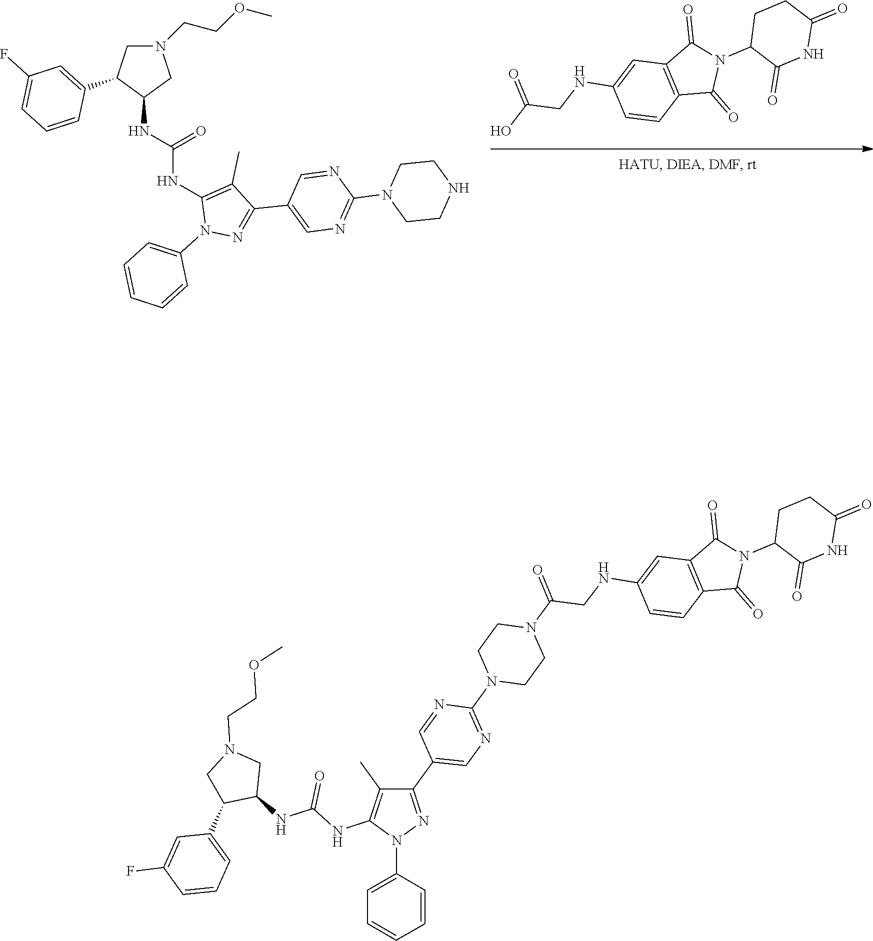 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CNc1ccc2c(c1)C(=O)N(C1CCC(=O)NC1=O)C2=O ; COCCN1C[C@@H](NC(=O)Nc2c(C)c(-c3cnc(N4CCN(C(=O)CNc5ccc6c(c5)C(=O)N(C5CCC(=O)NC5=O)C6=O)CC4)nc3)nn2-c2ccccc2)[C@H](c2cccc(F)c2)C1]

CPD-019 was synthesized following the same procedure as CPD-001 (3 mg, yield: 37%). MS (ESI) m/z=913.8 [M+H]⁺.

### Example 20: (2S,4R)-1-((S)-2-(1-Fluorocyclopropane-1-carboxamido)-3,3-dimethylbutanoyl)-N-(2-((6-(4-(5-(5-(3-((3S,4R)-4-(3-fluorophenyl)-1-(2-methoxyethyl)pyrrolidin-3-yl)ureido)-4-methyl-1-phenyl-1H-pyrazol-3-yl)pyrimidin-2-yl)piperazin-1-yl)-6-oxohexyl)oxy)-4-(4-methylthiazol-5-yl)benzyl)-4-hydroxypyrrolidine-2-carboxamide (CPD-020)

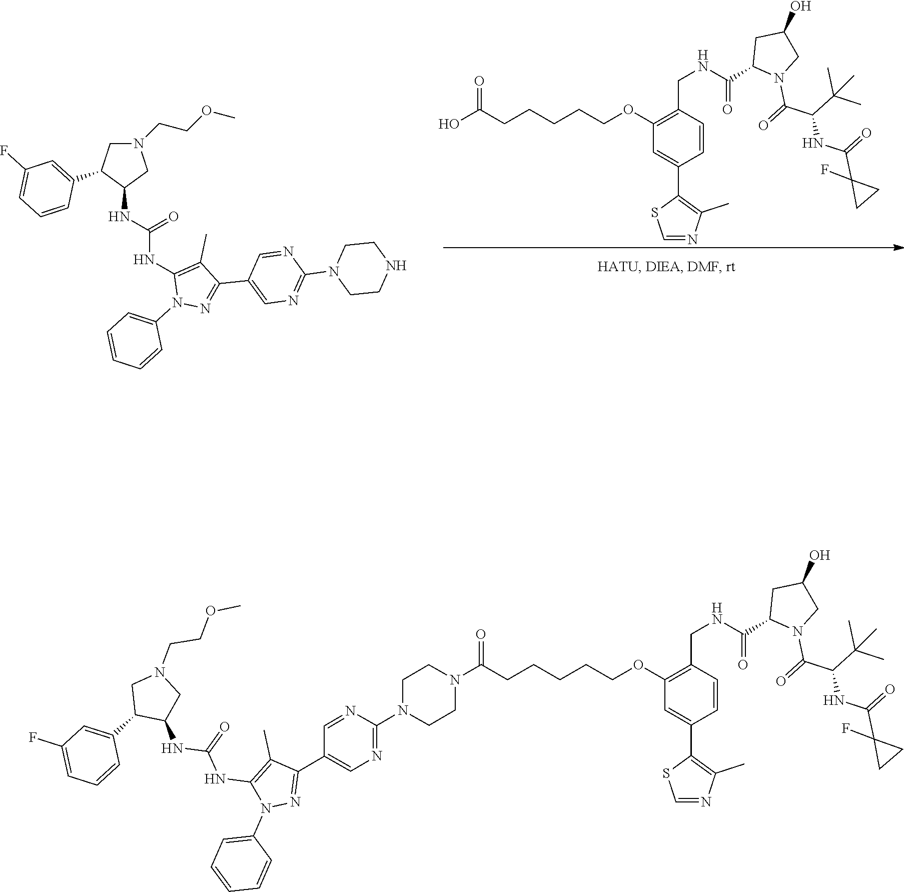 [SMILES: COCCN1C[C@@H](NC(=O)Nc2c(C)c(-c3cnc(N4CCNCC4)nc3)nn2-c2ccccc2)[C@H](c2cccc(F)c2)C1 ; Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)O)c1 ; COCCN1C[C@@H](NC(=O)Nc2c(C)c(-c3cnc(N4CCN(C(=O)CCCCCOc5cc(-c6scnc6C)ccc5CNC(=O)[C@@H]5C[C@@H](O)CN5C(=O)[C@@H](NC(=O)C5(F)CC5)C(C)(C)C)CC4)nc3)nn2-c2ccccc2)[C@H](c2cccc(F)c2)C1]

CPD-020 was synthesized following the same procedure as CPD-001 (3 mg, yield: 29%). MS (ESI) m/z=1229.3 [M+H]⁺.

### Example 21: 1-(3-(2-(4-(2-((2-(2,6-Dioxopiperidin-3-yl)-1,3-dioxoisoindolin-5-yl)oxy)acetyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-023)

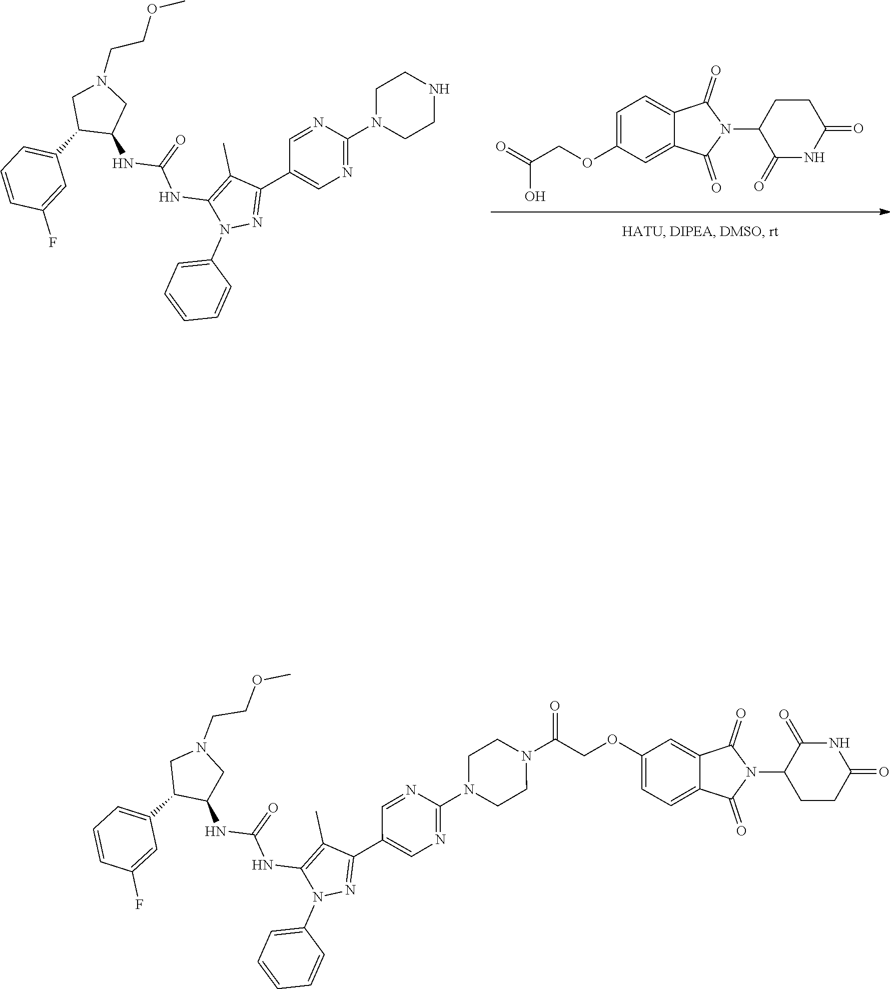 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)COc1ccc2c(c1)C(=O)N(C1CCC(=O)NC1=O)C2=O ; COCCN1C[C@@H](NC(=O)Nc2c(C)c(-c3cnc(N4CCN(C(=O)COc5ccc6c(c5)C(=O)N(C5CCC(=O)NC5=O)C6=O)CC4)nc3)nn2-c2ccccc2)[C@H](c2cccc(F)c2)C1]

CPD-023 was synthesized following the same procedure as CPD-001 (1.1 mg, yield: 14%) as a white solid. MS (ESI) m/z=914.9 [M+H]⁺.

### Example 22: 1-(3-(2-(4-(2-((2-(2,6-Dioxopiperidin-3-yl)-1,3-dioxoisoindolin-4-yl)oxy)acetyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-024)

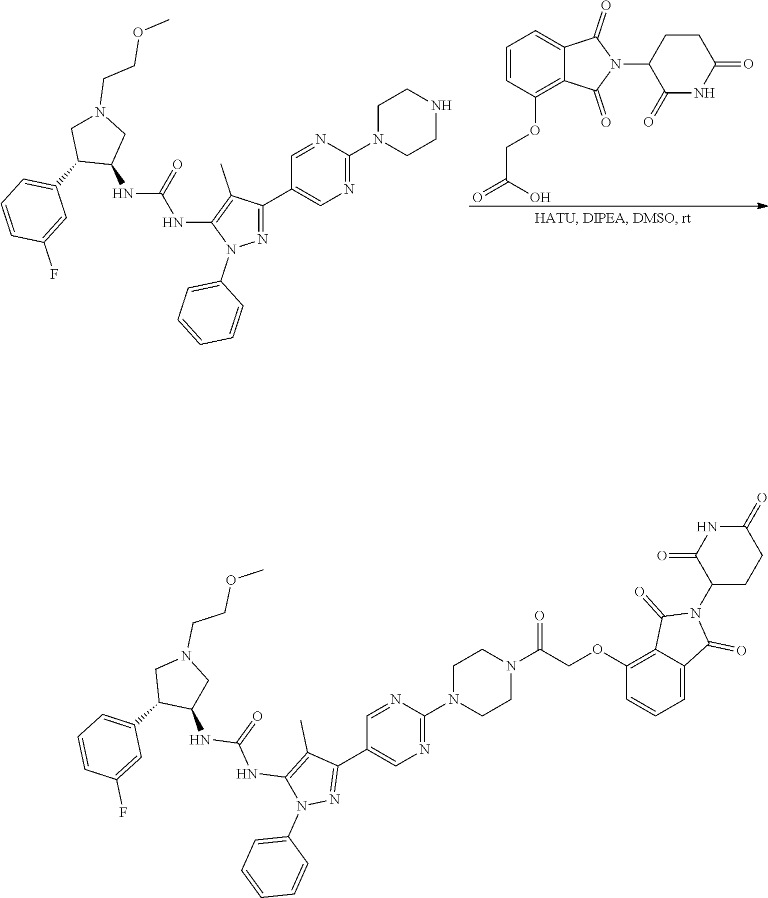 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)COc1cccc2c1C(=O)N(C1CCC(=O)NC1=O)C2=O ; COCCN1C[C@@H](NC(=O)Nc2c(C)c(-c3cnc(N4CCN(C(=O)COc5cccc6c5C(=O)N(C5CCC(=O)NC5=O)C6=O)CC4)nc3)nn2-c2ccccc2)[C@H](c2cccc(F)c2)C1]

CPD-024 was synthesized following the same procedure as CPD-001 (3.9 mg, yield: 51%) as a white solid. MS (ESI) m/z=915.0 [M+H]⁺.

### Example 23: 1-(3-(2-(4-(3-(2-(2,6-Dioxopiperidin-3-yl)-1,3-dioxoisoindolin-4-yl)propanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-025)

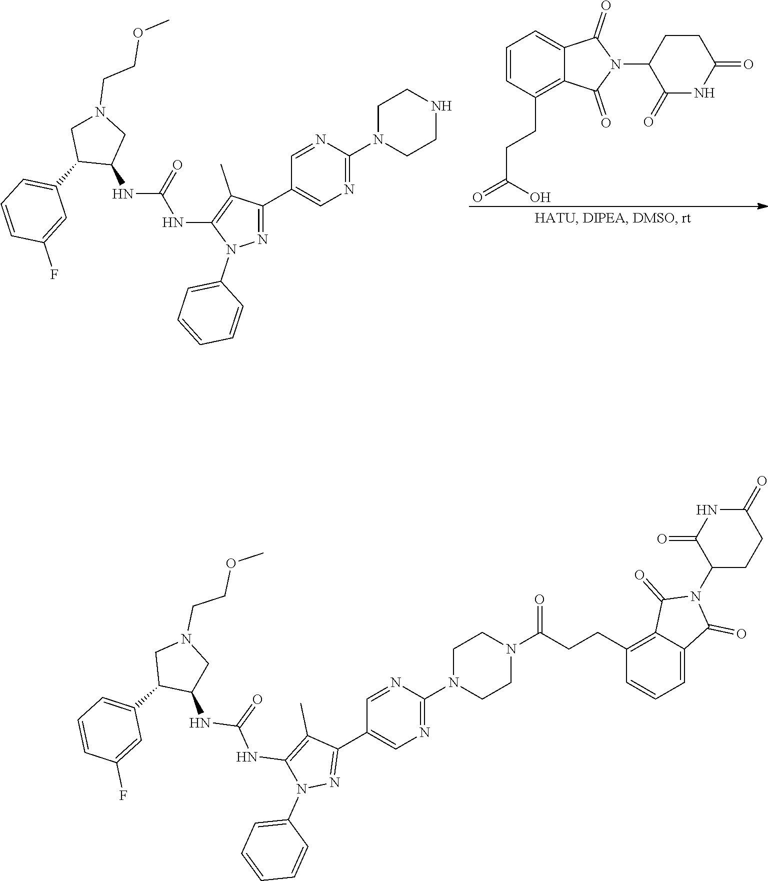 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCc1cccc2c1C(=O)N(C1CCC(=O)NC1=O)C2=O ; COCCN1C[C@@H](NC(=O)Nc2c(C)c(-c3cnc(N4CCN(C(=O)CCc5cccc6c5C(=O)N(C5CCC(=O)NC5=O)C6=O)CC4)nc3)nn2-c2ccccc2)[C@H](c2cccc(F)c2)C1]

CPD-025 was synthesized following the same procedure as CPD-001 (3.7 mg, yield: 49%) as a white solid. MS (ESI) m/z=913.0 [M+H]⁺.

### Example 24: 1-(3-(2-(4-(3-(2-(2,6-Dioxopiperidin-3-yl)-1,3-dioxoisoindolin-5-yl)propanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-026)

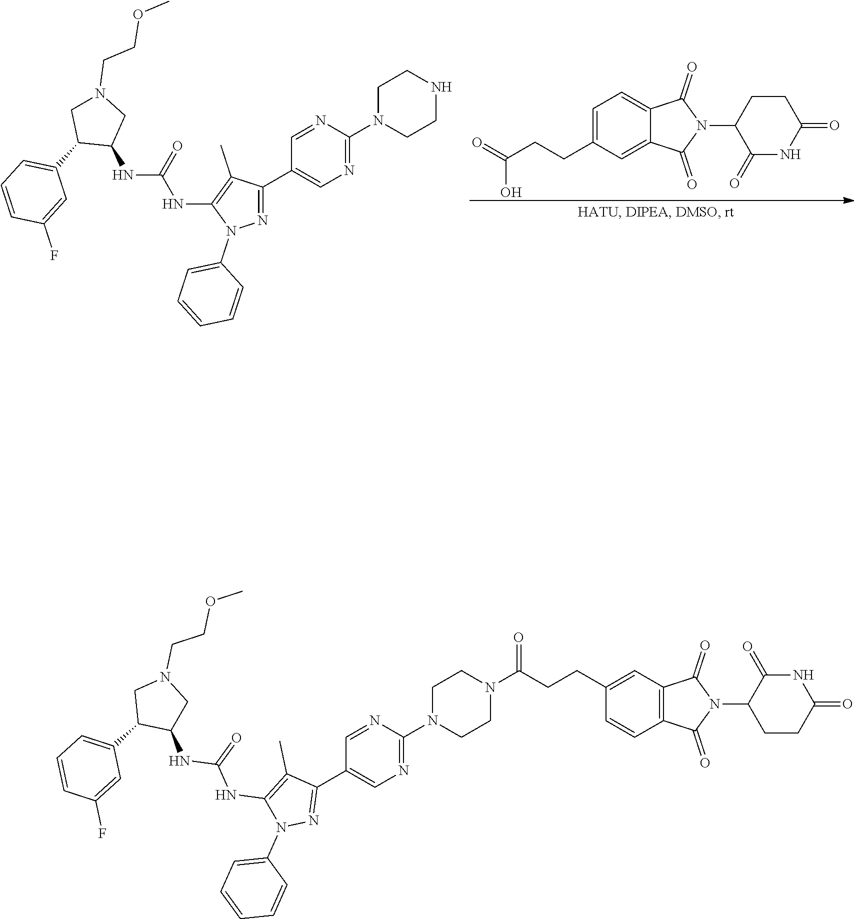 [SMILES: COCCN1C[C@@H](NC(=O)Nc2c(C)c(-c3cnc(N4CCNCC4)nc3)nn2-c2ccccc2)[C@H](c2cccc(F)c2)C1 ; O=C(O)CCc1ccc2c(c1)C(=O)N(C1CCC(=O)NC1=O)C2=O ; COCCN1C[C@@H](NC(=O)Nc2c(C)c(-c3cnc(N4CCN(C(=O)CCc5ccc6c(c5)C(=O)N(C5CCC(=O)NC5=O)C6=O)CC4)nc3)nn2-c2ccccc2)[C@H](c2cccc(F)c2)C1]

CPD-026 was synthesized following the same procedure as CPD-001 (3.5 mg, yield: 46%) as a white solid. MS (ESI) m/z=912.8 [M+H]⁺.

### Example 25: 1-(3-(2-(4-(2-((2-(2,6-Dioxopiperidin-3-yl)-1,3-dioxoisoindolin-4-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-027)

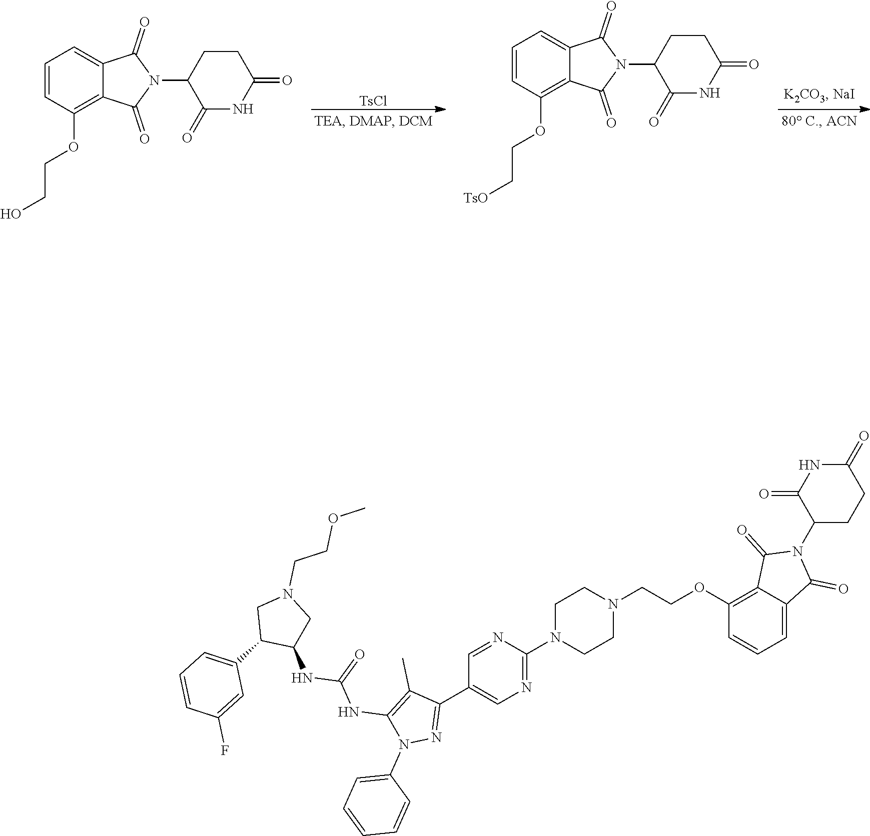 [SMILES: O=C1CCC(N2C(=O)c3cccc(OCCO)c3C2=O)C(=O)N1 ; "TsCl" ; Cc1ccc(S(=O)(=O)OCCOc2cccc3c2C(=O)N(C2CCC(=O)NC2=O)C3=O)cc1 ; COCCN1C[C@@H](NC(=O)Nc2c(C)c(-c3cnc(N4CCN(CCOc5cccc6c5C(=O)N(C5CCC(=O)NC5=O)C6=O)CC4)nc3)nn2-c2ccccc2)[C@H](c2cccc(F)c2)C1]

### Step 1. Synthesis of 2-((2-(2,6-dioxopiperidin-3-yl)-1,3-dioxoisoindolin-4-yl)oxy)ethyl 4-methylbenzenesulfonate

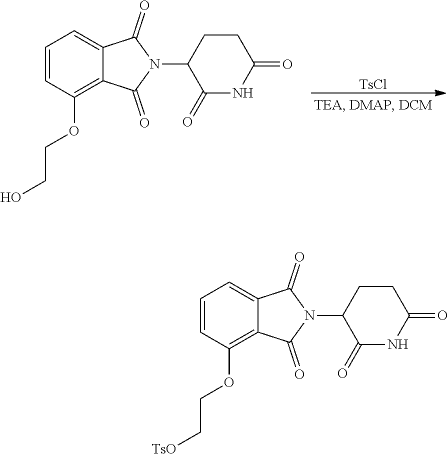 [SMILES: O=C1CCC(N2C(=O)c3cccc(OCCO)c3C2=O)C(=O)N1 ; "TsCl" ; Cc1ccc(S(=O)(=O)OCCOc2cccc3c2C(=O)N(C2CCC(=O)NC2=O)C3=O)cc1]

To a mixture of 2-(2,6-dioxopiperidin-3-yl)-4-(2-hydroxyethoxy)isoindoline-1,3-dione (50 mg, 157 umol) and TsCl (45 mg, 235 umol) in TEA (0.5 mL) and DCM (1 mL) was added DMAP (2 mg, 15 umol). The reaction mixture was stirred at rt for 2 h, before it was quenched with water. The resulting mixture was extracted with DCM. The organic layer was washed with brine and concentrated. The residue was purified by reverse phase chromatography (0-70% MeCN in H₂O) to afford the title compound (30 mg, yield: 40%) as a white solid. MS (ESI) m/z=473.5 [M+H]⁺.

### Step 2. Synthesis of 1-(3-(2-(4-(2-((2-(2,6-dioxopiperidin-3-yl)-1,3-dioxoisoindolin-4-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea

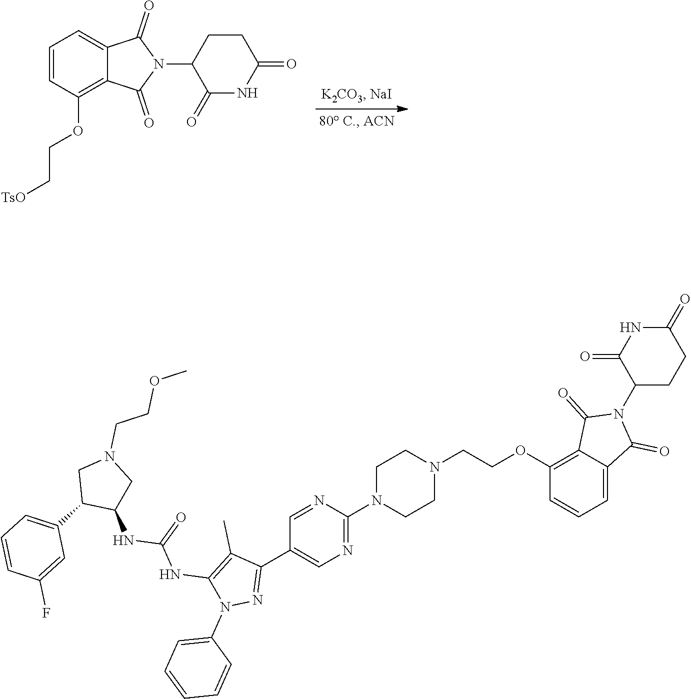 [SMILES: Cc1ccc(S(=O)(=O)OCCOc2cccc3c2C(=O)N(C2CCC(=O)NC2=O)C3=O)cc1 ; COCCN1C[C@@H](NC(=O)Nc2c(C)c(-c3cnc(N4CCN(CCOc5cccc6c5C(=O)N(C5CCC(=O)NC5=O)C6=O)CC4)nc3)nn2-c2ccccc2)[C@H](c2cccc(F)c2)C1]

To a mixture of 1-((3S,4R)-4-(3-fluorophenyl)-1-(2-methoxyethyl)pyrrolidin-3-yl)-3-(4-methyl-1-phenyl-3-(2-(piperazin-1-yl)pyrimidin-5-yl)-1H-pyrazol-5-yl)urea (10 mg, 17 μmol) and 2-((2-(2,6-dioxopiperidin-3-yl)-1,3-dioxoisoindolin-4-yl)oxy)ethyl 4-methylbenzenesulfonate (12 mg, 25 umol) in MeCN (1 mL) were added K₂CO₃ (5 mg, 33 mol) and NaI (4 mg, 25 μmol). The reaction mixture was stirred at 80° C. overnight, before it was purified by reverse phase chromatography (0-70% MeCN in H₂O) to afford the title compound (5.1 mg, yield: 34%) as a white solid. MS (ESI) m/z=900.5 [M+H]⁺.

### Example 26: 1-(3-(2-(4-(2-((2-(2,6-Dioxopiperidin-3-yl)-1,3-dioxoisoindolin-5-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-028)

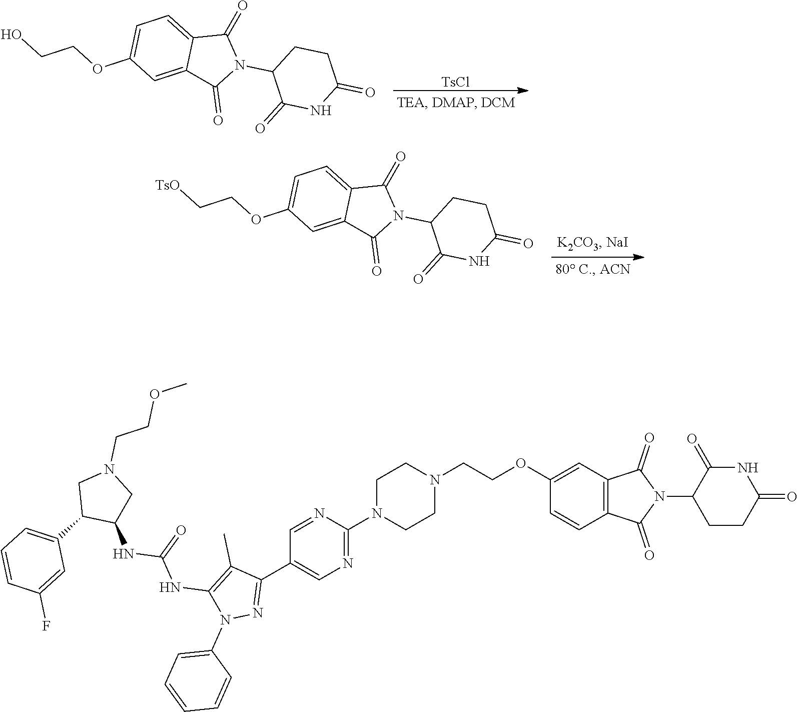 [SMILES: O=C1CCC(N2C(=O)c3ccc(OCCO)cc3C2=O)C(=O)N1 ; "TsCl" ; Cc1ccc(S(=O)(=O)OCCOc2ccc3c(c2)C(=O)N(C2CCC(=O)NC2=O)C3=O)cc1 ; COCCN1C[C@@H](NC(=O)Nc2c(C)c(-c3cnc(N4CCN(CCOc5ccc6c(c5)C(=O)N(C5CCC(=O)NC5=O)C6=O)CC4)nc3)nn2-c2ccccc2)[C@H](c2cccc(F)c2)C1]

CPD-028 was synthesized following the same procedure as CPD-027 (5.3 mg, yield: 14% over 2 steps) as a white solid. MS (ESI) m/z=900.6 [M+H]⁺.

### Example 27: 1-(3-(2-(4-(3-(2-(2,6-Dioxopiperidin-3-yl)-1,3-dioxoisoindolin-4-yl)propyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-029)

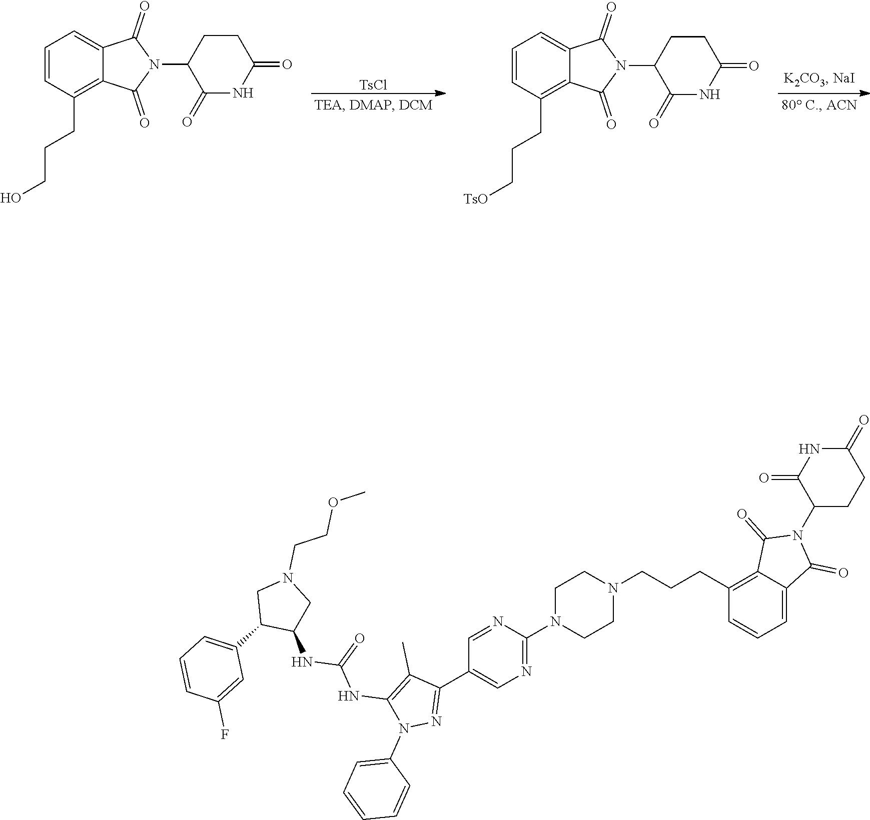 [SMILES: O=C1CCC(N2C(=O)c3cccc(CCCO)c3C2=O)C(=O)N1 ; "TsCl" ; Cc1ccc(S(=O)(=O)OCCCc2cccc3c2C(=O)N(C2CCC(=O)NC2=O)C3=O)cc1 ; COCCN1C[C@@H](NC(=O)Nc2c(C)c(-c3cnc(N4CCN(CCCc5cccc6c5C(=O)N(C5CCC(=O)NC5=O)C6=O)CC4)nc3)nn2-c2ccccc2)[C@H](c2cccc(F)c2)C1]

CPD-029 was synthesized following the same procedure as CPD-027 (3.0 mg, yield: 9% over 2 steps) as a white solid. MS (ESI) m/z=898.6 [M+H]⁺.

### Example 28: 1-(3-(2-(4-(2-((2-(2,6-Dioxopiperidin-3-yl)-1,3-dioxoisoindolin-4-yl)amino)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-030)

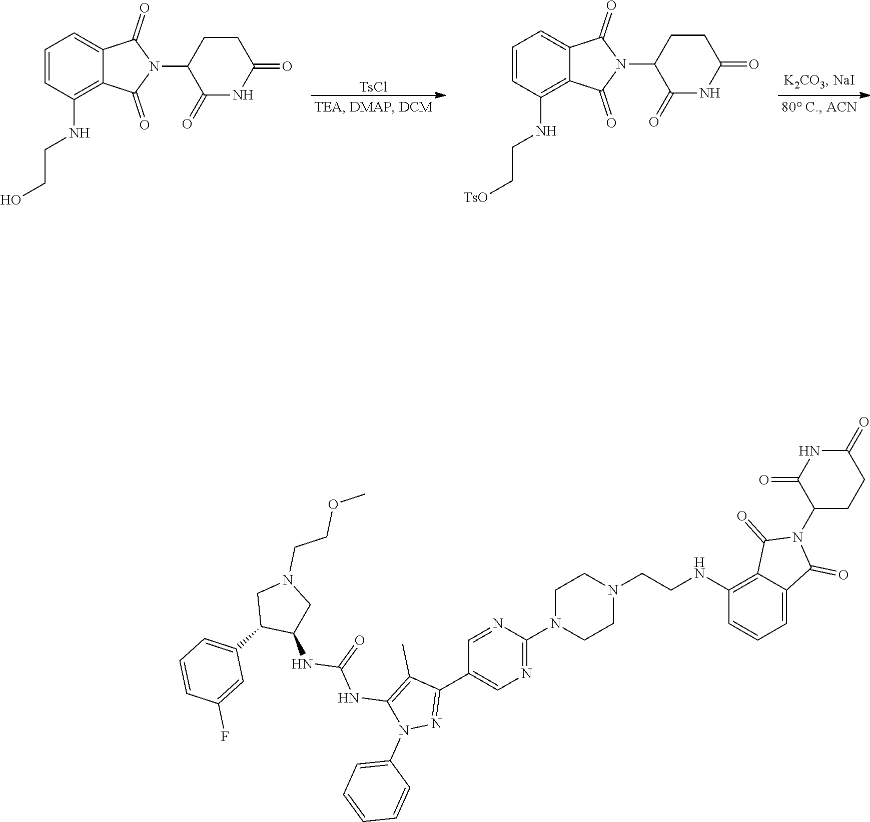 [SMILES: O=C1CCC(N2C(=O)c3cccc(NCCO)c3C2=O)C(=O)N1 ; "TsCl" ; Cc1ccc(S(=O)(=O)OCCNc2cccc3c2C(=O)N(C2CCC(=O)NC2=O)C3=O)cc1 ; COCCN1C[C@@H](NC(=O)Nc2c(C)c(-c3cnc(N4CCN(CCNc5cccc6c5C(=O)N(C5CCC(=O)NC5=O)C6=O)CC4)nc3)nn2-c2ccccc2)[C@H](c2cccc(F)c2)C1]

CPD-030 was synthesized following the same procedure as CPD-027 (5.0 mg, yield: 11% over 2 steps) as a white solid. MS (ESI) m/z=899.6 [M+H]⁺.

### Example 29: 1-(3-(2-(4-(2-((2-(2,6-Dioxopiperidin-3-yl)-1,3-dioxoisoindolin-5-yl)amino)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-031)

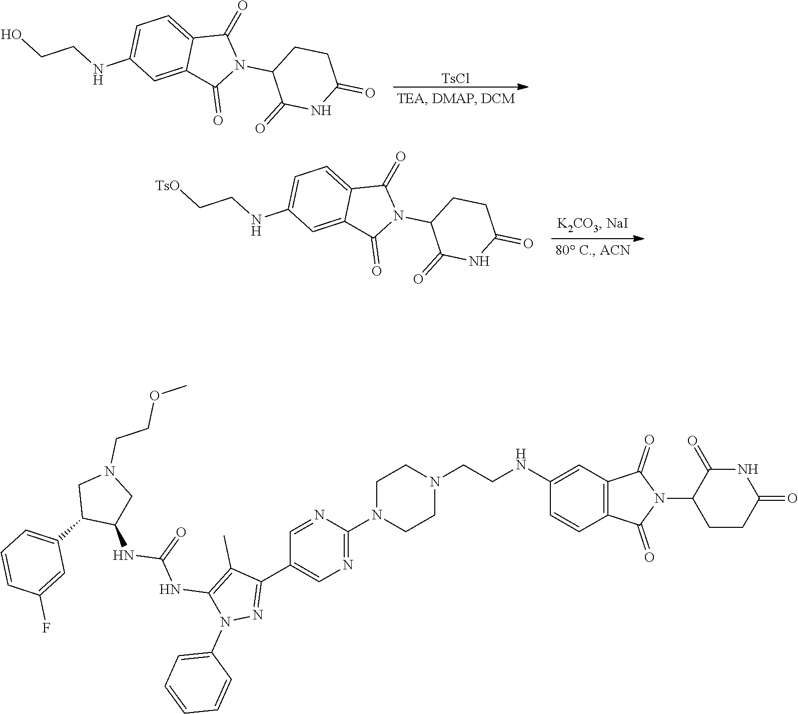 [SMILES: O=C1CCC(N2C(=O)c3ccc(NCCO)cc3C2=O)C(=O)N1 ; "TsCl" ; Cc1ccc(S(=O)(=O)OCCNc2ccc3c(c2)C(=O)N(C2CCC(=O)NC2=O)C3=O)cc1 ; COCCN1C[C@@H](NC(=O)Nc2c(C)c(-c3cnc(N4CCN(CCNc5ccc6c(c5)C(=O)N(C5CCC(=O)NC5=O)C6=O)CC4)nc3)nn2-c2ccccc2)[C@H](c2cccc(F)c2)C1]

CPD-031 was synthesized following the same procedure as CPD-027 (2.5 mg, yield: 6% over 2 steps) as a white solid. MS (ESI) m/z=899.8 [M+H]⁺.

### Example 30: 1-(3-(2-(4-(3-(2-(2,6-Dioxopiperidin-3-yl)-1,3-dioxoisoindolin-5-yl)propyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-032)

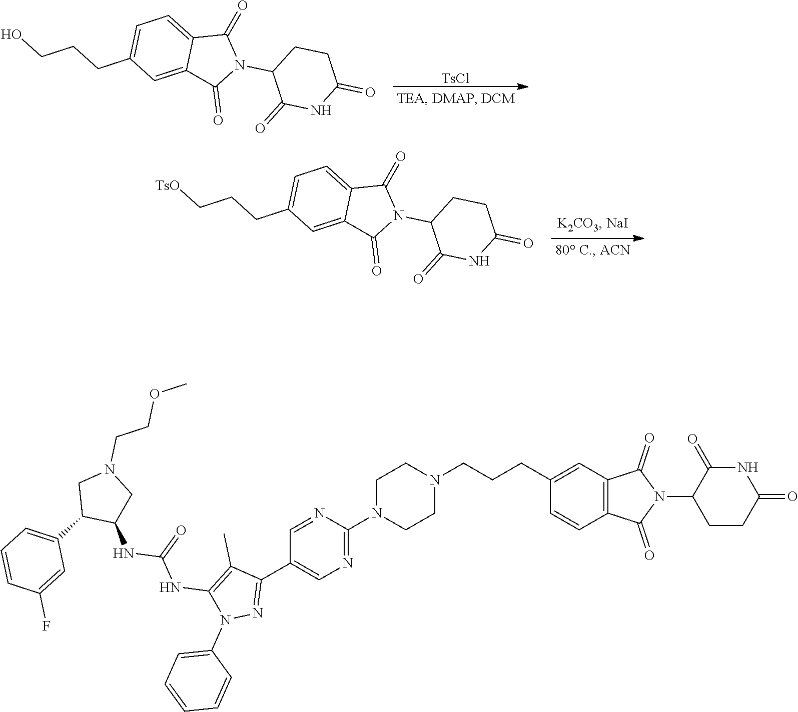 [SMILES: O=C1CCC(N2C(=O)c3ccc(CCCO)cc3C2=O)C(=O)N1 ; "TsCl" ; Cc1ccc(S(=O)(=O)OCCCc2ccc3c(c2)C(=O)N(C2CCC(=O)NC2=O)C3=O)cc1 ; COCCN1C[C@@H](NC(=O)Nc2c(C)c(-c3cnc(N4CCN(CCCc5ccc6c(c5)C(=O)N(C5CCC(=O)NC5=O)C6=O)CC4)nc3)nn2-c2ccccc2)[C@H](c2cccc(F)c2)C1]

CPD-032 was synthesized following the same procedure as CPD-027 (1.1 mg, yield: 3% over 2 steps) as a white solid. MS (ESI) m/z=898.9 [M+H]⁺.

### Example 31: 1-(3-(2-(4-(2-(2-(2,6-Dioxopiperidin-3-yl)-1,3-dioxoisoindolin-5-yl)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl urea (CPD-033)

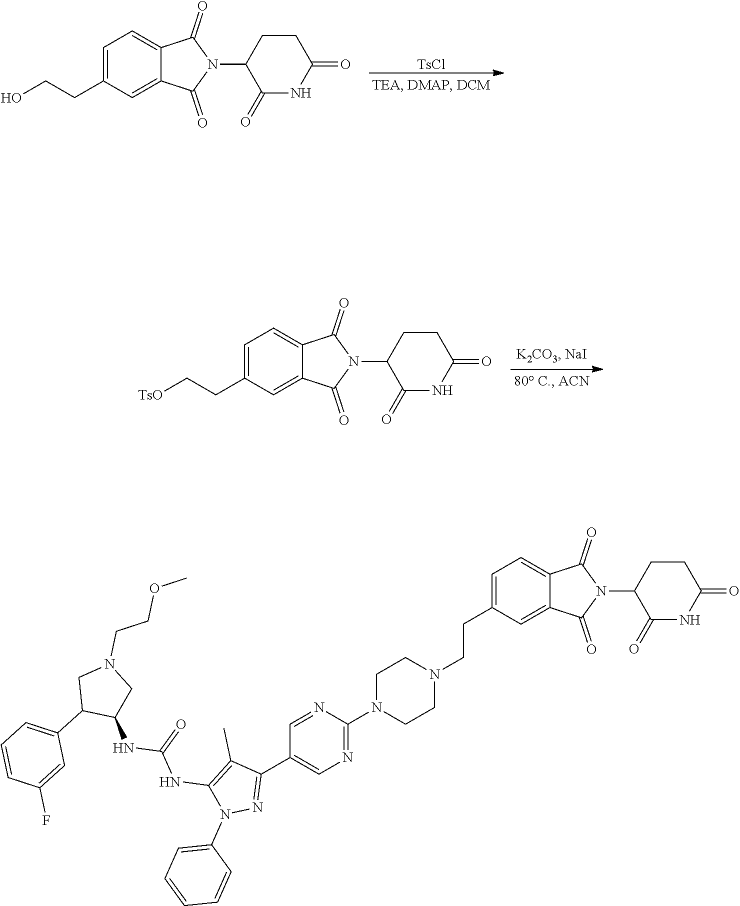 [SMILES: O=C1CCC(N2C(=O)c3ccc(CCO)cc3C2=O)C(=O)N1 ; "TsCl" ; Cc1ccc(S(=O)(=O)OCCc2ccc3c(c2)C(=O)N(C2CCC(=O)NC2=O)C3=O)cc1 ; COCCN1CC(c2cccc(F)c2)[C@H](NC(=O)Nc2c(C)c(-c3cnc(N4CCN(CCc5ccc6c(c5)C(=O)N(C5CCC(=O)NC5=O)C6=O)CC4)nc3)nn2-c2ccccc2)C1]

CPD-033 was synthesized following the same procedure as CPD-027 (2.1 mg, yield: 4% over 2 steps) as a white solid. MS (ESI) m/z=884.8 [M+H]⁺.

### Example 32: 1-(3-(2-(4-Acetylpiperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (WH-1)

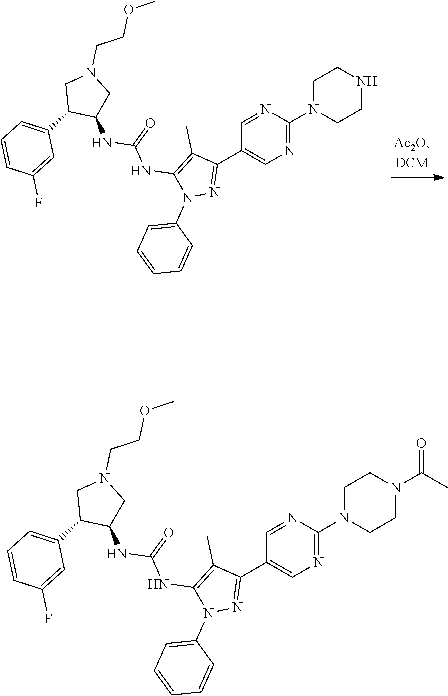 [SMILES: COCCN1C[C@@H](NC(=O)Nc2c(C)c(-c3cnc(N4CCNCC4)nc3)nn2-c2ccccc2)[C@H](c2cccc(F)c2)C1 ; COCCN1C[C@@H](NC(=O)Nc2c(C)c(-c3cnc(N4CCN(C(C)=O)CC4)nc3)nn2-c2ccccc2)[C@H](c2cccc(F)c2)C1]

To a solution of 1-((3S,4R)-4-(3-fluorophenyl)-1-(2-methoxyethyl)pyrrolidin-3-yl)-3-(4-methyl-1-phenyl-3-(2-(piperazin-1-yl)pyrimidin-5-yl)-1H-pyrazol-5-yl)urea (15 mg, 24.76 mol) in DCM (3 mL) was added Ac₂O (0.3 mL) at rt. The mixture was stirred at this temperature overnight, before it was concentrated. The residue was purified by reverse phase chromatography to afford the title compound (8 mg, yield: 50%) as a white solid.

Certain compounds disclosed herein have the structures shown in Table 1.

TABLE 1

| Cpd. | Structure | Chemical Name |
|---|---|---|
| CPD- 001 | 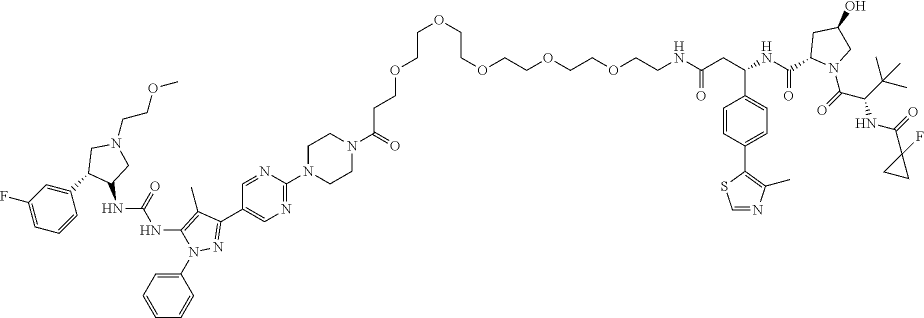 | (2S,4R)-1-((S)-2-(1- fluorocyclopropane-1-carboxamido)- 3,3-dimethylbutanoyl)-N-((S)-22-(4-(5- (5-(3-((3S,4R)-4-(3-fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-1-(4- (4-methylthiazol-5-yl)phenyl)-3,22- dioxo-7,10,13,16,19-pentaoxa-4- azadocosyl)-4-hydroxypyrrolidine-2- carboxamide |
| CPD- 002 | 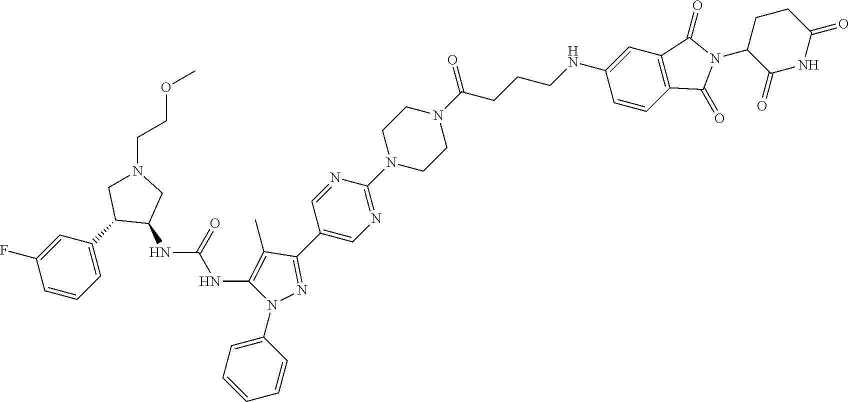 [SMILES: COCCN1C[C@@H](NC(=O)Nc2c(C)c(-c3cnc(N4CCN(C(=O)CCCNc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(4-((2-(2,6-dioxopiperidin-3- yl)-1,3-dioxoisoindolin-5- yl)amino)butanoyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 003 | 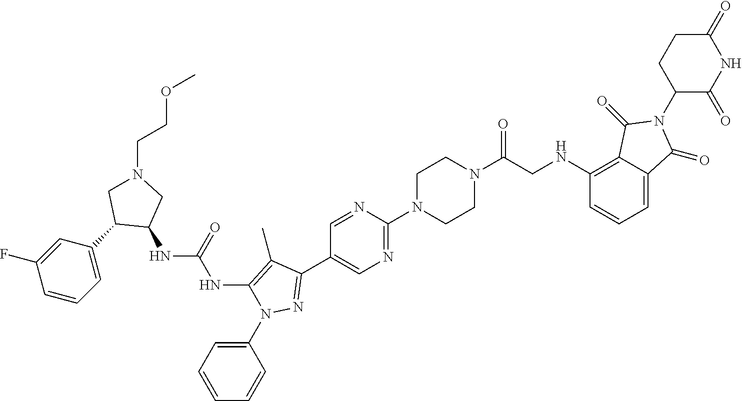 [SMILES: COCCN1C[C@@H](NC(=O)Nc2c(C)c(-c3cnc(N4CCN(C(=O)CNc5cccc6c5C(=O)N(C5CCC(=O)NC5=O)C6=O)CC4)nc3)nn2-c2ccccc2)[C@H](c2cccc(F)c2)C1] | 1-(3-(2-(4-((2-(2,6-dioxopiperidin-3- yl)-1,3-dioxoisoindolin-4- yl)glycyl)piperazin-1-yl)pyrimidin-5- yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 004 | 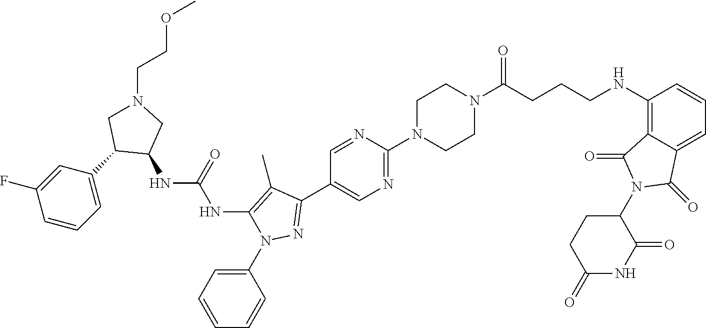 [SMILES: COCCN1C[C@@H](NC(=O)Nc2c(C)c(-c3cnc(N4CCN(C(=O)CCCNc5cccc6c5C(=O)N(C5CCC(=O)NC5=O)C6=O)CC4)nc3)nn2-c2ccccc2)[C@H](c2cccc(F)c2)C1] | 1-(3-(2-(4-(4-((2-(2,6-dioxopiperidin-3- yl)-1,3-dioxoisoindolin-4- yl)amino)butanoyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 005 | 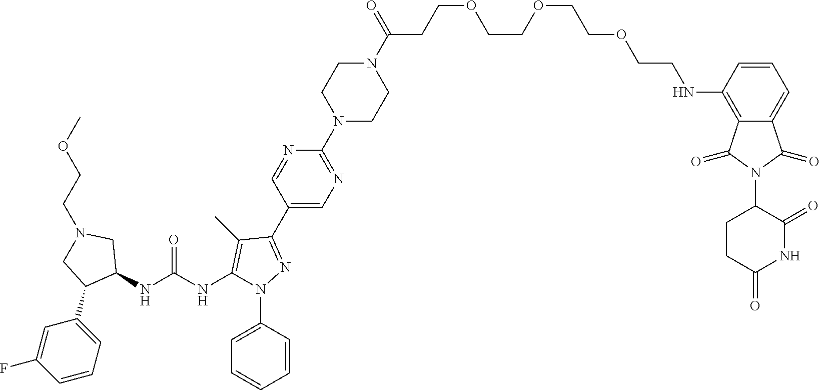 [SMILES: COCCN1C[C@@H](NC(=O)Nc2c(C)c(-c3cnc(N4CCN(C(=O)CCOCCOCCOCCNc5cccc6c5C(=O)N(C5CCC(=O)NC5=O)C6=O)CC4)nc3)nn2-c2ccccc2)[C@H](c2cccc(F)c2)C1] | 1-(3-(2-(4-(3-(2-(2-(2-((2-(2,6- dioxopiperidin-3-yl)-1,3- dioxoisoindolin-4- yl)amino)ethoxy)ethoxy)ethoxy) propanoyl)piperazin-1-yl)pyrimidin-5-yl)-4- methyl-1-phenyl-1H-pyrazol-5-yl)-3- ((3S,4R)-4-(3-fluorophenyl)-1-(2- methoxyethyl)pyrrolidin-3-yl)urea |
| CPD- 006 | 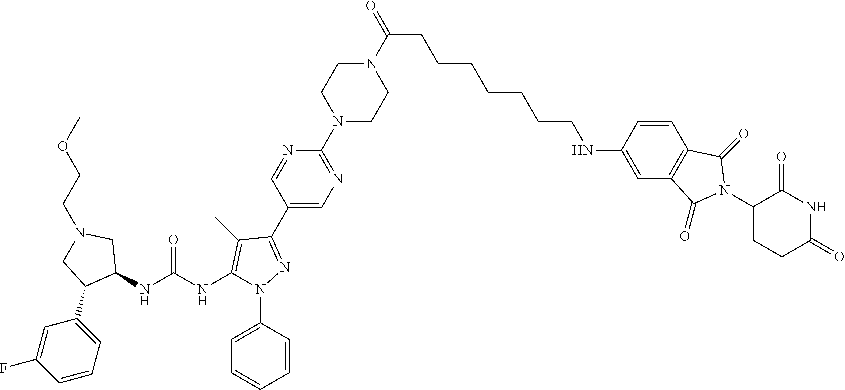 [SMILES: COCCN1C[C@@H](NC(=O)Nc2c(C)c(-c3cnc(N4CCN(C(=O)CCCCCCCNc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(8-((2-(2,6-dioxopiperidin-3- yl)-1,3-dioxoisoindolin-5- yl)amino)octanoyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 007 | 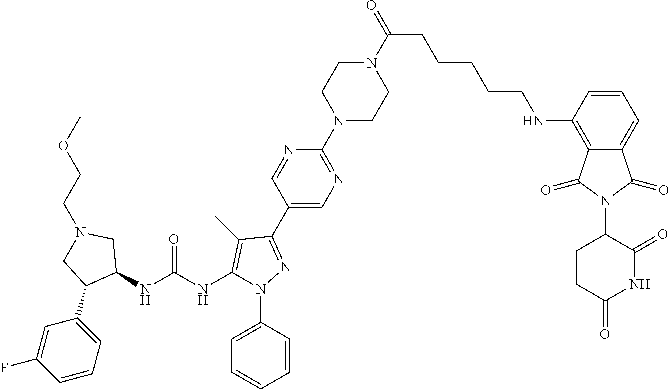 [SMILES: COCCN1C[C@@H](NC(=O)Nc2c(C)c(-c3cnc(N4CCN(C(=O)CCCCCNc5cccc6c5C(=O)N(C5CCC(=O)NC5=O)C6=O)CC4)nc3)nn2-c2ccccc2)[C@H](c2cccc(F)c2)C1] | 1-(3-(2-(4-(6-((2-(2,6-dioxopiperidin-3- yl)-1,3-dioxoisoindolin-4- yl)amino)hexanoyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 008 | 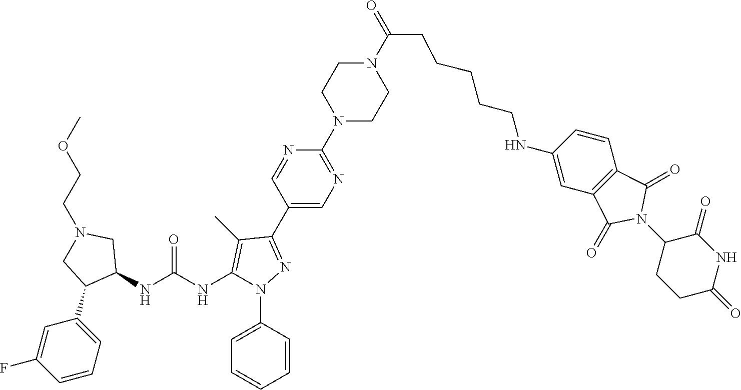 [SMILES: COCCN1C[C@@H](NC(=O)Nc2c(C)c(-c3cnc(N4CCN(C(=O)CCCCCNc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(6-((2-(2,6-dioxopiperidin-3- yl)-1,3-dioxoisoindolin-5- yl)amino)hexanoyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 009 | 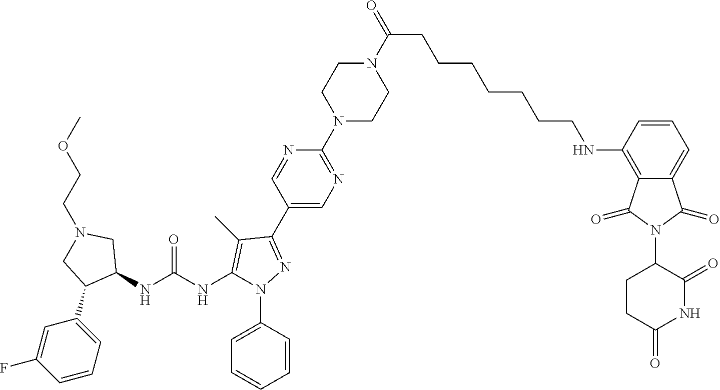 [SMILES: COCCN1C[C@@H](NC(=O)Nc2c(C)c(-c3cnc(N4CCN(C(=O)CCCCCCCNc5cccc6c5C(=O)N(C5CCC(=O)NC5=O)C6=O)CC4)nc3)nn2-c2ccccc2)[C@H](c2cccc(F)c2)C1] | 1-(3-(2-(4-(8-((2-(2,6-dioxopiperidin-3- yl)-1,3-dioxoisoindolin-4- yl)amino)octanoyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 010 | 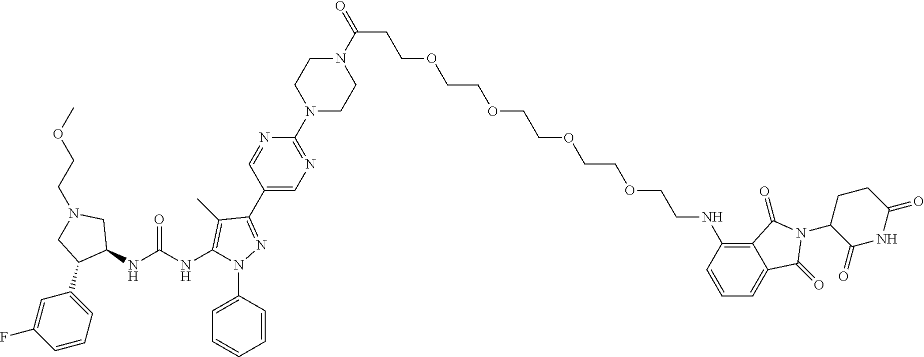 [SMILES: COCCN1C[C@@H](NC(=O)Nc2c(C)c(-c3cnc(N4CCN(C(=O)CCOCCOCCOCCOCCNc5cccc6c5C(=O)N(C5CCC(=O)NC5=O)C6=O)CC4)nc3)nn2-c2ccccc2)[C@H](c2cccc(F)c2)C1] | 1-(3-(2-(4-(1-((2-(2,6-dioxopiperidin-3- yl)-1,3-dioxoisoindolin-4-yl)amino)- 3,6,9,12-tetraoxapentadecan-15- oyl)piperazin-1-yl)pyrimidin-5-yl)-4- methyl-1-phenyl-1H-pyrazol-5-yl)-3- ((3S,4R)-4-(3-fluorophenyl)-1-(2- methoxyethyl)pyrrolidin-3-yl)urea |
| CPD- 011 | 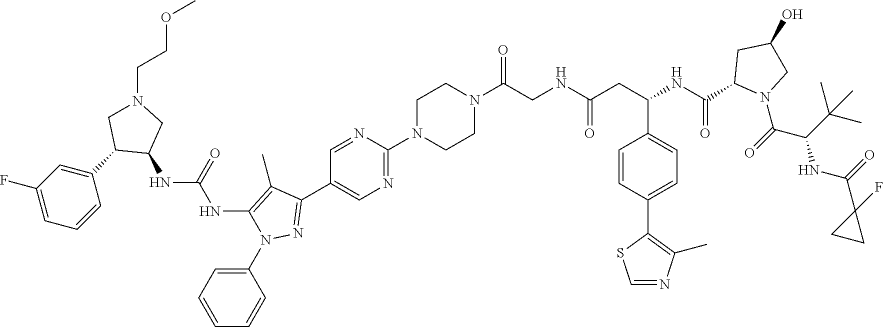 [SMILES: COCCN1C[C@@H](NC(=O)Nc2c(C)c(-c3cnc(N4CCN(C(=O)CNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-3-((2-(4- (5-(5-(3-((3S,4R)-4-(3-fluorophenyl)-1- (2-methoxyethyl)pyrrolidin-3- yl)ureido)-4-methyl-1-phenyl-1H- pyrazol-3-yl)pyrimidin-2-yl)piperazin- 1-yl)-2-oxoethyl)amino)-1-(4-(4- methylthiazol-5-yl)phenyl)-3- oxopropyl)-4-hydroxypyrrolidine-2- carboxamide |
| CPD- 012 | 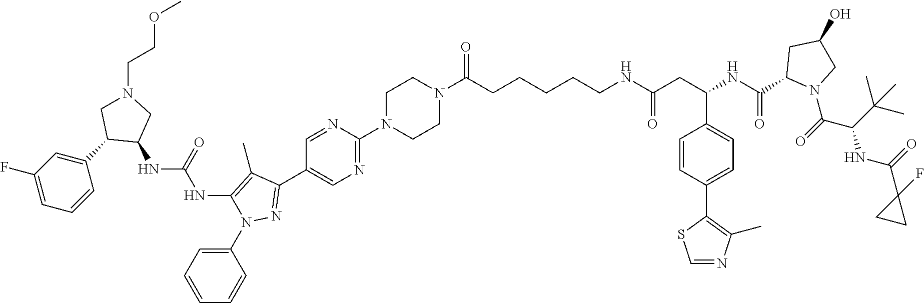 [SMILES: COCCN1C[C@@H](NC(=O)Nc2c(C)c(-c3cnc(N4CCN(C(=O)CCCCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-3-((6-(4- (5-(5-(3-((3S,4R)-4-(3-fluorophenyl)-1- (2-methoxyethyl)pyrrolidin-3- yl)ureido)-4-methyl-1-phenyl-1H- pyrazol-3-yl)pyrimidin-2-yl)piperazin- 1-yl)-6-oxohexyl)amino)-1-(4-(4- methylthiazol-5-yl)phenyl)-3- oxopropyl)-4-hydroxypyrrolidine-2- carboxamide |
| CPD- 013 | 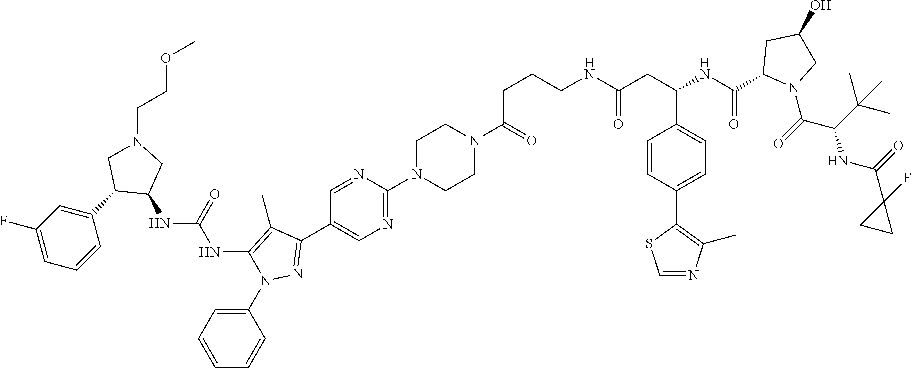 [SMILES: COCCN1C[C@@H](NC(=O)Nc2c(C)c(-c3cnc(N4CCN(C(=O)CCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-3-((4-(4- (5-(5-(3-((3S,4R)-4-(3-fluorophenyl)-1- (2-methoxyethyl)pyrrolidin-3- yl)ureido)-4-methyl-1-phenyl-1H- pyrazol-3-yl)pyrimidin-2-yl)piperazin- 1-yl)-4-oxobutyl)amino)-1-(4-(4- methylthiazol-5-yl)phenyl)-3- oxopropyl)-4-hydroxypyrrolidine-2- carboxamide |
| CPD- 014 | 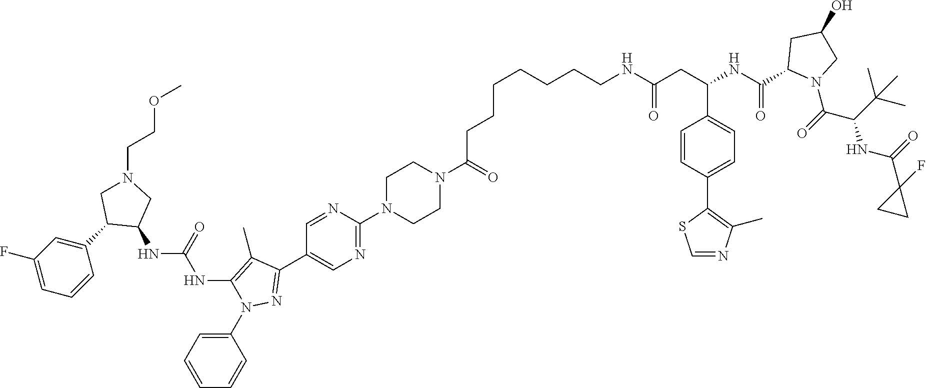 [SMILES: COCCN1C[C@@H](NC(=O)Nc2c(C)c(-c3cnc(N4CCN(C(=O)CCCCCCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-3-((8-(4- (5-(5-(3-((3S,4R)-4-(3-fluorophenyl)-1- (2-methoxyethyl)pyrrolidin-3- yl)ureido)-4-methyl-1-phenyl-1H- pyrazol-3-yl)pyrimidin-2-yl)piperazin- 1-yl)-8-oxooctyl)amino)-1-(4-(4- methylthiazol-5-yl)phenyl)-3- oxopropyl)-4-hydroxypyrrolidine-2- carboxamide |
| CPD- 015 | 167 | (2S,4R)-1-((S)-2-(1- fluorocyclopropane-1-carboxamido)- 3,3-dimethylbutanoyl)-N-((S)-3-((10- (4-(5-(5-(3-((3S,4R)-4-(3- fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-10- oxodecyl)amino)-1-(4-(4- methylthiazol-5-yl)phenyl)-3- oxopropyl)-4-hydroxypyrrolidine-2- carboxamide |
| CPD- 016 | 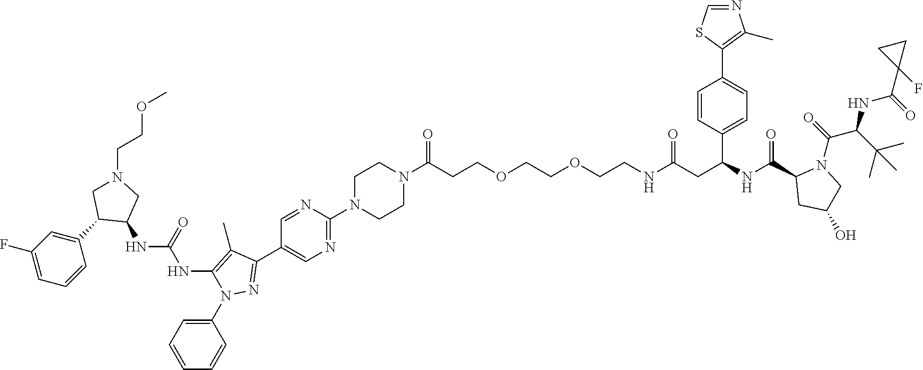 [SMILES: COCCN1C[C@@H](NC(=O)Nc2c(C)c(-c3cnc(N4CCN(C(=O)CCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-3-((2-(2- (3-(4-(5-(5-(3-((3S,4R)-4-(3- fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-3- oxopropoxy)ethoxy)ethyl)amino)-1-(4- (4-methylthiazol-5-yl)phenyl)-3- oxopropyl)-4-hydroxypyrrolidine-2- carboxamide |
| CPD- 017 | 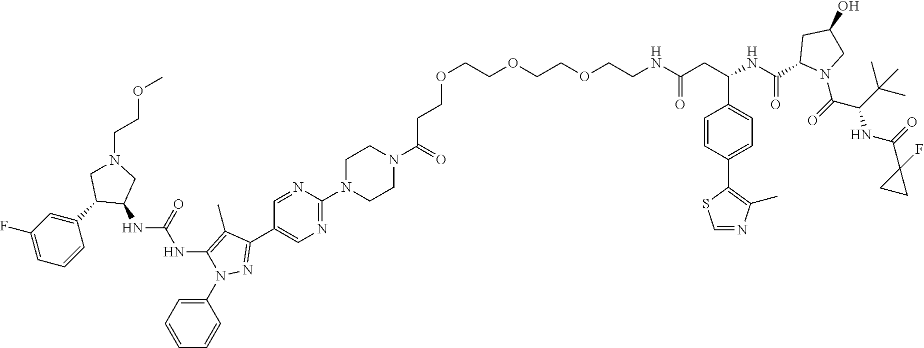 [SMILES: COCCN1C[C@@H](NC(=O)Nc2c(C)c(-c3cnc(N4CCN(C(=O)CCOCCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-16-(4-(5- (5-(3-((3S,4R)-4-(3-fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-1-(4- (4-methylthiazol-5-yl)phenyl)-3,16- dioxo-7,10,13-trioxa-4-azahexadecyl)- 4-hydroxypyrrolidine-2-carboxamide |
| CPD- 018 | 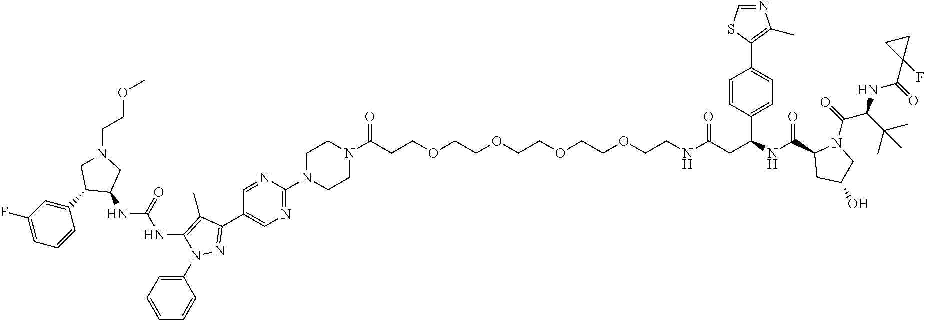 [SMILES: COCCN1C[C@@H](NC(=O)Nc2c(C)c(-c3cnc(N4CCN(C(=O)CCOCCOCCOCCOCCNC(=O)C[C@H](NC(=O)[C@@H]5C[C@@H](O)CN5C(=O)[C@@H](NC(=O)C5(F)CC5)C(C)(C)C)c5ccc(-c6scnc6C)cc5)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-((S)-19-(4-(5- (5-(3-((3S,4R)-4-(3-fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-1-(4- (4-methylthiazol-5-yl)phenyl)-3,19- dioxo-7,10,13,16-tetraoxa-4- azanonadecyl)-4-hydroxypyrrolidine-2- carboxamide |
| CPD- 019 | 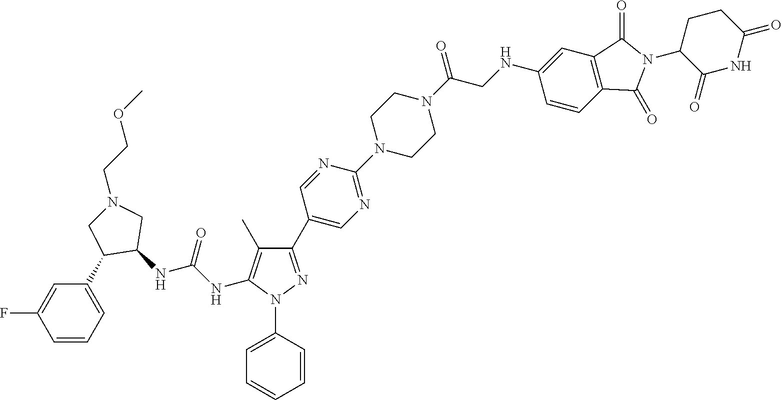 [SMILES: COCCN1C[C@@H](NC(=O)Nc2c(C)c(-c3cnc(N4CCN(C(=O)CNc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-((2-(2,6-dioxopiperidin-3- yl)-1,3-dioxoisoindolin-5- yl)glycyl)piperazin-1-yl)pyrimidin-5- yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 020 | 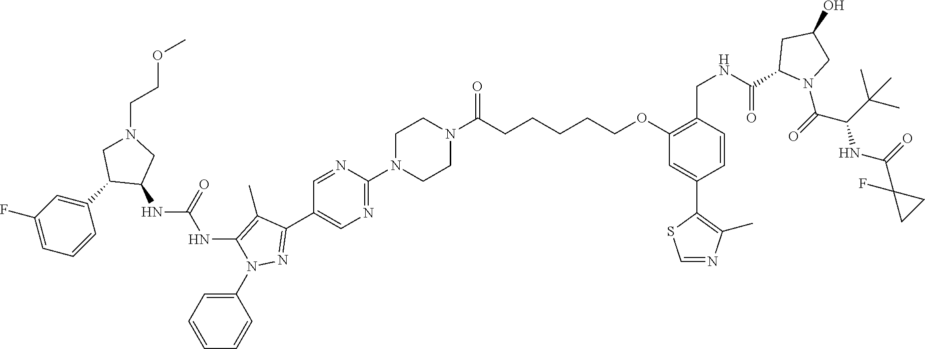 [SMILES: COCCN1C[C@@H](NC(=O)Nc2c(C)c(-c3cnc(N4CCN(C(=O)CCCCCOc5cc(-c6scnc6C)ccc5CNC(=O)[C@@H]5C[C@@H](O)CN5C(=O)[C@@H](NC(=O)C5(F)CC5)C(C)(C)C)CC4)nc3)nn2-c2ccccc2)[C@H](c2cccc(F)c2)C1] | (2S,4R)-1-((S)-2-(1- fluorocyclopropane-1-carboxamido)- 3,3-dimethylbutanoyl)-N-(2-((6-(4-(5- (5-(3-((3S,4R)-4-(3-fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-6- oxohexyl)oxy)-4-(4-methylthiazol-5- yl)benzyl)-4-hydroxypyrrolidine-2- carboxamide |
| CPD- 021 | 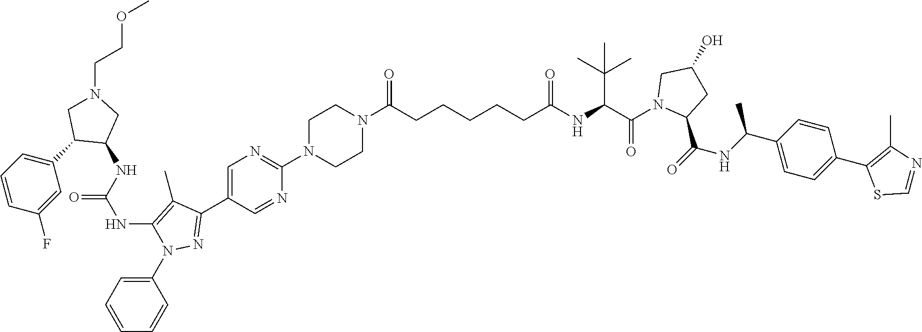 [SMILES: COCCN1C[C@@H](NC(=O)Nc2c(C)c(-c3cnc(N4CCN(C(=O)CCCCCC(=O)N[C@H](C(=O)N5C[C@H](O)C[C@H]5C(=O)N[C@@H](C)c5ccc(-c6scnc6C)cc5)C(C)(C)C)CC4)nc3)nn2-c2ccccc2)[C@H](c2cccc(F)c2)C1] | (2S,4R)-1-((S)-2-(7-(4-(5-(5-(3- ((3S,4R)-4-(3-fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-7- oxoheptanamido)-3,3- dimethylbutanoyl)-4-hydroxy-N-((S)-1- (4-(4-methylthiazol-5- yl)phenyl)ethyl)pyrrolidine-2- carboxamide |
| CPD- 022 | 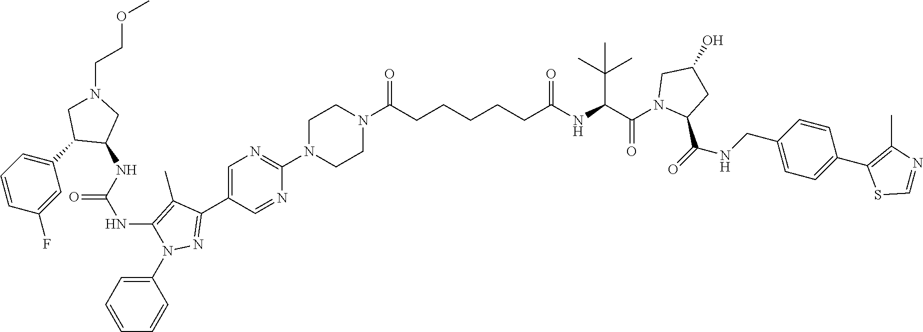 [SMILES: COCCN1C[C@@H](NC(=O)Nc2c(C)c(-c3cnc(N4CCN(C(=O)CCCCCC(=O)N[C@H](C(=O)N5C[C@H](O)C[C@H]5C(=O)NCc5ccc(-c6scnc6C)cc5)C(C)(C)C)CC4)nc3)nn2-c2ccccc2)[C@H](c2cccc(F)c2)C1] | (2S,4R)-1-((S)-2-(7-(4-(5-(5-(3- ((3S,4R)-4-(3-fluorophenyl)-1-(2- methoxyethyl)pyrrolidin-3-yl)ureido)- 4-methyl-1-phenyl-1H-pyrazol-3- yl)pyrimidin-2-yl)piperazin-1-yl)-7- oxoheptanamido)-3,3- dimethylbutanoyl)-4-hydroxy-N-(4-(4- methylthiazol-5-yl)benzyl)pyrrolidine- 2-carboxamide |
| CPD- 023 | 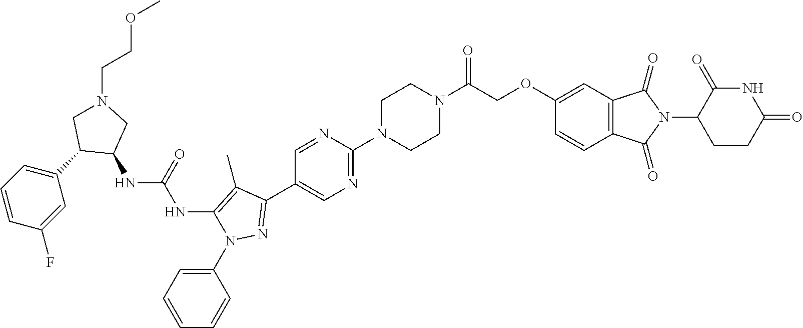 [SMILES: COCCN1C[C@@H](NC(=O)Nc2c(C)c(-c3cnc(N4CCN(C(=O)COc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(2-((2-(2,6-dioxopiperidin-3- yl)-1,3-dioxoisoindolin-5- yl)oxy)acetyl)piperazin-1-yl)pyrimidin- 5-yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 024 | 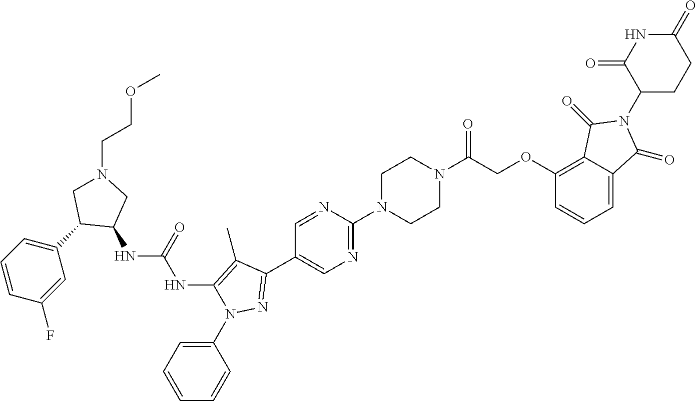 [SMILES: COCCN1C[C@@H](NC(=O)Nc2c(C)c(-c3cnc(N4CCN(C(=O)COc5cccc6c5C(=O)N(C5CCC(=O)NC5=O)C6=O)CC4)nc3)nn2-c2ccccc2)[C@H](c2cccc(F)c2)C1] | 1-(3-(2-(4-(2-((2-(2,6-dioxopiperidin-3- yl)-1,3-dioxoisoindolin-4- yl)oxy)acetyl)piperazin-1-yl)pyrimidin- 5-yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 025 | 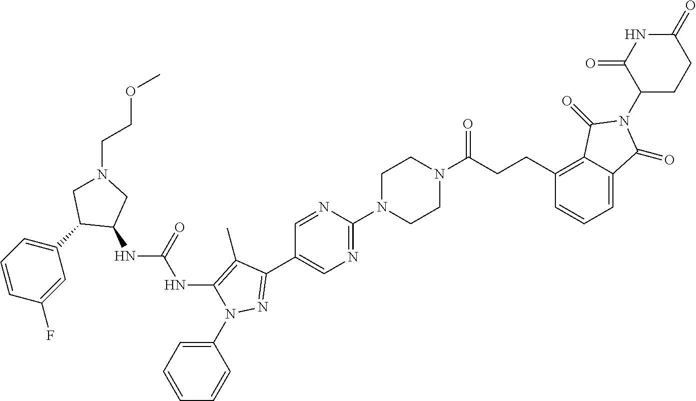 [SMILES: COCCN1C[C@@H](NC(=O)Nc2c(C)c(-c3cnc(N4CCN(C(=O)CCc5cccc6c5C(=O)N(C5CCC(=O)NC5=O)C6=O)CC4)nc3)nn2-c2ccccc2)[C@H](c2cccc(F)c2)C1] | 1-(3-(2-(4-(3-(2-(2,6-dioxopiperidin-3- yl)-1,3-dioxoisoindolin-4- yl)propanoyl)piperazin-1-yl)pyrimidin- 5-yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 026 | 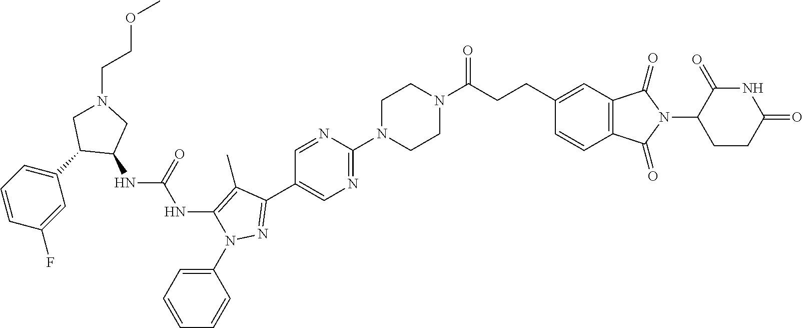 [SMILES: COCCN1C[C@@H](NC(=O)Nc2c(C)c(-c3cnc(N4CCN(C(=O)CCc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(3-(2-(2,6-dioxopiperidin-3- yl)-1,3-dioxoisoindolin-5- yl)propanoyl)piperazin-1-yl)pyrimidin- 5-yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 027 | 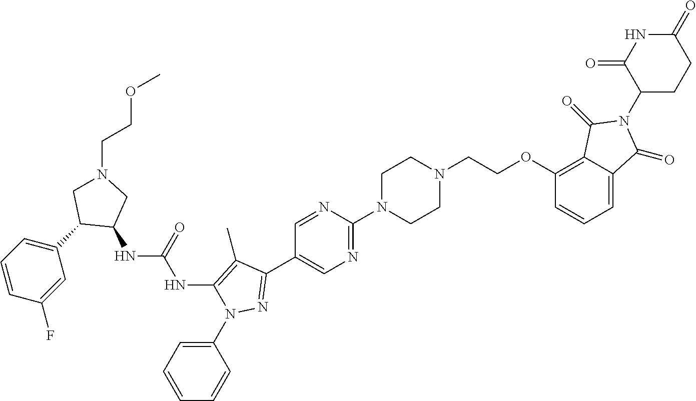 [SMILES: COCCN1C[C@@H](NC(=O)Nc2c(C)c(-c3cnc(N4CCN(CCOc5cccc6c5C(=O)N(C5CCC(=O)NC5=O)C6=O)CC4)nc3)nn2-c2ccccc2)[C@H](c2cccc(F)c2)C1] | 1-(3-(2-(4-(2-((2-(2,6-dioxopiperidin-3- yl)-1,3-dioxoisoindolin-4- yl)oxy)ethyl)piperazin-1-yl)pyrimidin- 5-yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 028 | 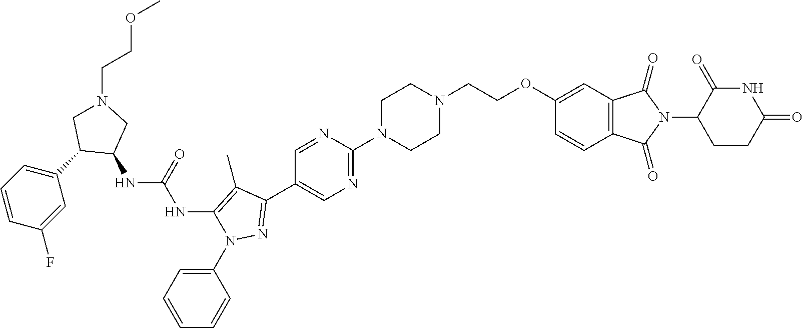 [SMILES: COCCN1C[C@@H](NC(=O)Nc2c(C)c(-c3cnc(N4CCN(CCOc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(2-((2-(2,6-dioxopiperidin-3- yl)-1,3-dioxoisoindolin-5- yl)oxy)ethyl)piperazin-1-yl)pyrimidin- 5-yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 029 | 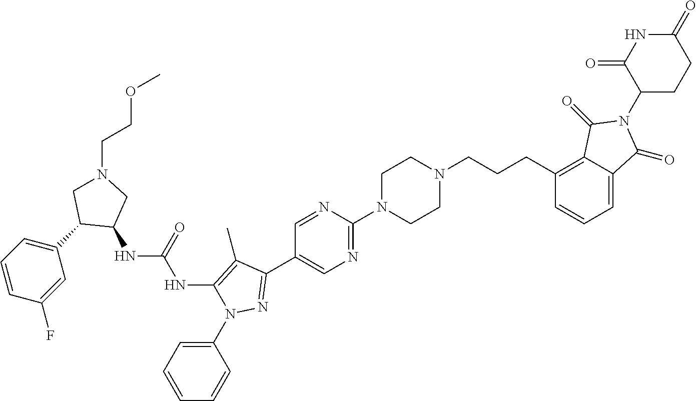 [SMILES: COCCN1C[C@@H](NC(=O)Nc2c(C)c(-c3cnc(N4CCN(CCCc5cccc6c5C(=O)N(C5CCC(=O)NC5=O)C6=O)CC4)nc3)nn2-c2ccccc2)[C@H](c2cccc(F)c2)C1] | 1-(3-(2-(4-(3-(2-(2,6-dioxopiperidin-3- yl)-1,3-dioxoisoindolin-4- yl)propyl)piperazin-1-yl)pyrimidin-5- yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 030 | 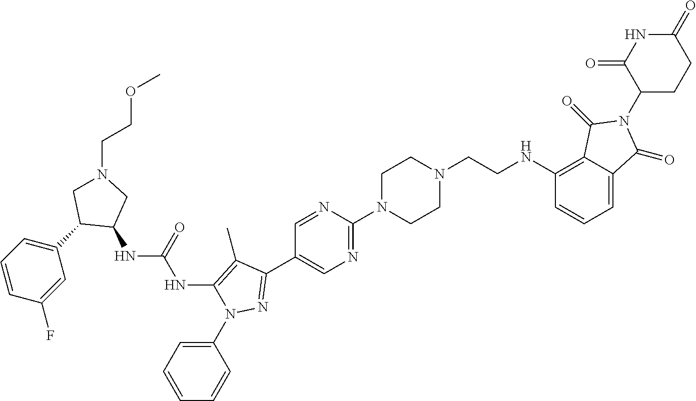 [SMILES: COCCN1C[C@@H](NC(=O)Nc2c(C)c(-c3cnc(N4CCN(CCNc5cccc6c5C(=O)N(C5CCC(=O)NC5=O)C6=O)CC4)nc3)nn2-c2ccccc2)[C@H](c2cccc(F)c2)C1] | 1-(3-(2-(4-(2-((2-(2,6-dioxopiperidin-3- yl)-1,3-dioxoisoindolin-4- yl)amino)ethyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 031 | 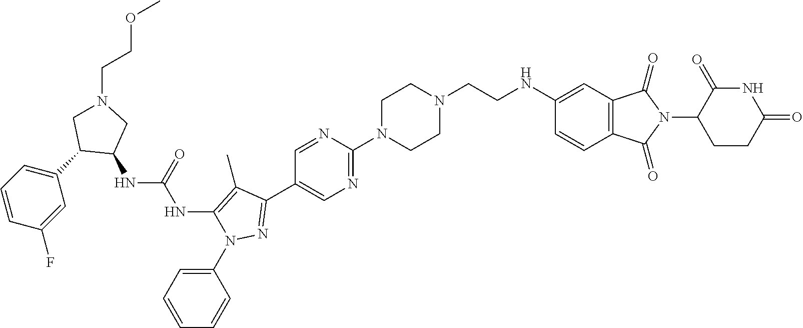 [SMILES: COCCN1C[C@@H](NC(=O)Nc2c(C)c(-c3cnc(N4CCN(CCNc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(2-((2-(2,6-dioxopiperidin-3- yl)-1,3-dioxoisoindolin-5- yl)amino)ethyl)piperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |
| CPD- 032 | 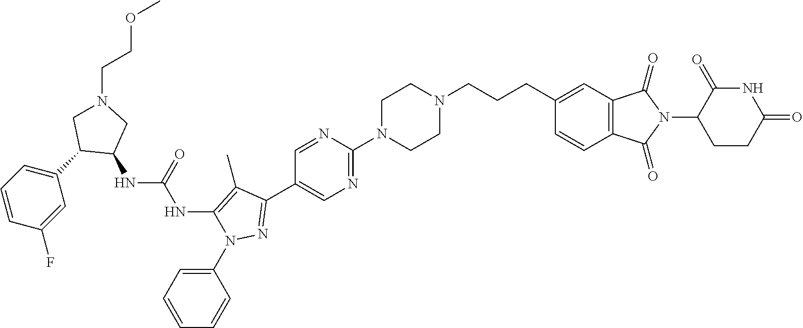 [SMILES: COCCN1C[C@@H](NC(=O)Nc2c(C)c(-c3cnc(N4CCN(CCCc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(3-(2-(2,6-dioxopiperidin-3- yl)-1,3-dioxoisoindolin-5- yl)propyl)piperazin-1-yl)pyrimidin-5- yl)-4-methyl-1-phenyl-1H-pyrazol-5- yl)-3-((3S,4R)-4-(3-fluorophenyl)-1-(2- methoxyethyl)pyrrolidin-3-yl)urea |
| CPD- 033 | 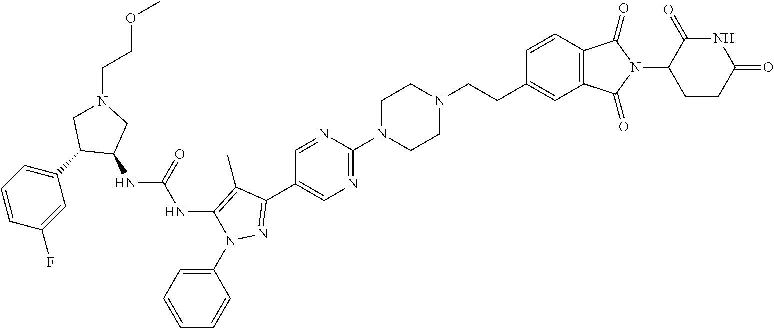 [SMILES: COCCN1C[C@@H](NC(=O)Nc2c(C)c(-c3cnc(N4CCN(CCc5ccc6c(c5)C(=O)N(C5CCC(=O)NC5=O)C6=O)CC4)nc3)nn2-c2ccccc2)[C@H](c2cccc(F)c2)C1] | 1-(3-(2-(4-(2-(2-(2,6-dioxopiperidin-3- yl)-1,3-dioxoisoindolin-5- yl)ethyl)piperazin-1-yl)pyrimidin-5-yl)- 4-methyl-1-phenyl-1H-pyrazol-5-yl)-3- ((3S,4R)-4-(3-fluorophenyl)-1-(2- methoxyethyl)pyrrolidin-3-yl)urea |
| WH-1 | 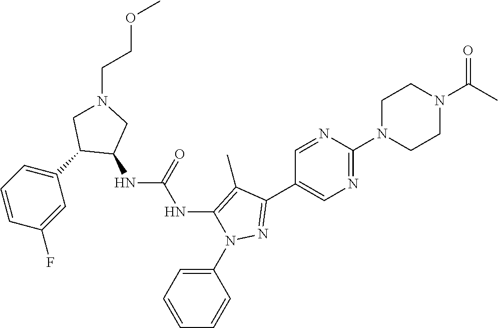 [SMILES: COCCN1C[C@@H](NC(=O)Nc2c(C)c(-c3cnc(N4CCN(C(C)=O)CC4)nc3)nn2-c2ccccc2)[C@H](c2cccc(F)c2)C1] | 1-(3-(2-(4-acetylpiperazin-1- yl)pyrimidin-5-yl)-4-methyl-1-phenyl- 1H-pyrazol-5-yl)-3-((3S,4R)-4-(3- fluorophenyl)-1-(2- methoxyethyl)pyrrolidin-3-yl)urea |

As used herein, in case of discrepancy between the structure and chemical name provided for a particular compound, the structure shall control.

### Example 33. Heterobifunctional Compounds Reduced the TPM3-TRKA Fusion Protein Levels in KM12 Colon Cancer Cells (FIG. 1)

KM12 cells were treated with heterobifunctional compounds at 10 nM or 100 nM for 16 hours. The Western blot results (FIG. 1A-1C) showed that multiple heterobifunctional compounds significantly reduced TPM3-TRKA protein levels even at 10 nM.

### Example 34. Heterobifunctional Compounds Reduced the Wildtype TRKA Protein Levels in HEL Erythroleukemia Cells (FIG. 2)

HEL cells were treated with heterobifunctional compounds CPD-019 and CPD-002 at 10 nM or 100 nM for 16 hours. The Western blot results showed that some heterobifunctional compounds significantly reduced TRKA protein levels.

### Example 35. Heterobifunctional Compounds Suppressed Viability of KM12 Colorectal Cancer Cells (FIG. 3)

KM12 cells seeded in 96-well plates were treated with heterobifunctional compounds following a 11-point 3-fold serial dilution for 3 days. Selected compounds CPD-019, CPD-002 and CPD-003 inhibited the viability of KM12 cells (FIG. 3). A summary of IC₅₀ values of selected compounds in KM12 cells and in TRKA G595R mutated KM12 cells is presented in Table 2.

### Example 36. Heterobifunctional Compound CPD-031 Reduced Wildtype TRKA Protein Levels in HEL Erythroleukemia Cells

NIH3T3 cells were infected with lentivirus directing expression of human full length TRKB or TRKC. Cells were then selected using 1 ug/ml puromycin to establish NIH3T3-TRKB and NIH3T3-TRKC cell lines that stably expressed human TRKB or TRKC, respectively.

HEL cells expressing endogenous TRKA, and NIH3T3-TRKB and NIH3T3-TRKC cells prepared as described above were treated with heterobifunctional compound CPD-031 at 0.1 nM, 1 nM, 10 nM, 100 nM and 1000 nM for 16 hours. The Western blot results showed that CPD-031 significantly reduced wildtype TRKA protein levels in HEL cells as shown in FIG. 4(A). CPD-031 treatment showed minimal reduction of TRKB or TRKC protein levels in NIH3T3-TRKB and NIH3T3-TRKC cells, FIG. 4(B) and 4(C), respectively.

### Materials and Methods:

### General Chemistry Methods:

All chemicals and reagents were purchased from commercial suppliers and used without further purification. LCMS spectra for all compounds were acquired using a Waters LC-MS AcQuity H UPLC class system. The Waters LC-MS AcQuity H UPLC class system comprising a pump (Quaternary Solvent Manager) with degasser, an autosampler (FTN), a column oven (40° C., unless otherwise indicated), a photo-diode array PDA detector. Chromatography was performed on an AcQuity UPLC BEH C18 (1.7 μm, 2.1×50 mm) with water containing 0.1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance 111400 spectrometer. Chemical shifts are expressed in parts per million (ppm) and reported as 6 value (chemical shift 6). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 m, 30×75 mm,) at room temperature. The flow rate was 40 mL/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm or 280 nm. The flow rate was 40 mL/min. A linear gradient was used with H₂O containing 0.05% TFA as solvent A and 100% of MeOH containing 0.05% TFA as solvent B. All compounds showed >95% purity using the LCMS methods described above.

### Cell Culture

KM12, TRKA G595R mutated KM12, HEL, and other cells were cultured at 37° C. with 5% CO₂ in DMEM or RPMI 1640 Medium supplemented with 10% fetal bovine serum. Cells were authenticated using the short tandem repeat (STR) assays. Mycoplasma test results were negative.

### Antibodies and Reagents

Rabbit anti-TRK antibody (92991S) was purchased from Cell Signaling Technology. HRP-conjugated anti-β-actin and anti-α-tubulin antibodies were purchased from GNI. Media and other cell culture reagents were purchased from Thermo Fisher. The CellTiter-Glo Assay kit was purchased from Promega.

### Immunoblotting

Cultured cells were washed with cold PBS once and lysed in cold RIPA buffer supplemented with protease inhibitors and phosphatase inhibitors (Beyotime Biotechnology). The solutions were then incubated at 4° C. for 30 minutes with gentle agitation to fully lyse cells. Cell lysates were centrifuged at 13,000 rpm for 10 minutes at 4° C. and pellets were discarded. Total protein concentrations in the lysates were determined by BCA assays (Beyotime Biotechnology). Cell lysates were mixed with Laemmli loading buffer to 1× and heated at 99° C. for 5 min. Proteins were resolved on SDS-PAGE and visualized by chemiluminescence. Images were taken by a ChemiDoc MP Imaging system (Bio-Rad). Protein bands were quantitated using the software provided by Bio-Rad.

### Cell Viability Assays

Cells were seeded at a density of 5000 cells per well in 96-well assay plates and treated with test compounds following a 11-point 3-fold serial dilution. Three days later, cell viability was determined using the CellTiter-Glo assay kit (Promega) according to the manufacturer's instructions. The dose-response curves were determined and IC₅₀ values were calculated using the GraphPad Prism software following a nonlinear regression (least squares fit) method.

The activities of selected compounds on cell viability (IC₅₀ values) and TMP3-TRKA degradation (percentage degradation at 10 nM) in KM12 cells or KM12-G595R mutated KM12 cells (KM12-G595R) are listed in Table 2.

TABLE 2

| | | | Degradation | |
|---|---|---|---|---|
| | IC₅₀ (nM) | IC₅₀ (nM) | (KM12, 10 | Degradation |
| Cmpd. | KM12 | KM12-G595R | nM) | (KM12, 100 nM) |
| CPD-001 | 517.5 | N/A | N/A | N/A |
| CPD-002 | 9.7 | N/A | +++ | +++ |
| CPD-003 | 5.1 | N/A | +++ | +++ |
| CPD-004 | 25.8 | N/A | ++ | ++ |
| CPD-005 | 47.27 | N/A | +++ | +++ |
| CPD-006 | 10.31 | N/A | +++ | +++ |
| CPD-007 | 10.57 | N/A | +++ | +++ |
| CPD-008 | 10.55 | N/A | +++ | +++ |
| CPD-009 | 13.58 | N/A | +++ | +++ |
| CPD-010 | 86.1 | N/A | ++ | +++ |
| CPD-011 | 118.7 | N/A | + | ++ |
| CPD-012 | 131.5 | N/A | + | ++ |
| CPD-013 | 79.74 | N/A | + | ++ |
| CPD-014 | 17.8 | N/A | ++ | +++ |
| CPD-015 | 35.52 | N/A | ++ | ++ |
| CPD-016 | 189.5 | N/A | + | ++ |
| CPD-017 | 444.7 | N/A | - | ++ |
| CPD-018 | 852.4 | N/A | - | ++ |
| CPD-019 | 9.2 | N/A | +++ | +++ |
| CPD-020 | 293.3 | N/A | — | ++ |
| CPD-023 | 6.46 | 44.00 | +++ | +++ |
| CPD-024 | 86.94 | 313.60 | + | ++ |
| CPD-025 | 30.02 | 158.00 | + | ++ |
| CPD-026 | 42.15 | 301.20 | +++ | +++ |
| CPD-027 | 3.13 | 4.47 | ++ | ++ |
| CPD-028 | 4.10 | 30.38 | +++ | ++ |
| CPD-029 | 13.43 | 277.30 | + | ++ |
| CPD-030 | 1.09 | 5.13 | ++ | ++ |
| CPD-031 | 2.04 | 6.11 | +++ | +++ |
| CPD-032 | 13.71 | 18.25 | +++ | +++ |
| CPD-033 | 14.38 | 94.56 | ++ | ++ |

The percentage of TRK protein degradation of each compound at 10 nM and 100 nM was determined in KM12 cells as described in Methods. The percentage of degradation of each compound was indicated in the following categories: N/A: data not available; −: no degradation; +: degradation less than 50%; ++: degradation equal or greater than 50% but less than 800%; +++: degradation equal or greater than 80%.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-40. (canceled)

41. A heterobifunctional compound of FORMULA I:
M_{TRK}-M_{L}-M_{DT} (FORMULA I)
or a pharmaceutically acceptable salt thereof, wherein M_{TRK} is a tropomyosin receptor kinase (TRK) ligand; M_{L} is a linker moiety; and M_{DT} is a degradation tag;
and wherein
a) the TRK ligand is a moiety of FORMULA 1-5A: 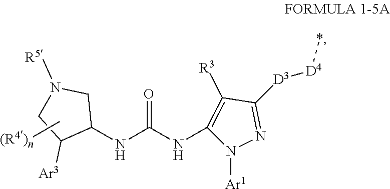
wherein
* indicates the connection to the linker moiety of the heterobifunctional compound;
-D³-D⁴- is selected from the group consisting of:
—CONH—, 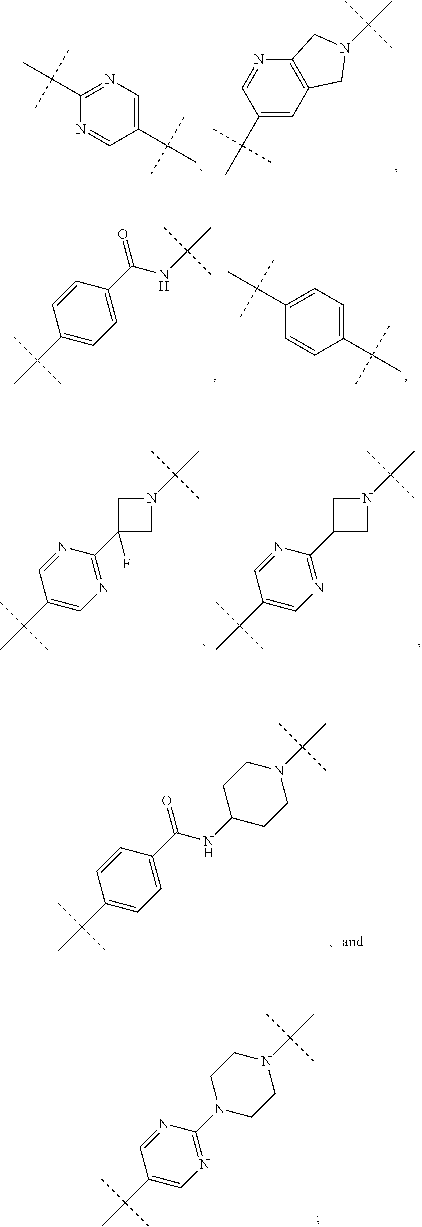
Ar³ is selected from optionally substituted aryl, and optionally substituted heteroaryl; Ar is selected from optionally substituted aryl and optionally substituted heteroaryl;
R³ is selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₁-C₈alkyl-NHC(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted aryl, and optionally substituted heteroaryl;
R^{4′}, at each occurrence, is independently selected from the group consisting of null, H, F, OH, optionally substituted C₁-C₃ alkyl, and optionally substituted 1-3 membered heteroalkyl;
n is selected from 0, 1, 2, 3, 4, 5, 6 and 7;
R^{5′} is selected from the group consisting of hydrogen, optionally substituted C₁-C₈ alkyl, optionally substituted C₂-C₈ alkenyl, optionally substituted C₂-C₈ alkynyl, optionally substituted 2-8 membered heteroalkyl, optionally substituted 3-8 membered heteroalkenyl, optionally substituted 3-8 membered heteroalkynyl, optionally substituted C₁-C₈ alkyl-C(O)—, optionally substituted C₁-C₈ alkyl-NHC(O)—, optionally substituted C₁-C₈alkoxyC₁-C₈alkylene, optionally substituted C₁-C₈ haloalkyl, optionally substituted C₁-C₈ hydroxyalkyl, optionally substituted C₁-C₈alkylaminoC₁-C₈alkylene, optionally substituted C₃-C₈ cycloalkyl, optionally substituted 3-8 membered carbocyclyl, optionally substituted 3-8 membered heterocyclyl, optionally substituted 3-8 membered carbocyclyl-C₁-C₈alkylene, optionally substituted 3-8 membered heterocyclyl-C₁-C₈alkylene, optionally substituted C₂-C₈ alkenyl, optionally substituted aryl, optionally substituted heteroaryl, optionally substituted aryl-C₁-C₈alkylene, and optionally substituted heteroaryl-C₁-C₈alkylene;
b) the degradation tag is a moiety of FORMULA 8A, 8B, 8C, 8D, 8E, 8F, 8G, 8H, 8I, 8J, 8K, 8L, 8M, 8N, 80, 8P, 8Q, 8R, 8S, 8T, 8U, 8V, 8W, 8X, 8Y, 8Z, 8AA, 8AB, 8AC, or 8AD: 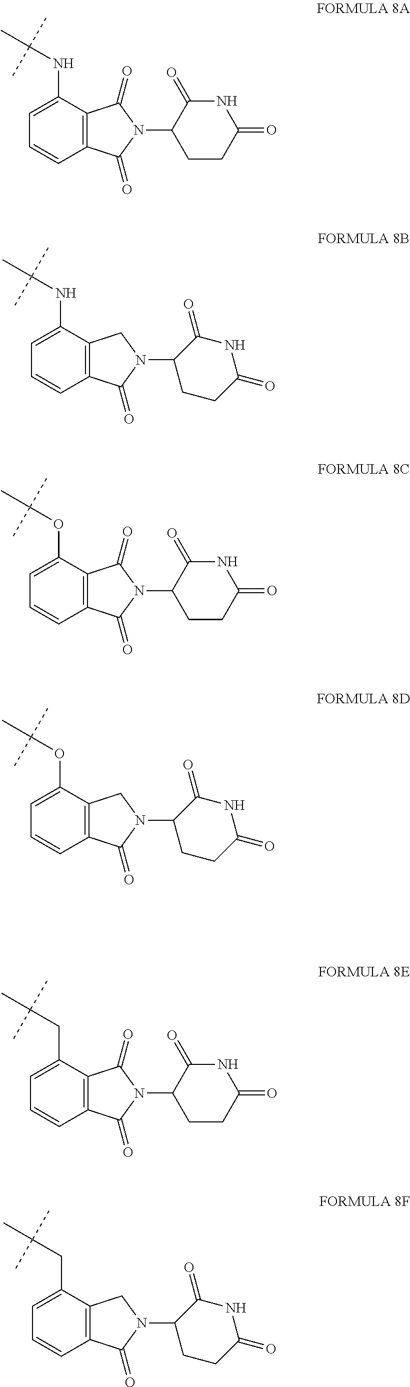 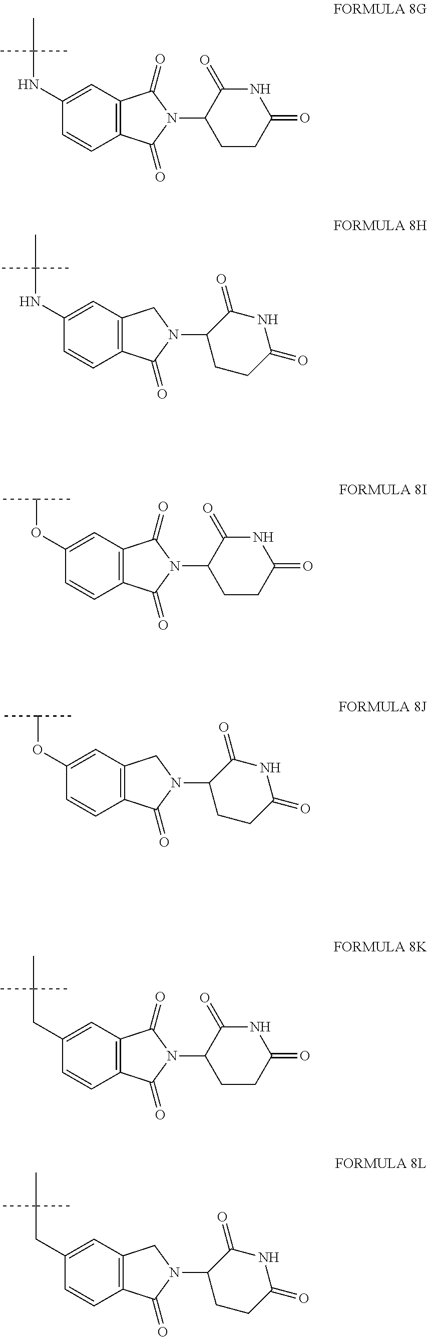 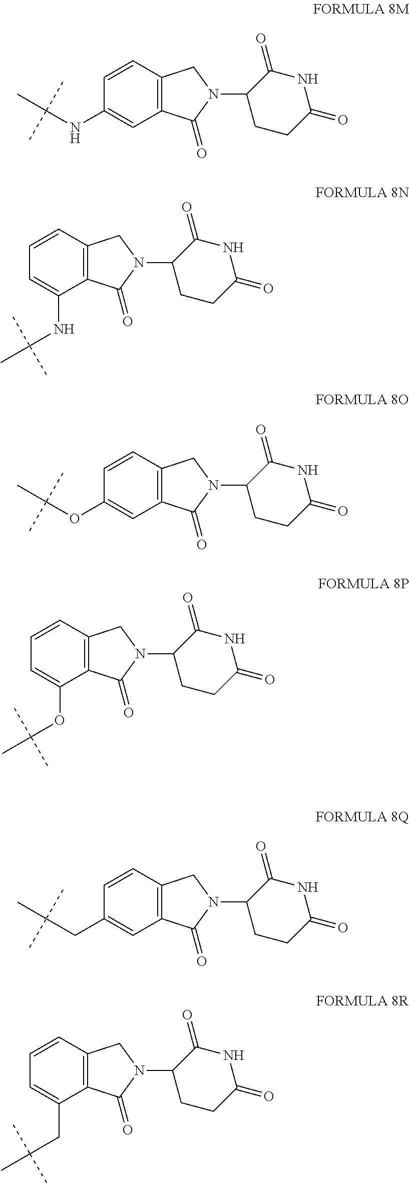 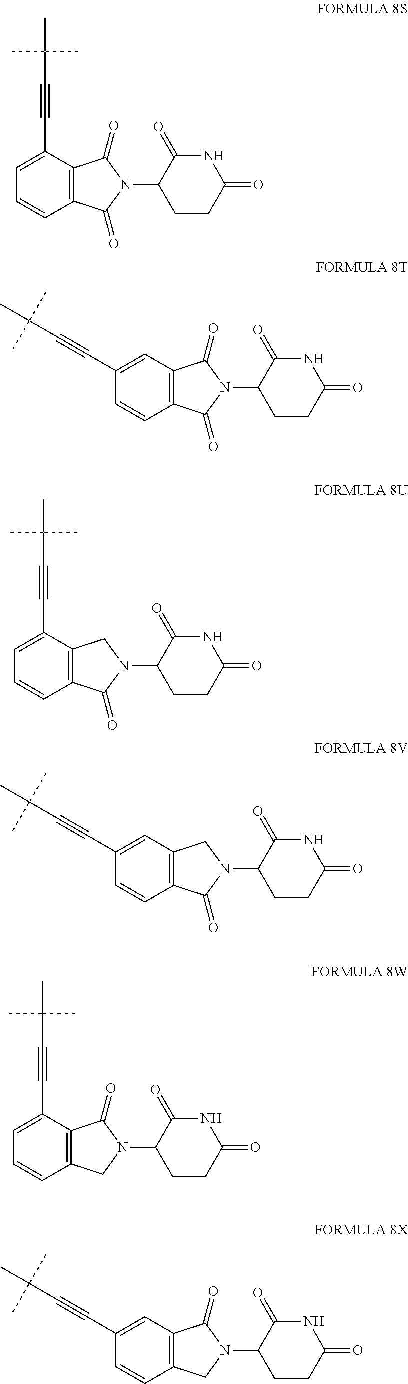 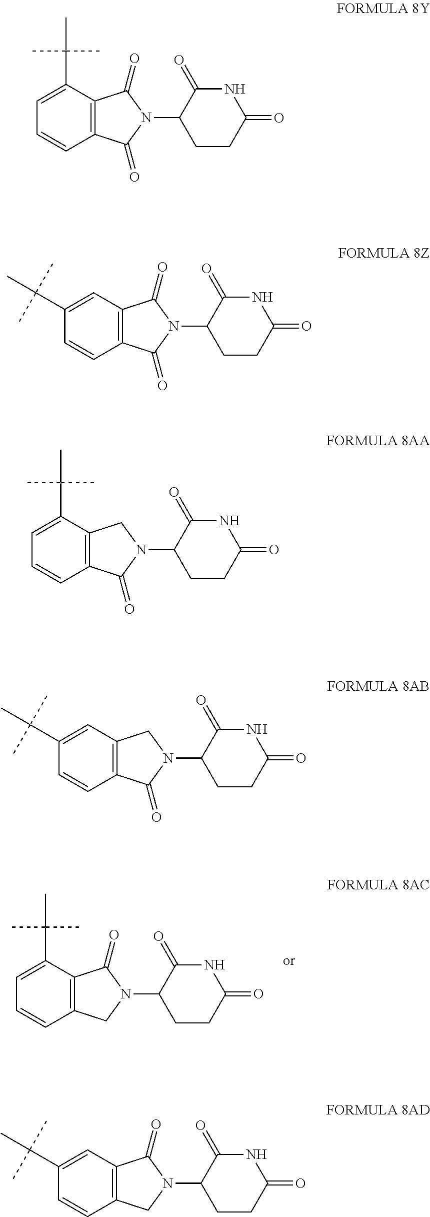
and
c) the linker moiety is of FORMULA 9: 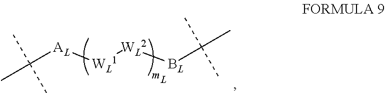
wherein:
A_{L} is selected from the group consisting of R_{L}^{d}—R_{L}^{e}, R_{L}^{d}C(O)R_{L}^{e}, R_{L}^{d}C(O)NHR_{L}^{e} and R_{L}^{d}NHC(O)R_{L}^{e};
B_{L} is selected from the group consisting of null, R_{L}^{d}C(O)NHR_{L}^{e}, R_{L}^{d}C(O)R_{L}^{e}, R_{L}^{d}OR_{L}^{e}, R_{L}^{d}NHC(O)R_{L}^{e} and R_{L}^{d}NHR_{L}^{e}
R_{L}^{d} and R_{L}^{e}, at each occurrence, are independently selected from the group consisting of null, optionally substituted C₁, C₂ or C₃ alkylene, R_{L}^{r}, R_{L}^{r}—(C₁, C₂ or C₃ alkylene), (C₁, C₂ or C₃ alkylene)-R_{L}^{r} and (C₁, C₂ or C₃ alkylene)-R_{L}^{r}—(C₁, C₂ or C₃ alkylene);
W_{L}², at each occurrence, is independently selected from the group consisting of null, O and NH;
W_{L}¹, at each occurrence, is independently selected from the group consisting of R_{L}^{r} and optionally substituted C₁, C₂ or C₃ alkylene; and
m_{L} is 1, 2, 3, 4, 5, 6, 7, 8, 9, or 10.

42. The heterobifunctional compound of claim 41, or a pharmaceutically acceptable salt thereof, wherein the TRK ligand is a moiety of FORMULA 1-6A:

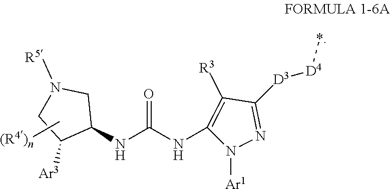

43. The heterobifunctional compound of claim 41, or a pharmaceutically acceptable salt thereof, wherein Ar³ is an optionally substituted aryl or optionally substituted heteroaryl selected from the group consisting of:

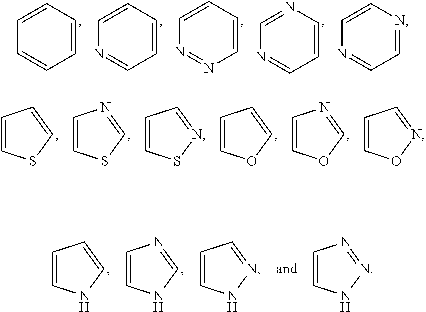

44. The heterobifunctional compound of claim 43, or a pharmaceutically acceptable salt thereof, wherein Ar³ is selected from the group consisting of

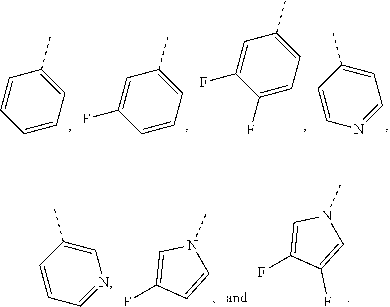

45. The heterobifunctional compound of claim 41, or a pharmaceutically acceptable salt thereof, wherein the TRK ligand is a moiety of FORMULA 1-7A: 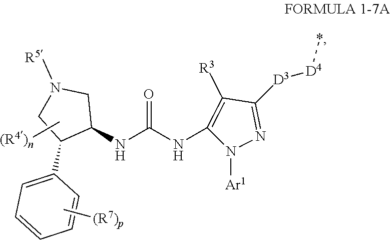
wherein: R⁷, at each occurrence, is independently selected from the group consisting of hydrogen, halogen, CN, NO₂, optionally substituted C₁-C₈ alkyl, optionally substituted C₂-C₈ alkenyl, optionally substituted C₂-C₈ alkynyl, optionally substituted 3-8 membered heteroalkyl, optionally substituted 3-8 membered heteroalkenyl, optionally substituted 3-8 membered heteroalkynyl, optionally substituted C₁-C₈alkoxy, optionally substituted C₁-C₈alkyl-S—, optionally substituted C₁-C₈alkyl-OC(O)—, optionally substituted C₁-C₈alkyl-NH—C(O)—, optionally substituted C₁-C₈alkylamino, optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₈ carbocyclyl, optionally substituted 3-8 membered heterocyclyl, optionally substituted C₃-C₈ carbocyclyl-C₁-C₈alkyl, and optionally substituted 3-8 membered heterocyclyl-C₁-C₈alkyl, optionally substituted aryl, optionally substituted heteroaryl, optionally substituted aryl-C₁-C₈alkyl, and optionally substituted heteroaryl-C₁-C₈alkyl; and
p is selected from 1, 2, 3, 4, and 5.

46. The heterobifunctional compound of claim 41, or a pharmaceutically acceptable salt thereof, wherein R^{4′}, at each occurrence, is independently selected from the group consisting of null, H, F, Cl, OH, CH₃, CHF₂, CF₃, isopropyl, and cyclopropyl.

47. The heterobifunctional compound of claim 41, or a pharmaceutically acceptable salt thereof, wherein R^{5′} is selected from the group consisting of methoxyethyl and difluoroethyl.

48. The heterobifunctional compound of claim 45, or a pharmaceutically acceptable salt thereof, wherein R⁷, at each occurrence, is independently selected from the group consisting of H, F, Cl, CN, CH₃, CHF₂, CF₃, isopropyl, and cyclopropyl.

49. The heterobifunctional compound of claim 41, or a pharmaceutically acceptable salt thereof, wherein R³ is selected from H, F, Cl, CN, CH₃, CHF₂, CF₃, isopropyl, and cyclopropyl.

50. The heterobifunctional compound of claim 41, or a pharmaceutically acceptable salt thereof, wherein Ar¹ is an optionally substituted aryl or an optionally substituted heteroaryl selected from the group consisting of

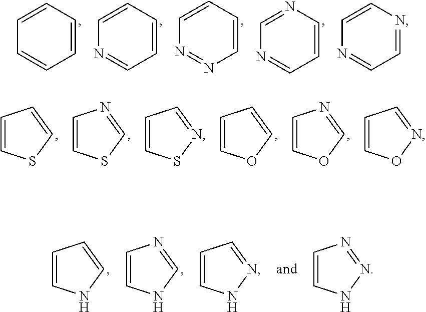

51. The heterobifunctional compound of claim 41, or a pharmaceutically acceptable salt thereof, wherein Ar¹ is selected from

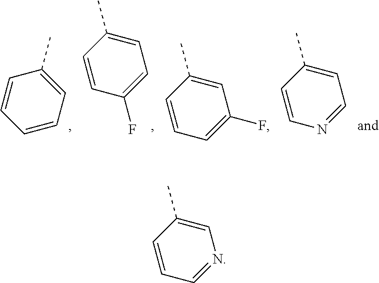

52. The heterobifunctional compound of claim 41, or a pharmaceutically acceptable salt thereof, wherein -D³-D⁴- is:

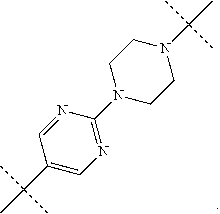

53. The heterobifunctional compound of claim 41, or a pharmaceutically acceptable salt thereof, wherein the TRK ligand is a moiety of FORMULA 1-8A:

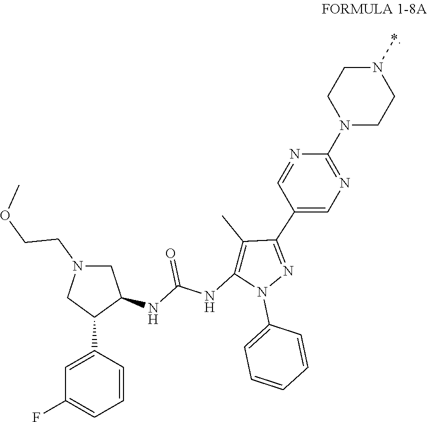

54. The heterobifunctional compound of claim 41, or a pharmaceutically acceptable salt thereof, wherein:
the bivalent moiety A_{L} is the point of attachment to the TRK ligand;
A_{L} is R_{L}^{d}C(O)R_{L}^{e};
R_{L}^{d} and R_{L}^{e}, at each occurrence, are independently selected from the group consisting of null, and optionally substituted C₁, C₂ or C₃ alkylene;
W_{L}², at each occurrence, is independently selected from null or O;
W_{L}¹, at each occurrence, is independently optionally substituted C₁, C₂ or C₃ alkylene.

55. The heterobifunctional compound of claim 41, wherein the heterobifunctional compound is selected from the group consisting of CPD-001 to CPD-033, or their enantiomers, or their racemates, or a pharmaceutically acceptable salt, or analog thereof.

56. The heterobifunctional compound of claim 41, wherein the heterobifunctional compound is selected from the group consisting of:
1-(3-(2-(4-(4-((2-(2,6-dioxopiperidin-3-yl)-1,3-dioxoisoindolin-5-yl)amino)butanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-002);
1-(3-(2-(4-((2-(2,6-dioxopiperidin-3-yl)-1,3-dioxoisoindolin-4-yl)glycyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-003);
1-(3-(2-(4-(8-((2-(2,6-dioxopiperidin-3-yl)-1,3-dioxoisoindolin-5-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-006);
1-(3-(2-(4-(6-((2-(2,6-dioxopiperidin-3-yl)-1,3-dioxoisoindolin-4-yl)amino)hexanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-007);
1-(3-(2-(4-(6-((2-(2,6-dioxopiperidin-3-yl)-1,3-dioxoisoindolin-5-yl)amino)hexanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-008);
1-(3-(2-(4-(8-((2-(2,6-dioxopiperidin-3-yl)-1,3-dioxoisoindolin-4-yl)amino)octanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-009);
1-(3-(2-(4-((2-(2,6-dioxopiperidin-3-yl)-1,3-dioxoisoindolin-5-yl)glycyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-019);
1-(3-(2-(4-(2-((2-(2,6-dioxopiperidin-3-yl)-1,3-dioxoisoindolin-5-yl)oxy)acetyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-023);
1-(3-(2-(4-(3-(2-(2,6-dioxopiperidin-3-yl)-1,3-dioxoisoindolin-5-yl)propanoyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-026);
1-(3-(2-(4-(2-((2-(2,6-dioxopiperidin-3-yl)-1,3-dioxoisoindolin-4-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-027);
1-(3-(2-(4-(2-((2-(2,6-dioxopiperidin-3-yl)-1,3-dioxoisoindolin-5-yl)oxy)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-028);
1-(3-(2-(4-(2-((2-(2,6-dioxopiperidin-3-yl)-1,3-dioxoisoindolin-4-yl)amino)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-030);
1-(3-(2-(4-(2-((2-(2,6-dioxopiperidin-3-yl)-1,3-dioxoisoindolin-5-yl)amino)ethyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-031); and
1-(3-(2-(4-(3-(2-(2,6-dioxopiperidin-3-yl)-1,3-dioxoisoindolin-5-yl)propyl)piperazin-1-yl)pyrimidin-5-yl)-4-methyl-1-phenyl-1H-pyrazol-5-yl)-3-((3S,4R)-4-(3-fluorophenyl)-1-(2-methoxyethyl)pyrrolidin-3-yl)urea (CPD-032);
or a pharmaceutically acceptable salt thereof.

57. A pharmaceutical composition comprising the heterobifunctional compound of claim 41, or a pharmaceutically acceptable salt thereof, and one or more pharmaceutically acceptable carriers.

58. The pharmaceutical composition of claim 57, further comprising one or more additional therapeutic agents.

59. A method of treating a TRK-mediated disease, wherein the method comprises administering to a subject in need thereof an effective amount of the heterobifunctional compound of claim 41, or a pharmaceutically acceptable salt thereof.

60. The method of claim 59, wherein the TRK-mediated disease is selected from the group consisting of cancer, pain, an inflammatory disorder, an immune disease, or a combination thereof.
